(19) United States
(12) Patent Application Publication
(10) Pub. No.: US 2025/0074922 A1
(43) Pub. Date: Mar. 6, 2025

(54) COMPOUNDS AND METHODS USEFUL FOR STABILIZING PHENYLALANINE HYDROXYLASE MUTATIONS

(71) Applicant: Agios Pharmaceuticals, Inc., Cambridge, MA (US)

(72) Inventors: Lucrezia De Pascalis, Cambridge, MA (US); Peng Liu, Belmont, MA (US); Brian C. Shook, Holliston, MA (US)

(21) Appl. No.: 18/818,695

(22) Filed: Aug. 29, 2024

Related U.S. Application Data
(60) Provisional application No. 63/535,435, filed on Aug. 30, 2023.

(51) Int. Cl. C07D 519/00; A61K 31/444; A61K 31/497; A61K 31/501; A61K 31/506; A61K 31/5377

(52) U.S. Cl. CPC C07D 519/00; A61K 31/444; A61K 31/497; A61K 31/501; A61K 31/506; A61K 31/5377

(57) ABSTRACT

The disclosure relates to compounds of Formula I, or a pharmaceutically acceptable salt thereof, wherein R^{a}, R^{b}, and x are defined herein, and pharmaceutical compositions comprising compounds of Formula I, or a pharmaceutically acceptable salt thereof.

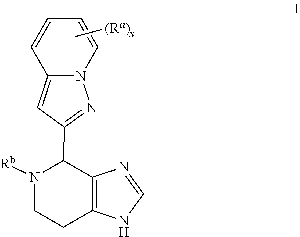

These compounds and pharmaceutical compositions are useful in methods for stabilizing a mutant PAH protein or reducing blood phenylalanine concentration in a subject suffering from phenylketonuria. In some embodiments, the mutant PAH protein contains at least one R408W, R261Q, R243Q, Y414C, L48S, A403V, I65T, R241C, L348V, R408Q, or V388M mutation. In other embodiments, the mutant PAH protein contains at least one R408W, Y414C, I65T, F39L, R408Q, L348V, R261Q, A300S, or L48S mutation.

### RELATED APPLICATIONS

This application claims the benefit of priority to U.S. Provisional Application No. 63/535,435, filed Aug. 30, 2023, the entire contents of which are incorporated herein by reference.

### TECHNICAL FIELD

This application pertains to compounds that stabilize phenylalanine hydroxylase (PAH) mutations, pharmaceutical compositions comprising those compounds, and methods of using those compounds for treating phenylketonuria.

### BACKGROUND

Phenylketonuria (PKU) is an autosomal recessive disorder affecting approximately 1:10,000 people worldwide (approx. 1:25,000 in the U.S.). The number of patients varies globally depending on region. PKU arises in patients who have mutations in the gene encoding the phenylalanine hydroxylase (PAH) enzyme responsible for converting phenylalanine to tyrosine. PAH is a tetrameric enzyme expressed in the liver requiring BH4 cofactor for activity. Reduction or loss of PAH activity results in toxic accumulation of phenylalanine (Phe) in the blood and brain. High levels of Phe damage brain white matter and interfere with neurotransmitter production. If untreated, high levels of Phe can result in mental retardation and decreased IQ in children and neurocognitive and psychiatric issues in adults, such as executive function deficits (for example, difficulty with attention, memory, flexible thinking, and organization/time management), psychological issues (for example, depression, anxiety, and mood swings), psychiatric and/or behavioral issues (for example, attention deficit hyperactivity disorder, self-harm, schizophrenia, agoraphobia, and agitation) and neurological abnormalities (for example, spasticity, tremor, gait disturbances, and seizures).

PKU is characterized by elevated blood Phe concentration or hyperphenylalaninemia (HPA). Normal blood Phe concentrations range from 50 to 110 μM. PKU phenotypes can vary from mild hyperphenylalaninemia (HPA) to more severe phenotypes that result in untreated blood Phe concentrations exceeding 1200 μM. American medical guidelines currently recommend maintaining blood Phe concentration in the range of 120 to 360 μM in both adults and children under the age of 12 years. European medical guidelines currently recommend maintaining blood Phe concentration below 360 μM in children under the age of 12 years and in pregnant women and below 600 μM in non-pregnant patients older than 12 years.

A standard of care for treating PKU is a Phe-restricted diet that severely limits the intake of natural protein, supplemented with medical foods to provide protein equivalents and prevent nutritional deficiencies. Such diets are very strict diets and challenging to adhere to. Two medications are currently approved for treating PKU, each having its own challenges. Kuvan (sapropterin dihydrochloride) is a synthetic BH4 cofactor approved in 2007 for use in infants to adults. Kuvan is not effective for all PKU patients, and the current guidelines suggest response testing in patients unless the patient is known to have two null mutations. Pegvaliase is an enzyme substitution therapy approved in 2018 for adults with a blood Phe concentration greater than 600 μM, despite prior management with available treatment options. Pegvaliase typically involves injection of a purified PEGylated form of phenylalanine ammonia lyase that reduces Phe by converting it to ammonia and trans-cinnamic acid instead of tyrosine. One of the main complications with enzyme substitution therapy is the attainment and maintenance of therapeutically effective amounts of protein in vivo due to rapid degradation or inactivation of the infused protein. A current approach to overcome this problem is to perform numerous costly high dose injections.

Pharmaceutical agents that enable PKU patients to increase their intake of natural protein are desired.

### SUMMARY

In some aspects, the disclosure provides compounds as disclosed in Tables 1 and 2 herein, or a pharmaceutically acceptable salt thereof.

In further aspects, the disclosure provides racemates of compounds disclosed in Tables 1 and 2 herein, or a pharmaceutically acceptable salt thereof.

In other aspects, the disclosure provides compounds of Formula I:

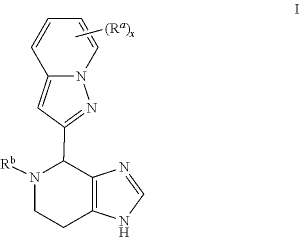

or a pharmaceutically acceptable salt thereof, wherein:
x is 0 to 5;
each R^{a} independently is halo, C₁₋₆alkyl, C₃₋₆cycloalkyl, C₁₋₆haloalkyl, C₁₋₆alkoxy or C₁₋₆haloalkoxy;
R^{b} is

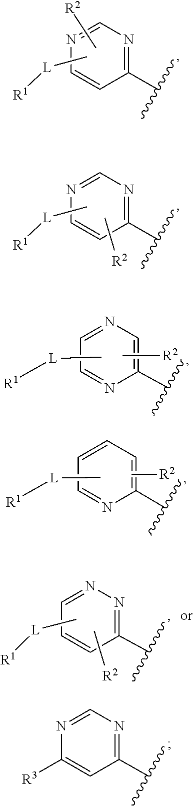

L is —C(O)—, —CH(OH)—, or —C(O)NH—;
R¹ is optionally substituted phenyl, optionally substituted 4-, 5-, or 6-membered heterocyclyl, —NR⁴R⁵, or optionally substituted C₃₋₆cycloalkyl;
R² is hydrogen, C₁₋₆alkyl, C₁₋₆haloalkyl, or halo;
R³ is optionally substituted 5- or 6-membered heterocyclyl; and
R⁴ and R⁵ are each independently hydrogen or C₁₋₆alkyl.

In further aspects, the disclosure provides pharmaceutical compositions comprising one or more compound described herein or a pharmaceutically acceptable salt thereof, and a pharmaceutically acceptable excipient.

In yet other aspects, the disclosure provides methods for stabilizing a mutant PAH protein, comprising contacting the protein with one or more compound as described herein or a pharmaceutically acceptable salt thereof. In some embodiments, the mutant PAH protein contains at least one R408W, R261Q, R243Q, Y414C, L48S, A403V, I65T, R241C, L348V, R408Q, or V388M mutation. In other embodiments, the mutant PAH protein contains at least one R408W, Y414C, I65T, F39L, R408Q, L348V, R261Q, A300S, or L48S mutation.

In still further aspects, the disclosure provides methods for reducing phenylalanine levels in a subject suffering from phenylketonuria comprising administering a therapeutically effective amount of one or more compound as described herein or a pharmaceutically acceptable salt thereof.

### DETAILED DESCRIPTION OF ILLUSTRATIVE EMBODIMENTS

### Definitions

Unless otherwise defined, all technical and scientific terms used herein have the same meaning as commonly understood by one of ordinary skill in the art to which this disclosure pertains. The terminology used in the description is for describing particular embodiments only and is not intended to be limiting of the disclosure.

As used in structures herein, “ [symbol] ” indicates the point of attachment of the particular depicted structure or substituent group to the appropriate atom(s) in the remainder of the molecule.

The articles “a” and “an” as used herein and in the appended claims are used herein to refer to one or to more than one (e.g., to at least one) of the grammatical object of the article unless the context clearly indicates otherwise. By way of example, “an element” means one element or more than one element.

“Pharmaceutically acceptable” means approved or approvable by a regulatory agency of the Federal or a state government or the corresponding agency in a country other than the United States, or that is listed in the U.S. Pharmacopoeia or other generally recognized pharmacopoeia for use in animals, e.g., in humans.

“Pharmaceutically acceptable salt” refers to a salt of a compound of the disclosure that is pharmaceutically acceptable and that possesses the desired pharmacological activity of the parent compound. In particular, such salts are non-toxic and may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, fumarate, tartrate, mesylate, acetate, maleate, oxalate and the like.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith.

The term “alkyl,” when used alone or as part of a substituent group, refers to a straight- or branched-chain hydrocarbon group having from 1 to 12 carbon atoms (“C₁₋₁₂”), for example 1 to 6 carbons atoms (“C₁₋₆”), in the group. Examples of alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentyl, amyl, neopentyl, 3-methyl-2-butanyl, tertiary amyl), hexyl (C₆) (e.g., n-hexyl), heptyl (C₇) (e.g., n-heptyl), octyl (C₈) (e.g., n-octyl), and the like. In some embodiments, the alkyl group is a C₁₋₆alkyl; in other embodiments, it is a C₁₋₄alkyl; and in other embodiments, it is a C₁₋₃alkyl.

The term “alkylene,” when used alone or as part of a substituent group, refers to an alkyl diradical, i.e., a straight- or branched-chain hydrocarbon group that is attached to two other groups. For example, one embodiment of a C₂alkylene is the diradical —CH₂CH₂—. In some embodiments, the alkylene group is C₁₋₆alkylene; in other embodiments, it is C₁₋₄alkylene.

When a range of carbon atoms is used herein, for example, C₁₋₆, all ranges, as well as individual numbers of carbon atoms are encompassed. For example, “C₁₋₃” includes C₁₋₃, C₁₋₂, C₂₋₃, C₁, C₂, and C₃.

The term “cycloalkyl” when used alone or as part of a substituent group refers to cyclic-containing, non-aromatic hydrocarbon groups having from 3 to 10 carbon atoms (“C₃₋₁₀”), for example from 3 to 7 carbon atoms (“C₃₋₇”) or from 3 to 6 carbon atoms (“C₃₋₆”). Examples of cycloalkyl groups include cyclopropyl (C₃), cyclobutyl (C₄), cyclopentyl (C₅), cyclohexyl (C₆), cycloheptyl (C₇), and the like. In some embodiments, the cycloalkyl group is a C₃₋₄cycloalkyl; in other embodiments, it is a C₃₋₆cycloalkyl; and in other embodiments, it is C₃₋₈ cycloalkyl. The cycloalkyl may be unsubstituted or substituted. In some embodiments, the cycloalkyl is substituted with one substituent. In other embodiments, the cycloalkyl is substituted with two substituents. In yet other embodiments, the cycloalkyl is substituted with three substituents. In still further embodiments, the cycloalkyl is unsubstituted.

The term “aryl” when used alone or as part of a substituent group also refers to a mono- or bicyclic-aromatic hydrocarbon ring structure having 6 or 10 carbon atoms in the ring, wherein one or more of the carbon atoms in the ring is optionally substituted. The term “aryl” also includes a mono- or bicyclic-aromatic hydrocarbon ring structure having 6 or 10 carbon atoms in the ring, wherein two adjacent carbon atoms in the ring are optionally substituted such that said two adjacent carbon atoms and their respective substituents form a cycloalkyl or heterocyclyl ring. Examples of aryl groups include phenyl, indenyl, naphthyl, 1,2,3,4-tetrahydronaphthyl, and the like. The aryl may be unsubstituted or substituted. In other embodiments, the optionally substituted phenyl has four substituents. In further embodiments, the optionally substituted phenyl has three substituents. In yet other embodiments, the optionally substituted phenyl has two substituents. In still further embodiments, the optionally substituted phenyl has one substituent. In other embodiments, the optionally substituted phenyl is unsubstituted.

As used herein, the term “alkenyl” refers to a straight- or branched-chain group having from 2 to 12 carbon atoms (“C₂₋₁₂”) in the group, wherein the group includes at least one carbon-carbon double bond. Examples of alkenyl groups include vinyl (—CH═CH₂; C₂alkenyl), allyl (—CH₂—CH═CH₂; C₃alkenyl), propenyl (—CH═CHCH₃; C₃alkenyl), isopropenyl (—C(CH₃)═CH₂; C₃alkenyl), butenyl (—CH═CHCH₂CH₃; C₄alkenyl), sec-butenyl (—C(CH₃)═CHCH₃; C₄alkenyl), iso-butenyl (—CH═C(CH₃)₂; C₄alkenyl), 2-butenyl (—CH₂CH═CHCH₃; C₄alkyl), pentenyl (—CH═CHCH₂CH₂CH₃; C₅alkenyl), and the like. In some embodiments, the alkenyl group is a C₂₋₆ alkenyl group; in other embodiments, it is C₂₋₄alkenyl.

As used herein, the term “alkynyl” refers to a straight- or branched-chain group having from 2 to 12 carbon atoms (“C₂₋₁₂”) in the group, and wherein the group includes at least one carbon-carbon triple bond. Examples of alkynyl groups include ethynyl (—C≡CH; C₂alkynyl), propargyl (—CH₂—C≡CH; C₃alkynyl), propynyl (—C≡CCH₃; C₃alkynyl), butynyl (—C≡CCH₂CH₃; C₄alkynyl), pentynyl (—C≡CCH₂CH₂CH₃; C₅alkynyl), and the like. In some embodiments, the alkynyl group is a C₂₋₆alkynyl group; in other embodiments, it is C₂₋₄alkynyl.

The term “carbonyl” as used by itself or as part of another group refers to C(O) or C(═O).

The term “halo” or “halogen,” as used by itself or as part of another group refers to a fluorine, chlorine, bromine, or iodine atom.

As used herein, the term “haloalkyl” refers to an alkyl group wherein one or more of the hydrogen atoms has been replaced with one or more halogen atoms which may be the same or different. In some embodiments, the alkyl is substituted by at least one halogen. In other embodiments, the alkyl is substituted by one, two, or three F and/or Cl. Examples of haloalkyl groups include fluoromethyl (CH₂F), 1-fluoroethyl (CH(CH₃)F), 2-fluoroethyl, difluoromethyl (CHF₂), trifluoromethyl (CF₃), pentafluoroethyl, 1,1-difluoroethyl (C(CH₃)F₂), 2,2-difluoroethyl (CH₂CHF₂), 2,2,2-trifluoroethyl (CH₂CF₃), 2-fluoropropan-2-yl (C(CH₃)₂F), 3,3,3-trifluoropropyl, 4,4,4-trifluorobutyl, trichloromethyl and the like. In some embodiments, the haloalkyl group is a C₁₋₆haloalkyl; in other embodiments, it is a C₁₋₄haloalkyl; and in other embodiments, it is a C₁₋₃haloalkyl.

The term “cyanoalkyl” as used by itself or as part of another group refers to an alkyl as defined herein that is substituted by one or more CN. In some embodiments, the alkyl is substituted by at least one CN. In other embodiments, the alkyl is substituted by one, two, or three CN. In further embodiments, the cyanoalkyl group is a C₁₋₆cyanoalkyl. In yet other embodiments, the cyanoalkyl is a C₁₋₄cyanoalkyl. Examples of cyanoalkyl groups include CH₂CN, CH₂CH₂CN, CH(CN)CH₃, CH₂CH₂CH₂CN, C(CH₃)₂CN, CH₂CH(CN)CH₃, CH(CN)CH₂CH₃, and the like.

The term “hydroxyalkyl” as used by itself or as part of another group refers to an alkyl group as defined herein wherein one or more of the hydrogen atoms has been replaced with one or more hydroxyl (i.e., —OH). In some embodiments, the hydroxyalkyl contains one OH. In other embodiments, the hydroxyalkyl contains two OH. In further embodiments, the hydroxyalkyl contains three OH. Examples of hydroxyalkyl groups include hydroxymethyl, hydroxyethyl (e.g., 1-hydroxyethyl, 2-hydroxyethyl), 1,2-dihydroxyethyl, hydroxypropyl (e.g., 2-hydroxypropyl, 3-hydroxypropyl), hydroxybutyl (e.g., 3-hydroxybutyl, 4-hydroxybutyl), 2-hydroxy-1-methylpropyl, 1,3-dihydroxyprop-2-yl, and the like. In some embodiments, the hydroxyalkyl group is C₁₋₆hydroxyalkyl; in other embodiments, it is C₁₋₄hydroxyalkyl; and in other embodiments, it is C₁₋₃hydroxyalkyl.

The term “cycloalkylsulfonyl” as used by itself or as part of another group refers to a cycloalkyl as defined herein that is bound to a sulfonyl, i.e., —SO₂—, and the sulfonyl group forms the point of attachment to the remainder of the molecule. In some embodiments, the cycloalkylsulfonyl is a C₃₋₈cycloalkylsulfonyl; in other embodiments, it is a C₃₋₆cycloalkylsulfonyl. Examples of cycloalkylsulfonyl groups include —SO₂-cyclopropyl, —SO₂-cyclobutyl, —SO₂-cyclopentyl, and the like.

The term “alkylsulfonyl” as used by itself or as part of another group refers to an alkyl as defined herein that is bound to a sulfonyl, i.e., —SO₂—, and the sulfonyl group forms the point of attachment to the remainder of the molecule. In some embodiments, the alkylsulfonyl is C₁₋₆alkylsulfonyl; in other embodiments, it is a C₁₋₄alkylsulfonyl. Examples of alkylsulfonyl groups include —SO₂CH₃, —SO₂CH₂CH₃, —SO₂CH(CH₃)₂, and the like.

The term “alkoxy” as used by itself or as part of another group refers to an oxygen radical attached to an alkyl group by a single bond. Examples of alkoxyl groups include methoxy (OCH₃), ethoxy (OCH₂CH₃), propoxy (e.g., —OⁿPr, —OⁱPr), or butoxy (e.g., —OⁿBu, —OⁱBu, —O^{s}Bu, —O^{t}Bu), and the like. In other embodiments, the alkoxy group is a C₁₋₆alkoxy. In further embodiments, the alkoxy group is a C₁₋₄alkoxy.

The term “alkoxy(alkylene)” as used by itself or as part of another group refers to an alkylene group as defined herein that is bound to an alkoxy group as defined herein. Examples of alkoxy(alkylene) groups include —CH₂OCH₃, —CH₂CH₂OCH₃, and the like.

The term “haloalkoxy” as used by itself or as part of another group refers to an oxygen radical attached to a haloalkyl group by a single bond, wherein haloalkyl is defined herein. Examples of haloalkoxy groups include fluoromethoxy (OCH₂F), 2-fluoroethoxy, difluoromethoxy (OCHF₂), trifluoromethoxy (OCF₃), pentafluoroethoxy, 1,1-difluoroethoxy (OC(CH₃)F₂), 2,2-difluoroethoxy, 2,2,2-trifluoroethoxy (OCH₂CF₃), 3,3,3-trifluoropropoxy, 4,4,4-trifluorobutoxy, trichloromethoxy groups, and the like. In some embodiments, the haloalkoxy group is a C₁₋₆haloalkoxy; in other embodiments, it is C₁₋₄haloalkoxy; and in other embodiments, it is C₁₋₃haloalkoxy.

The term “heteroaryl” when used alone or as part of a substituent group refers to a mono- or bicyclic-aromatic ring structure including carbon atoms as well as up to four heteroatoms that are each independently nitrogen, oxygen, or sulfur. Heteroaryl rings can include a total of 5, 6, 9, or 10 ring atoms. In some embodiments, heteroaryl rings are characterized by the number of ring atoms in the heteroaryl group. For example, a 6-membered heteroaryl group refers to a heteroaryl group having 6 ring atoms in the group. Similarly, a 5-membered heteroaryl group refers to a heteroaryl group having 5 ring atoms in the group. Examples of heteroaryl groups include pyridinyl, pyrrolyl, pyrazinyl, pyrimidinyl, pyridazinyl, pyrazolyl, thienyl, indolyl, isoindolyl, imidazolyl, oxazolyl, isoxazolyl, thiazolyl, furyl, oxadiazolyl, thiadiazolyl, quinolyl, isoquinolyl, benzothiazolyl, benzoisothiazolyl, benzoxazolyl, benzoisoxazolyl, benzofuranyl, isobenzofuranyl, benzothiophenyl, benzoimidazolyl, indazolyl, quinoxalyl, quinazolinyl, triazolyl, tetrazolyl, isothiazolyl, pyranyl, purinyl, naphthyridinyl, phthalazinyl, cinnolinyl, pteridinyl, benzoxazinyl, chromenyl, 1H-pyrrolo[2,3-b]pyridinyl, oxazolo[4,5-b]pyridinyl, oxazolo[5,4-b]pyridinyl, thiazolo[5,4-b]pyridinyl, thiazolo[4,5-b]pyridinyl, 3H-imidazo[4,5-b]pyridinyl, furo[2,3-b]pyridinyl, thieno[2,3-b]pyridinyl, pyrazolo[1,5-a]pyridinyl, imidazol[1,5-a]pyridinyl, and pyrrolo[1,2]pyridazinyl and the like. The term “heteroaryl” also includes N-oxides. The heteroaryl may be unsubstituted or substituted. In some embodiments, the heteroaryl is substituted with one substituent. In other embodiments, the heteroaryl is substituted with two substituents. In yet other embodiments, the heteroaryl is substituted with three substituents. In still further embodiments, the heteroaryl is unsubstituted. Substitution may occur on any available carbon or heteroatom (e.g., nitrogen), or both, as permitted by substituent valency.

In some embodiments, the heteroaryl is a 5- or 6-membered heteroaryl. In some embodiments, the heteroaryl is a 5-membered heteroaryl, i.e., the heteroaryl is a monocyclic aromatic ring system having 5 ring atoms wherein at least one carbon atom of the ring is replaced with a heteroatom independently selected from nitrogen, oxygen, and sulfur. Examples of 5-membered heteroaryl groups include thienyl (e.g., thien-2-yl and thien-3-yl), furyl (e.g., 2-furanyl, 3-furanyl, 4-furanyl), pyrrolyl (e.g., pyrrol-2-yl, pyrrol-3-yl), oxazolyl (e.g., oxazol-2-yl, oxazol-4-yl, and oxazol-5-yl), pyrazolyl (e.g., pyrazol-1-yl, pyrazol-3-yl, pyrazol-4-yl, pyrazol-5-yl), imidazolyl (e.g., imidazol-2-yl, imidazol-4-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), isoxazolyl (e.g., isoxazol-3-yl, isoxazol-4-yl, and isoxazol-5-yl), triazolyl (e.g., 1,2,3-triazol-2-yl, 1,2,4-triazol-1-yl, 1,2,4-triazol-3-yl, 1,2,4-triazol-5-yl), tetrazolyl, thiadiazolyl (e.g., 1,2,3-thiadiazolyl, 1,2,4-thiadiazolyl, 1,2,5-thidiazolyl, 1,3,4-thiadiazolyl), oxadiazolyl (e.g., 1,2,3-oxadiazolyl, 1,2,4-oxadiazolyl, 1,2,5-oxadiazolyl, 1,3,4-oxadiazolyl), and the like. In other embodiments, the heteroaryl is a 6-membered heteroaryl, e.g., the heteroaryl is a monocyclic aromatic ring system having 6 ring atoms wherein at least one carbon atom of the ring is replaced with a nitrogen atom. Examples of 6-membered heteroaryl groups include pyridinyl (e.g., pyridin-1-yl, pyridin-2-yl, pyridin-3-yl, and pyridin-4-yl), pyrazinyl (e.g., pyrazin-2-yl, pyrazin-3-yl, pyrazin-5-yl, pyrazin-6-yl), pyrimidinyl (e.g., pyrimidin-2-yl, pyrimidin-4-yl, and pyrimidin-5-yl), pyridazinyl (e.g., pyridazin-3-yl, pyridazin-4-yl, pyridazin-6-yl) and the like.

The term “heterocyclyl” as used by itself or as part of another group refers to non-aromatic, saturated or partially unsaturated, e.g., containing one or two double bonds, cyclic groups containing one, two, or three rings having from three to fourteen ring members, i.e., a 3-14-membered heterocyclyl, wherein at least one carbon atom of one of the rings is replaced with a heteroatom. Each heteroatom is independently selected from oxygen, sulfur, including sulfoxide and sulfone, and/or nitrogen atoms, which can be oxidized or quaternized. The term “heterocyclyl” also includes groups having fused optionally substituted aryl groups, e.g., indolinyl or chroman-4-yl and groups having fused optionally substituted cycloalkyl groups, e.g., 6-azaspiro[2.5]octanyl. The heterocyclyl group may be attached to another group or substituent through any heteroatom or carbon atom of the ring that results in a stable structure. In some embodiments, heterocyclyl rings are characterized by the number of ring atoms in the heterocyclyl group. For example, a 6-membered heterocyclyl group refers to a heterocyclyl group having 6 ring atoms in the group. Similarly, a 5-membered heterocyclyl group refers to a heterocyclyl group having 5 ring atoms in the group. Similarly, a 4-membered heterocyclyl group refers to a heterocyclyl group having 4 ring atoms in the group. Examples of heterocyclyl groups include aziridinyl, azetidinyl, pyrrolidinyl, dioxolanyl, imidazolidinyl, pyrazolidinyl, piperazinyl, piperidinyl, dioxanyl, morpholinyl, thianyl, thianyl sulfoxide, 1-oxo-1-imino-1thiacyclohexyl, dithianyl, thiomorpholinyl, azepanyl, oxazepanyl, oxiranyl, oxetanyl, tetrahydrofuranyl, tetrahydropyranyl, tetrahydrothiophenyl, pyranyl, and the like. In some embodiments, the heterocyclyl is a three to twelve-membered monocyclic, saturated ring containing at least one heteroatom that is oxygen, nitrogen, or sulfur. In some embodiments, the heterocyclyl group is a 4-, 5- or 6-membered heterocyclyl group. In other embodiments, the heterocyclyl group is a 5- or 6-membered heterocyclyl group. In further embodiments, the heterocyclyl group is a 4-, 5- or 6-membered cyclic group containing one nitrogen atom. In yet other embodiments, the heterocyclyl group is a 5- or 6-membered cyclic group containing one or two nitrogen atoms or one nitrogen atom and one oxygen atom. In some embodiments, the heterocyclyl group includes azetidinyl, pyrrolidinyl, piperidinyl, piperazinyl, and morpholinyl. The heterocyclyl moiety can be unsubstituted, or one or more of the carbon atoms, nitrogen, or sulfur atoms in the ring can be substituted. In some embodiments, the heterocyclyl is substituted with one substituent. In other embodiments, the heterocyclyl is substituted with two substituents. In yet other embodiments, the heterocyclyl is substituted with three substituents. In still further embodiments, the heterocyclyl is unsubstituted.

The term “optionally substituted,” as used herein to describe a chemical moiety defined herein, means that the moiety may, but is not required to be, substituted with one or more suitable functional groups or other substituents as provided herein. For example, a substituent may be optionally substituted with one or more of: halo, cyano, —NO₂, —N₃, —OH, —SH, C₁₋₆alkyl, C₃₋₈cycloalkyl, C₃₋₈cycloalkenyl, C₂₋₆alkenyl, C₂₋₆alkynyl, C₁₋₆haloalkyl, C₁₋₆alkoxy, C₁₋₆alkoxy(alkylene), C₁₋₆haloalkoxy, C₁₋₆haloalkoxy(alkylene), C₁₋₆alkylcarbonyl, C₁₋₆cyanoalkyl, C₁₋₆hydroxyalkyl, C₁₋₆alkylenethio, (CR^{v}R^{x})_{q}NR^{y}R^{z} (wherein R^{v} and R^{x} are, independently, H or C₁₋₆alkyl; R^{v} and R^{z}, are independently, H, C₁₋₆alkyl, C₃₋₆cycloalkyl, C₁₋₆hydroxyalkyl, C₁₋₆haloalkyl, C₁₋₆alkoxy(alkylene), or C(O)OC₁₋₆alkyl, and q is 0, 1, 2, or 3), —C(O)NH₂, —C(O)NHC₁₋₆alkyl, —C(O)N(C₁₋₆alkyl)₂, —C(O)NHC₃₋₆cycloalkyl, —C(O)N(C₃₋₆cycloalkyl)₂, —COOH, —C₁₋₆alkyleneCOOH, —C₃₋₆cycloalkylCOOH, —C₁₋₆alkyleneCONH₂, C₃₋₆ cycloalkylCONH₂, —C₁₋₆alkyleneCONHC₁₋₆alkyl, —C₁₋₆alkyleneCON(C₁₋₆alkyl)₂, —C(O)OC₁₋₆alkyl, —NHCO(C₁₋₆alkyl), —N(C₁₋₆alkyl)C(O)(C₁₋₆alkyl), —S(O)C₁₋₆alkyl, —S(O)C₃₋₆cycloalkyl, C₁₋₆alkylsulfonyl, C₃₋₈cycloalkylsulfonyl, C₁₋₆alkylsulfonyl(alkylene), oxo (═O), 3-7-membered heterocyclyl, heterocyclyl(alkylene), aryl, aryl(alkylene), or heteroaryl groups. In some embodiments, the C₁₋₆alkyl group in any of the substituent groups in this paragraph is a C₁₋₄alkyl; in other embodiments it is C₁₋₃alkyl. In some embodiments, the C₁₋₆alkylene group in any of the substituent groups in this paragraph is a C₁₋₄alkylene. In some embodiments, the C₁₋₆haloalkyl substituent is a C₁₋₄haloalkyl; in other embodiments, it is C₁₋₃haloalkyl. In some embodiments, the C₃₋₆cycloalkyl substituent is a C₃₋₄cycloalkyl substituent. In some embodiments, the C₁₋₆alkoxy substituent is a C₁₋₃alkoxy; in other embodiments, it is C₁₋₄alkoxy. In some embodiments, the C₁₋₆haloalkoxy substituent is a C₁₋₃haloalkoxy; in other embodiments, it is C₁₋₄haloalkoxy.

In some embodiments, “optionally substituted,” refers to the following substituents: halo, CN, NO₂, N₃, alkyl, alkenyl, alkynyl, cycloalkyl, aryl, heterocyclyl, heteroaryl, OR^{o1}, SR^{s1}, N(Rⁿ¹)₂, C(═O)N(Rⁿ¹)₂, N(Rⁿ¹)C(═O)R^{c1}, C(═O)R^{c1}, C(═O)OR^{o1}, OC(═O)R^{c1}, S(═O)R^{s1}, S(═O)₂R^{s1}, S(═O)(C₃-C₆cycloalkyl), S(═O)₂(C₃-C₆cycloalkyl), S(═O)OR^{o1}, OS(═O)R^{c1}, S(═O)₂OR^{o1}, OS(═O)₂R^{c1}, S(═O)N(Rⁿ¹)₂, S(═O)₂N(Rⁿ¹)₂, N(Rⁿ¹)S(═O)R^{s1}, N(Rⁿ¹)S(═O)₂R^{s1}, N(Rⁿ¹)C(═O)OR^{o1}, OC(═O)N(Rⁿ¹)₂, N(Rⁿ¹)C(═O)N(Rⁿ¹)₂, N(Rⁿ¹)S(═O)N(Rⁿ¹)₂, N(Rⁿ¹)S(═O)₂N(Rⁿ¹)₂, N(Rⁿ¹)S(═O)OR^{o1}, N(Rⁿ¹)S(═O)₂OR^{o1}, OS(═O)N(Rⁿ¹)₂, or OS(═O)₂N(Rⁿ¹)₂; wherein each instance of Rⁿ¹ is independently hydrogen, an optionally substituted C₁-C₆alkyl, or a nitrogen protecting group; each instance of R^{o1} is independently hydrogen, an optionally substituted C₁-C₆alkyl, or an oxygen protecting group; and each instance of R^{c1} is an optionally substituted C₁-C₆alkyl; and each instance of R^{s1} is independently an optionally substituted C₁-C₆alkyl or a sulfur protecting group.

The term “nitrogen protecting group” refers to a moiety that is attached to a nitrogen atom to prevent reaction at that nitrogen atom. Nitrogen protecting groups will be known by those skilled in the art and include those described in Wuts, P. G., Greene's Protective Groups in Organic Synthesis. Wiley; 5th edition (Oct. 27, 2014), which is incorporated by reference herein.

The term “oxygen protecting group” refers to a moiety that is attached to an oxygen atom to prevent reaction at that oxygen atom. Oxygen protecting groups will be known by those skilled in the art and include those described in Wuts, P. G., Greene's Protective Groups in Organic Synthesis. Wiley; 5th edition (Oct. 27, 2014), which is incorporated by reference herein.

The term “sulfur protecting group” refers to a moiety that is attached to a sulfur atom to prevent reaction at that sulfur atom. Sulfur protecting groups will be known by those skilled in the art and include those described in Wuts, P. G., Greene's Protective Groups in Organic Synthesis. Wiley: 5th edition (Oct. 27, 2014), which is incorporated by reference herein.

Recitation of ranges of values herein are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

The term “about” when used in combination with a numeric value or range of values means the value or range of values may deviate to an extent deemed reasonable to one of ordinary skill in the art.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including supercritical fluid chromatography (SFC), chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, IN 1972).

Exemplary compounds of the disclosure including a chiral center may be depicted herein as having particular stereochemistries, but for which absolute stereochemistry has not been obtained. Absolute configurations can be obtained using methods known in the art.

As used herein, the term “stereoisomers” refers to compounds which have identical chemical constitution and connectivity but differ with regard to the arrangement of the atoms or groups in space, e.g., enantiomers or diastereomers.

When the stereochemical configuration at a chiral center in a compound having one or more chiral centers is depicted by its chemical name (e.g., where the configuration is indicated in the chemical name by “R” or “S”) or structure (e.g., the configuration is indicated by dashed or wedge bonds), the enrichment of the indicated configuration relative to the opposite configuration is greater than 50%, 60%, 70%, 80%, 90%, 99% or 99.9%. “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

When a disclosed compound is named or depicted by structure without indicating stereochemistry, it is understood that the name or the structure encompasses one of the possible stereoisomers or geometric isomers free of the others, or a mixture of the encompassed stereoisomers or geometric isomers.

It will be understood that certain compounds disclosed herein may exist in tautomeric forms. Such forms are included as part of the present disclosure. Thus, when a compound herein is represented by a structural formula or designated by a chemical name herein, all tautomeric forms which may exist for the compound are encompassed by the structural formula.

When the compounds described herein contain olefinic double bonds or other centers of geometric asymmetry, and unless specified otherwise, it is intended that they include both E and Z geometric isomers.

In some embodiments, the compounds described herein are isotopically enriched compound, e.g., an isotopologue. The term “isotopically enriched” refers to an atom having an isotopic composition other than the naturally abundant isotopic composition of that atom. “Isotopically enriched” may also refer to a compound containing at least one atom having an isotopic composition other than the natural isotopic composition of that atom. In an isotopologue, “isotopic enrichment” refers to the percentage of incorporation of an amount of a specific isotope of a given atom in a molecule in the place of that atom's natural isotopic composition. For example, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%. In one embodiment, one or more hydrogen atoms on a described compound may be replaced by deuterium.

Thus, as used herein, and unless otherwise indicated, the term “isotopic enrichment factor” refers to the ratio between the isotopic composition and the natural isotopic composition of a specified isotope.

With regard to the compounds provided herein, when a particular atom's position is designated as having deuterium or “D” or “²H”, it is understood that the abundance of deuterium at that position is substantially greater than the natural abundance of deuterium, which is about 0.015%. A position designated as having deuterium typically has a minimum isotopic enrichment factor of, in particular embodiments, at least 1000 (15% deuterium incorporation), at least 2000 (30% deuterium incorporation), at least 3000 (45% deuterium incorporation), at least 3500 (52.5% deuterium incorporation), at least 4000 (60% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation) at each designated deuterium atom. The isotopic enrichment and isotopic enrichment factor of the compounds provided herein can be determined using conventional analytical methods known to one of ordinary skill in the art, including mass spectrometry and nuclear magnetic resonance spectroscopy.

The term “patient” or “subject” is used throughout the specification to describe an animal, preferably a human or a domesticated animal, to whom treatment, including prophylactic treatment, with the compounds or compositions according to the present disclosure is provided. For treatment of those conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term.

The terms “therapeutically effective amount” or “effective amount” means an amount or dose of a compound of the disclosure (or a pharmaceutically acceptable salt thereof) sufficient to generally bring about the desired therapeutic benefit in subjects in need of such treatment for the designated disease or disorder. Further, a therapeutically effective amount with respect to a compound of the disclosure means that amount of therapeutic agent alone, or in combination with other therapies, that provides a therapeutic benefit in the treatment or prevention of a disease.

“Treating” or “treatment” of any disease or disorder refers, in one embodiment, to ameliorating the disease or disorder (e.g., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

The terms “prevent,” “preventing,” and “prevention” refer to the prevention of the onset, recurrence, or spread of the disease in a subject resulting from the administration of a prophylactic or therapeutic agent.

### Compounds

The present disclosure provides compounds as described in Tables 1 or 2 herein, and their pharmaceutically acceptable salts and/or isotopologues. In some embodiments, the compound is one or more compounds in Tables 1 or 2, or a pharmaceutically acceptable salt thereof. In other embodiments, the compound is one or more S-enantiomers in Tables 1 or 2 (e.g., wherein the stereogenic carbon atom of the imidazopyridinyl is in the S-configuration), or a pharmaceutically acceptable salt thereof. In further embodiments, the compound is one or more R-enantiomers in Tables 1 or 2 (e.g., wherein the stereogenic carbon atom of the imidazopyridinyl is in the R-configuration), or a pharmaceutically acceptable salt thereof. In still further embodiments, the compound is a racemate of one or more of the compounds in Tables 1 or 2, or a pharmaceutically acceptable salt thereof. Compounds having Formula I are further disclosed in the Exemplification and are included in the present disclosure. Pharmaceutically acceptable salts thereof as well as the neutral forms are included for any of the compounds described herein.

The present disclosure provides compounds of Formula I:

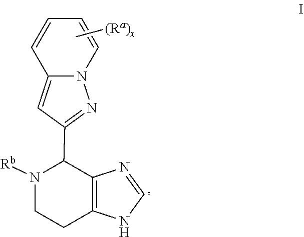

or a pharmaceutically acceptable salt thereof, wherein R^{a}, R^{b}, and x are defined herein.

In the structure of Formula I, x is 0 to 5. In some embodiments, x is 0. In other embodiments, x is 1. In further embodiments, x is 2. In yet other embodiments, x is 3. In still further embodiments, x is 4. In other embodiments, x is 5.

In further embodiments, x is 0 or 1, such that the pyrazolo[1,5-a]pyridinyl moiety is

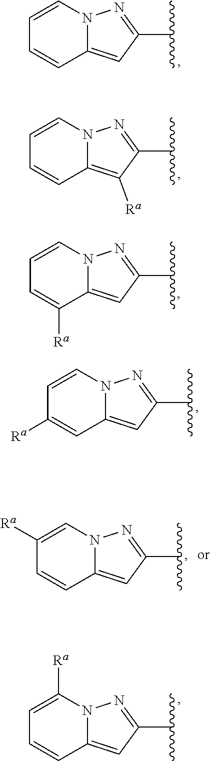

In other embodiments, the pyrazolo[1,5-a]pyridinyl moiety is

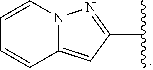 [SMILES: *c1cc2ccccn2n1]

In yet other embodiments, the pyrazolo[1,5-a]pyridinyl moiety is

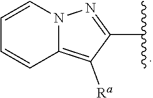

In still other embodiments, the pyrazolo[1,5-a]pyridinyl moiety is

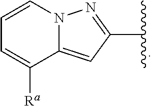

In yet other embodiment, the pyrazolo[1,5-a]pyridinyl moiety is

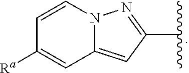

In still further embodiments, the pyrazolo[1,5-a]pyridinyl moiety is

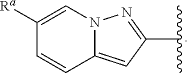

In other embodiments, the pyrazolo[1,5-a]pyridinyl moiety is

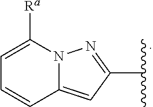

In some embodiments, the compound is a single enantiomer and the pyrazolo[1,5-a]pyridinyl moiety is in an alpha (a) configuration. In some embodiments, the compound is a single enantiomer and the pyrazolo[1,5-a]pyridinyl moiety is in an beta ((3) configuration.

In the structure of Formula I, each R^{a} is independently halo, C₁₋₆alkyl, C₃₋₆cycloalkyl, C₁₋₆haloalkyl, C₁₋₆alkoxy or C₁₋₆haloalkoxy. In some embodiments, R^{a} is halo such as F, Cl, Br, or I. In other embodiments, R^{a} is F, Br, or Cl. In still other embodiments, R^{a} is F. In further embodiments, R^{a} is Br. In yet other embodiments, R^{a} is Cl. In still further embodiments, R^{a} is I. In other embodiments, R^{a} is C₁₋₆alkyl such as methyl, ethyl, propyl, butyl, pentyl, or hexyl. In further embodiments, R^{a} is methyl, ethyl, or isopropyl. In yet other embodiments, R^{a} is methyl. In further embodiments, R^{a} is C₃₋₆cycloalkyl such as cyclopropyl, cyclobutyl, cyclopentyl, or cyclohexyl. In still further embodiments, R^{a} is cyclopropyl. In yet other embodiments, R^{a} is C₁₋₆haloalkyl such as CF₃, CHF₂, CH₂F, CH₂CF₃, C(CH₃)₂F, or C(CH₃)F₂. In still other embodiments, R^{a} is CF₃ or CHF₂. In further embodiments, R^{a} is C₁₋₆alkoxy such as methoxy, ethoxy, propoxy, butoxy, pentoxy, or hexoxy. In yet other embodiments, R^{a} is methoxy or ethoxy. In still further embodiments, R^{a} is methoxy. In yet other embodiments, R^{a} is C₁₋₆haloalkoxy such as OCF₃ or OCH₂CF₃. In further embodiments, R^{a} is OCF₃. In other embodiments, R^{a} is F, Cl, methyl, ethyl, isopropyl, cyclopropyl, CF₃, CHF₂, methoxy, or OCF₃. In still other embodiments, one R^{a} is halo or C₁₋₆alkyl and the second R^{a} is C₁₋₆alkyl. In further embodiments, one R^{a} is F or methyl, and the second R^{a} is methyl.

In the structure of Formula I, R^{b} is

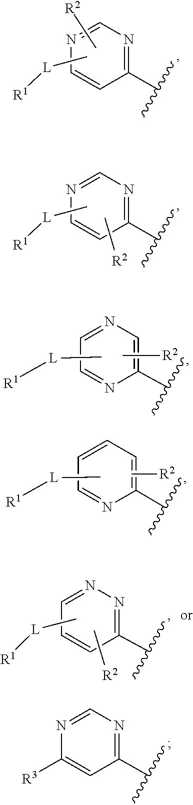

In some embodiments, R^{b} is

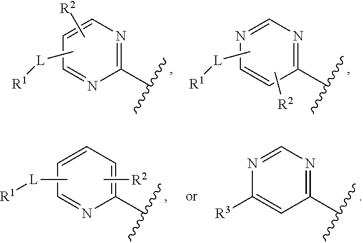

In some embodiments, R^{b} is

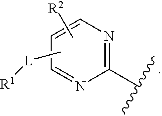

In other embodiments, R^{b} is

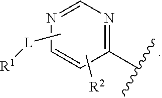

In yet other embodiments, R^{b} is

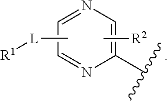

In other embodiments, R^{b} is

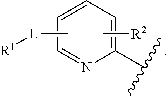

In further embodiments, R^{b} is

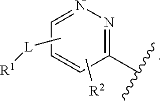

In still further embodiments, R^{b} is

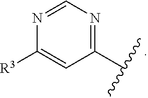

In some embodiments, R^{b} is

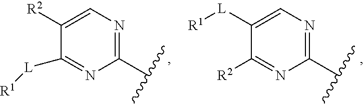

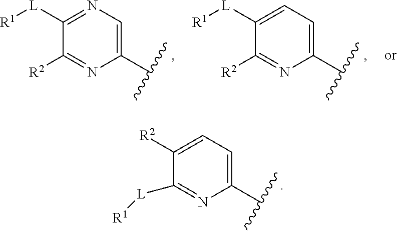

In other embodiments, R^{b} is

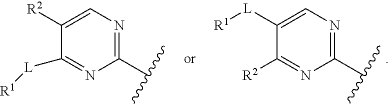

In yet other embodiments, R^{b} is

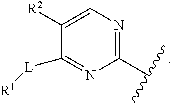

In still other embodiments, R^{b} is

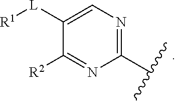

In some embodiments, R^{b} is

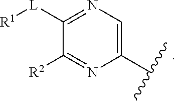

In other embodiments, R^{b} is

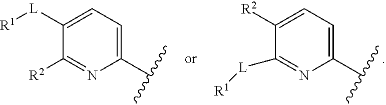

In yet other embodiments, R^{b} is

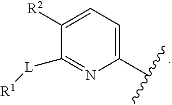

In further embodiments, R^{b} is

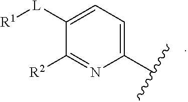

In the structures of R^{b}, L is —C(O)—, —CH(OH)—, or —C(O)NH—. In some embodiments, L is —C(O)— or —CH(OH)—. In other embodiments, L is —C(O)—. In yet other embodiments, L is —CH(OH)—. In further embodiments, L is —C(O)NH—. In some embodiments when L is —C(O)NH—, the carbonyl moiety is attached to the heteroaryl moiety, and the amino moiety is attached to R¹. In other embodiments, the carbonyl moiety is attached to R¹, and the amino moiety is attached to the heteroaryl moiety.

In the structures of R^{b}, R¹ is optionally substituted phenyl, optionally substituted 4-, 5-, or 6-membered heterocyclyl, —NR⁴R⁵, or optionally substituted C₃₋₆cycloalkyl. In some embodiments, R¹ is optionally substituted phenyl, optionally substituted 4-, 5-, or 6-membered heterocyclyl, or optionally substituted C₃₋₆cycloalkyl.

In some embodiments, R¹ is phenyl, optionally substituted with one or more of C₁₋₆alkoxy, halo, C₁₋₆alkyl, or C₁₋₆haloalkyl. In further embodiments, the phenyl is unsubstituted. In other embodiments, the phenyl group is optionally substituted with C₁₋₆alkoxy, such as methoxy, ethoxy, or propoxy. In yet other embodiments, the phenyl group is optionally substituted with halo such as F, Cl, or Br. In still further embodiments, the phenyl group is optionally substituted with C₁₋₆alkyl such as methyl, ethyl, or isopropyl. In yet other embodiments, the phenyl group is optionally substituted with C₁₋₆haloalkyl such as CF₃, CH₂CF₃, or CHF₂. In some embodiments, R¹ is

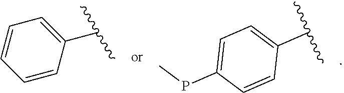

In some embodiments, R¹ is 4-, 5-, or 6-membered heterocyclyl, optionally substituted with one or more of one or more of halo, C₁₋₆alkyl, OH, C₁₋₆hydroxyalkyl, or C₃₋₆cycloalkyl. In further embodiments, R¹ is optionally substituted azetidinyl, optionally substituted pyrrolidinyl, optionally substituted piperidinyl, optionally substituted piperazinyl, or optionally substituted morpholinyl. In other embodiments, R¹ is optionally substituted pyrrolidinyl or optionally substituted piperidinyl. In yet other embodiments, R¹ is optionally substituted azetidinyl. In still other embodiments, the heterocyclyl group is unsubstituted. In yet other embodiments, R¹ is optionally substituted pyrrolidinyl. In further embodiments, R¹ is optionally substituted piperidinyl. In yet other embodiments, R¹ is optionally substituted piperazinyl. In still further embodiments, R¹ is optionally substituted morpholinyl. In other embodiments, the 4-, 5-, or 6-membered heterocyclyl group is unsubstituted. In further embodiments, R¹ is unsubstituted azetidinyl, pyrrolidinyl, piperidinyl, piperazinyl, or morpholinyl. In still further embodiments, R¹ is unsubstituted pyrrolidinyl or piperidinyl. In other embodiments, the 4-, 5-, or 6-membered heterocyclyl group is optionally substituted with one or more halo such as F, Cl, or Br. In still further embodiments, the 4-, 5-, or 6-membered heterocyclyl group is optionally substituted with one or more C₁₋₆alkyl such as methyl, ethyl, or isopropyl. In other embodiments, the 4-, 5-, or 6-membered heterocyclyl group is optionally substituted with OH. In further embodiments, the 4-, 5-, or 6-membered heterocyclyl group is optionally substituted with C₁₋₆hydroxyalkyl such as C(CH₃)₂OH. In further embodiments, the 4-, 5-, or 6-membered heterocyclyl group is optionally substituted with C₃₋₆cycloalkyl such as cyclopropyl or cyclobutyl. In some embodiments, R¹ is

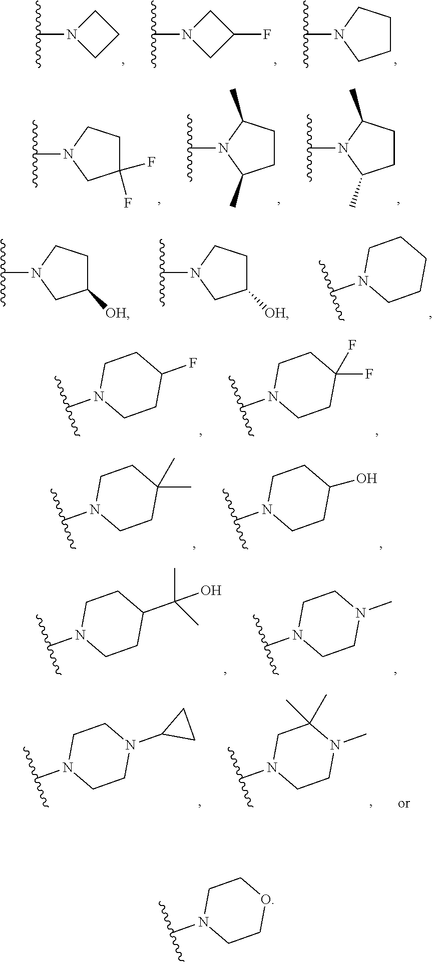

In further embodiments R¹ is

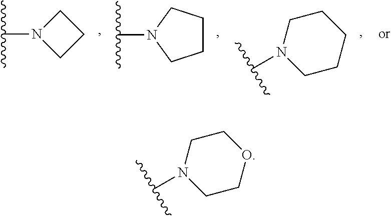

In still further embodiments, R¹ is

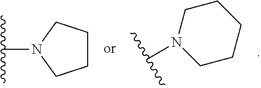 [SMILES: *N1CCCC1 ; *N1CCCCC1]

In yet other embodiments, R¹ is

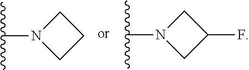

In other embodiments, R¹ is

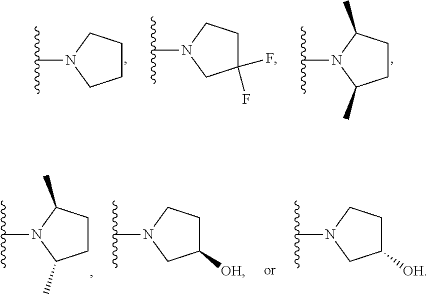

In still other embodiments, R¹ is

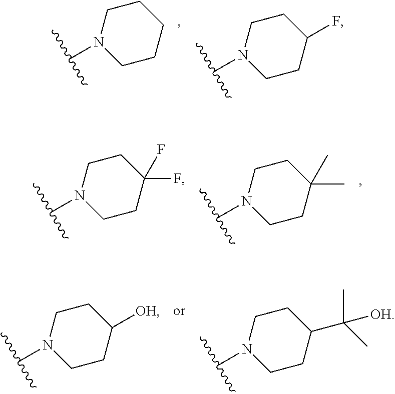

In yet other embodiments, R¹ is

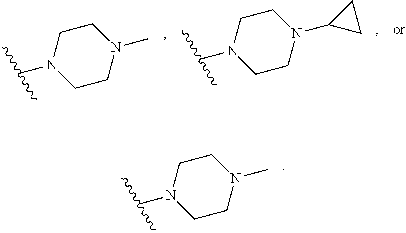

In other embodiments, R¹ is

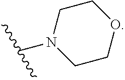 [SMILES: *N1CCOCC1]

In some embodiments, R¹ is C₃₋₆cycloalkyl, optionally substituted with one or more of halo, OH, C₁₋₆alkyl, or C₁₋₆haloalkyl. In further embodiments, R¹ is optionally substituted cyclopropyl or optionally substituted cyclobutyl. In other embodiments, the C₃₋₆cycloalkyl group is unsubstituted. In further embodiments, R¹ is unsubstituted cyclopropyl or unsubstituted cyclobutyl. In other embodiments, the cycloalkyl group is optionally substituted with one or more halo such as F, Cl, or Br. In yet other embodiments, the cycloalkyl group is optionally substituted with OH. In still further embodiments, the cycloalkyl group is optionally substituted with one or more C₁₋₆alkyl such as methyl, ethyl, or isopropyl. In further embodiments, the cycloalkyl group is optionally substituted with C₁₋₆haloalkyl such as CF₃, CH₂CF₃, CH₂F, or CHF₂. In still further embodiments, R² is

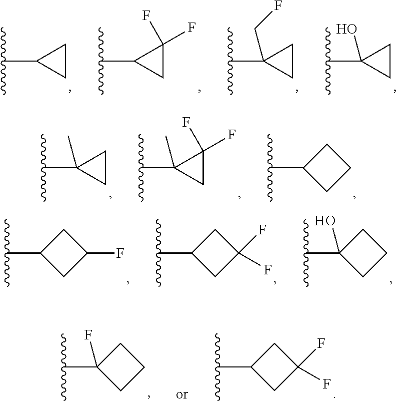

In some embodiments, R¹ is

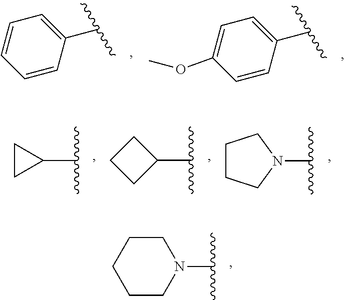

or —N(CH₃)₂. In other embodiments, R¹ is

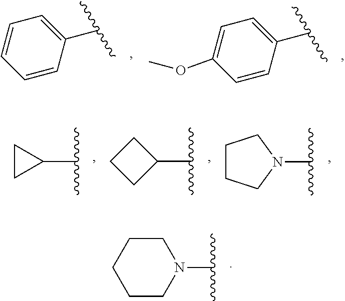

In some embodiments, R¹ is —NR⁴R⁵, wherein R⁴ and R⁵ are each independently hydrogen or C₁₋₆alkyl. In some embodiments, R⁴ and R⁵ are each independently hydrogen or methyl.

In the structures of R^{b}, R² is hydrogen, C₁₋₆alkyl, C₁₋₆haloalkyl, or halo. In some embodiments, R² is hydrogen or C₁₋₆alkyl. In other embodiments, R² is hydrogen. In yet other embodiments, R² is halo such as F, Cl, or Br. In further embodiments, R² is C₁₋₆alkyl such as methyl, ethyl, or isopropyl. In other embodiments, R² is methyl. In still further embodiments, R² is C₁₋₆haloalkyl such as CF₃, CH₂CF₃, or CHF₂. In other embodiments, R² is hydrogen or methyl.

In the structures of R^{b}, R³ is optionally substituted 5- or 6-membered heterocyclyl. In some embodiments, R³ is optionally substituted pyrrolidinyl, optionally substituted piperidinyl, optionally substituted piperazinyl, or optionally substituted morpholinyl. In some embodiments, R³ is optionally substituted 5-membered heterocyclyl. In further embodiments, R³ is optionally substituted pyrrolidinyl. In other embodiments, R³ is optionally substituted 6-membered heterocyclyl. In further embodiments, the optionally substituted 6-membered heterocyclyl is optionally substituted piperidinyl, optionally substituted piperazinyl, or optionally substituted morpholinyl. In still further embodiments, R³ is optionally substituted piperidinyl. In yet other embodiments, R³ is optionally substituted piperazinyl. In still other embodiments, R³ is optionally substituted morpholinyl. In further embodiments, R³ is unsubstituted 5- or 6-membered heterocyclyl.

In other embodiments, the 5- or 6-membered heterocyclyl group is optionally substituted with one or more of halo, C₁₋₆alkyl, OH, C₁₋₆hydroxyalkyl, or optionally substituted phenyl. In other embodiments, the 5- or 6-membered heterocyclyl group is optionally substituted with one or more halo such as F, Cl, or Br. In still further embodiments, the 5- or 6-membered heterocyclyl group is optionally substituted with one or more C₁₋₆alkyl such as methyl, ethyl, or isopropyl. In yet other embodiments, the 5- or 6-membered heterocyclyl group is optionally substituted with OH. In further embodiments, the 5- or 6-membered heterocyclyl group is optionally substituted with C₁₋₆hydroxyalkyl such as C(CH₃)₂OH. In some embodiments, the 5- or 6-membered heterocyclyl group is optionally substituted with a phenyl group wherein the phenyl group is optionally substituted with one or more of halo, C₁₋₆alkyl, C₁₋₆haloalkyl, or cyano. In some embodiments, R³ is morpholinyl, optionally substituted with one phenyl group. In further embodiments, R³ is

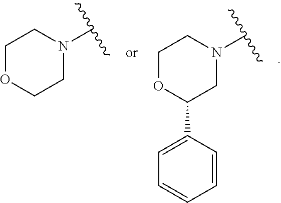 [SMILES: *N1CCOCC1 ; *N1CCO[C@@H](c2ccccc2)C1]

The disclosure further provides R-enantiomers, S-enantiomers, or racemic mixtures of any of the compounds described herein. In some embodiments, the compound is an S-enantiomer. In other embodiments, the compound is the R-enantiomer. In further embodiments, the compound is racemic.

In another embodiment, the compounds of the disclosure may be enantiomerically enriched, e.g., the enantiomeric excess or “ee” of the compound is greater than about 5% as measured by chiral HPLC. In some embodiments, the ee is greater than about 10%. In other embodiments, the ee is greater than about 20%. In further embodiments, the ee is greater than about 30%. In yet other embodiments, the ee is greater than about 40%. In still further embodiments, the ee is greater than about 50%. In other embodiments, the ee is greater than about 60%. In further embodiments, the ee is greater than about 70%. In still other embodiments, the ee is greater than about 80%. In yet further embodiments, the ee is greater than about 85%. In other embodiments, the ee is greater than about 90%. In further embodiments, the ee is greater than about 91%. In yet other embodiments, the ee is greater than about 92%. In still further embodiments, the ee is greater than about 93%. In other embodiments, the ee is greater than about 94%. In further embodiments, the ee is greater than about 95%. In still other embodiments, the ee is greater than about 96%. In yet further embodiments, the ee is greater than about 97%. In other embodiments, the ee is greater than about 98%. In further embodiments, the ee is greater than about 99%.

The present disclosure encompasses the preparation and use of salts of compounds of the disclosure. Salts of compounds of the disclosure can be prepared during the final isolation and purification of the compounds or separately by reacting the compound with an acid or base as appropriate.

### EXAMPLES

In some embodiments, the disclosure provides specific examples as set forth in Table 1 below, and their pharmaceutically acceptable salts and/or isotopologues.

TABLE 1

| Ex. No. | Structure | Name |
|---|---|---|
| 89 | 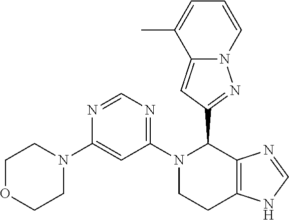 [SMILES: Cc1cccn2nc([C@H]3c4nc[nH]c4CCN3c3cc(N4CCOCC4)ncn3)cc12] | (R)-4-(6-(4-(4-methylpyrazolo[1,5-a]pyridin-2- yl)-1,4,6,7-tetrahydro-5H-imidazo[4,5- c]pyridin-5-yl)pyrimidin-4-yl)morpholine |
| 90 | 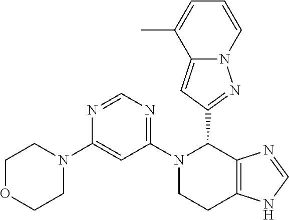 [SMILES: Cc1cccn2nc([C@@H]3c4nc[nH]c4CCN3c3cc(N4CCOCC4)ncn3)cc12] | (S)-4-(6-(4-(4-methylpyrazolo[1,5-a]pyridin-2- yl)-1,4,6,7-tetrahydro-5H-imidazo[4,5- c]pyridin-5-yl)pyrimidin-4-yl)morpholine |
| 91 | 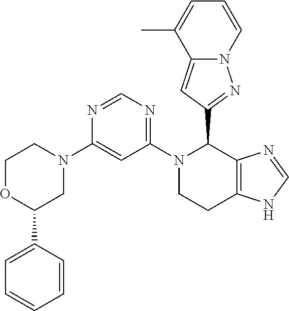 [SMILES: Cc1cccn2nc([C@H]3c4nc[nH]c4CCN3c3cc(N4CCO[C@@H](c5ccccc5)C4)ncn3)cc12] | (S)-4-(6-((R)-4-(4-methylpyrazolo[1,5- a]pyridin-2-yl)-1,4,6,7-tetrahydro-5H- imidazo[4,5-c]pyridin-5-yl)pyrimidin-4-yl)-2- phenylmorpholine |
| 92 | 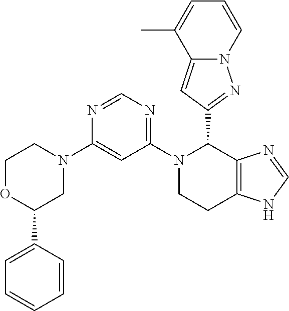 [SMILES: Cc1cccn2nc([C@@H]3c4nc[nH]c4CCN3c3cc(N4CCO[C@@H](c5ccccc5)C4)ncn3)cc12] | (S)-4-(6-((S)-4-(4-methylpyrazolo[1,5- a]pyridin-2-yl)-1,4,6,7-tetrahydro-5H- imidazo[4,5-c]pyridin-5-yl)pyrimidin-4-yl)-2- phenylmorpholine |
| 93 | 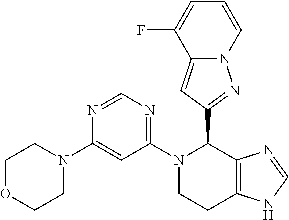 [SMILES: Fc1cccn2nc([C@H]3c4nc[nH]c4CCN3c3cc(N4CCOCC4)ncn3)cc12] | (R)-4-(6-(4-(4-fluoropyrazolo[1,5-a]pyridin-2- yl)-1,4,6,7-tetrahydro-5H-imidazo[4,5- c]pyridin-5-yl)pyrimidin-4-yl)morpholine |
| 94 | 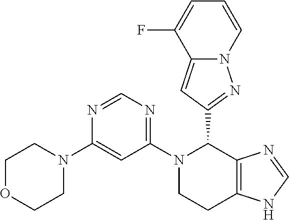 [SMILES: Fc1cccn2nc([C@@H]3c4nc[nH]c4CCN3c3cc(N4CCOCC4)ncn3)cc12] | (S)-4-(6-(4-(4-fluoropyrazolo[1,5-a]pyridin-2- yl)-1,4,6,7-tetrahydro-5H-imidazo[4,5- c]pyridin-5-yl)pyrimidin-4-yl)morpholine |
| 95 | 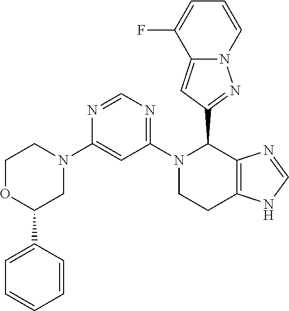 [SMILES: Fc1cccn2nc([C@H]3c4nc[nH]c4CCN3c3cc(N4CCO[C@@H](c5ccccc5)C4)ncn3)cc12] | (S)-4-(6-((R)-4-(4-fluoropyrazolo[1,5-a]pyridin- 2-yl)-1,4,6,7-tetrahydro-5H-imidazo[4,5- c]pyridin-5-yl)pyrimidin-4-yl)-2- phenylmorpholine |
| 96 | 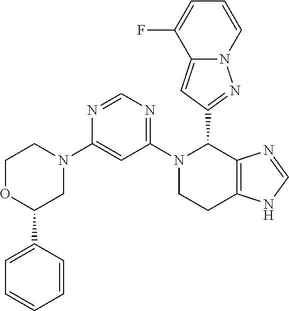 [SMILES: Fc1cccn2nc([C@@H]3c4nc[nH]c4CCN3c3cc(N4CCO[C@@H](c5ccccc5)C4)ncn3)cc12] | (S)-4-(6-((S)-4-(4-fluoropyrazolo[1,5-a]pyridin- 2-yl)-1,4,6,7-tetrahydro-5H-imidazo[4,5- c]pyridin-5-yl)pyrimidin-4-yl)-2- phenylmorpholine |
| 97 | 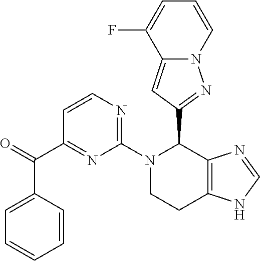 [SMILES: O=C(c1ccccc1)c1ccnc(N2CCc3[nH]cnc3[C@@H]2c2cc3c(F)cccn3n2)n1] | (R)-(2-(4-(4-fluoropyrazolo[1,5-a]pyridin-2-yl)- 1,4,6,7-tetrahydro-5H-imidazo[4,5-c]pyridin-5- yl)pyrimidin-4-yl)(phenyl)methanone |
| 98 | 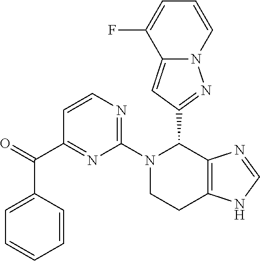 [SMILES: O=C(c1ccccc1)c1ccnc(N2CCc3[nH]cnc3[C@H]2c2cc3c(F)cccn3n2)n1] | (S)-(2-(4-(4-fluoropyrazolo[1,5-a]pyridin-2-yl)- 1,4,6,7-tetrahydro-5H-imidazo[4,5-c]pyridin-5- yl)pyrimidin-4-yl)(phenyl)methanone |
| 99 | 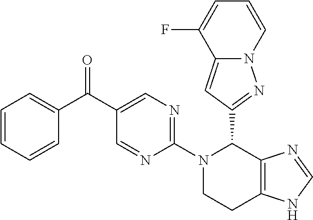 [SMILES: O=C(c1ccccc1)c1cnc(N2CCc3[nH]cnc3[C@H]2c2cc3c(F)cccn3n2)nc1] | (S)-(2-(4-(4-fluoropyrazolo[1,5-a]pyridin-2-yl)- 1,4,6,7-tetrahydro-5H-imidazo[4,5-c]pyridin-5- yl)pyrimidin-5-yl)(phenyl)methanone |
| 100 | 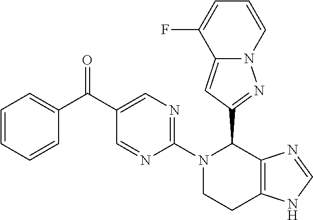 [SMILES: O=C(c1ccccc1)c1cnc(N2CCc3[nH]cnc3[C@@H]2c2cc3c(F)cccn3n2)nc1] | (R)-(2-(4-(4-fluoropyrazolo[1,5-a]pyridin-2-yl)- 1,4,6,7-tetrahydro-5H-imidazo[4,5-c]pyridin-5- yl)pyrimidin-5-yl)(phenyl)methanone |
| 101 | 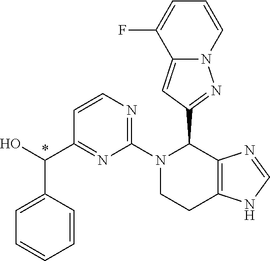 [SMILES: OC(c1ccccc1)c1ccnc(N2CCc3[nH]cnc3[C@@H]2c2cc3c(F)cccn3n2)n1] | (2-((R)-4-(4-fluoropyrazolo[1,5-a]pyridin-2-yl)- 1,4,6,7-tetrahydro-5H-imidazo[4,5-c]pyridin-5- yl)pyrimidin-4-yl)(phenyl)methanol Diastereomer #1 (Rt = 1.038 min) |
| 102 | 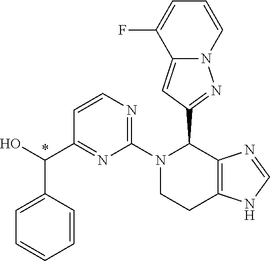 [SMILES: OC(c1ccccc1)c1ccnc(N2CCc3[nH]cnc3[C@@H]2c2cc3c(F)cccn3n2)n1] | (2-((R)-4-(4-fluoropyrazolo[1,5-a]pyridin-2-yl)- 1,4,6,7-tetrahydro-5H-imidazo[4,5-c]pyridin-5- yl)pyrimidin-4-yl)(phenyl)methanol Diastereomer #2 (Rt = 1.557 min) |
| 103 | 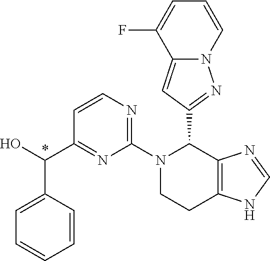 [SMILES: OC(c1ccccc1)c1ccnc(N2CCc3[nH]cnc3[C@H]2c2cc3c(F)cccn3n2)n1] | (2-((S)-4-(4-fluoropyrazolo[1,5-a]pyridin-2-yl)- 1,4,6,7-tetrahydro-5H-imidazo[4,5-c]pyridin-5- yl)pyrimidin-4-yl)(phenyl)methanol Diastereomer #3 (Rt = 2.838 min) |
| 104 | 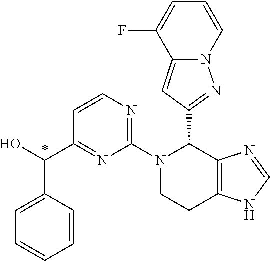 [SMILES: OC(c1ccccc1)c1ccnc(N2CCc3[nH]cnc3[C@H]2c2cc3c(F)cccn3n2)n1] | (2-((S)-4-(4-fluoropyrazolo[1,5-a]pyridin-2-yl)- 1,4,6,7-tetrahydro-5H-imidazo[4,5-c]pyridin-5- yl)pyrimidin-4-yl)(phenyl)methanol Diastereomer #4 (Rt = 4.738 min) |
| 105 | 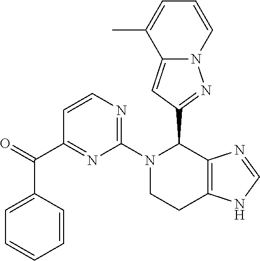 [SMILES: Cc1cccn2nc([C@H]3c4nc[nH]c4CCN3c3nccc(C(=O)c4ccccc4)n3)cc12] | (R)-(2-(4-(4-methylpyrazolo[1,5-a]pyridin-2- yl)-1,4,6,7-tetrahydro-5H-imidazo[4,5- c]pyridin-5-yl)pyrimidin-4- yl)(phenyl)methanone |
| 106 | 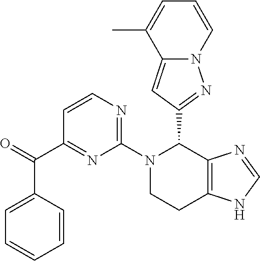 [SMILES: Cc1cccn2nc([C@@H]3c4nc[nH]c4CCN3c3nccc(C(=O)c4ccccc4)n3)cc12] | (S)-(2-(4-(4-methylpyrazolo[1,5-a]pyridin-2- yl)-1,4,6,7-tetrahydro-5H-imidazo[4,5- c]pyridin-5-yl)pyrimidin-4- yl)(phenyl)methanone |
| 107 | 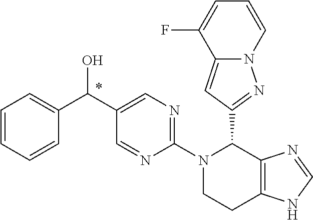 [SMILES: OC(c1ccccc1)c1cnc(N2CCc3[nH]cnc3[C@H]2c2cc3c(F)cccn3n2)nc1] | (2-((S)-4-(4-fluoropyrazolo[1,5-a]pyridin-2-yl)- 1,4,6,7-tetrahydro-5H-imidazo[4,5-c]pyridin-5- yl)pyrimidin-5-yl)(phenyl)methanol Diastereomer #1 |
| 108 | 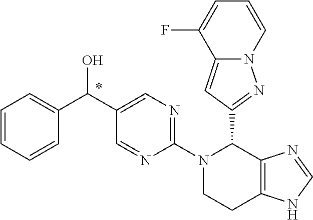 [SMILES: OC(c1ccccc1)c1cnc(N2CCc3[nH]cnc3[C@H]2c2cc3c(F)cccn3n2)nc1] | (2-((S)-4-(4-fluoropyrazolo[1,5-a]pyridin-2-yl)- 1,4,6,7-tetrahydro-5H-imidazo[4,5-c]pyridin-5- yl)pyrimidin-5-yl)(phenyl)methanol Diastereomer #2 |
| 109 | 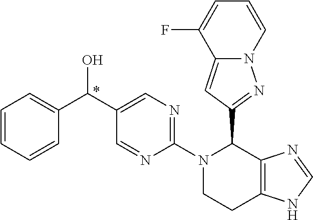 [SMILES: OC(c1ccccc1)c1cnc(N2CCc3[nH]cnc3[C@@H]2c2cc3c(F)cccn3n2)nc1] | (2-((R)-4-(4-fluoropyrazolo[1,5-a]pyridin-2-yl)- 1,4,6,7-tetrahydro-5H-imidazo[4,5-c]pyridin-5- yl)pyrimidin-5-yl)(phenyl)methanol Diastereomer #3 |
| 110 | 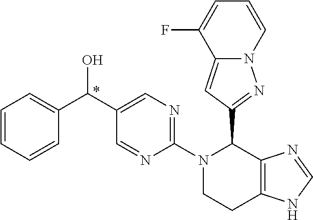 [SMILES: OC(c1ccccc1)c1cnc(N2CCc3[nH]cnc3[C@@H]2c2cc3c(F)cccn3n2)nc1] | (2-((R)-4-(4-fluoropyrazolo[1,5-a]pyridin-2-yl)- 1,4,6,7-tetrahydro-5H-imidazo[4,5-c]pyridin-5- yl)pyrimidin-5-yl)(phenyl)methanol Diastereomer #4 |
| 111 | 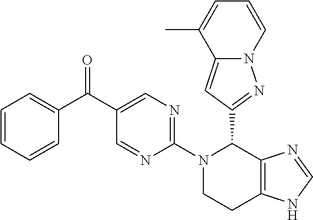 [SMILES: Cc1cccn2nc([C@@H]3c4nc[nH]c4CCN3c3ncc(C(=O)c4ccccc4)cn3)cc12] | (S)-(2-(4-(4-methylpyrazolo[1,5-a]pyridin-2- yl)-1,4,6,7-tetrahydro-5H-imidazo[4,5- c]pyridin-5-yl)pyrimidin-5- yl)(phenyl)methanone |
| 112 | 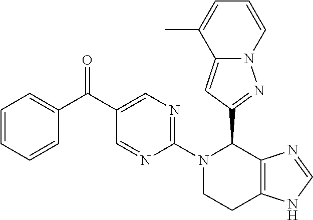 [SMILES: Cc1cccn2nc([C@H]3c4nc[nH]c4CCN3c3ncc(C(=O)c4ccccc4)cn3)cc12] | (R)-(2-(4-(4-methylpyrazolo[1,5-a]pyridin-2- yl)-1,4,6,7-tetrahydro-5H-imidazo[4,5- c]pyridin-5-yl)pyrimidin-5- yl)(phenyl)methanone |
| 113 | 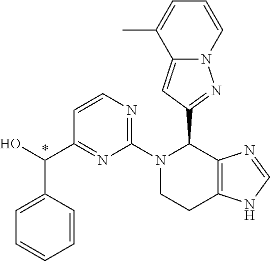 [SMILES: Cc1cccn2nc([C@H]3c4nc[nH]c4CCN3c3nccc(C(O)c4ccccc4)n3)cc12] | (2-((R)-4-(4-methylpyrazolo[1,5-a]pyridin-2- yl)-1,4,6,7-tetrahydro-5H-imidazo[4,5- c]pyridin-5-yl)pyrimidin-4-yl)(phenyl)methanol Diastereomer #1 (Rt = 2.737 min) |
| 114 | 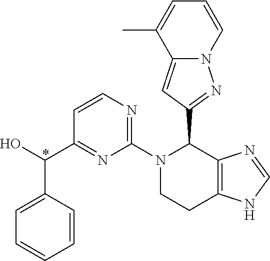 [SMILES: Cc1cccn2nc([C@H]3c4nc[nH]c4CCN3c3nccc(C(O)c4ccccc4)n3)cc12] | (2-((R)-4-(4-methylpyrazolo[1,5-a]pyridin-2- yl)-1,4,6,7-tetrahydro-5H-imidazo[4,5- c]pyridin-5-yl)pyrimidin-4-yl)(phenyl)methanol Diastereomer #2 (Rt = 3.621 min) |
| 115 | 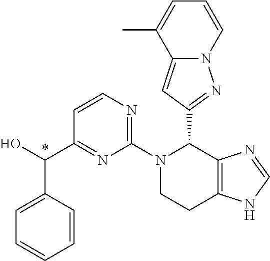 [SMILES: Cc1cccn2nc([C@@H]3c4nc[nH]c4CCN3c3nccc(C(O)c4ccccc4)n3)cc12] | (2-((S)-4-(4-methylpyrazolo[1,5-a]pyridin-2- yl)-1,4,6,7-tetrahydro-5H-imidazo[4,5- c]pyridin-5-yl)pyrimidin-4-yl)(phenyl)methanol Diastereomer #3 (Rt = 4.930 min) |
| 116 | 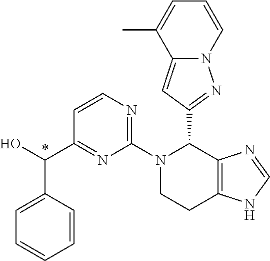 [SMILES: Cc1cccn2nc([C@@H]3c4nc[nH]c4CCN3c3nccc(C(O)c4ccccc4)n3)cc12] | (2-((S)-4-(4-methylpyrazolo[1,5-a]pyridin-2- yl)-1,4,6,7-tetrahydro-5H-imidazo[4,5- c]pyridin-5-yl)pyrimidin-4-yl)(phenyl)methanol Diastereomer #4 (Rt = 7.601 min) |
| 117 | 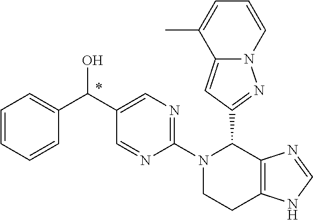 [SMILES: Cc1cccn2nc([C@@H]3c4nc[nH]c4CCN3c3ncc(C(O)c4ccccc4)cn3)cc12] | (2-((S)-4-(4-methylpyrazolo[1,5-a]pyridin-2- yl)-1,4,6,7-tetrahydro-5H-imidazo[4,5- c]pyridin-5-yl)pyrimidin-5-yl)(phenyl)methanol Diastereomer #1 |
| 118 | 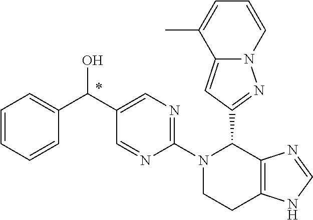 [SMILES: Cc1cccn2nc([C@@H]3c4nc[nH]c4CCN3c3ncc(C(O)c4ccccc4)cn3)cc12] | (2-((S)-4-(4-methylpyrazolo[1,5-a]pyridin-2- yl)-1,4,6,7-tetrahydro-5H-imidazo[4,5- c]pyridin-5-yl)pyrimidin-5-yl)(phenyl)methanol Diastereomer #2 |
| 119 | 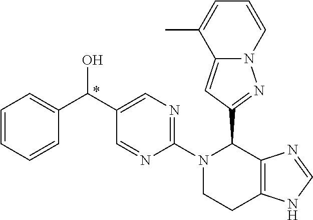 [SMILES: Cc1cccn2nc([C@H]3c4nc[nH]c4CCN3c3ncc(C(O)c4ccccc4)cn3)cc12] | (2-((R)-4-(4-methylpyrazolo[1,5-a]pyridin-2- yl)-1,4,6,7-tetrahydro-5H-imidazo[4,5- c]pyridin-5-yl)pyrimidin-5-yl)(phenyl)methanol Diastereomer #3 |
| 120 | 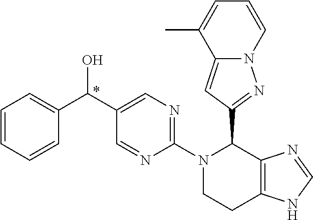 [SMILES: Cc1cccn2nc([C@H]3c4nc[nH]c4CCN3c3ncc(C(O)c4ccccc4)cn3)cc12] | (2-((R)-4-(4-methylpyrazolo[1,5-a]pyridin-2- yl)-1,4,6,7-tetrahydro-5H-imidazo[4,5- c]pyridin-5-yl)pyrimidin-5-yl)(phenyl)methanol Diastereomer #4 |
| 121 | 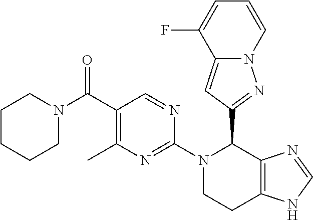 [SMILES: Cc1nc(N2CCc3[nH]cnc3[C@@H]2c2cc3c(F)cccn3n2)ncc1C(=O)N1CCCCC1] | (R)-(2-(4-(4-fluoropyrazolo[1,5-a]pyridin-2-yl)- 1,4,6,7-tetrahydro-5H-imidazo[4,5-c]pyridin-5- yl)-4-methylpyrimidin-5-yl)(piperidin-1- yl)methanone |
| 122 | 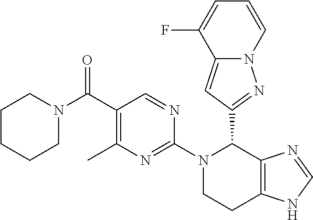 [SMILES: Cc1nc(N2CCc3[nH]cnc3[C@H]2c2cc3c(F)cccn3n2)ncc1C(=O)N1CCCCC1] | (S)-(2-(4-(4-fluoropyrazolo[1,5-a]pyridin-2-yl)- 1,4,6,7-tetrahydro-5H-imidazo[4,5-c]pyridin-5- yl)-4-methylpyrimidin-5-yl)(piperidin-1- yl)methanone |
| 123 | 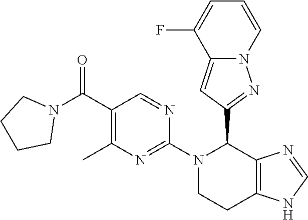 [SMILES: Cc1nc(N2CCc3[nH]cnc3[C@@H]2c2cc3c(F)cccn3n2)ncc1C(=O)N1CCCC1] | (R)-(2-(4-(4-fluoropyrazolo[1,5-a]pyridin-2-yl)- 1,4,6,7-tetrahydro-5H-imidazo[4,5-c]pyridin-5- yl)-4-methylpyrimidin-5-yl)(pyrrolidin-1- yl)methanone |
| 124 | 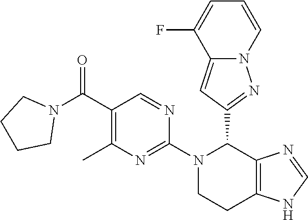 [SMILES: Cc1nc(N2CCc3[nH]cnc3[C@H]2c2cc3c(F)cccn3n2)ncc1C(=O)N1CCCC1] | (S)-(2-(4-(4-fluoropyrazolo[1,5-a]pyridin-2-yl)- 1,4,6,7-tetrahydro-5H-imidazo[4,5-c]pyridin-5- yl)-4-methylpyrimidin-5-yl)(pyrrolidin-1- yl)methanone |
| 125 | 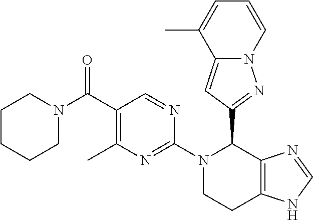 [SMILES: Cc1nc(N2CCc3[nH]cnc3[C@@H]2c2cc3c(C)cccn3n2)ncc1C(=O)N1CCCCC1] | (R)-(4-methyl-2-(4-(4-methylpyrazolo[1,5- a]pyridin-2-yl)-1,4,6,7-tetrahydro-5H- imidazo[4,5-c]pyridin-5-yl)pyrimidin-5- yl)(piperidin-1-yl)methanone |
| 126 | 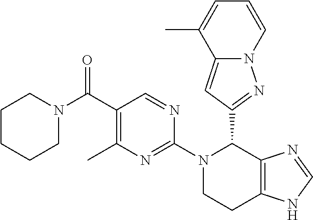 [SMILES: Cc1nc(N2CCc3[nH]cnc3[C@H]2c2cc3c(C)cccn3n2)ncc1C(=O)N1CCCCC1] | (S)-(4-methyl-2-(4-(4-methylpyrazolo[1,5- a]pyridin-2-yl)-1,4,6,7-tetrahydro-5H- imidazo[4,5-c]pyridin-5-yl)pyrimidin-5- yl)(piperidin-1-yl)methanone |
| 127 | 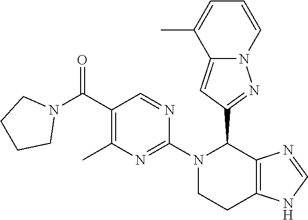 [SMILES: Cc1nc(N2CCc3[nH]cnc3[C@@H]2c2cc3c(C)cccn3n2)ncc1C(=O)N1CCCC1] | (R)-(4-methyl-2-(4-(4-methylpyrazolo[1,5- a]pyridin-2-yl)-1,4,6,7-tetrahydro-5H- imidazo[4,5-c]pyridin-5-yl)pyrimidin-5- yl)(pyrrolidin-1-yl)methanone |
| 128 | 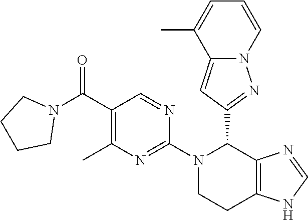 [SMILES: Cc1nc(N2CCc3[nH]cnc3[C@H]2c2cc3c(C)cccn3n2)ncc1C(=O)N1CCCC1] | (S)-(4-methyl-2-(4-(4-methylpyrazolo[1,5- a]pyridin-2-yl)-1,4,6,7-tetrahydro-5H- imidazo[4,5-c]pyridin-5-yl)pyrimidin-5- yl)(pyrrolidin-1-yl)methanone |
| 129 | 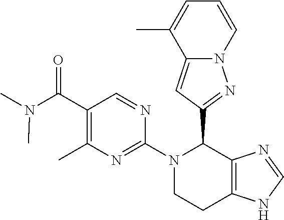 [SMILES: Cc1nc(N2CCc3[nH]cnc3[C@@H]2c2cc3c(C)cccn3n2)ncc1C(=O)N(C)C] | (R)-N,N,4-trimethyl-2-(4-(4- methylpyrazolo[1,5-a]pyridin-2-yl)-1,4,6,7- tetrahydro-5H-imidazo[4,5-c]pyridin-5- yl)pyrimidine-5-carboxamide |
| 130 | 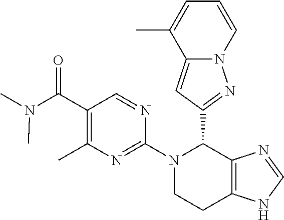 [SMILES: Cc1nc(N2CCc3[nH]cnc3[C@H]2c2cc3c(C)cccn3n2)ncc1C(=O)N(C)C] | (S)-N,N,4-trimethyl-2-(4-(4- methylpyrazolo[1,5-a]pyridin-2-yl)-1,4,6,7- tetrahydro-5H-imidazo[4,5-c]pyridin-5- yl)pyrimidine-5-carboxamide |
| 131 | 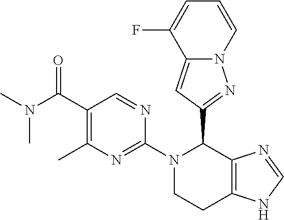 [SMILES: Cc1nc(N2CCc3[nH]cnc3[C@@H]2c2cc3c(F)cccn3n2)ncc1C(=O)N(C)C] | (R)-2-(4-(4-fluoropyrazolo[1,5-a]pyridin-2-yl)- 1,4,6,7-tetrahydro-5H-imidazo[4,5-c]pyridin-5- yl)-N,N,4-trimethylpyrimidine-5-carboxamide |
| 132 | 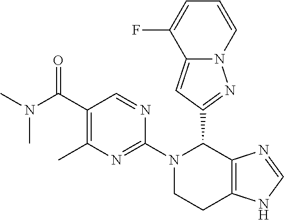 [SMILES: Cc1nc(N2CCc3[nH]cnc3[C@H]2c2cc3c(F)cccn3n2)ncc1C(=O)N(C)C] | (S)-2-(4-(4-fluoropyrazolo[1,5-a]pyridin-2-yl)- 1,4,6,7-tetrahydro-5H-imidazo[4,5-c]pyridin-5- yl)-N,N,4-trimethylpyrimidine-5-carboxamide |
| 204 | 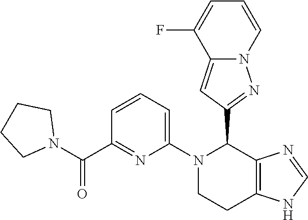 [SMILES: O=C(c1cccc(N2CCc3[nH]cnc3[C@@H]2c2cc3c(F)cccn3n2)n1)N1CCCC1] | (R)-(6-(4-(4-fluoropyrazolo[1,5-a]pyridin-2-yl)- 1,4,6,7-tetrahydro-5H-imidazo[4,5-c]pyridin-5- yl)pyridin-2-yl)(pyrrolidin-1-yl)methanone |
| 205 | 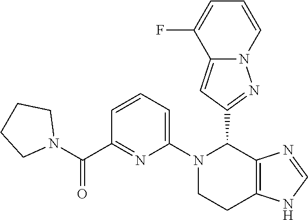 [SMILES: O=C(c1cccc(N2CCc3[nH]cnc3[C@H]2c2cc3c(F)cccn3n2)n1)N1CCCC1] | (S)-(6-(4-(4-fluoropyrazolo[1,5-a]pyridin-2-yl)- 1,4,6,7-tetrahydro-5H-imidazo[4,5-c]pyridin-5- yl)pyridin-2-yl)(pyrrolidin-1-yl)methanone |
| 216 | 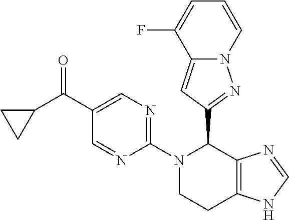 [SMILES: O=C(c1cnc(N2CCc3[nH]cnc3[C@@H]2c2cc3c(F)cccn3n2)nc1)C1CC1] | (R)-cyclopropyl(2-(4-(4-fluoropyrazolo[1,5- a]pyridin-2-yl)-1,4,6,7-tetrahydro-5H- imidazo[4,5-c]pyridin-5-yl)pyrimidin-5- yl)methanone |
| 217 | 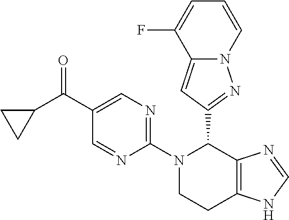 [SMILES: O=C(c1cnc(N2CCc3[nH]cnc3[C@H]2c2cc3c(F)cccn3n2)nc1)C1CC1] | (S)-cyclopropyl(2-(4-(4-fluoropyrazolo[1,5- a]pyridin-2-yl)-1,4,6,7-tetrahydro-5H- imidazo[4,5-c]pyridin-5-yl)pyrimidin-5- yl)methanone |
| 218 | 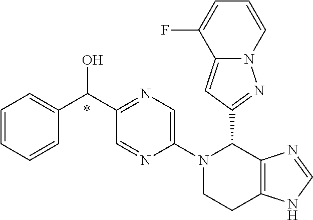 [SMILES: OC(c1ccccc1)c1cnc(N2CCc3[nH]cnc3[C@H]2c2cc3c(F)cccn3n2)cn1] | (5-((S)-4-(4-fluoropyrazolo[1,5-a]pyridin-2-yl)- 1,4,6,7-tetrahydro-5H-imidazo[4,5-c]pyridin-5- yl)pyrazin-2-yl)(phenyl)methanol Diastereomer #2 (Rt = 3.528 min) |
| 219 | 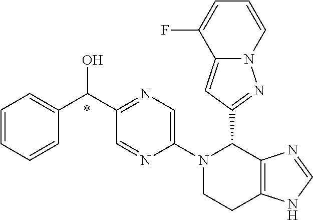 [SMILES: OC(c1ccccc1)c1cnc(N2CCc3[nH]cnc3[C@H]2c2cc3c(F)cccn3n2)cn1] | (5-((S)-4-(4-fluoropyrazolo[1,5-a]pyridin-2-yl)- 1,4,6,7-tetrahydro-5H-imidazo[4,5-c]pyridin-5- yl)pyrazin-2-yl)(phenyl)methanol Diastereomer #1 (Rt = 3.131 min) |
| 220 | 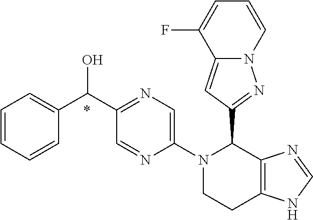 [SMILES: OC(c1ccccc1)c1cnc(N2CCc3[nH]cnc3[C@@H]2c2cc3c(F)cccn3n2)cn1] | (5-((R)-4-(4-fluoropyrazolo[1,5-a]pyridin-2-yl)- 1,4,6,7-tetrahydro-5H-imidazo[4,5-c]pyridin-5- yl)pyrazin-2-yl)(phenyl)methanol Diastereomer #3 (Rt = 5.655 min) |
| 221 | 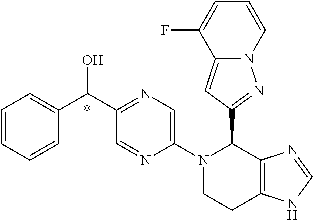 [SMILES: OC(c1ccccc1)c1cnc(N2CCc3[nH]cnc3[C@@H]2c2cc3c(F)cccn3n2)cn1] | (5-((R)-4-(4-fluoropyrazolo[1,5-a]pyridin-2-yl)- 1,4,6,7-tetrahydro-5H-imidazo[4,5-c]pyridin-5- yl)pyrazin-2-yl)(phenyl)methanol Diastereomer #4 (Rt = 6.153 min) |
| 222 | 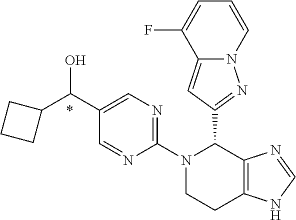 [SMILES: OC(c1cnc(N2CCc3[nH]cnc3[C@H]2c2cc3c(F)cccn3n2)nc1)C1CCC1] | cyclobutyl(2-((S)-4-(4-fluoropyrazolo[1,5- a]pyridin-2-yl)-1,4,6,7-tetrahydro-5H- imidazo[4,5-c]pyridin-5-yl)pyrimidin-5- yl)methanol Diastereomer #2 (Rt = 2.309 min) |
| 223 | 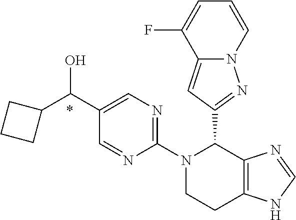 [SMILES: OC(c1cnc(N2CCc3[nH]cnc3[C@H]2c2cc3c(F)cccn3n2)nc1)C1CCC1] | cyclobutyl(2-((S)-4-(4-fluoropyrazolo[1,5- a]pyridin-2-yl)-1,4,6,7-tetrahydro-5H- imidazo[4,5-c]pyridin-5-yl)pyrimidin-5- yl)methanol Diastereomer #3 (Rt = 2.904 min) |
| 224 | 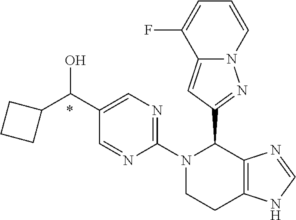 [SMILES: OC(c1cnc(N2CCc3[nH]cnc3[C@@H]2c2cc3c(F)cccn3n2)nc1)C1CCC1] | cyclobutyl(2-((R)-4-(4-fluoropyrazolo[1,5- a]pyridin-2-yl)-1,4,6,7-tetrahydro-5H- imidazo[4,5-c]pyridin-5-yl)pyrimidin-5- yl)methanol Diastereomer #1 (Rt = 2.303 min) |
| 225 | 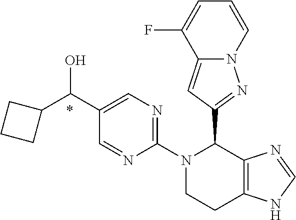 [SMILES: OC(c1cnc(N2CCc3[nH]cnc3[C@@H]2c2cc3c(F)cccn3n2)nc1)C1CCC1] | cyclobutyl(2-((R)-4-(4-fluoropyrazolo[1,5- a]pyridin-2-yl)-1,4,6,7-tetrahydro-5H- imidazo[4,5-c]pyridin-5-yl)pyrimidin-5- yl)methanol Diastereomer #4 (Rt = 2.924 min) |
| 226 | 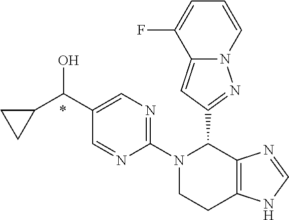 [SMILES: OC(c1cnc(N2CCc3[nH]cnc3[C@H]2c2cc3c(F)cccn3n2)nc1)C1CC1] | cyclopropyl(2-((S)-4-(4-fluoropyrazolo[1,5- a]pyridin-2-yl)-1,4,6,7-tetrahydro-5H- imidazo[4,5-c]pyridin-5-yl)pyrimidin-5- yl)methanol Diastereomer #3 (Rt = 3.738 min) |
| 227 | 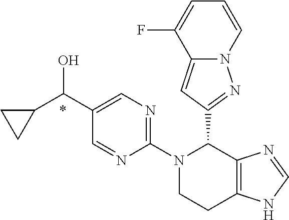 [SMILES: OC(c1cnc(N2CCc3[nH]cnc3[C@H]2c2cc3c(F)cccn3n2)nc1)C1CC1] | cyclopropyl(2-((S)-4-(4-fluoropyrazolo[1,5- a]pyridin-2-yl)-1,4,6,7-tetrahydro-5H- imidazo[4,5-c]pyridin-5-yl)pyrimidin-5- yl)methanol Diastereomer #4 (Rt = 4.254 min) |
| 228 | 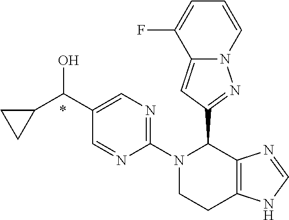 [SMILES: OC(c1cnc(N2CCc3[nH]cnc3[C@@H]2c2cc3c(F)cccn3n2)nc1)C1CC1] | cyclopropyl(2-((R)-4-(4-fluoropyrazolo[1,5- a]pyridin-2-yl)-1,4,6,7-tetrahydro-5H- imidazo[4,5-c]pyridin-5-yl)pyrimidin-5- yl)methanol Diastereomer #1 (Rt = 1.943 min) |
| 229 | 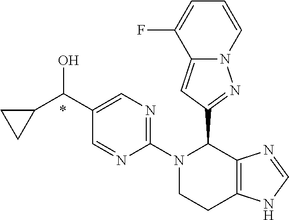 [SMILES: OC(c1cnc(N2CCc3[nH]cnc3[C@@H]2c2cc3c(F)cccn3n2)nc1)C1CC1] | cyclopropyl(2-((R)-4-(4-fluoropyrazolo[1,5- a]pyridin-2-yl)-1,4,6,7-tetrahydro-5H- imidazo[4,5-c]pyridin-5-yl)pyrimidin-5- yl)methanol Diastereomer #2 (Rt = 2.539 min) |
| 230 | 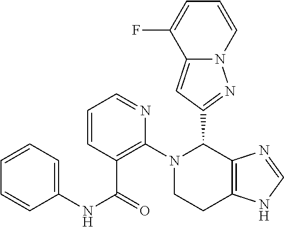 [SMILES: O=C(Nc1ccccc1)c1cccnc1N1CCc2[nH]cnc2[C@H]1c1cc2c(F)cccn2n1] | (S)-2-(4-(4-fluoropyrazolo[1,5-a]pyridin-2-yl)- 1,4,6,7-tetrahydro-5H-imidazo[4,5-c]pyridin-5- yl)-N-phenylnicotinamide |
| 231 | 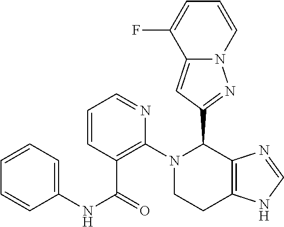 [SMILES: O=C(Nc1ccccc1)c1cccnc1N1CCc2[nH]cnc2[C@@H]1c1cc2c(F)cccn2n1] | (R)-2-(4-(4-fluoropyrazolo[1,5-a]pyridin-2-yl)- 1,4,6,7-tetrahydro-5H-imidazo[4,5-c]pyridin-5- yl)-N-phenylnicotinamidee |
| 232 | 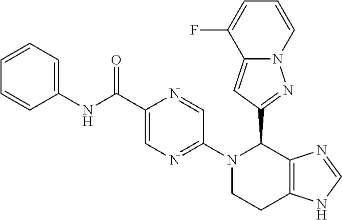 [SMILES: O=C(Nc1ccccc1)c1cnc(N2CCc3[nH]cnc3[C@@H]2c2cc3c(F)cccn3n2)cn1] | (R)-5-(4-(4-fluoropyrazolo[1,5-a]pyridin-2-yl)- 1,4,6,7-tetrahydro-5H-imidazo[4,5-c]pyridin-5- yl)-N-phenylpyrazine-2-carboxamide |
| 233 | 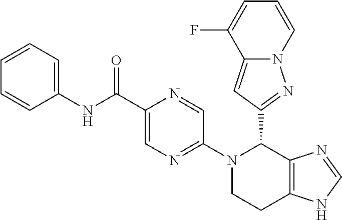 [SMILES: O=C(Nc1ccccc1)c1cnc(N2CCc3[nH]cnc3[C@H]2c2cc3c(F)cccn3n2)cn1] | (S)-5-(4-(4-fluoropyrazolo[1,5-a]pyridin-2-yl)- 1,4,6,7-tetrahydro-5H-imidazo[4,5-c]pyridin-5- yl)-N-phenylpyrazine-2-carboxamide |
| 234 | 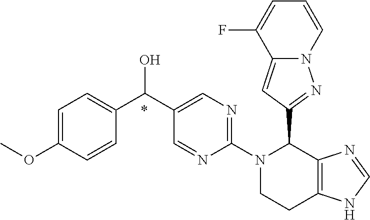 [SMILES: COc1ccc(C(O)c2cnc(N3CCc4[nH]cnc4[C@@H]3c3cc4c(F)cccn4n3)nc2)cc1] | (2-((R)-4-(4-fluoropyrazolo[1,5-a]pyridin-2-yl)- 1,4,6,7-tetrahydro-5H-imidazo[4,5-c]pyridin-5- yl)pyrimidin-5-yl)(4-methoxyphenyl)methanol Diastereomer #1 (Rt = 2.236 min) |
| 235 | 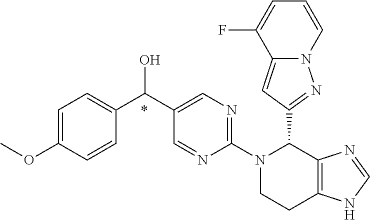 [SMILES: COc1ccc(C(O)c2cnc(N3CCc4[nH]cnc4[C@H]3c3cc4c(F)cccn4n3)nc2)cc1] | (2-((S)-4-(4-fluoropyrazolo[1,5-a]pyridin-2-yl)- 1,4,6,7-tetrahydro-5H-imidazo[4,5-c]pyridin-5- yl)pyrimidin-5-yl)(4-methoxyphenyl)methanol Diastereomer #2 (Rt = 2.858 min) |
| 236 | 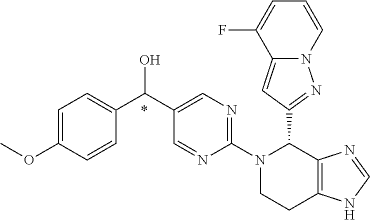 [SMILES: COc1ccc(C(O)c2cnc(N3CCc4[nH]cnc4[C@H]3c3cc4c(F)cccn4n3)nc2)cc1] | (2-((S)-4-(4-fluoropyrazolo[1,5-a]pyridin-2-yl)- 1,4,6,7-tetrahydro-5H-imidazo[4,5-c]pyridin-5- yl)pyrimidin-5-yl)(4-methoxyphenyl)methanol Diastereomer #3 (Rt = 3.784) |
| 237 | 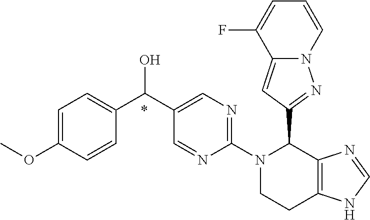 [SMILES: COc1ccc(C(O)c2cnc(N3CCc4[nH]cnc4[C@@H]3c3cc4c(F)cccn4n3)nc2)cc1] | (2-((R)-4-(4-fluoropyrazolo[1,5-a]pyridin-2-yl)- 1,4,6,7-tetrahydro-5H-imidazo[4,5-c]pyridin-5- yl)pyrimidin-5-yl)(4-methoxyphenyl)methanol Diastereomer #4 (Rt = 5.022 min) |
| 238 | 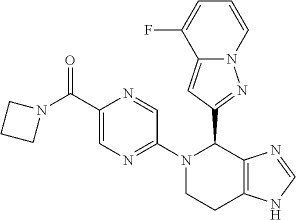 [SMILES: O=C(c1cnc(N2CCc3[nH]cnc3[C@@H]2c2cc3c(F)cccn3n2)cn1)N1CCC1] | (R)-azetidin-1-yl(5-(4-(4-fluoropyrazolo[1,5- a]pyridin-2-yl)-1,4,6,7-tetrahydro-5H- imidazo[4,5-c]pyridin-5-yl)pyrazin-2- yl)methanone |
| 239 | 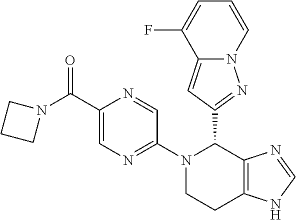 [SMILES: O=C(c1cnc(N2CCc3[nH]cnc3[C@H]2c2cc3c(F)cccn3n2)cn1)N1CCC1] | (S)-azetidin-1-yl(5-(4-(4-fluoropyrazolo[1,5- a]pyridin-2-yl)-1,4,6,7-tetrahydro-5H- imidazo[4,5-c]pyridin-5-yl)pyrazin-2- yl)methanone |
| 240 | 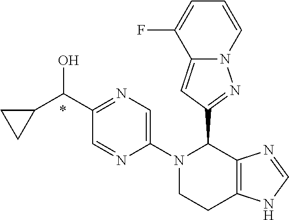 [SMILES: OC(c1cnc(N2CCc3[nH]cnc3[C@@H]2c2cc3c(F)cccn3n2)cn1)C1CC1] | cyclopropyl(5-((4R)-4-(4-fluoropyrazolo[1,5- a]pyridin-2-yl)-1,4,6,7-tetrahydro-5H- imidazo[4,5-c]pyridin-5-yl)pyrazin-2- yl)methanol Diastereomer #1 |
| 241 | 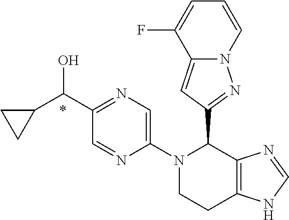 [SMILES: OC(c1cnc(N2CCc3[nH]cnc3[C@@H]2c2cc3c(F)cccn3n2)cn1)C1CC1] | cyclopropyl(5-((4R)-4-(4-fluoropyrazolo[1,5- a]pyridin-2-yl)-1,4,6,7-tetrahydro-5H- imidazo[4,5-c]pyridin-5-yl)pyrazin-2- yl)methanol Diastereomer #2 |
| 242 | 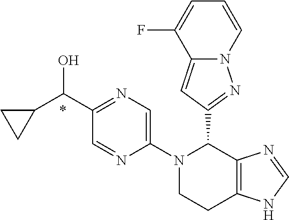 [SMILES: OC(c1cnc(N2CCc3[nH]cnc3[C@H]2c2cc3c(F)cccn3n2)cn1)C1CC1] | cyclopropyl(5-((4S)-4-(4-fluoropyrazolo[1,5- a]pyridin-2-yl)-1,4,6,7-tetrahydro-5H- imidazo[4,5-c]pyridin-5-yl)pyrazin-2- yl)methanol Diastereomer #3 |
| 243 | 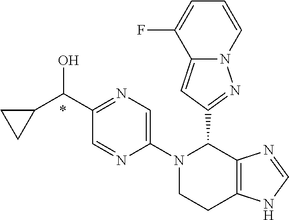 [SMILES: OC(c1cnc(N2CCc3[nH]cnc3[C@H]2c2cc3c(F)cccn3n2)cn1)C1CC1] | cyclopropyl(5-((4S)-4-(4-fluoropyrazolo[1,5- a]pyridin-2-yl)-1,4,6,7-tetrahydro-5H- imidazo[4,5-c]pyridin-5-yl)pyrazin-2- yl)methanol Diastereomer #4 |
| 244 | 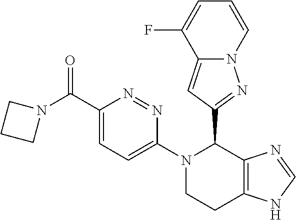 [SMILES: O=C(c1ccc(N2CCc3[nH]cnc3[C@@H]2c2cc3c(F)cccn3n2)nn1)N1CCC1] | (R)-azetidin-1-yl(6-(4-(4-fluoropyrazolo[1,5- a]pyridin-2-yl)-1,4,6,7-tetrahydro-5H- imidazo[4,5-c]pyridin-5-yl)pyridazin-3- yl)methanone |
| 245 | 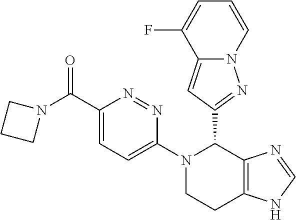 [SMILES: O=C(c1ccc(N2CCc3[nH]cnc3[C@H]2c2cc3c(F)cccn3n2)nn1)N1CCC1] | (S)-azetidin-1-yl(6-(4-(4-fluoropyrazolo[1,5- a]pyridin-2-yl)-1,4,6,7-tetrahydro-5H- imidazo[4,5-c]pyridin-5-yl)pyridazin-3- yl)methanone |
| 246 | 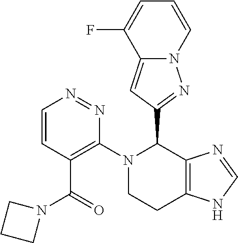 [SMILES: O=C(c1ccnnc1N1CCc2[nH]cnc2[C@@H]1c1cc2c(F)cccn2n1)N1CCC1] | (R)-azetidin-1-yl(3-(4-(4-fluoropyrazolo[1,5- a]pyridin-2-yl)-1,4,6,7-tetrahydro-5H- imidazo[4,5-c]pyridin-5-yl)pyridazin-4- yl)methanone |
| 247 | 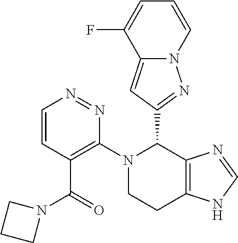 [SMILES: O=C(c1ccnnc1N1CCc2[nH]cnc2[C@H]1c1cc2c(F)cccn2n1)N1CCC1] | (S)-azetidin-1-yl(3-(4-(4-fluoropyrazolo[1,5- a]pyridin-2-yl)-1,4,6,7-tetrahydro-5H- imidazo[4,5-c]pyridin-5-yl)pyridazin-4- yl)methanone |
| 248 | 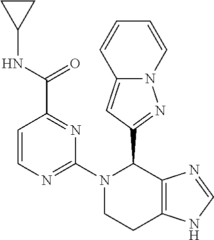 [SMILES: O=C(NC1CC1)c1ccnc(N2CCc3[nH]cnc3[C@@H]2c2cc3ccccn3n2)n1] | (R)-N-cyclopropyl-2-(4-(pyrazolo[1,5- a]pyridin-2-yl)-1,4,6,7-tetrahydro-5H- imidazo[4,5-c]pyridin-5-yl)pyrimidine-4- carboxamide |
| 249 | 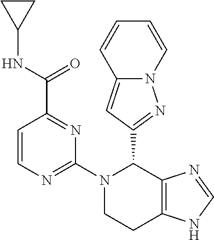 [SMILES: O=C(NC1CC1)c1ccnc(N2CCc3[nH]cnc3[C@H]2c2cc3ccccn3n2)n1] | (S)-N-cyclopropyl-2-(4-(pyrazolo[1,5- a]pyridin-2-yl)-1,4,6,7-tetrahydro-5H- imidazo[4,5-c]pyridin-5-yl)pyrimidine-4- carboxamide |
| 250 | 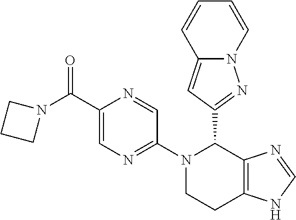 [SMILES: O=C(c1cnc(N2CCc3[nH]cnc3[C@H]2c2cc3ccccn3n2)cn1)N1CCC1] | (S)-azetidin-1-yl(5-(4-(pyrazolo[1,5-a]pyridin- 2-yl)-1,4,6,7-tetrahydro-5H-imidazo[4,5- c]pyridin-5-yl)pyrazin-2-yl)methanone |
| 251 | 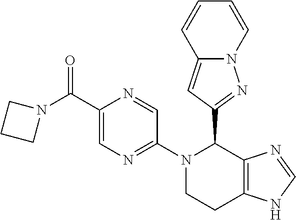 [SMILES: O=C(c1cnc(N2CCc3[nH]cnc3[C@@H]2c2cc3ccccn3n2)cn1)N1CCC1] | (R)-azetidin-1-yl(5-(4-(pyrazolo[1,5-a]pyridin- 2-yl)-1,4,6,7-tetrahydro-5H-imidazo[4,5- c]pyridin-5-yl)pyrazin-2-yl)methanone |
| 252 | 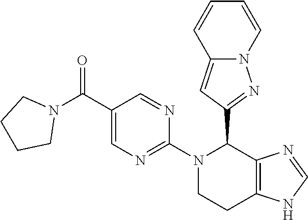 [SMILES: O=C(c1cnc(N2CCc3[nH]cnc3[C@@H]2c2cc3ccccn3n2)nc1)N1CCCC1] | (R)-(2-(4-(pyrazolo[1,5-a]pyridin-2-yl)-1,4,6,7- tetrahydro-5H-imidazo[4,5-c]pyridin-5- yl)pyrimidin-5-yl)(pyrrolidin-1-yl)methanone |
| 253 | 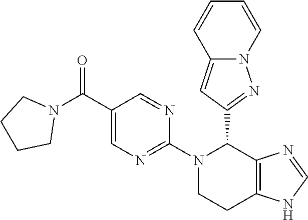 [SMILES: O=C(c1cnc(N2CCc3[nH]cnc3[C@H]2c2cc3ccccn3n2)nc1)N1CCCC1] | (S)-(2-(4-(pyrazolo[1,5-a]pyridin-2-yl)-1,4,6,7- tetrahydro-5H-imidazo[4,5-c]pyridin-5- yl)pyrimidin-5-yl)(pyrrolidin-1-yl)methanone |
| 254 | 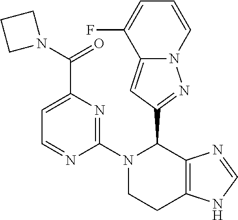 [SMILES: O=C(c1ccnc(N2CCc3[nH]cnc3[C@@H]2c2cc3c(F)cccn3n2)n1)N1CCC1] | (R)-azetidin-1-yl(2-(4-(4-fluoropyrazolo[1,5- a]pyridin-2-yl)-1,4,6,7-tetrahydro-5H- imidazo[4,5-c]pyridin-5-yl)pyrimidin-4- yl)methanone |
| 255 | 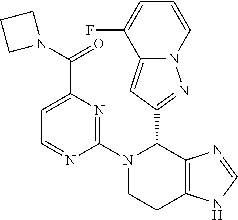 [SMILES: O=C(c1ccnc(N2CCc3[nH]cnc3[C@H]2c2cc3c(F)cccn3n2)n1)N1CCC1] | (S)-azetidin-1-yl(2-(4-(4-fluoropyrazolo[1,5- a]pyridin-2-yl)-1,4,6,7-tetrahydro-5H- imidazo[4,5-c]pyridin-5-yl)pyrimidin-4- yl)methanone |
| 256 | 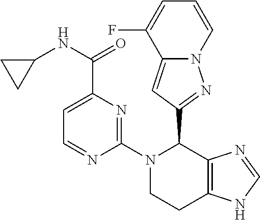 [SMILES: O=C(NC1CC1)c1ccnc(N2CCc3[nH]cnc3[C@@H]2c2cc3c(F)cccn3n2)n1] | (R)-N-cyclopropyl-2-(4-(4-fluoropyrazolo[1,5- a]pyridin-2-yl)-1,4,6,7-tetrahydro-5H- imidazo[4,5-c]pyridin-5-yl)pyrimidine-4- carboxamide |
| 257 | 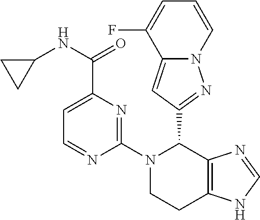 [SMILES: O=C(NC1CC1)c1ccnc(N2CCc3[nH]cnc3[C@H]2c2cc3c(F)cccn3n2)n1] | S)-N-cyclopropyl-2-(4-(4-fluoropyrazolo[1,5- a]pyridin-2-yl)-1,4,6,7-tetrahydro-5H- imidazo[4,5-c]pyridin-5-yl)pyrimidine-4- carboxamide |
| 258 | 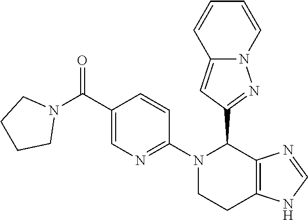 [SMILES: O=C(c1ccc(N2CCc3[nH]cnc3[C@@H]2c2cc3ccccn3n2)nc1)N1CCCC1] | (R)-(6-(4-(pyrazolo[1,5-a]pyridin-2-yl)-1,4,6,7- tetrahydro-5H-imidazo[4,5-c]pyridin-5- yl)pyridin-3-yl)(pyrrolidin-1-yl)methanone |
| 259 | 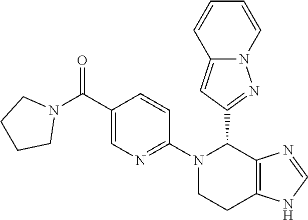 [SMILES: O=C(c1ccc(N2CCc3[nH]cnc3[C@H]2c2cc3ccccn3n2)nc1)N1CCCC1] | (S)-(6-(4-(pyrazolo[1,5-a]pyridin-2-yl)-1,4,6,7- tetrahydro-5H-imidazo[4,5-c]pyridin-5- yl)pyridin-3-yl)(pyrrolidin-1-yl)methanone |
| 260 | 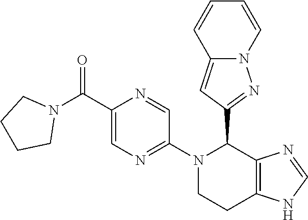 [SMILES: O=C(c1cnc(N2CCc3[nH]cnc3[C@@H]2c2cc3ccccn3n2)cn1)N1CCCC1] | (R)-(5-(4-(pyrazolo[1,5-a]pyridin-2-yl)-1,4,6,7- tetrahydro-5H-imidazo[4,5-c]pyridin-5- yl)pyrazin-2-yl)(pyrrolidin-1-yl)methanone |
| 261 | 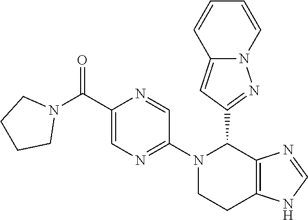 [SMILES: O=C(c1cnc(N2CCc3[nH]cnc3[C@H]2c2cc3ccccn3n2)cn1)N1CCCC1] | (S)-(5-(4-(pyrazolo[1,5-a]pyridin-2-yl)-1,4,6,7- tetrahydro-5H-imidazo[4,5-c]pyridin-5- yl)pyrazin-2-yl)(pyrrolidin-1-yl)methanone |
| 262 | 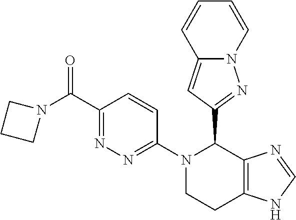 [SMILES: O=C(c1ccc(N2CCc3[nH]cnc3[C@@H]2c2cc3ccccn3n2)nn1)N1CCC1] | (R)-azetidin-1-yl(6-(4-(pyrazolo[1,5-a]pyridin- 2-yl)-1,4,6,7-tetrahydro-5H-imidazo[4,5- c]pyridin-5-yl)pyridazin-3-yl)methanone |
| 263 | 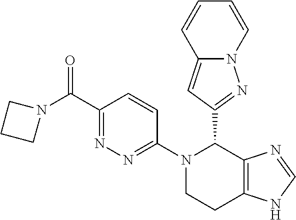 [SMILES: O=C(c1ccc(N2CCc3[nH]cnc3[C@H]2c2cc3ccccn3n2)nn1)N1CCC1] | (S)-azetidin-1-yl(6-(4-(pyrazolo[1,5-a]pyridin- 2-yl)-1,4,6,7-tetrahydro-5H-imidazo[4,5- c]pyridin-5-yl)pyridazin-3-yl)methanone |
| 264 | 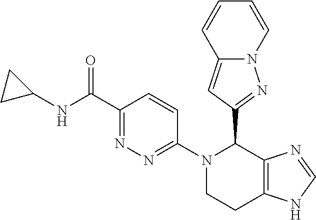 [SMILES: O=C(NC1CC1)c1ccc(N2CCc3[nH]cnc3[C@@H]2c2cc3ccccn3n2)nn1] | (R)-N-cyclopropyl-6-(4-(pyrazolo[1,5- a]pyridin-2-yl)-1,4,6,7-tetrahydro-5H- imidazo[4,5-c]pyridin-5-yl)pyridazine-3- carboxamide |
| 265 | 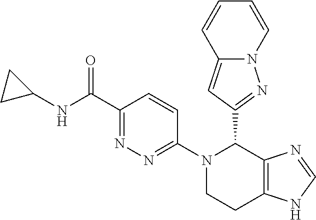 [SMILES: O=C(NC1CC1)c1ccc(N2CCc3[nH]cnc3[C@H]2c2cc3ccccn3n2)nn1] | (S)-N-cyclopropyl-6-(4-(pyrazolo[1,5- a]pyridin-2-yl)-1,4,6,7-tetrahydro-5H- imidazo[4,5-c]pyridin-5-yl)pyridazine-3- carboxamide |
| 266 | 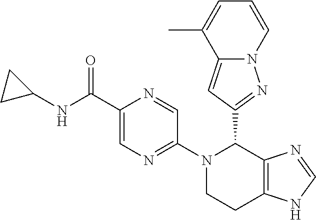 [SMILES: Cc1cccn2nc([C@@H]3c4nc[nH]c4CCN3c3cnc(C(=O)NC4CC4)cn3)cc12] | (S)-N-cyclopropyl-5-(4-(4-methylpyrazolo[1,5- a]pyridin-2-yl)-1,4,6,7-tetrahydro-5H- imidazo[4,5-c]pyridin-5-yl)pyrazine-2- carboxamide |
| 267 | 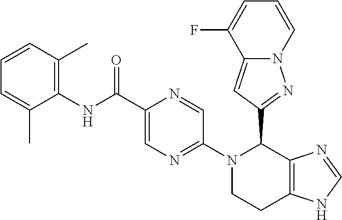 [SMILES: Cc1cccc(C)c1NC(=O)c1cnc(N2CCc3[nH]cnc3[C@@H]2c2cc3c(F)cccn3n2)cn1] | (R)-N-(2,6-dimethylphenyl)-5-(4-(4- fluoropyrazolo[1,5-a]pyridin-2-yl)-1,4,6,7- tetrahydro-5H-imidazo[4,5-c]pyridin-5- yl)pyrazine-2-carboxamide |
| 268 | 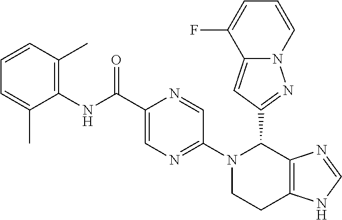 [SMILES: Cc1cccc(C)c1NC(=O)c1cnc(N2CCc3[nH]cnc3[C@H]2c2cc3c(F)cccn3n2)cn1] | (S)-N-(2,6-dimethylphenyl)-5-(4-(4- fluoropyrazolo[1,5-a]pyridin-2-yl)-1,4,6,7- tetrahydro-5H-imidazo[4,5-c]pyridin-5- yl)pyrazine-2-carboxamide |
| 269 | 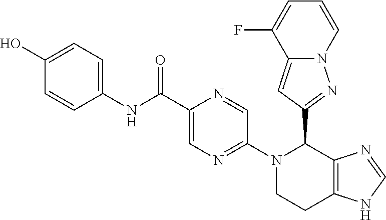 [SMILES: O=C(Nc1ccc(O)cc1)c1cnc(N2CCc3[nH]cnc3[C@@H]2c2cc3c(F)cccn3n2)cn1] | (R)-5-(4-(4-fluoropyrazolo[1,5-a]pyridin-2-yl)- 1,4,6,7-tetrahydro-5H-imidazo[4,5-c]pyridin-5-yl)- N-(4-hydroxyphenyl)pyrazine-2-carboxamide |
| 270 | 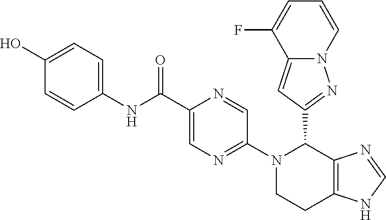 [SMILES: O=C(Nc1ccc(O)cc1)c1cnc(N2CCc3[nH]cnc3[C@H]2c2cc3c(F)cccn3n2)cn1] | (S)-5-(4-(4-fluoropyrazolo[1,5-a]pyridin-2-yl)- 1,4,6,7-tetrahydro-5H-imidazo[4,5-c]pyridin-5-yl)- N-(4-hydroxyphenyl)pyrazine-2-carboxamide |
| 271 | 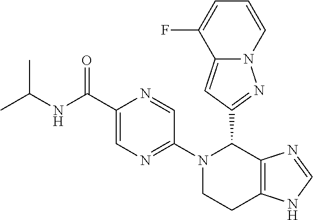 [SMILES: CC(C)NC(=O)c1cnc(N2CCc3[nH]cnc3[C@H]2c2cc3c(F)cccn3n2)cn1] | (S)-5-(4-(4-fluoropyrazolo[1,5-a]pyridin-2-yl)- 1,4,6,7-tetrahydro-5H-imidazo[4,5-c]pyridin-5- yl)-N-isopropylpyrazine-2-carboxamide |
| 272 | 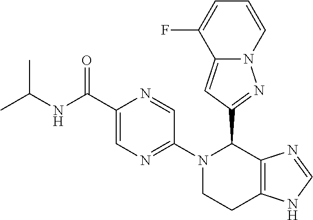 [SMILES: CC(C)NC(=O)c1cnc(N2CCc3[nH]cnc3[C@@H]2c2cc3c(F)cccn3n2)cn1] | (R)-5-(4-(4-fluoropyrazolo[1,5-a]pyridin-2-yl)- 1,4,6,7-tetrahydro-5H-imidazo[4,5-c]pyridin-5- yl)-N-isopropylpyrazine-2-carboxamide |
| 273 | 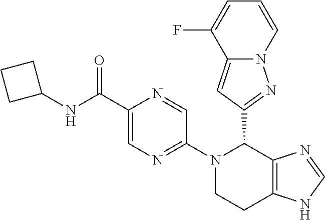 [SMILES: O=C(NC1CCC1)c1cnc(N2CCc3[nH]cnc3[C@H]2c2cc3c(F)cccn3n2)cn1] | (S)-N-cyclobutyl-5-(4-(4-fluoropyrazolo[1,5- a]pyridin-2-yl)-1,4,6,7-tetrahydro-5H- imidazo[4,5-c]pyridin-5-yl)pyrazine-2- carboxamide |
| 274 | 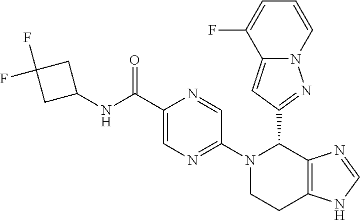 [SMILES: O=C(NC1CC(F)(F)C1)c1cnc(N2CCc3[nH]cnc3[C@H]2c2cc3c(F)cccn3n2)cn1] | (S)-N-(3,3-difluorocyclobutyl)-5-(4-(4- fluoropyrazolo[1,5-a]pyridin-2-yl)-1,4,6,7- tetrahydro-5H-imidazo[4,5-c]pyridin-5- yl)pyrazine-2-carboxamide |
| 275 | 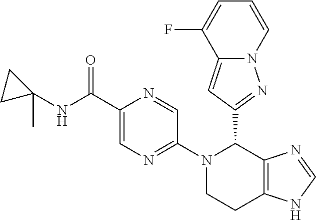 [SMILES: CC1(NC(=O)c2cnc(N3CCc4[nH]cnc4[C@H]3c3cc4c(F)cccn4n3)cn2)CC1] | (S)-5-(4-(4-fluoropyrazolo[1,5-a]pyridin-2-yl)- 1,4,6,7-tetrahydro-5H-imidazo[4,5-c]pyridin-5- yl)-N-(1-methylcyclopropyl)pyrazine-2- carboxamide |
| 276 | 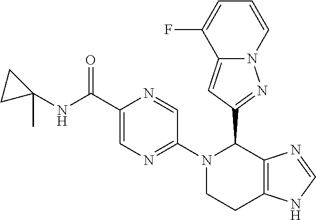 [SMILES: CC1(NC(=O)c2cnc(N3CCc4[nH]cnc4[C@@H]3c3cc4c(F)cccn4n3)cn2)CC1] | (R)-5-(4-(4-fluoropyrazolo[1,5-a]pyridin-2-yl)- 1,4,6,7-tetrahydro-5H-imidazo[4,5-c]pyridin-5- yl)-N-(1-methylcyclopropyl)pyrazine-2- carboxamide |
| 277 | 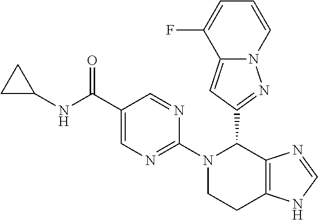 [SMILES: O=C(NC1CC1)c1cnc(N2CCc3[nH]cnc3[C@H]2c2cc3c(F)cccn3n2)nc1] | (S)-N-cyclopropyl-2-(4-(4-fluoropyrazolo[1,5- a]pyridin-2-yl)-1,4,6,7-tetrahydro-5H- imidazo[4,5-c]pyridin-5-yl)pyrimidine-5- carboxamide |
| 278 | 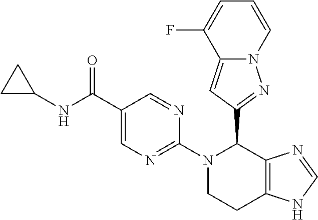 [SMILES: O=C(NC1CC1)c1cnc(N2CCc3[nH]cnc3[C@@H]2c2cc3c(F)cccn3n2)nc1] | (R)-N-cyclopropyl-2-(4-(4-fluoropyrazolo[1,5- a]pyridin-2-yl)-1,4,6,7-tetrahydro-5H- imidazo[4,5-c]pyridin-5-yl)pyrimidine-5- carboxamide |
| 279 | 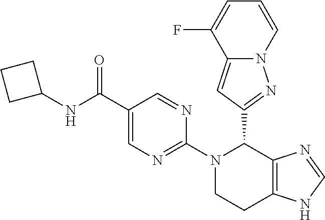 [SMILES: O=C(NC1CCC1)c1cnc(N2CCc3[nH]cnc3[C@H]2c2cc3c(F)cccn3n2)nc1] | (S)-N-cyclobutyl-2-(4-(4-fluoropyrazolo[1,5- a]pyridin-2-yl)-1,4,6,7-tetrahydro-5H- imidazo[4,5-c]pyridin-5-yl)pyrimidine-5- carboxamide |
| 280 | 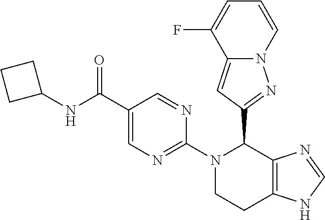 [SMILES: O=C(NC1CCC1)c1cnc(N2CCc3[nH]cnc3[C@@H]2c2cc3c(F)cccn3n2)nc1] | (R)-N-cyclobutyl-2-(4-(4-fluoropyrazolo[1,5- a]pyridin-2-yl)-1,4,6,7-tetrahydro-5H- imidazo[4,5-c]pyridin-5-yl)pyrimidine-5- carboxamide |
| 281 | 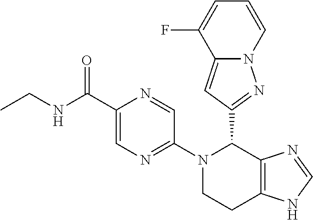 [SMILES: CCNC(=O)c1cnc(N2CCc3[nH]cnc3[C@H]2c2cc3c(F)cccn3n2)cn1] | (S)-N-ethyl-5-(4-(4-fluoropyrazolo[1,5- a]pyridin-2-yl)-1,4,6,7-tetrahydro-5H- imidazo[4,5-c]pyridin-5-yl)pyrazine-2- carboxamide |
| 282 | 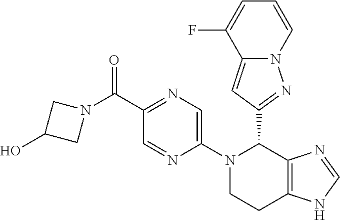 [SMILES: O=C(c1cnc(N2CCc3[nH]cnc3[C@H]2c2cc3c(F)cccn3n2)cn1)N1CC(O)C1] | (S)-(5-(4-(4-fluoropyrazolo[1,5-a]pyridin-2-yl)- 1,4,6,7-tetrahydro-5H-imidazo[4,5-c]pyridin-5- yl)pyrazin-2-yl)(3-hydroxyazetidin-1- yl)methanone |
| 283 | 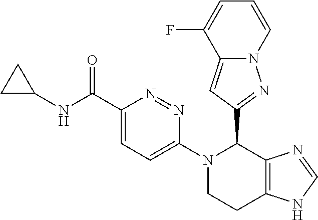 [SMILES: O=C(NC1CC1)c1ccc(N2CCc3[nH]cnc3[C@@H]2c2cc3c(F)cccn3n2)nn1] | (R)-N-cyclopropyl-6-(4-(4-fluoropyrazolo[1,5- a]pyridin-2-yl)-1,4,6,7-tetrahydro-5H- imidazo[4,5-c]pyridin-5-yl)pyridazine-3- carboxamide |
| 284 | 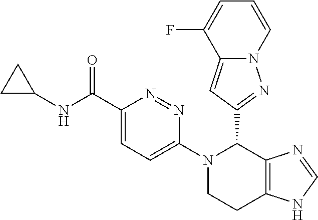 [SMILES: O=C(NC1CC1)c1ccc(N2CCc3[nH]cnc3[C@H]2c2cc3c(F)cccn3n2)nn1] | (S)-N-cyclopropyl-6-(4-(4-fluoropyrazolo[1,5- a]pyridin-2-yl)-1,4,6,7-tetrahydro-5H- imidazo[4,5-c]pyridin-5-yl)pyridazine-3- carboxamide |
| 285 | 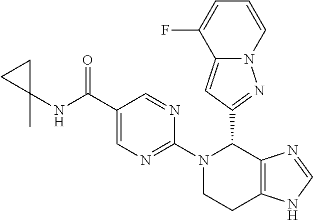 [SMILES: CC1(NC(=O)c2cnc(N3CCc4[nH]cnc4[C@H]3c3cc4c(F)cccn4n3)nc2)CC1] | (S)-2-(4-(4-fluoropyrazolo[1,5-a]pyridin-2-yl)- 1,4,6,7-tetrahydro-5H-imidazo[4,5-c]pyridin-5- yl)-N-(1-methylcyclopropyl)pyrimidine-5- carboxamide |
| 286 | 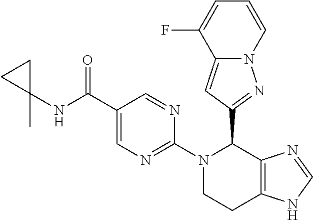 [SMILES: CC1(NC(=O)c2cnc(N3CCc4[nH]cnc4[C@@H]3c3cc4c(F)cccn4n3)nc2)CC1] | (R)-2-(4-(4-fluoropyrazolo[1,5-a]pyridin-2-yl)- 1,4,6,7-tetrahydro-5H-imidazo[4,5-c]pyridin-5- yl)-N-(1-methylcyclopropyl)pyrimidine-5- carboxamide |
| 287 | 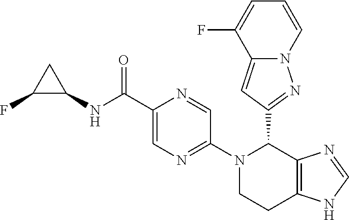 [SMILES: O=C(N[C@@H]1C[C@@H]1F)c1cnc(N2CCc3[nH]cnc3[C@H]2c2cc3c(F)cccn3n2)cn1] | N-((1R,2S)-2-fluorocyclopropyl)-5-((S)-4-(4- fluoropyrazolo[1,5-a]pyridin-2-yl)-1,4,6,7- tetrahydro-5H-imidazo[4,5-c]pyridin-5- yl)pyrazine-2-carboxamide |
| 288 | 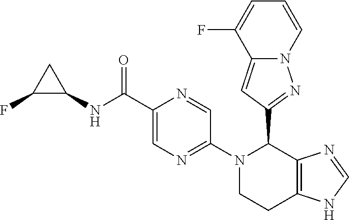 [SMILES: O=C(N[C@@H]1C[C@@H]1F)c1cnc(N2CCc3[nH]cnc3[C@@H]2c2cc3c(F)cccn3n2)cn1] | N-((1R,2S)-2-fluorocyclopropyl)-5-((R)-4-(4- fluoropyrazolo[1,5-a]pyridin-2-yl)-1,4,6,7- tetrahydro-5H-imidazo[4,5-c]pyridin-5- yl)pyrazine-2-carboxamide |
| 289 | 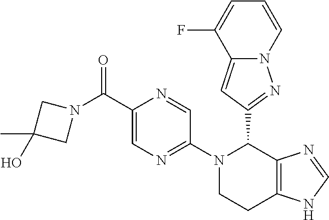 [SMILES: CC1(O)CN(C(=O)c2cnc(N3CCc4[nH]cnc4[C@H]3c3cc4c(F)cccn4n3)cn2)C1] | (S)-(5-(4-(4-fluoropyrazolo[1,5-a]pyridin-2-yl)- 1,4,6,7-tetrahydro-5H-imidazo[4,5-c]pyridin-5- yl)pyrazin-2-yl)(3-hydroxy-3-methylazetidin-1- yl)methanone |
| 290 | 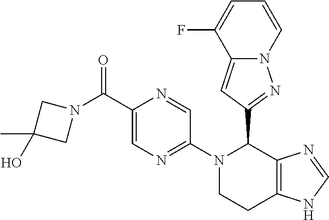 [SMILES: CC1(O)CN(C(=O)c2cnc(N3CCc4[nH]cnc4[C@@H]3c3cc4c(F)cccn4n3)cn2)C1] | (R)-(5-(4-(4-fluoropyrazolo[1,5-a]pyridin-2-yl)- 1,4,6,7-tetrahydro-5H-imidazo[4,5-c]pyridin-5- yl)pyrazin-2-yl)(3-hydroxy-3-methylazetidin-1- yl)methanone |
| 291 | 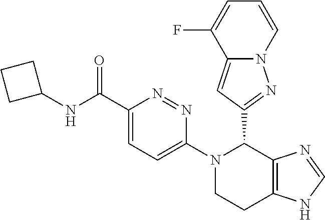 [SMILES: O=C(NC1CCC1)c1ccc(N2CCc3[nH]cnc3[C@H]2c2cc3c(F)cccn3n2)nn1] | (S)-N-cyclobutyl-6-(4-(4-fluoropyrazolo[1,5- a]pyridin-2-yl)-1,4,6,7-tetrahydro-5H- imidazo[4,5-c]pyridin-5-yl)pyridazine-3- carboxamide |
| 292 | 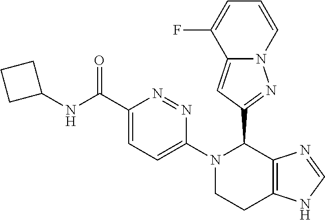 [SMILES: O=C(NC1CCC1)c1ccc(N2CCc3[nH]cnc3[C@@H]2c2cc3c(F)cccn3n2)nn1] | (R)-N-cyclobutyl-6-(4-(4-fluoropyrazolo[1,5- a]pyridin-2-yl)-1,4,6,7-tetrahydro-5H- imidazo[4,5-c]pyridin-5-yl)pyridazine-3- carboxamide |
| 293 | 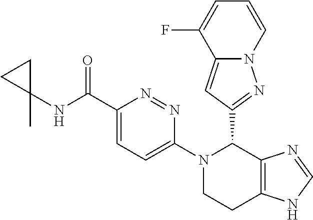 [SMILES: CC1(NC(=O)c2ccc(N3CCc4[nH]cnc4[C@H]3c3cc4c(F)cccn4n3)nn2)CC1] | (S)-6-(4-(4-fluoropyrazolo[1,5-a]pyridin-2-yl)- 1,4,6,7-tetrahydro-5H-imidazo[4,5-c]pyridin-5- yl)-N-(1-methylcyclopropyl)pyridazine-3- carboxamide |
| 294 | 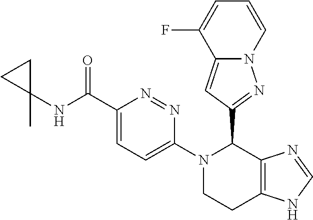 [SMILES: CC1(NC(=O)c2ccc(N3CCc4[nH]cnc4[C@@H]3c3cc4c(F)cccn4n3)nn2)CC1] | (R)-6-(4-(4-fluoropyrazolo[1,5-a]pyridin-2-yl)- 1,4,6,7-tetrahydro-5H-imidazo[4,5-c]pyridin-5- yl)-N-(1-methylcyclopropyl)pyridazine-3- carboxamide |
| 295 | 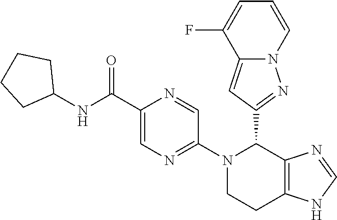 [SMILES: O=C(NC1CCCC1)c1cnc(N2CCc3[nH]cnc3[C@H]2c2cc3c(F)cccn3n2)cn1] | (S)-N-cyclopentyl-5-(4-(4-fluoropyrazolo[1,5- a]pyridin-2-yl)-1,4,6,7-tetrahydro-5H- imidazo[4,5-c]pyridin-5-yl)pyrazine-2- carboxamide |
| 296 | 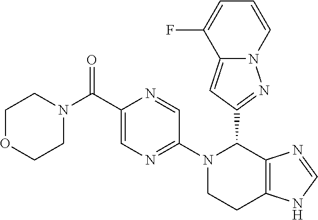 [SMILES: O=C(c1cnc(N2CCc3[nH]cnc3[C@H]2c2cc3c(F)cccn3n2)cn1)N1CCOCC1] | (S)-(5-(4-(4-fluoropyrazolo[1,5-a]pyridin-2-yl)- 1,4,6,7-tetrahydro-5H-imidazo[4,5-c]pyridin-5- yl)pyrazin-2-yl)(morpholino)methanone |
| 297 | 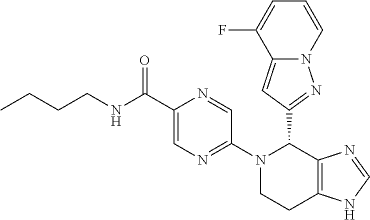 [SMILES: CCCCNC(=O)c1cnc(N2CCc3[nH]cnc3[C@H]2c2cc3c(F)cccn3n2)cn1] | (S)-N-butyl-5-(4-(4-fluoropyrazolo[1,5- a]pyridin-2-yl)-1,4,6,7-tetrahydro-5H- imidazo[4,5-c]pyridin-5-yl)pyrazine-2- carboxamide |
| 298 | 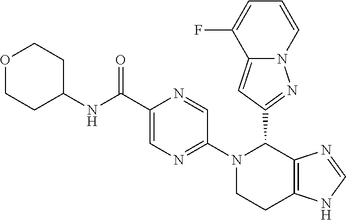 [SMILES: O=C(NC1CCOCC1)c1cnc(N2CCc3[nH]cnc3[C@H]2c2cc3c(F)cccn3n2)cn1] | (S)-5-(4-(4-fluoropyrazolo[1,5-a]pyridin-2-yl)- 1,4,6,7-tetrahydro-5H-imidazo[4,5-c]pyridin-5- yl)-N-(tetrahydro-2H-pyran-4-yl)pyrazine-2- carboxamide |
| 299 | 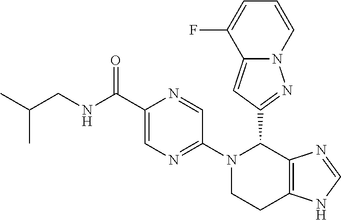 [SMILES: CC(C)CNC(=O)c1cnc(N2CCc3[nH]cnc3[C@H]2c2cc3c(F)cccn3n2)cn1] | (S)-5-(4-(4-fluoropyrazolo[1,5-a]pyridin-2-yl)- 1,4,6,7-tetrahydro-5H-imidazo[4,5-c]pyridin-5- yl)-N-isobutylpyrazine-2-carboxamide |
| 300 | 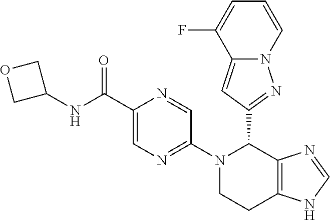 [SMILES: O=C(NC1COC1)c1cnc(N2CCc3[nH]cnc3[C@H]2c2cc3c(F)cccn3n2)cn1] | (S)-5-(4-(4-fluoropyrazolo[1,5-a]pyridin-2-yl)- 1,4,6,7-tetrahydro-5H-imidazo[4,5-c]pyridin-5- yl)-N-(oxetan-3-yl)pyrazine-2-carboxamide |
| 301 | 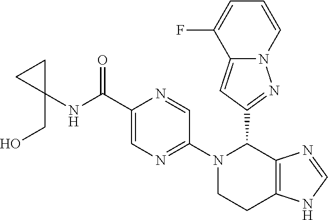 [SMILES: O=C(NC1(CO)CC1)c1cnc(N2CCc3[nH]cnc3[C@H]2c2cc3c(F)cccn3n2)cn1] | (S)-5-(4-(4-fluoropyrazolo[1,5-a]pyridin-2-yl)- 1,4,6,7-tetrahydro-5H-imidazo[4,5-c]pyridin-5- yl)-N-(1-(hydroxymethyl)cyclopropyl)pyrazine- 2-carboxamide |
| 302 | 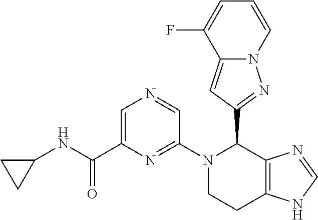 [SMILES: O=C(NC1CC1)c1cncc(N2CCc3[nH]cnc3[C@@H]2c2cc3c(F)cccn3n2)n1] | (R)-N-cyclopropyl-6-(4-(4-fluoropyrazolo[1,5- a]pyridin-2-yl)-1,4,6,7-tetrahydro-5H- imidazo[4,5-c]pyridin-5-yl)pyrazine-2- carboxamide |
| 303 | 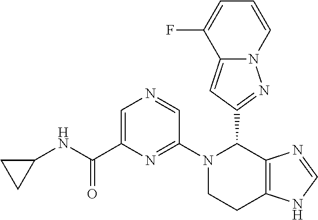 [SMILES: O=C(NC1CC1)c1cncc(N2CCc3[nH]cnc3[C@H]2c2cc3c(F)cccn3n2)n1] | (S)-N-cyclopropyl-6-(4-(4-fluoropyrazolo[1,5- a]pyridin-2-yl)-1,4,6,7-tetrahydro-5H- imidazo[4,5-c]pyridin-5-yl)pyrazine-2- carboxamide |
| 304 | 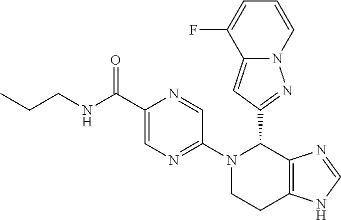 [SMILES: CCCNC(=O)c1cnc(N2CCc3[nH]cnc3[C@H]2c2cc3c(F)cccn3n2)cn1] | (S)-5-(4-(4-fluoropyrazolo[1,5-a]pyridin-2-yl)- 1,4,6,7-tetrahydro-5H-imidazo[4,5-c]pyridin-5- yl)-N-propylpyrazine-2-carboxamide |
| 305 | 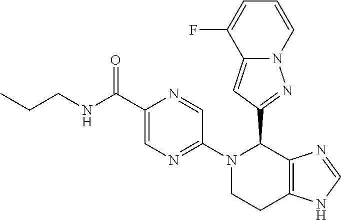 [SMILES: CCCNC(=O)c1cnc(N2CCc3[nH]cnc3[C@@H]2c2cc3c(F)cccn3n2)cn1] | (R)-5-(4-(4-fluoropyrazolo[1,5-a]pyridin-2-yl)- 1,4,6,7-tetrahydro-5H-imidazo[4,5-c]pyridin-5- yl)-N-propylpyrazine-2-carboxamide |
| 306 | 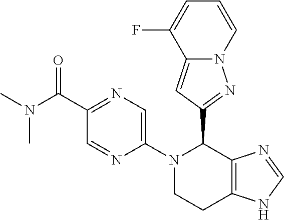 [SMILES: CN(C)C(=O)c1cnc(N2CCc3[nH]cnc3[C@@H]2c2cc3c(F)cccn3n2)cn1] | (R)-5-(4-(4-fluoropyrazolo[1,5-a]pyridin-2-yl)- 1,4,6,7-tetrahydro-5H-imidazo[4,5-c]pyridin-5- yl)-N,N-dimethylpyrazine-2-carboxamide |
| 307 | 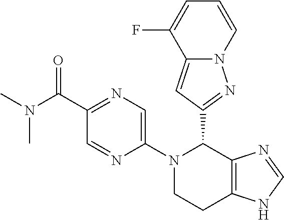 [SMILES: CN(C)C(=O)c1cnc(N2CCc3[nH]cnc3[C@H]2c2cc3c(F)cccn3n2)cn1] | (S)-5-(4-(4-fluoropyrazolo[1,5-a]pyridin-2-yl)- 1,4,6,7-tetrahydro-5H-imidazo[4,5-c]pyridin-5- yl)-N,N-dimethylpyrazine-2-carboxamide |
| 308 | 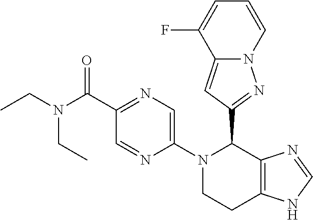 [SMILES: CCN(CC)C(=O)c1cnc(N2CCc3[nH]cnc3[C@@H]2c2cc3c(F)cccn3n2)cn1] | (R)-N,N-diethyl-5-(4-(4-fluoropyrazolo[1,5- a]pyridin-2-yl)-1,4,6,7-tetrahydro-5H- imidazo[4,5-c]pyridin-5-yl)pyrazine-2- carboxamide |
| 309 | 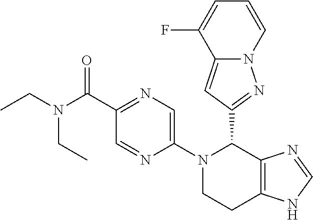 [SMILES: CCN(CC)C(=O)c1cnc(N2CCc3[nH]cnc3[C@H]2c2cc3c(F)cccn3n2)cn1] | (S)-N,N-diethyl-5-(4-(4-fluoropyrazolo[1,5- a]pyridin-2-yl)-1,4,6,7-tetrahydro-5H- imidazo[4,5-c]pyridin-5-yl)pyrazine-2- carboxamide |
| 310 | 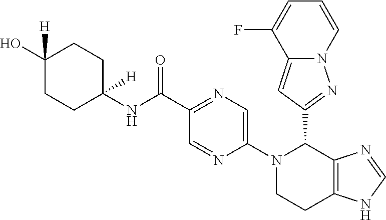 [SMILES: O=C(N[C@H]1CC[C@H](O)CC1)c1cnc(N2CCc3[nH]cnc3[C@H]2c2cc3c(F)cccn3n2)cn1] | 5-((S)-4-(4-fluoropyrazolo[1,5-a]pyridin-2-yl)- 1,4,6,7-tetrahydro-5H-imidazo[4,5-c]pyridin-5- yl)-N-((1r,4S)-4-hydroxycyclohexyl)pyrazine- 2-carboxamide |
| 311 | 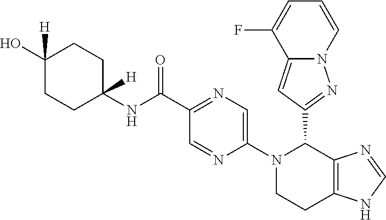 [SMILES: O=C(N[C@H]1CC[C@@H](O)CC1)c1cnc(N2CCc3[nH]cnc3[C@H]2c2cc3c(F)cccn3n2)cn1] | 5-((S)-4-(4-fluoropyrazolo[1,5-a]pyridin-2-yl)- 1,4,6,7-tetrahydro-5H-imidazo[4,5-c]pyridin-5- yl)-N-((1s,4R)-4-hydroxycyclohexyl)pyrazine- 2-carboxamide |
| 312 | 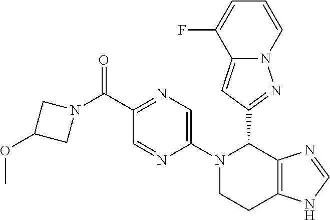 [SMILES: COC1CN(C(=O)c2cnc(N3CCc4[nH]cnc4[C@H]3c3cc4c(F)cccn4n3)cn2)C1] | (S)-(5-(4-(4-fluoropyrazolo[1,5-a]pyridin-2-yl)- 1,4,6,7-tetrahydro-5H-imidazo[4,5-c]pyridin-5- yl)pyrazin-2-yl)(3-methoxyazetidin-1- yl)methanone |
| 313 | 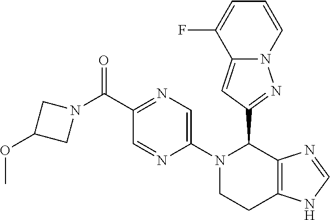 [SMILES: COC1CN(C(=O)c2cnc(N3CCc4[nH]cnc4[C@@H]3c3cc4c(F)cccn4n3)cn2)C1] | (R)-(5-(4-(4-fluoropyrazolo[1,5-a]pyridin-2-yl)- 1,4,6,7-tetrahydro-5H-imidazo[4,5-c]pyridin-5- yl)pyrazin-2-yl)(3-methoxyazetidin-1- yl)methanone |
| 314 | 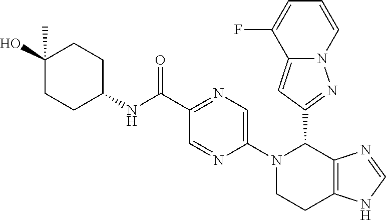 [SMILES: C[C@]1(O)CC[C@H](NC(=O)c2cnc(N3CCc4[nH]cnc4[C@H]3c3cc4c(F)cccn4n3)cn2)CC1] | 5-((S)-4-(4-fluoropyrazolo[1,5-a]pyridin-2-yl)- 1,4,6,7-tetrahydro-5H-imidazo[4,5-c]pyridin-5- yl)-N-((1r,4S)-4-hydroxy-4- methylcyclohexyl)pyrazine-2-carboxamide |
| 315 | 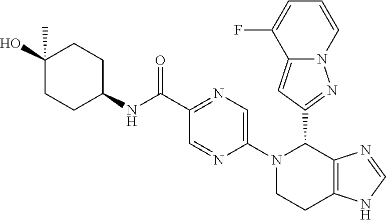 [SMILES: C[C@]1(O)CC[C@@H](NC(=O)c2cnc(N3CCc4[nH]cnc4[C@H]3c3cc4c(F)cccn4n3)cn2)CC1] | 5-((S)-4-(4-fluoropyrazolo[1,5-a]pyridin-2-yl)- 1,4,6,7-tetrahydro-5H-imidazo[4,5-c]pyridin-5- yl)-N-((1s,4R)-4-hydroxy-4- methylcyclohexyl)pyrazine-2-carboxamide |
| 316 | 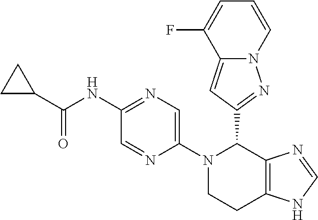 [SMILES: O=C(Nc1cnc(N2CCc3[nH]cnc3[C@H]2c2cc3c(F)cccn3n2)cn1)C1CC1] | (S)-N-(5-(4-(4-fluoropyrazolo[1,5-a]pyridin-2- yl)-1,4,6,7-tetrahydro-5H-imidazo[4,5- c]pyridin-5-yl)pyrazin-2- yl)cyclopropanecarboxamide |
| 317 | 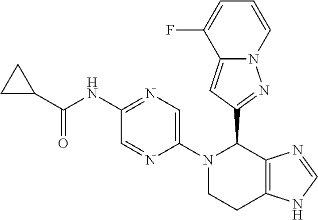 [SMILES: O=C(Nc1cnc(N2CCc3[nH]cnc3[C@@H]2c2cc3c(F)cccn3n2)cn1)C1CC1] | (R)-N-(5-(4-(4-fluoropyrazolo[1,5-a]pyridin-2- yl)-1,4,6,7-tetrahydro-5H-imidazo[4,5- c]pyridin-5-yl)pyrazin-2- yl)cyclopropanecarboxamide |
| 318 | 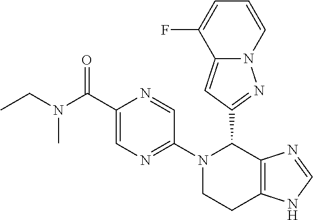 [SMILES: CCN(C)C(=O)c1cnc(N2CCc3[nH]cnc3[C@H]2c2cc3c(F)cccn3n2)cn1] | (S)-N-ethyl-5-(4-(4-fluoropyrazolo[1,5- a]pyridin-2-yl)-1,4,6,7-tetrahydro-5H- imidazo[4,5-c]pyridin-5-yl)-N-methylpyrazine- 2-carboxamide |
| 319 | 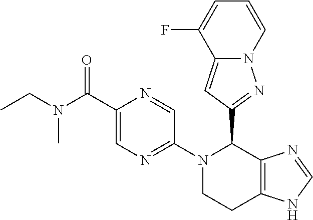 [SMILES: CCN(C)C(=O)c1cnc(N2CCc3[nH]cnc3[C@@H]2c2cc3c(F)cccn3n2)cn1] | (R)-N-ethyl-5-(4-(4-fluoropyrazolo[1,5- a]pyridin-2-yl)-1,4,6,7-tetrahydro-5H- imidazo[4,5-c]pyridin-5-yl)-N-methylpyrazine- 2-carboxamide |
| 320 | 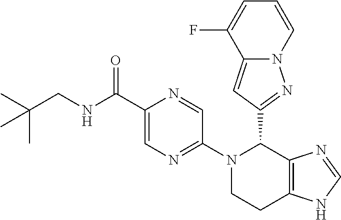 [SMILES: CC(C)(C)CNC(=O)c1cnc(N2CCc3[nH]cnc3[C@H]2c2cc3c(F)cccn3n2)cn1] | (S)-5-(4-(4-fluoropyrazolo[1,5-a]pyridin-2-yl)- 1,4,6,7-tetrahydro-5H-imidazo[4,5-c]pyridin-5- yl)-N-neopentylpyrazine-2-carboxamide |
| 321 | 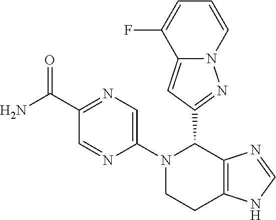 [SMILES: NC(=O)c1cnc(N2CCc3[nH]cnc3[C@H]2c2cc3c(F)cccn3n2)cn1] | (S)-5-(4-(4-fluoropyrazolo[1,5-a]pyridin-2-yl)- 1,4,6,7-tetrahydro-5H-imidazo[4,5-c]pyridin-5- yl)pyrazine-2-carboxamide |
| 322 | 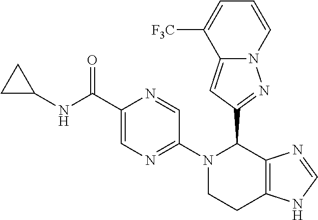 [SMILES: O=C(NC1CC1)c1cnc(N2CCc3[nH]cnc3[C@@H]2c2cc3c(C(F)(F)F)cccn3n2)cn1] | (R)-N-cyclopropyl-5-(4-(4- (trifluoromethyl)pyrazolo[1,5-a]pyridin-2-yl)- 1,4,6,7-tetrahydro-5H-imidazo[4,5-c]pyridin-5- yl)pyrazine-2-carboxamide |
| 323 | 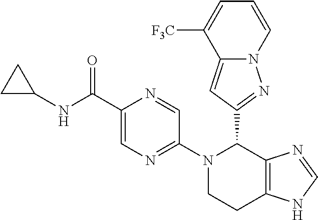 [SMILES: O=C(NC1CC1)c1cnc(N2CCc3[nH]cnc3[C@H]2c2cc3c(C(F)(F)F)cccn3n2)cn1] | (S)-N-cyclopropyl-5-(4-(4- (trifluoromethyl)pyrazolo[1,5-a]pyridin-2-yl)- 1,4,6,7-tetrahydro-5H-imidazo[4,5-c]pyridin-5- yl)pyrazine-2-carboxamide |
| 324 | 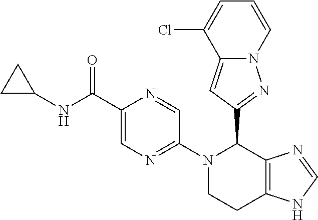 [SMILES: O=C(NC1CC1)c1cnc(N2CCc3[nH]cnc3[C@@H]2c2cc3c(Cl)cccn3n2)cn1] | (R)-5-(4-(4-chloropyrazolo[1,5-a]pyridin-2-yl)- 1,4,6,7-tetrahydro-5H-imidazo[4,5-c]pyridin-5- yl)-N-cyclopropylpyrazine-2-carboxamide |
| 325 | 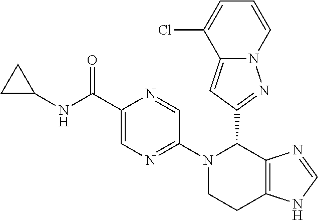 [SMILES: O=C(NC1CC1)c1cnc(N2CCc3[nH]cnc3[C@H]2c2cc3c(Cl)cccn3n2)cn1] | (S)-5-(4-(4-chloropyrazolo[1,5-a]pyridin-2-yl)- 1,4,6,7-tetrahydro-5H-imidazo[4,5-c]pyridin-5- yl)-N-cyclopropylpyrazine-2-carboxamide |
| 326 | 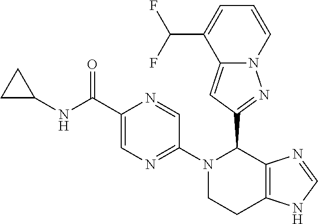 [SMILES: O=C(NC1CC1)c1cnc(N2CCc3[nH]cnc3[C@@H]2c2cc3c(C(F)F)cccn3n2)cn1] | (R)-N-cyclopropyl-5-(4-(4- (difluoromethyl)pyrazolo[1,5-a]pyridin-2-yl)- 1,4,6,7-tetrahydro-5H-imidazo[4,5-c]pyridin-5- yl)pyrazine-2-carboxamide |
| 327 | 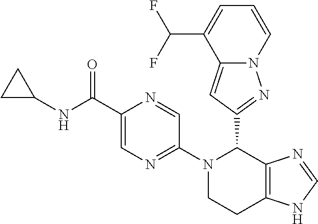 [SMILES: O=C(NC1CC1)c1cnc(N2CCc3[nH]cnc3[C@H]2c2cc3c(C(F)F)cccn3n2)cn1] | (S)-N-cyclopropyl-5-(4-(4- (difluoromethyl)pyrazolo[1,5-a]pyridin-2-yl)- 1,4,6,7-tetrahydro-5H-imidazo[4,5-c]pyridin-5- yl)pyrazine-2-carboxamide |
| 328 | 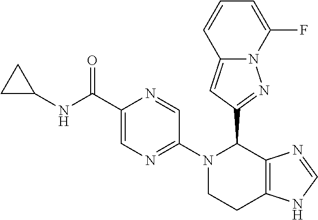 [SMILES: O=C(NC1CC1)c1cnc(N2CCc3[nH]cnc3[C@@H]2c2cc3cccc(F)n3n2)cn1] | (R)-N-cyclopropyl-5-(4-(7-fluoropyrazolo[1,5- a]pyridin-2-yl)-1,4,6,7-tetrahydro-5H- imidazo[4,5-c]pyridin-5-yl)pyrazine-2- carboxamide |
| 329 | 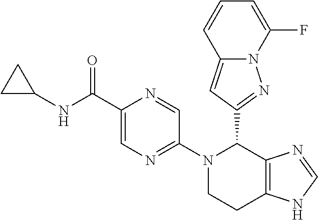 [SMILES: O=C(NC1CC1)c1cnc(N2CCc3[nH]cnc3[C@H]2c2cc3cccc(F)n3n2)cn1] | (S)-N-cyclopropyl-5-(4-(7-fluoropyrazolo[1,5- a]pyridin-2-yl)-1,4,6,7-tetrahydro-5H- imidazo[4,5-c]pyridin-5-yl)pyrazine-2- carboxamide |
| 330 | 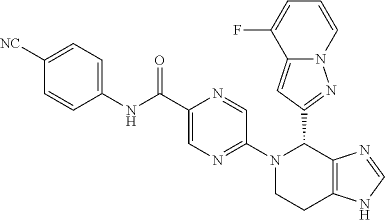 [SMILES: N#Cc1ccc(NC(=O)c2cnc(N3CCc4[nH]cnc4[C@H]3c3cc4c(F)cccn4n3)cn2)cc1] | (S)-N-(4-cyanophenyl)-5-(4-(4- fluoropyrazolo[1,5-a]pyridin-2-yl)-1,4,6,7- tetrahydro-5H-imidazo[4,5-c]pyridin-5- yl)pyrazine-2-carboxamide |
| 331 | 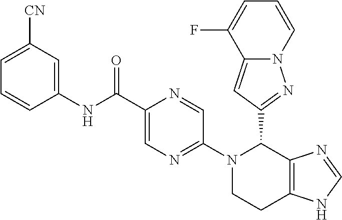 [SMILES: N#Cc1cccc(NC(=O)c2cnc(N3CCc4[nH]cnc4[C@H]3c3cc4c(F)cccn4n3)cn2)c1] | (S)-N-(3-cyanophenyl)-5-(4-(4- fluoropyrazolo[1,5-a]pyridin-2-yl)-1,4,6,7- tetrahydro-5H-imidazo[4,5-c]pyridin-5- yl)pyrazine-2-carboxamide |
| 332 | 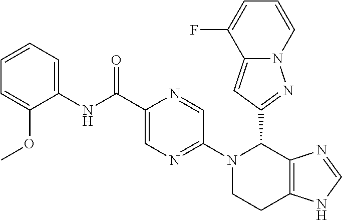 [SMILES: COc1ccccc1NC(=O)c1cnc(N2CCc3[nH]cnc3[C@H]2c2cc3c(F)cccn3n2)cn1] | (S)-5-(4-(4-fluoropyrazolo[1,5-a]pyridin-2-yl)- 1,4,6,7-tetrahydro-5H-imidazo[4,5-c]pyridin-5- yl)-N-(2-methoxyphenyl)pyrazine-2- carboxamide |
| 333 | 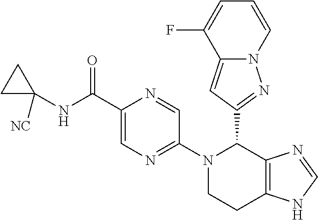 [SMILES: N#CC1(NC(=O)c2cnc(N3CCc4[nH]cnc4[C@H]3c3cc4c(F)cccn4n3)cn2)CC1] | (S)-N-(1-cyanocyclopropyl)-5-(4-(4- fluoropyrazolo[1,5-a]pyridin-2-yl)-1,4,6,7- tetrahydro-5H-imidazo[4,5-c]pyridin-5- yl)pyrazine-2-carboxamide |
| 334 | 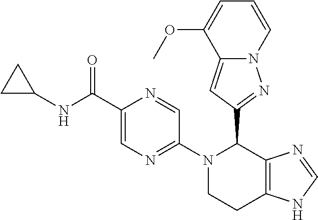 [SMILES: COc1cccn2nc([C@H]3c4nc[nH]c4CCN3c3cnc(C(=O)NC4CC4)cn3)cc12] | (R)-N-cyclopropyl-5-(4-(4- methoxypyrazolo[1,5-a]pyridin-2-yl)-1,4,6,7- tetrahydro-5H-imidazo[4,5-c]pyridin-5- yl)pyrazine-2-carboxamide |
| 335 | 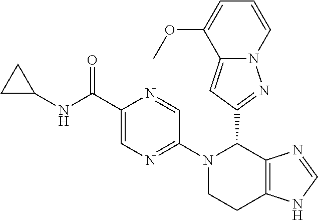 [SMILES: COc1cccn2nc([C@@H]3c4nc[nH]c4CCN3c3cnc(C(=O)NC4CC4)cn3)cc12] | (S)-N-cyclopropyl-5-(4-(4- methoxypyrazolo[1,5-a]pyridin-2-yl)-1,4,6,7- tetrahydro-5H-imidazo[4,5-c]pyridin-5- yl)pyrazine-2-carboxamide |
| 336 | 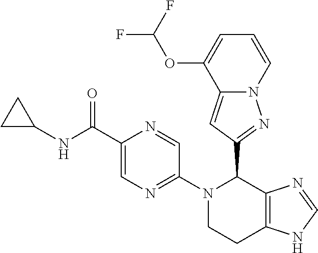 [SMILES: O=C(NC1CC1)c1cnc(N2CCc3[nH]cnc3[C@@H]2c2cc3c(OC(F)F)cccn3n2)cn1] | (R)-N-cyclopropyl-5-(4-(4- (difluoromethoxy)pyrazolo[1,5-a]pyridin-2-yl)- 1,4,6,7-tetrahydro-5H-imidazo[4,5-c]pyridin-5- yl)pyrazine-2-carboxamide |
| 337 | 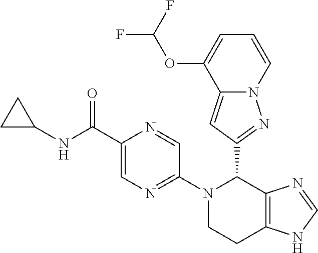 [SMILES: O=C(NC1CC1)c1cnc(N2CCc3[nH]cnc3[C@H]2c2cc3c(OC(F)F)cccn3n2)cn1] | (S)-N-cyclopropyl-5-(4-(4- (difluoromethoxy)pyrazolo[1,5-a]pyridin-2-yl)- 1,4,6,7-tetrahydro-5H-imidazo[4,5-c]pyridin-5- yl)pyrazine-2-carboxamide |
| 338 | 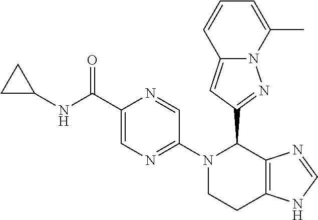 [SMILES: Cc1cccc2cc([C@H]3c4nc[nH]c4CCN3c3cnc(C(=O)NC4CC4)cn3)nn12] | (R)-N-cyclopropyl-5-(4-(7-methylpyrazolo[1,5- a]pyridin-2-yl)-1,4,6,7-tetrahydro-5H- imidazo[4,5-c]pyridin-5-yl)pyrazine-2- carboxamide |
| 339 | 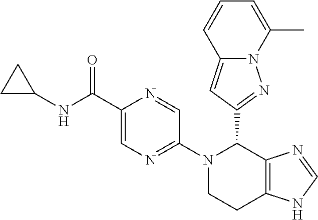 [SMILES: Cc1cccc2cc([C@@H]3c4nc[nH]c4CCN3c3cnc(C(=O)NC4CC4)cn3)nn12] | (S)-N-cyclopropyl-5-(4-(7-methylpyrazolo[1,5- a]pyridin-2-yl)-1,4,6,7-tetrahydro-5H- imidazo[4,5-c]pyridin-5-yl)pyrazine-2- carboxamide |
| 340 | 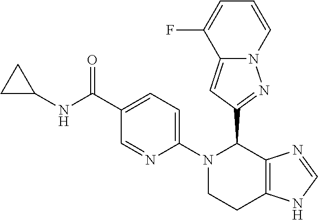 [SMILES: O=C(NC1CC1)c1ccc(N2CCc3[nH]cnc3[C@@H]2c2cc3c(F)cccn3n2)nc1] | (R)-N-cyclopropyl-6-(4-(4-fluoropyrazolo[1,5- a]pyridin-2-yl)-1,4,6,7-tetrahydro-5H- imidazo[4,5-c]pyridin-5-yl)nicotinamide |
| 341 | 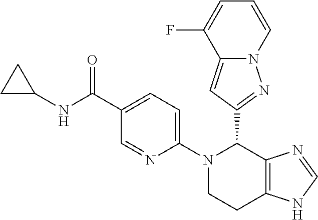 [SMILES: O=C(NC1CC1)c1ccc(N2CCc3[nH]cnc3[C@H]2c2cc3c(F)cccn3n2)nc1] | (S)-N-cyclopropyl-6-(4-(4-fluoropyrazolo[1,5- a]pyridin-2-yl)-1,4,6,7-tetrahydro-5H- imidazo[4,5-c]pyridin-5-yl)nicotinamide |
| 342 | 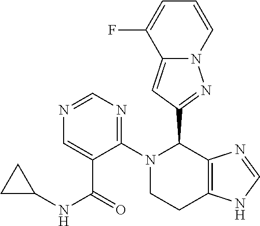 [SMILES: O=C(NC1CC1)c1cncnc1N1CCc2[nH]cnc2[C@@H]1c1cc2c(F)cccn2n1] | (R)-N-cyclopropyl-4-(4-(4-fluoropyrazolo[1,5- a]pyridin-2-yl)-1,4,6,7-tetrahydro-5H- imidazo[4,5-c]pyridin-5-yl)pyrimidine-5- carboxamide |
| 343 | 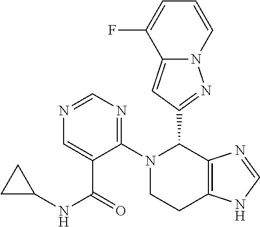 [SMILES: O=C(NC1CC1)c1cncnc1N1CCc2[nH]cnc2[C@H]1c1cc2c(F)cccn2n1] | (S)-N-cyclopropyl-4-(4-(4-fluoropyrazolo[1,5- a]pyridin-2-yl)-1,4,6,7-tetrahydro-5H- imidazo[4,5-c]pyridin-5-yl)pyrimidine-5- carboxamide |
| 344 | 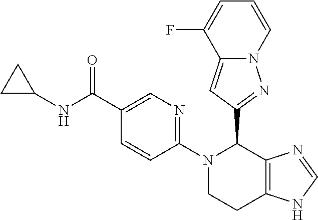 [SMILES: O=C(NC1CC1)c1ccc(N2CCc3[nH]cnc3[C@@H]2c2cc3c(F)cccn3n2)nc1] | (R)-N-cyclopropyl-5-(4-(4-fluoropyrazolo[1,5- a]pyridin-2-yl)-1,4,6,7-tetrahydro-5H- imidazo[4,5-c]pyridin-5-yl)picolinamide |
| 345 | 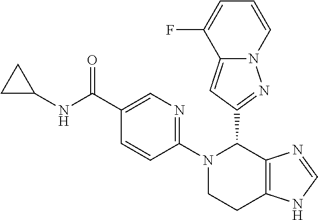 [SMILES: O=C(NC1CC1)c1ccc(N2CCc3[nH]cnc3[C@H]2c2cc3c(F)cccn3n2)nc1] | (S)-N-cyclopropyl-5-(4-(4-fluoropyrazolo[1,5- a]pyridin-2-yl)-1,4,6,7-tetrahydro-5H- imidazo[4,5-c]pyridin-5-yl)picolinamide |
| 346 | 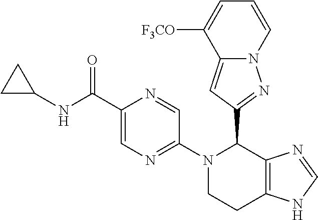 [SMILES: O=C(NC1CC1)c1cnc(N2CCc3[nH]cnc3[C@@H]2c2cc3c(OC(F)(F)F)cccn3n2)cn1] | (R)-N-cyclopropyl-5-(4-(4- (trifluoromethoxy)pyrazolo[1,5-a]pyridin-2-yl)- 1,4,6,7-tetrahydro-5H-imidazo[4,5-c]pyridin-5- yl)pyrazine-2-carboxamide |
| 347 | 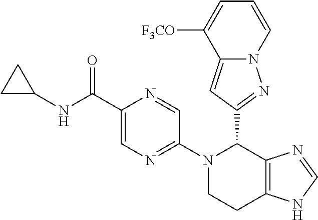 [SMILES: O=C(NC1CC1)c1cnc(N2CCc3[nH]cnc3[C@H]2c2cc3c(OC(F)(F)F)cccn3n2)cn1] | (S)-N-cyclopropyl-5-(4-(4- (trifluoromethoxy)pyrazolo[1,5-a]pyridin-2-yl)- 1,4,6,7-tetrahydro-5H-imidazo[4,5-c]pyridin-5- yl)pyrazine-2-carboxamide |
| 348 | 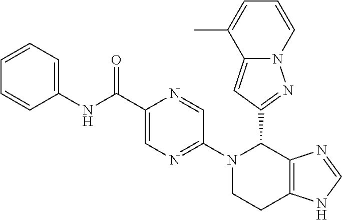 [SMILES: Cc1cccn2nc([C@@H]3c4nc[nH]c4CCN3c3cnc(C(=O)Nc4ccccc4)cn3)cc12] | (S)-5-(4-(4-methylpyrazolo[1,5-a]pyridin-2-yl)- 1,4,6,7-tetrahydro-5H-imidazo[4,5-c]pyridin-5- yl)-N-phenylpyrazine-2-carboxamide |
| 349 | 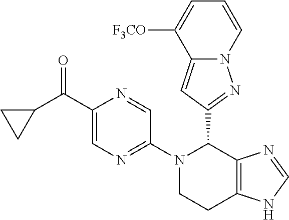 [SMILES: O=C(c1cnc(N2CCc3[nH]cnc3[C@H]2c2cc3c(OC(F)(F)F)cccn3n2)cn1)C1CC1] | (S)-cyclopropyl(5-(4-(4- (trifluoromethyl)pyrazolo[1,5-a]pyridin-2-yl)- 1,4,6,7-tetrahydro-5H-imidazo[4,5-c]pyridin-5- yl)pyrazin-2-yl)methanone |
| 350 | 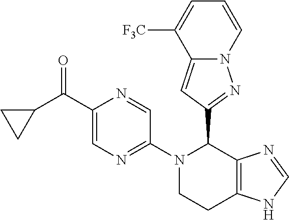 [SMILES: O=C(c1cnc(N2CCc3[nH]cnc3[C@@H]2c2cc3c(C(F)(F)F)cccn3n2)cn1)C1CC1] | (R)-cyclopropyl(5-(4-(4- (trifluoromethyl)pyrazolo[1,5-a]pyridin-2-yl)- 1,4,6,7-tetrahydro-5H-imidazo[4,5-c]pyridin-5- yl)pyrazin-2-yl)methanone |
| 351 | 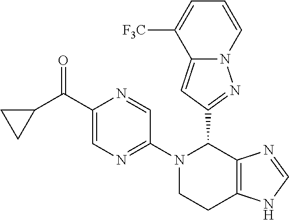 [SMILES: O=C(c1cnc(N2CCc3[nH]cnc3[C@H]2c2cc3c(C(F)(F)F)cccn3n2)cn1)C1CC1] | (S)-cyclopropyl(5-(4-(4- (trifluoromethoxy)pyrazolo[1,5-a]pyridin-2-yl)- 1,4,6,7-tetrahydro-5H-imidazo[4,5-c]pyridin-5- yl)pyrazin-2-yl)methanone |
| 352 | 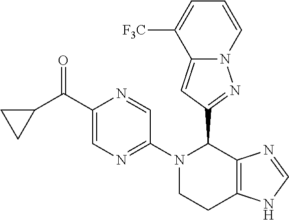 [SMILES: O=C(c1cnc(N2CCc3[nH]cnc3[C@@H]2c2cc3c(C(F)(F)F)cccn3n2)cn1)C1CC1] | (R)-cyclopropyl(5-(4-(4- (trifluoromethoxy)pyrazolo[1,5-a]pyridin-2-yl)- 1,4,6,7-tetrahydro-5H-imidazo[4,5-c]pyridin-5- yl)pyrazin-2-yl)methanone |
| 353 | 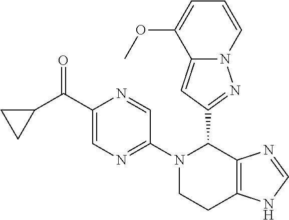 [SMILES: COc1cccn2nc([C@@H]3c4nc[nH]c4CCN3c3cnc(C(=O)C4CC4)cn3)cc12] | (S)-cyclopropyl(5-(4-(4-methoxypyrazolo[1,5- a]pyridin-2-yl)-1,4,6,7-tetrahydro-5H- imidazo[4,5-c]pyridin-5-yl)pyrazin-2- yl)methanone |
| 354 | 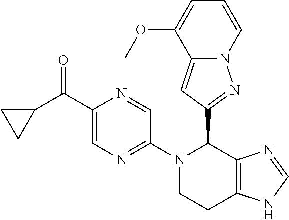 [SMILES: COc1cccn2nc([C@H]3c4nc[nH]c4CCN3c3cnc(C(=O)C4CC4)cn3)cc12] | (R)-cyclopropyl(5-(4-(4-methoxypyrazolo[1,5- a]pyridin-2-yl)-1,4,6,7-tetrahydro-5H- imidazo[4,5-c]pyridin-5-yl)pyrazin-2- yl)methanone |
| 355 | 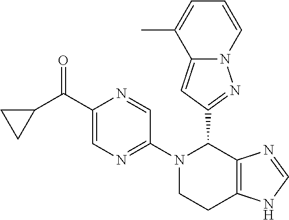 [SMILES: Cc1cccn2nc([C@@H]3c4nc[nH]c4CCN3c3cnc(C(=O)C4CC4)cn3)cc12] | (S)-cyclopropyl(5-(4-(4-methylpyrazolo[1,5- a]pyridin-2-yl)-1,4,6,7-tetrahydro-5H- imidazo[4,5-c]pyridin-5-yl)pyrazin-2- yl)methanone |
| 356 | 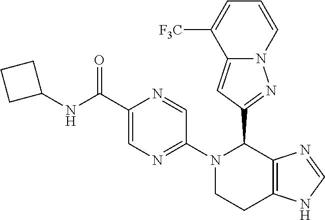 [SMILES: O=C(NC1CCC1)c1cnc(N2CCc3[nH]cnc3[C@@H]2c2cc3c(C(F)(F)F)cccn3n2)cn1] | (R)-N-cyclobutyl-5-(4-(4- (trifluoromethyl)pyrazolo[1,5-a]pyridin-2-yl)- 1,4,6,7-tetrahydro-5H-imidazo[4,5-c]pyridin-5- yl)pyrazine-2-carboxamide |
| 357 | 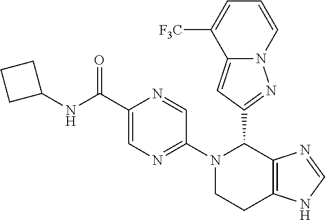 [SMILES: O=C(NC1CCC1)c1cnc(N2CCc3[nH]cnc3[C@H]2c2cc3c(C(F)(F)F)cccn3n2)cn1] | (S)-N-cyclobutyl-5-(4-(4- (trifluoromethyl)pyrazolo[1,5-a]pyridin-2-yl)- 1,4,6,7-tetrahydro-5H-imidazo[4,5-c]pyridin-5- yl)pyrazine-2-carboxamide |
| 358 | 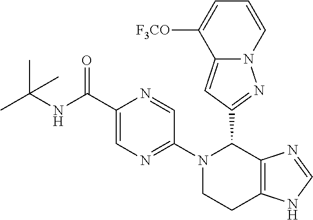 [SMILES: CC(C)(C)NC(=O)c1cnc(N2CCc3[nH]cnc3[C@H]2c2cc3c(OC(F)(F)F)cccn3n2)cn1] | (S)-N-(tert-butyl)-5-(4-(4- (trifluoromethoxy)pyrazolo[1,5-a]pyridin-2-yl)- 1,4,6,7-tetrahydro-5H-imidazo[4,5-c]pyridin-5- yl)pyrazine-2-carboxamide |
| 359 | 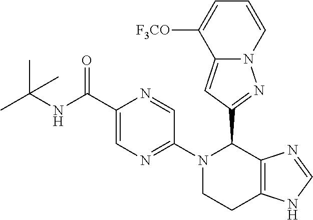 [SMILES: CC(C)(C)NC(=O)c1cnc(N2CCc3[nH]cnc3[C@@H]2c2cc3c(OC(F)(F)F)cccn3n2)cn1] | (R)-N-(tert-butyl)-5-(4-(4- (trifluoromethoxy)pyrazolo[1,5-a]pyridin-2-yl)- 1,4,6,7-tetrahydro-5H-imidazo[4,5-c]pyridin-5- yl)pyrazine-2-carboxamide |
| 360 | 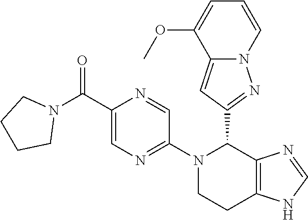 [SMILES: COc1cccn2nc([C@@H]3c4nc[nH]c4CCN3c3cnc(C(=O)N4CCCC4)cn3)cc12] | (S)-(5-(4-(4-methoxypyrazolo[1,5-a]pyridin-2- yl)-1,4,6,7-tetrahydro-5H-imidazo[4,5- c]pyridin-5-yl)pyrazin-2-yl)(pyrrolidin-1- yl)methanone |
| 361 | 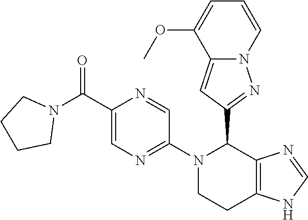 [SMILES: COc1cccn2nc([C@H]3c4nc[nH]c4CCN3c3cnc(C(=O)N4CCCC4)cn3)cc12] | (R)-(5-(4-(4-methoxypyrazolo[1,5-a]pyridin-2- yl)-1,4,6,7-tetrahydro-5H-imidazo[4,5- c]pyridin-5-yl)pyrazin-2-yl)(pyrrolidin-1- yl)methanone |

In further embodiments, the compound is one or more of the compounds in Table 1 that is the S-enantiomer (e.g., the stereogenic carbon atom of the imidazopyridinyl is in the S-configuration), or a pharmaceutically acceptable salt thereof. In yet other embodiments, the compound is one or more of the compounds in Table 1 that is the R-enantiomer (e.g., the stereogenic carbon atom of the imidazopyridinyl is in the R-configuration), or a pharmaceutically acceptable salt thereof. In still further embodiments, the compound is a racemate of one or more of the compounds in Table 1, or a pharmaceutically acceptable salt thereof.

In some embodiments, the disclosure provides additional prophetic examples as set forth in Table 2 below, and their pharmaceutically acceptable salts and/or isotopologues. The prophetic compounds in Table 2 may be made in accordance with the procedures described in the General Schemes, Intermediate Schemes, and Examples herein alone or in combination with knowledge of a person of ordinary skill in the art.

TABLE 2 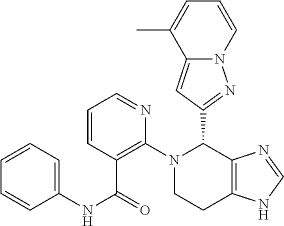 [SMILES: Cc1cccn2nc([C@@H]3c4nc[nH]c4CCN3c3ncccc3C(=O)Nc3ccccc3)cc12]
Ex. 362 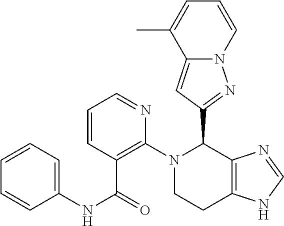 [SMILES: Cc1cccn2nc([C@H]3c4nc[nH]c4CCN3c3ncccc3C(=O)Nc3ccccc3)cc12]
Ex. 363 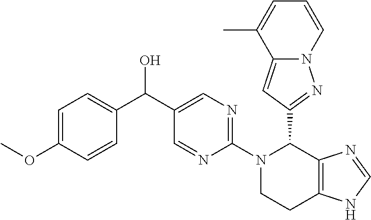 [SMILES: COc1ccc(C(O)c2cnc(N3CCc4[nH]cnc4[C@H]3c3cc4c(C)cccn4n3)nc2)cc1]
Ex. 364 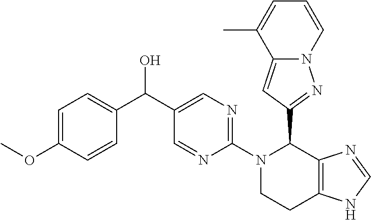 [SMILES: COc1ccc(C(O)c2cnc(N3CCc4[nH]cnc4[C@@H]3c3cc4c(C)cccn4n3)nc2)cc1]
Ex. 365 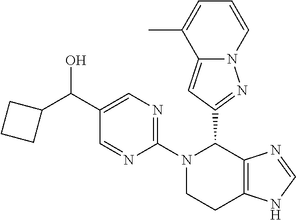 [SMILES: Cc1cccn2nc([C@@H]3c4nc[nH]c4CCN3c3ncc(C(O)C4CCC4)cn3)cc12]
Ex. 366 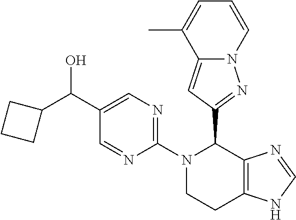 [SMILES: Cc1cccn2nc([C@H]3c4nc[nH]c4CCN3c3ncc(C(O)C4CCC4)cn3)cc12]
Ex. 367 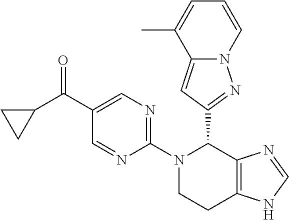 [SMILES: Cc1cccn2nc([C@@H]3c4nc[nH]c4CCN3c3ncc(C(=O)C4CC4)cn3)cc12]
Ex. 368 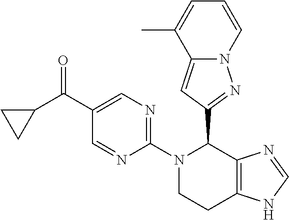 [SMILES: Cc1cccn2nc([C@H]3c4nc[nH]c4CCN3c3ncc(C(=O)C4CC4)cn3)cc12]
Ex. 369 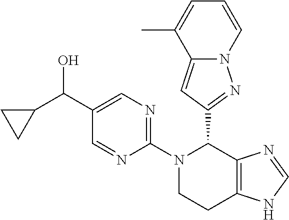 [SMILES: Cc1cccn2nc([C@@H]3c4nc[nH]c4CCN3c3ncc(C(O)C4CC4)cn3)cc12]
Ex. 370 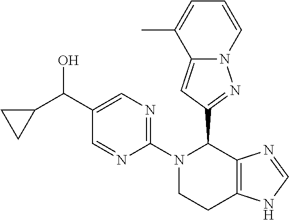 [SMILES: Cc1cccn2nc([C@H]3c4nc[nH]c4CCN3c3ncc(C(O)C4CC4)cn3)cc12]
Ex. 371 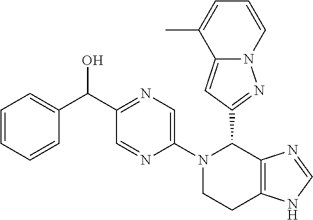 [SMILES: Cc1cccn2nc([C@@H]3c4nc[nH]c4CCN3c3cnc(C(O)c4ccccc4)cn3)cc12]
Ex. 372 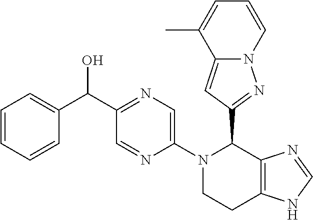 [SMILES: Cc1cccn2nc([C@H]3c4nc[nH]c4CCN3c3cnc(C(O)c4ccccc4)cn3)cc12]
Ex. 373

In further embodiments, the compound is one or more of the compounds in Table 2 that is the S-enantiomer (e.g., the stereogenic carbon atom of the imidazopyridinyl is in the S-configuration), or a pharmaceutically acceptable salt thereof. In yet other embodiments, the compound is one or more of the compounds in Table 2 that is the R-enantiomer (e.g., the stereogenic carbon atom of the imidazopyridinyl is in the R-configuration), or a pharmaceutically acceptable salt thereof. In still further embodiments, the compound is a racemate of one or more of the compounds in Table 2, or a pharmaceutically acceptable salt thereof.

Within Tables 1 and 2 containing example numbers 89 to 373, example numbers 133-203 and 206-215 are not used.

### Treatment Methods

Compounds and pharmaceutical compositions of the disclosure have several uses as described herein. In some embodiments, compounds and pharmaceutical compositions of the disclosure are useful in methods for stabilizing mutant PAH proteins. These methods comprise contacting the protein with one or more compounds described herein or a pharmaceutically acceptable salt thereof. The compounds and pharmaceutical compositions of the disclosure can provide for better Phe control for patients whose disease is not well-managed on diet alone and lessen the severity of a patient's phenylketonuria. Thus, patients administered a compound or pharmaceutical composition of the disclosure will have a better quality of life, e.g., a more normal lifestyle and/or none or fewer dietary restrictions, as compared with phenylketonuria patients who have not been administered a compound or pharmaceutical composition of the disclosure. In some embodiments, patients administered a compound of pharmaceutical composition of the disclosure may experience increases in executive function, decreases in anxiety symptoms, and/or decreases in attention deficit hyperactivity disorder symptoms.

The term “mutant PAH gene” as used herein refers to the full DNA sequence of PAH that differs in one or more ways from the canonically accepted sequence (“the basis gene”) that is published in any one of a variety of curated databases. As one example, the sequence described by GenBank Accession number NG_008690.2 describes the basis gene.

The term “mutant PAH protein” as used herein refers to a PAH protein that contains at least one mutation in the amino acid sequence relative to that encoded by the reference. The reference human PAH protein is described by Genbank Accession number NP_000268 and contains 452 amino acids. PAH protein mutations can be identified using methods known in the art. In some embodiments, the mutant PAH protein contains at least one R408W, R261Q, R243Q, Y414C, L48S, A403V, I65T, R241C, L348V, R408Q, or V388M mutation. In other embodiments, the mutant PAH protein contains at least one R408W, Y414C, I65T, F39L, R408Q, L348V, R261Q, A300S, or L48S mutation. In still other embodiments, the mutant PAH protein contains at least one R408W, R243Q, R408Q, V388M, or L348V mutation. In yet other embodiments, the mutant PAH protein contains at least one R408W mutation. In further embodiments, the mutant PAH protein contains at least two R408W mutations. In further embodiments, the mutant PAH protein contains at least one R261Q mutation. In yet other embodiments, the mutant PAH protein contains at least one R243Q mutation. In yet other embodiments, the mutant PAH protein contains at least one Y414C mutation. In still further embodiments, the mutant PAH protein contains at least one L48S mutation. In other embodiments, the mutant PAH protein contains at least one A403V mutation. In further embodiments, the mutant PAH protein contains at least one I65T mutation. In yet further embodiments, the mutant PAH protein contains at least one R241C mutation. In yet other embodiments, the mutant PAH protein contains at least one L348V mutation. In further embodiments, the mutant PAH protein contains at least one R408Q mutation. In other embodiments, the mutant PAH protein contains at least one V388M mutation. In other embodiments, the mutant PAH protein contains at least one F39L mutation. In still further embodiments, the mutant PAH protein contains at least one A300S mutation. In yet further embodiments, the mutant PAH protein contains at least one L48S mutation.

In other embodiments, the disclosure provides methods for stabilizing the activity of mutant phenylalanine hydroxylase (PAH) proteins as compared to wild type PAH. Such methods include contacting phenylalanine hydroxylase with one or more compounds described herein, or a pharmaceutically acceptable salt thereof, or pharmaceutical compositions comprising compounds of the disclosure. The term “stabilizing” as used herein refers to modulating the activity or quantity of a PAH enzyme so that it catalyzes hydroxylation of the aromatic side-chain of phenylalanine at a rate that is more similar to the PAH catalysis rate of a control population having wild type PAH, i.e., without a mutant PAH gene mutation, as compared to the baseline PAH catalysis rate. In some aspects, the term “stabilizing” refers to modulating the activity of a subject's PAH so that it catalyzes hydroxylation of the aromatic side-chain of phenylalanine at a flux more similar to the PAH catalytic flux of a control subject population without a mutant PAH gene mutation. In some embodiments, “stabilizing” activity of PAH includes increasing levels of the enzyme PAH as compared to baseline. By increasing the buildup of stabilized active PAH protein, a subject's toxic Phe levels can be reduced as compared to the subject's baseline levels of dietary Phe prior to administration of a compound of the disclosure or a pharmaceutical composition comprising compounds of the disclosure.

In some embodiments, the disclosure provides methods for reducing blood phenylalanine concentrations in a subject suffering from phenylketonuria to a concentration less than or equal to about 600 μM. In other embodiments, the blood Phe concentration is reduced to a concentration less than or equal to about 360 μM. In other embodiments, the disclosure provides methods for reducing blood Phe concentrations as compared to untreated baseline. In some embodiments, a subject's blood Phe concentration as compared to untreated baseline is reduced by a percentage including but not limited to at least about 10%, at least about 20%, at least about 30%, at least about 40%, at least about 50%, at least about 60%, at least about 70%, at least about 80%, or at least about 90%. In other embodiments, a subject's blood Phe concentration as compared to untreated baseline is reduced by at least about 10%. In further embodiments, a subject's blood Phe concentration as compared to untreated baseline is reduced by at least about 20%. In yet other embodiments, a subject's blood Phe concentration as compared to untreated baseline is reduced by at least about 30%. In still further embodiments, a subject's blood Phe concentration as compared to untreated baseline is reduced by at least about 40%. In other embodiments, a subject's blood Phe concentration as compared to untreated baseline is reduced by at least about 50%. In further embodiments, a subject's blood Phe concentration as compared to untreated baseline is reduced by at least about 60%. In yet other embodiments, a subject's blood Phe concentration as compared to untreated baseline is reduced by at least about 70%. In still further embodiments, a subject's blood Phe concentration as compared to untreated baseline is reduced by at least about 80%. In other embodiments, a subject's blood Phe concentration as compared to untreated baseline is reduced by at least about 90%. A subject's Phe concentration can be determined by blood tests and methods for measuring such levels are known in the art. In some embodiments, the reduction in Phe concentration achieved using compounds of the disclosure is obtained in conjunction with the subject actively managing their dietary Phe intake. In other embodiments, the reduction in Phe concentration is obtained in conjunction with the subject maintaining a Phe-restricted diet.

In some embodiments, a subject is treated with compounds of the disclosure or a pharmaceutical composition comprising compounds of the disclosure. The compound is administered in an amount sufficient for stabilizing the PAH protein, or for reducing blood phenylalanine concentration in a subject, or combinations thereof in the subject.

In further embodiments, the subject is a human patient, such as a human adult over 18 years old in need of treatment. In yet further embodiments, the human patient is a human child less than 18 years old. In still further embodiments, the human patient is a human child between 12 years and 18 years old. In yet other embodiments, the human patient is a human child less than 12 years old. In any of the embodiments, the subject has phenylketonuria (PKU), optionally classic PKU or severe PKU. In some embodiments, the subject has a blood Phe concentration greater than about 600 μM prior to administration of a compound of the disclosure or a pharmaceutical composition comprising compounds of the disclosure. In other embodiments, the subject's blood Phe concentration prior to administration is greater than about 700 μM. In further embodiments, the subject's blood Phe concentration prior to administration is greater than about 800 μM. In still further embodiments, the subject's blood Phe concentration prior to administration is greater than about 900 μM. In yet other embodiments, the subject's blood Phe concentration prior to administration is greater than about 1000 μM. In further embodiments, the subject's blood Phe concentration prior to administration is greater than about 1100 μM. In other embodiments, the subject's blood Phe concentration prior to administration is greater than about 1200 μM.

The present methods also encompass administering an additional therapeutic agent to the subject in addition to the compounds of the disclosure. In some embodiments, the additional therapeutic agent is selected from drugs known as useful in a stabilizing mutant PAH protein and/or reducing blood Phe concentrations. The additional therapeutic agent is different from the compounds of the disclosure. In some embodiments, the additional therapeutic agent is sapropterin or sepiapterin. In other embodiments, the additional therapeutic agent is a nutritional supplement. Nutritional supplements that may be used include those that contain amino acids and other nutrients. In further embodiments, the nutritional supplement contains large neutral amino acids such as leucine, tyrosine, tryptophan, methionine, histidine, isoleucine, valine, threonine. In other embodiments, the nutritional supplement contains tyrosine. In further embodiments, the nutritional supplement contains casein glycomacropeptide, i.e., a milk peptide naturally free of Phe in its pure form. In other embodiments, the additional therapeutic agent is an enzyme substrate or enzyme co-factor. In yet other embodiments, the enzyme substrate or co-factor is tetrahydrobiopterin. In other embodiments, the additional therapeutic agent is a biopterin analogue. In further embodiments, the additional therapeutic agent is a biotherapeutic, synthetic biotic, microbiota or probiotic. In yet other embodiments, the biotherapeutic, synthetic biotic, microbiota or probiotic contains a genetically modified phenylalanine ammonia lyase (PAL) gene, such as, for example, E. coli Nissle PAL. Examples of genetically modified E. coli Nissle PAL biotherapeutics include SYNB1934 and SYNB1618, and the like. In still further embodiments, the additional therapeutic agent is an inhibitor of an amino acid transporter. In some embodiments, the amino acid transporter is B⁰AT1 (also referred to as SLC6A19), and the additional therapeutic agent is a SLC6A19 inhibitor. Examples of SLC6A19 inhibitors include nimesulide, benztropine, NSC63912, NSC22789, cinromide, CB3, E62, JNT-517, and the like.

Compounds and pharmaceutical compositions of the disclosure and the additional therapeutic agents can be administered simultaneously or sequentially to achieve the desired effect. In addition, the compounds of the disclosure and additional therapeutic agent can be administered in a single composition or two separate compositions.

The additional therapeutic agent is administered in an amount to provide its desired therapeutic effect. The effective dosage range for each additional therapeutic agent is known in the art, and the additional therapeutic agent is administered to an individual in need thereof within such established ranges.

Compounds and pharmaceutical compositions of the disclosure and the additional therapeutic agents can be administered together as a single-unit dose or separately as multi-unit doses, wherein the compounds or pharmaceutical compositions of the disclosure are administered before the additional therapeutic agent or vice versa. One or more doses of the compounds or pharmaceutical compositions of the disclosure and/or one or more dose of the additional therapeutic agents can be administered.

The compounds and pharmaceutical compositions of the disclosure may also be administered sequentially or concurrently with non-pharmacological techniques. In some embodiments, the patient uses non-pharmacological techniques to maintain lower Phe levels. In other embodiments, the non-pharmacological technique is administering a diet that is low in Phe. One skilled in the art would be able to determine what type of diet to maintain appropriate levels of Phe. In some embodiments, a phenylamine diet containing about 200 to about 500 mg/day (patients 10 years or younger) of Phe or less than about 600 mg/day (patients over 10 years of age). In other embodiments, the diet may include restricting or eliminating one or more foods that are high in Phe, such as soybeans, egg whites, shrimp, chicken breast, spirulina, watercress, fish, nuts, crayfish, lobster, tuna, turkey, legumes, and low-fat cottage cheese.

An example of a dose is in the range of from about 0.001 to about 100 mg of compound per kg of subject's body weight per day, in single or divided dosage units (e.g., BID, TID, QID). For a 70-kg human, a suitable dosage amount is from about 0.05 to about 7 g/day.

In some embodiments, the therapeutically effective amount of one or more compounds or pharmaceutical compositions described herein is an amount that is effective in stabilizing a mutant PAH protein described herein. In other embodiments, the therapeutically effective amount of one or more compounds or pharmaceutical compositions described herein is an amount that is effective in reducing blood phenylalanine concentrations.

Unless otherwise noted, the amounts of the compounds described herein are set forth on a free base basis. That is, the amounts indicate that amount of the compound administered, exclusive of, for example, solvent or counterions (such as in pharmaceutically acceptable salts).

### Pharmaceutical Compositions

The disclosure also provides pharmaceutical compositions comprising compounds of the disclosure and a pharmaceutically acceptable carrier or excipient.

The methods of the present disclosure can be accomplished by administering compounds of the disclosure as the neat compound or as a pharmaceutical composition. Administration of a pharmaceutical composition, or neat compound of the disclosure, can be performed at any time period as determined by the attending physician. Typically, the pharmaceutical compositions contain no toxic, carcinogenic, or mutagenic compounds that would cause an adverse reaction when administered.

Pharmaceutical compositions include those wherein compounds of the disclosure are administered in an effective amount to achieve its intended purpose. The exact formulation, route of administration, and dosage is determined by an individual physician.

Compounds of the disclosure can be administered by any suitable route, e.g., by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high-pressure technique.

The above-mentioned additional therapeutically active agents, one or more of which can be used in combination with compounds of the disclosure are prepared and administered as described in the art.

Compounds of the disclosure may be administered in admixture with pharmaceutical carriers selected with regard to the intended route of administration and standard pharmaceutical practice. Pharmaceutical compositions for use in accordance with the present disclosure are formulated in a conventional manner using one or more physiologically acceptable carriers comprising excipients and/or auxiliaries that facilitate processing of compounds of the disclosure.

Administration of the compounds or pharmaceutical compositions of the disclosure can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. Compounds can also be administered intraadiposally or intrathecally.

The amount of the compound or pharmaceutical composition administered will be dependent on the subject being treated, the severity of the disorder or condition, the rate of administration, the disposition of the compound and the discretion of the prescribing physician. The desired dose can be administered in a single dose, or as multiple doses administered at appropriate intervals, e.g., as one, two, three, four or more sub doses per day. In some embodiments, the compounds and pharmaceutical compositions disclosed herein are effective over a wide dosage range. For example, in the treatment of adult humans, dosage forms containing from about 0.01 to 2000 mg of a compound disclosed herein per day are examples of dosage forms that may be used. The exact dosage will depend upon the route of administration, the form in which the compound is administered, the subject to be treated, the body weight of the subject to be treated, and the preference and experience of the attending physician. In some instances, dosage levels below the lower limit of the aforesaid range may be more than adequate, while in other cases still larger doses may be employed without causing any harmful side effect, e.g., by dividing such larger doses into several small doses for administration throughout the day.

In some embodiments, a compound of the disclosure is administered in a single dose.

Typically, such administration will be by a solid oral dosage form such as tablet or capsule. However, other routes may be used as appropriate. A single dose of a compound may also be used for treatment of an acute condition.

In some embodiments, a compound of the disclosure may be administered in multiple doses. Dosing may be about once, twice, three times, four times, five times, six times, or more than six times per day. In another embodiment, a compound described herein and another therapeutic agent are administered together about once per day to about 6 times per day. Administration of the compounds disclosed herein may continue as long as necessary. In some embodiments, a compound is administered chronically on an ongoing basis, e.g., for the treatment of chronic effects.

An effective amount of a compound or pharmaceutical composition of the disclosure may be administered in either single or multiple doses by any of the accepted modes of administration of therapeutic agents having similar utilities, including rectal, buccal, intranasal and transdermal routes, by intra-arterial injection, intravenously, intraperitoneally, parenterally, intramuscularly, subcutaneously, orally, topically, or as an inhalant.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include one or more conventional pharmaceutical carriers or excipients and a compound disclosed herein as an active ingredient. In addition, it may include other medicinal or pharmaceutical agents, carriers, adjuvants, etc.

Exemplary parenteral administration forms include solutions or suspensions of the compound of the disclosure in sterile aqueous solutions, for example, aqueous propylene glycol or dextrose solutions. Such dosage forms can be suitably buffered, if desired.

### Pharmaceutical Compositions for Oral Administration

In some embodiments, the disclosure provides a pharmaceutical composition for oral administration containing a compound of the disclosure and pharmaceutical excipients suitable for oral administration.

In some embodiments, the disclosure provides a solid pharmaceutical composition for oral administration containing: (i) an effective amount of a compound of the disclosure; optionally (ii) an effective amount of a second therapeutic agent; and (iii) a pharmaceutical excipient suitable for oral administration. In some embodiments, the composition further contains: (iv) an effective amount of a third therapeutic agent.

In some embodiments, the pharmaceutical composition may be a pharmaceutical composition suitable for oral consumption. Pharmaceutical compositions containing a compound of the disclosure suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the compound of the disclosure into association with the carrier, which constitutes one or more necessary ingredients. In general, the compositions are prepared by uniformly and intimately admixing the compound of the disclosure with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This disclosure further encompasses anhydrous pharmaceutical compositions and dosage forms comprising a compound of the disclosure as the active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms containing a compound of the disclosure can be prepared using anhydrous or low moisture containing ingredients and low moisture or low humidity conditions. Pharmaceutical compositions and dosage forms containing a compound of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

The compound of the disclosure can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, colloidal silicon dioxide, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 2 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical, and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-1000 succinate, PEG-24 cholesterol, polyglyceryl-10-oleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the disclosure and to minimize precipitation of the compound of the disclosure. This can be important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol (PEG), polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; polyethylene glycol 660 12-hydroxystearate; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, glycofurol, transcutol, propylene glycol, and dimethyl isosorbide. Particularly preferred solubilizers include sorbitol, glycerol, triacetin, ethyl alcohol, PEG having an average molecular weight of about 100 to about 8000 g/mole, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of less than about 10%, less than about 25%, less than about 50%, about 100%, or up to less than about 200% by weight, based on the combined weight of the drug, and other excipients. If desired, very small amounts of solubilizer may also be used, such as less than about 5%, less than about 2%, less than about 1% or even less. Typically, the solubilizer may be present in an amount of less than about 1% to about 100%, more typically less than about 5% to less than about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

### Pharmaceutical Compositions for Injection

In some embodiments, the disclosure provides a pharmaceutical composition for injection containing a compound described herein and pharmaceutical excipients suitable for injection. Components and amounts of agents in the compositions are as described herein.

The forms in which the compositions of the disclosure may be incorporated for administration by injection include aqueous or oil suspensions or emulsions. Such compositions may comprise sesame oil, corn oil, cottonseed oil, peanut oil, elixirs containing mannitol or dextrose, sterile water, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Other Pharmaceutical Compositions

Pharmaceutical compositions may also be prepared from compositions described herein and one or more pharmaceutically acceptable excipients suitable for topical, sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2004; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

### Synthesis of Compounds of the Disclosure

Compounds of the disclosure can be prepared by methods described in the General Schemes, procedures, and Examples set forth within, and by related methods known in the art, including those methods described in PCT patent application publication nos. WO2023/164235, WO2023/164234, WO2023/164236, WO2023/164233, and WO2023/164237.

General Scheme 1: Preparation of Intermediate of Formula 1.3

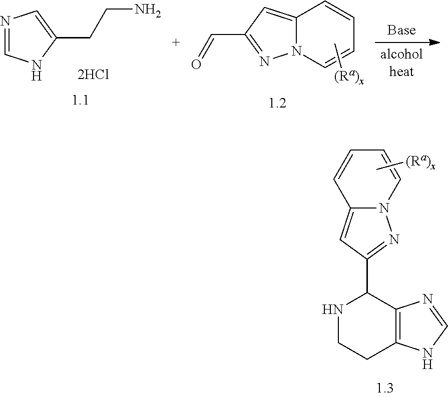

Intermediates of Formula 1.3 (wherein each R^{a} independently is halo, C₁₋₆alkyl, C₃₋₆cycloalkyl, C₁₋₆haloalkyl, C₁₋₆alkoxy or C₁₋₆haloalkoxy and x is 0 to 5) were obtained through the Pictet-Spengler reaction shown in General Scheme 1. An amine of formula 1.1 and an aldehyde of formula 1.2 were reacted in the presence of a base such as sodium carbonate, an alcoholic solvent such as ethanol or methanol, and heat to afford the core amine of formula 1.3. To the extent that an imine by-product is formed during the Pictet-Spengler reaction, the imine by-product can be converted to the core amine of formula 1.3 by reaction with sodium borohydride in an alcoholic solvent (for example, methanol or ethanol). The core amine of formula 1.3 can then be used in nucleophilic substitution reactions (see General Schemes 2, 4, 6, and 8) or Buchwald or other similar cross-coupling reactions (see General Scheme 3). The core amine of formula 1.3 can also be separated into its R- and S-enantiomers (e.g., the stereogenic carbon atom of the imidazopyridinyl is in either the R-configuration or the S-configuration) through SFC separation, and then either one or both enantiomers may be separately further reacted in nucleophilic substitution or Buchwald or other similar cross-coupling reactions in accordance with General Schemes 2 to 4, 6, and 8.

### General Scheme 2: Nucleophilic Substitution Reactions

As shown in Methods A through F, an optionally substituted heteroaryl halide was coupled to the amine of formula 1.3 using a nucleophilic substitution reaction under basic conditions, such as DIPEA or TEA, to afford a racemic compound which was then separated into its enantiomers through SFC separation. Alternatively, an amine of formula 3.3,

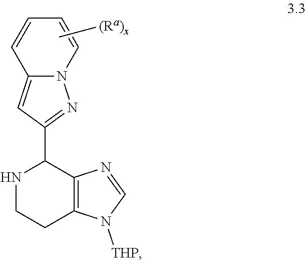

prepared in accordance with General Scheme 3 below, may also be used in the nucleophilic substitution reactions, followed by deprotection of the tetrahydropyranyl (THP) protecting group under acidic conditions. Alternatively, the R- or S-enantiomer of the amine of formulas 1.3 and 3.3 (e.g., the stereogenic carbon atom of the imidazopyridinyl is in either the R-configuration or the S-configuration) may also be separately used in the nucleophilic substitution reactions described herein.

As shown in Method A, the core amine of formula 1.3 may be reacted with an optionally substituted pyrazinyl of formula 2.1 (wherein each R^{m} independently can be cyano, alkyl, haloalkyl, alkoxy, haloalkoxy, alkoxy(alkylene), halo, cycloalkyl, cyanoalkyl, hydroxyalkyl, amino, hydroxy, oxo, —CO₂(C₁-C₆alkyl), or L-R¹ as defined herein; and y is 0, 1, or 2) in a nucleophilic substitution reaction, followed by SFC separation to afford enantiomers of formulas 2.2 and 2.3.

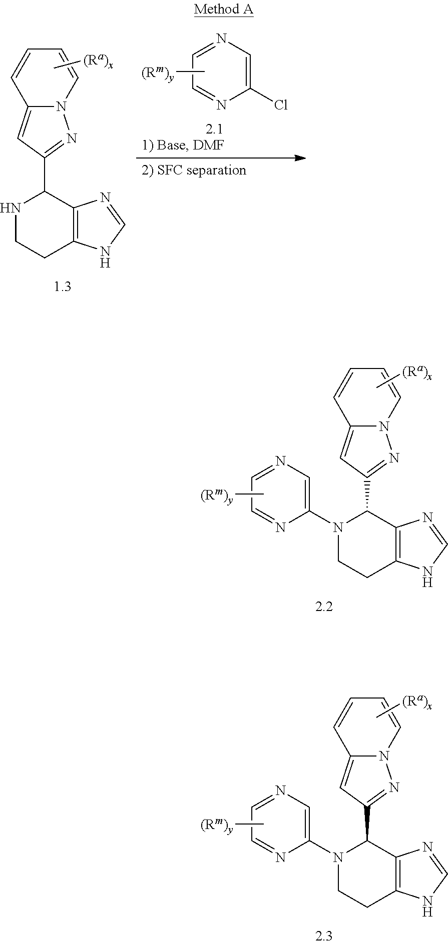

As shown in Method B, the core amine of formula 1.3 may be reacted with an optionally substituted pyridinyl of formula 2.4 (wherein each Rⁿ independently can be cyano, alkyl, haloalkyl, alkoxy, haloalkoxy, alkoxy(alkylene), halo, cycloalkyl, cyanoalkyl, hydroxyalkyl, amino, hydroxy, oxo, —CO₂(C₁-C₆alkyl), or L-R¹ as defined herein; y is 0, 1, or 2; and X is Cl or F) in a nucleophilic substitution reaction, followed by SFC separation to afford enantiomers of formulas 2.5 and 2.6.

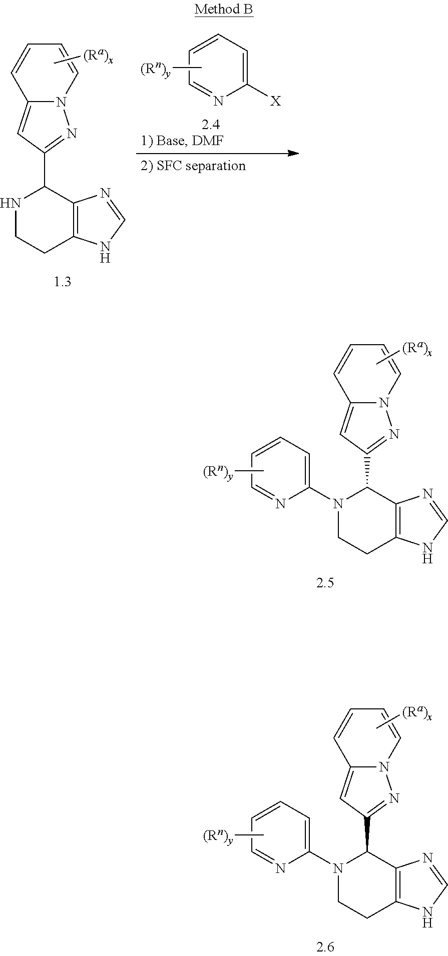

As shown in Methods C1 and C2, the core amine of formula 1.3 may be reacted with an optionally substituted pyrimidinyl of formula 2.7 or formula 2.10 (wherein each R^{o} independently can be cyano, alkyl, haloalkyl, alkoxy, haloalkoxy, halo, cycloalkyl, cyanoalkyl, hydroxyalkyl, amino, hydroxy, —CO₂(C₁-C₆alkyl), or L-R¹ as defined herein; y is 0, 1, or 2; and X is Cl or F) in a nucleophilic substitution reaction, followed by SFC separation to afford enantiomers of formulas 2.8, 2.9, 2.11, and 2.12.

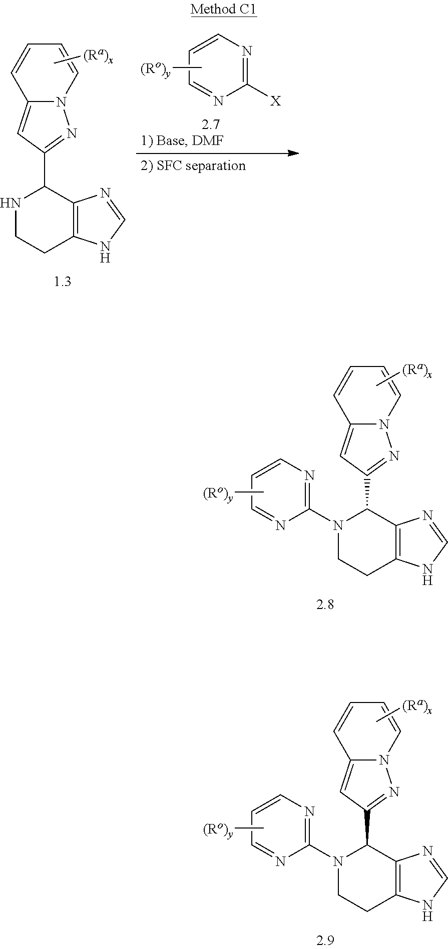

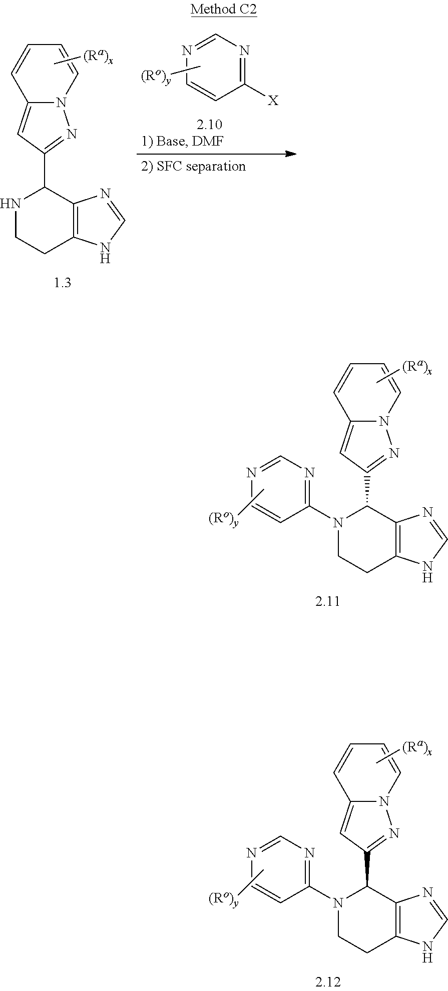

As shown in Method D, the core amine of formula 1.3 may be reacted with an optionally substituted pyridazinyl of formula 2.13 (wherein each RP independently can be cyano, alkyl, haloalkyl, alkoxy, haloalkoxy, halo, cycloalkyl, cyanoalkyl, hydroxyalkyl, amino, hydroxy, —CO₂(C₁-C₆alkyl), or L-R¹ as defined herein; and y is 0, 1, or 2) in a nucleophilic substitution reaction, followed by SFC separation to afford enantiomers of formulas 2.14 and 2.15.

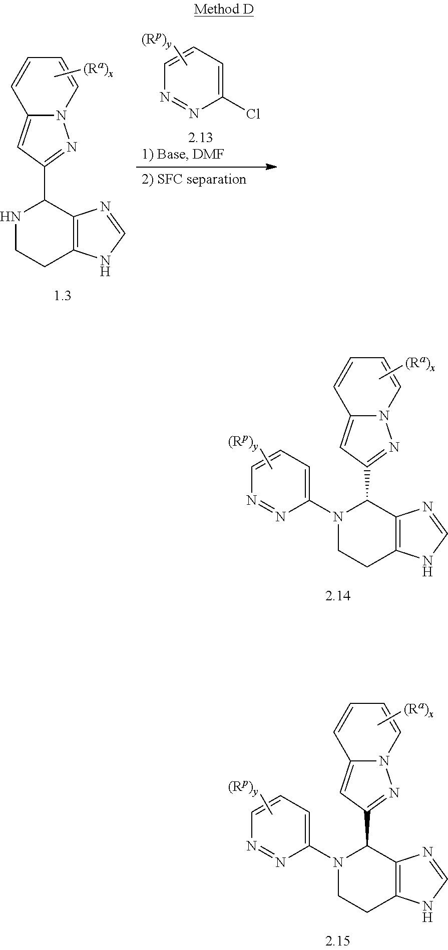

### General Scheme 3: Buchwald or Cross-Coupling Reactions

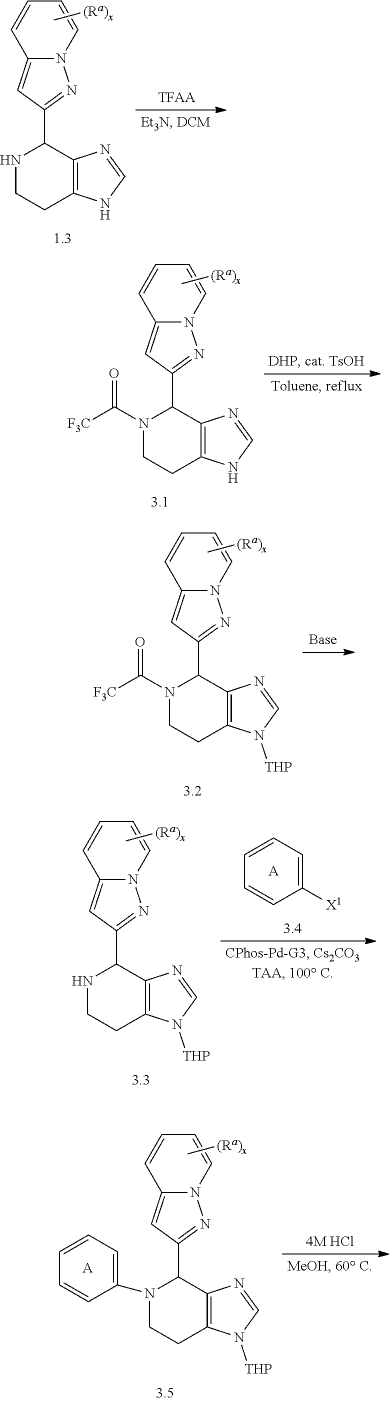

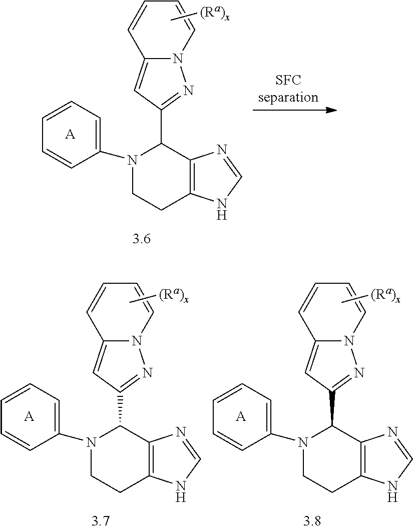

As shown in General Scheme 3, the core amine of formula 1.3 may be protected with a trifluoroacetyl (TFA) protecting group through reaction with trifluoroacetic anhydride and a base such as triethyl amine to afford compounds of formula 3.1. One of the imidazole nitrogen atoms in the compound of formula 3.1 is protected with a THP protecting group via reaction with DHP and a catalytic amount of an acid such as TsOH to afford compounds of formula 3.2. The TFA protecting group in a compound of formula 3.2 may then be removed by reaction with a base such as 1M NaOH in THF or K₂CO₃ in MeOH to afford a compound of formula 3.3. An optionally substituted 6-membered heteroaryl halide of formula 3.4 (wherein ring A represents a 6-membered heteroaryl such as a pyrazinyl, a pyridinyl, a pyrimidinyl, or a pyridazinyl and X¹ is Br, Cl, or I) may be installed by using Buchwald coupling or cross-coupling conditions known in the art, such as using a palladium catalyst (for example, CPhos-Pd-G3, Pd(OAc)₂, or Pd(dppf)Cl₂), and a base such as Cs₂CO₃, to afford compounds of formula 3.5. The THP protecting group in compounds of formula 3.5 can be removed under acidic conditions such as 4M HCl in MeOH, TsOH in THF/water, or PPTS in ethanol to afford racemic compounds of formula 3.6. Racemic compounds of formula 3.6 may be separated into enantiomers of formulas 3.7 and 3.8 by SFC separation.

### General Schemes 4A, 4B, and 4C: Nucleophilic Substitution Reactions

In addition to the nucleophilic substitution reactions described in General Scheme 2, compounds of Formula I were prepared as set forth in General Schemes 4A, 4B, and 4C.

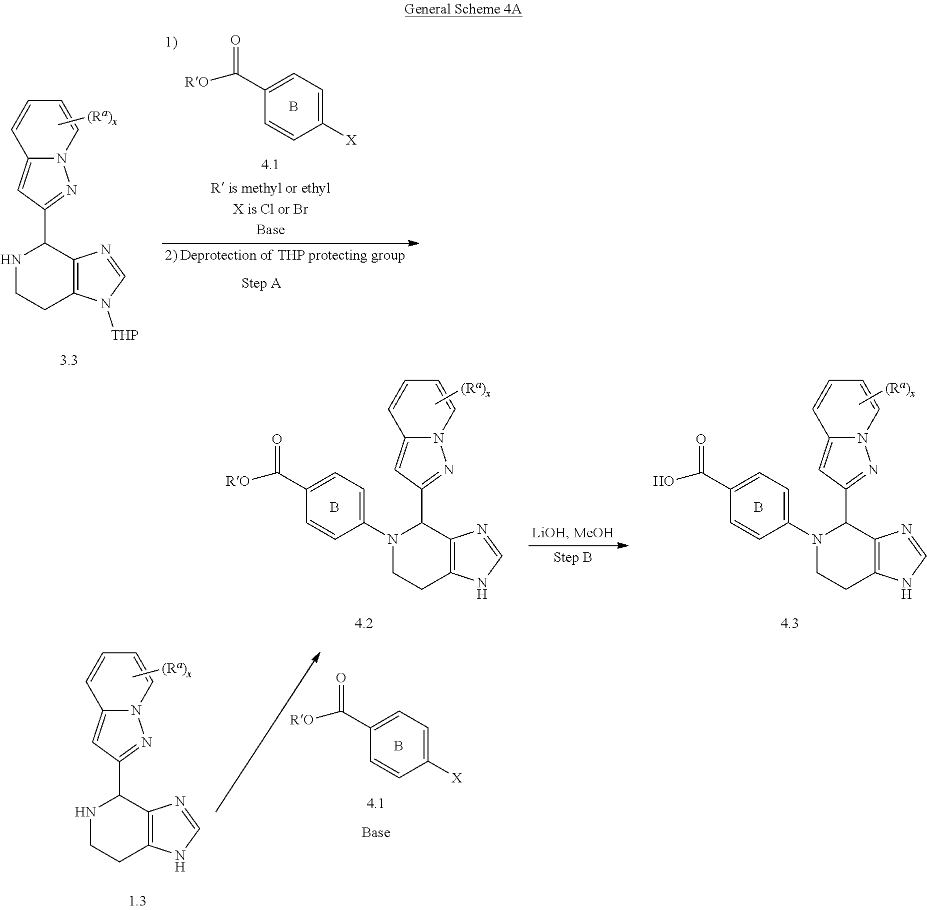

In Step A of General Scheme 4A, compounds of formula 4.2 may be obtained by reacting an amine of formula 1.3 or formula 3.3 with a halide-substituted heteroaryl alkyl carboxylate of formula 4.1 (wherein ring B is a pyrazinyl, pyridinyl, pyrimidinyl, or pyridazinyl heteroaryl) under nucleophilic substitution conditions using basic conditions, such as DIPEA or TEA, followed by deprotection of the THP protecting group when an amine of formula 3.3 is used under acid conditions, such as TsOH in THF/water, 4M HCL solution, or PPTS in ethanol. Examples of compounds of formula 4.1 that may be used in the reactions in General Scheme 4A include methyl 5-chloropyrazine-2-carboxylate, ethyl 4-chloropyrimidine-5-carboxylate, methyl 6-chloropicolinate, ethyl 2-chloro-4-methylpyrimidine-5-carboxylate, and methyl 6-bromopyridazine-3-carboxylate. In Step B of General Scheme 4A, the alkyl ester moiety of a compound of formula 4.2 is cleaved to a carboxylic acid (or lithium salt thereof) by reaction with lithium hydroxide in an alcoholic solvent such as methanol or ethanol to afford a compound of formula 4.3.

As shown in Routes A and B in General Scheme 4B, the carboxylic acid (or lithium salt thereof) of a compound of formula 4.3 may be reacted with various amines (including R¹- and R⁴-primary amines, (alkyl)₂NH, and heterocyclic amines) using acid coupling conditions known in the art, such as using HATU, T4P, T3P, or EDCI and HOBt along with a base, such as DIPEA or TEA, at room temperature or at elevated temperatures to afford racemic compounds of formulas 4.4, 4.5, and 4.6. In Route C of General Scheme 4B, substituted aminophenols may be reacted with N,N,N′,N′-tetramethylchloroformamidinium hexafluorophosphate and N-methylimidazole in order to form an amide bond and afford racemic compounds of formula 4.7. The racemic compounds of formulas 4.4, 4.5, 4.6, and 4.7 may be separated into their S- and R-enantiomers by SFC separation.

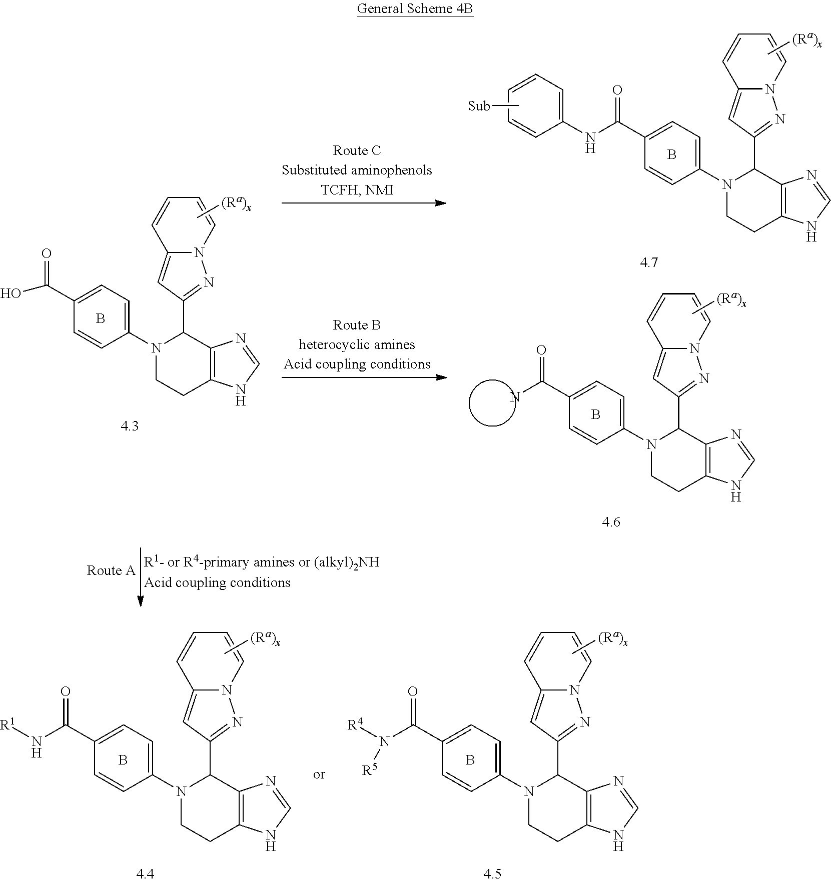

Alternatively, as shown in General Scheme 4C, carboxylate compounds of formula 4.2 or the S- or R-enantiomer thereof may be reacted with R¹-primary amines in ethanol at elevated temperatures to afford compounds of formula 4.4 or the S- or R-enantiomer thereof.

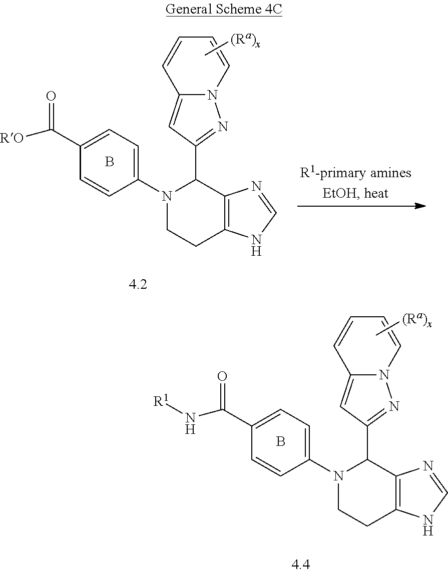

### General Scheme 5: Preparation of Intermediates of Formulas 5.1, 5.2, and 5.3

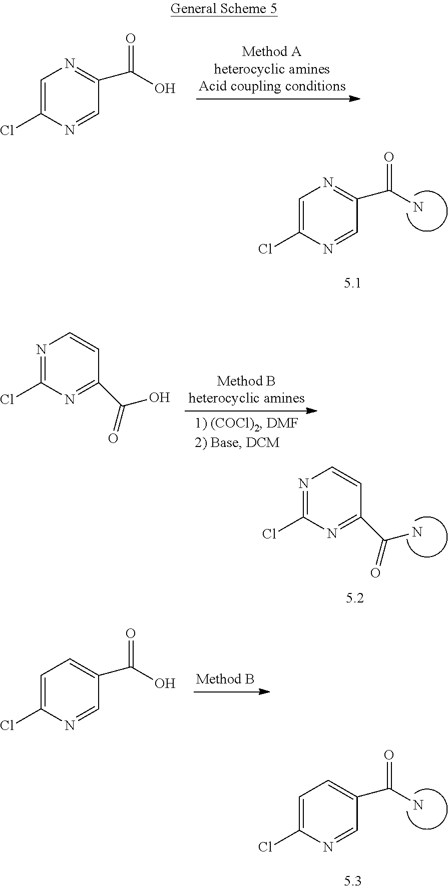 [SMILES: O=C(O)c1cnc(Cl)cn1 ; NC(=O)c1cnc(Cl)cn1 ; O=C(O)c1ccnc(Cl)n1 ; NC(=O)c1ccnc(Cl)n1 ; O=C(O)c1ccc(Cl)nc1 ; NC(=O)c1ccc(Cl)nc1]

As shown in General Scheme 5, chloro-substituted heteroaryl carboxylic acids may be reacted with heterocyclic amines to afford compounds of formulas 5.1, 5.2, and 5.3. Examples of chloro-substituted heteroaryl carboxylic acids that may be used include 5-chloropyrazine-2-carboxylic acid, 2-chloropyrimidine-4-carboxylic acid, and 6-chloropyridine-3-carboxylic acid. In Method A, 5-chloropyrazine-2-carboxylic acid may be reacted with heterocyclic amines using acid coupling conditions known in the art, such as using HATU, T4P, T3P, or EDCI and HOBt along with a base, such as DIPEA or TEA, at room temperature or at elevated temperatures to afford compounds of formula 5.1. In Method B, 2-chloropyrimidine-4-carboxylic acid may be reacted first with oxalyl chloride and then with a heterocyclic amine and a base such as DIPEA or TEA to afford compounds of formula 5.2. Similarly, 6-chloropyridine-3-carboxylic acid may be reacted as described in Method B to afford compounds of formula 5.3. Compounds of formulas 5.1, 5.2, and 5.3 can be used in a nucleophilic substitution reaction to afford racemic compounds of formula 4.6 or the S- or R-enantiomer thereof.

### General Scheme 6: Nucleophilic Substitution Reactions

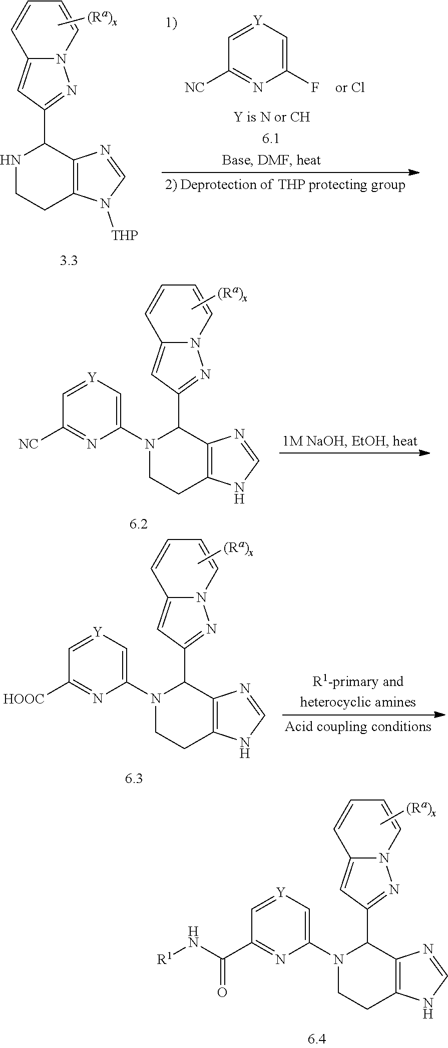

In addition to the nucleophilic substitution reactions described in General Schemes 2 and 4, compounds of formula 6.4 may be prepared in accordance with General Scheme 6. An amine of formula 3.3 may be reacted in a nucleophilic substitution reaction with a halide-substituted heteroaryl carbonitrile of formula 6.1 (wherein Y is N or CH) such as 6-fluoropicolinonitrile or 6-chloropyrazine-2-carbonitrile and a base such as DIPEA or TEA at elevated temperatures. The THP protecting group can then be removed under acidic conditions, such as TsOH in THF/water, 4M HCL solution, or PPTS in ethanol, to afford compounds of formula 6.2. The cyano moiety of compounds of formula 6.2 may be hydrolyzed to a carboxylic acid moiety by reaction with 1M sodium hydroxide solution in ethanol at elevated temperatures to afford compounds of formula 6.3. Compounds of formula 6.3 may be reacted with R¹ primary amines and heterocyclic amines using acid coupling conditions known in the art, such as using HATU, T4P, T3P, or EDCI and HOBt along with a base, such as DIPEA or TEA, to afford compounds of formula 6.4. Racemic compounds of formula 6.4 may be separated into their S- and R-enantiomers using SFC separation or alternatively, the S- or R-enantiomer of a compound of formula 3.3 may be used in General Scheme 6.

### General Scheme 7: Preparation of Compounds of Formulas 7.2 and 7.3

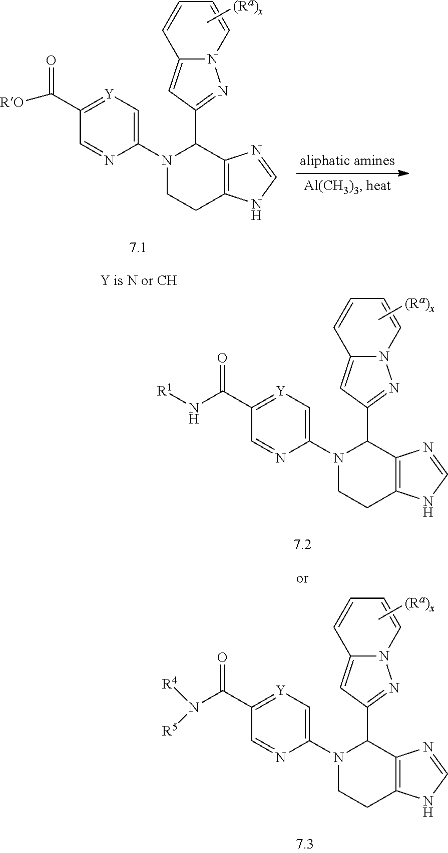

As shown in General Scheme 7, carboxylates of formula 7.1 (wherein Y is N or CH) may be reacted with aliphatic amines in the presence of trimethylaluminum and elevated temperatures to afford racemic compounds of formulas 7.2 and 7.3. Racemic compounds of formulas 7.2 and 7.3 may be separated into their S- and R-enantiomers using SFC separation or alternatively, the S- or R-enantiomer of a compound of formula 7.1 may be used in General Scheme 7.

### General Scheme 8: Preparation of Compounds of Formula 8.4

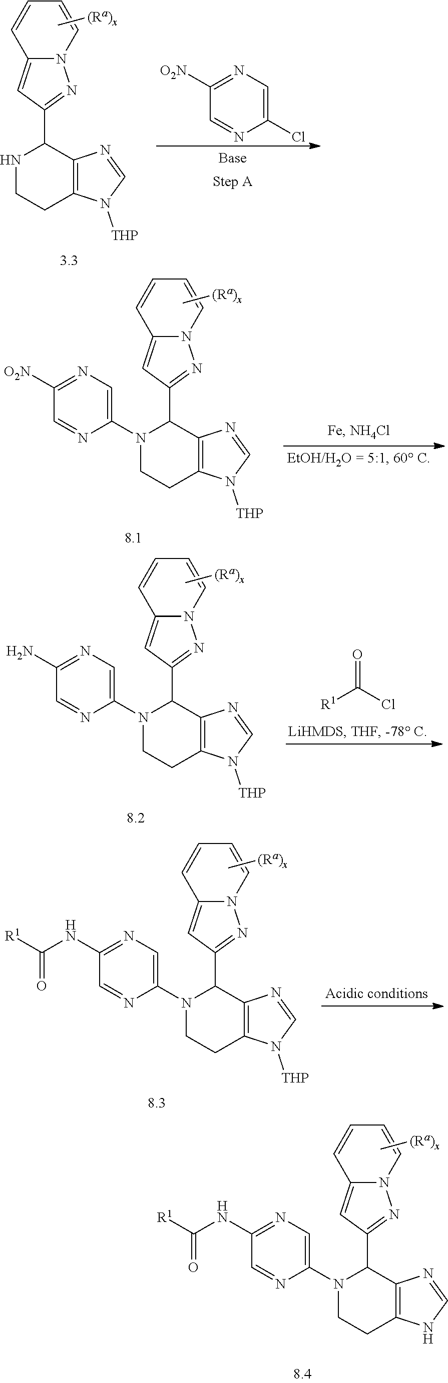

Compounds of Formula I wherein L is —C(O)NH— and R¹ is attached to the carbonyl moiety of L may be prepared as described in General Scheme 8. Amines of formula 3.3 may be reacted with 2-chloro-5-nitro-pyrazine in a nucleophilic substitution reaction using a base such as DIPEA or TEA to afford compounds of formula 8.1. The nitro moiety of compounds of formula 8.1 is reduced using iron and ammonium chloride or another reducing agent known in the art to afford a compound of formula 8.2. Compounds of formula 8.2 may be reacted with a R¹-acid chloride and lithium bis(trimethylsilyl)amide to afford compounds of formula 8.3. The THP protecting group in compounds of formula 8.3 may be removed under acidic conditions such as TsOH in THF/water, 4M HCL solution, or PPTS in ethanol to afford compounds of formula 8.4. Racemic compounds of formula 8.4 may be separated into their S- and R-enantiomers using SFC separation or alternatively, the S- or R-enantiomer of the amine of formula 3.3 may be used in General Scheme 8.

The present disclosure will be more fully understood by reference to the Examples described herein. The examples should not, however, be construed as limiting the scope of the present disclosure.

### Abbreviations and Terms List

| | |
|---|---|
| aq | aqueous |
| min | minute(s) |
| hrs | hours |
| mL | milliliter |
| μL | microliter |
| mmol | millimole(s) |
| μmol | micromole(s) |
| mol | mole(s) |
| M | molar |
| eq | equivalents |
| LCMS | Liquid chromatography mass spectrometry |
| NMR | nuclear magnetic resonance |
| TLC | thin layer chromatography |
| HPLC | high-performance liquid chromatography |
| SFC | supercritical fluid chromatography |
| sat | saturated |
| ° C. | degrees Celsius |
| rt | room temperature |
| N₂ | nitrogen gas |
| Hz | Hertz |
| δ | chemical shift |
| s | singlet |
| d | doublet |
| t | triplet |
| q | quartet |
| m | multiplet |
| br | broad |
| dd | doublet of doublets |
| ddd | doublet of doublet or doublets |
| td | triplet of doublets |
| dt | doublet of triplets |
| CDCl₃ | chloroform-d |
| CD₃OD | methanol-d₄ |
| DMSO-d₆ | dimethyl sulfoxide-d₆ |

### Solvents and Reagents:

| | |
|---|---|
| DCM | dichloromethane |
| DMAc | dimethylacetamide |
| DMF | dimethylformamide |
| DMSO | dimethyl sulfoxide |
| EtOH | ethyl alcohol |
| EtOAc | ethyl acetate |
| i-PrOH | isopropanol |
| or IPA | |
| MeOH | methanol |
| TAA | tert-amyl alcohol or 2-methylbutan-2-ol |
| MeCN | acetonitrile |
| or ACN | |
| PE | petroleum ether |
| TBME | tert-butyl methyl ether |
| H₂O | water |
| THF | tetrahydrofuran |
| NMP | N-methyl-2-pyrrolidone |
| HCl | hydrochloric acid |
| H₂SO₄ | sulfuric acid |
| TFA | trifluoroacetic acid |
| TFAA | trifluoroacetic anhydride |
| TosOH | p-toluenesulfonic acid |
| or TsOH | |
| PPTS | pyridinium p-toluenesulfonate |
| NH₄Cl | ammonium chloride |
| KOH | Potassium hydroxide |
| LiOH | lithium hydroxide |
| K₂CO₃ | potassium carbonate |
| Na₂CO₃ | sodium carbonate |
| Cs₂CO₃ | cesium carbonate |
| Na₂SO₄ | sodium sulfate |
| NaHCO₃ | sodium bicarbonate |
| Et₃N or TEA | triethylamine |
| DIPEA | N,N-diisopropylethylamine |
| or DIEA | |
| Py | pyridine |
| DHP | dihyropyran |
| CsF | cesium fluoride |
| NH₃ | ammonia |
| LiHMDS | lithium bis(trimethylsilyl)amide |
| MeMgBr | methylmagnesium bromide |
| DIBAL-H | diisobutylaluminium hydride |
| NaOMe | sodium methoxide |
| IBX | 2-iodoxybenzoic acid |
| t-BuONO | tert-butyl nitrite |
| LiBH₄ | lithium borohydride |
| NaBH₄ | sodium borohydride |
| EDCI | 1-ethyl-3-(3-dimethylaminopropyl)carbodiimide |
| HOBt | 1-hydroxybenzotriazole |
| HATU | 1-[Bis(dimethylamino)methylene]-1H-1,2,3- |
| | triazolo[4,5-b]pyridinium 3-oxid |
| | hexafluorophosphate |
| T3P | Propanephosphonic acid anhydride |
| T4P | 3-[2,6,8-trioxo-9-[(2R,3R,4R)-2,3,4,5- |
| | tetrahydroxypentyl]-3H-purin-7-yl]propyl |
| | dihydrogen phosphate |
| TCFH | N,N,N′,N′-tetramethylchloroformamidinium |
| | hexafluorophosphate |
| NMI | N-methylimidazole |
| Pd/C | palladium on carbon |
| Pd(PPh₃)₂Cl₂ | bis(triphenylphosphine)palladium(II) dichloride |
| Pd(dppf)Cl₂ | (1,1′-Bis(diphenylphosphino)ferrocene)palladium(II) |
| | dichloride |
| Pd(OAc)₂ | palladium(II) acetate |
| CPhos-Pd-G3 | [(2-Dicyclohexylphosphine-2′,6′-bis(N,N- |
| | dimethylamino)-1,1′-biphenyl)-2-(2′-amino-1,1′- |
| | biphenyl)] palladium(II) methanesulfonate |
| Pd₂(dba)₃ | Tris(dibenzylideneacetone)dipalladium (0) |
| Sphos | |
| CuI | copper iodide |
| CuBr₂ | copper (II) bromide |
| H₂ | hydrogen gas |
| SiO₂ | silica |

### General Experimental

In the following examples, the reagents and solvents were purchased from commercial sources (such as Alfa, Acros, AstaTech, CombiBlocks, Enamine, Sigma Aldrich, TCI, PharmaBock, Bide Pharmatech Ltd., Accela ChemBio, Aladdin, Shanghai Haohong Pharmaceutical Co., Ltd, Amkchem, Beijing Ouhe Technology Co., Ltd, Haoyuan Chemexpress Co., Ltd, Hualun, Coolpharm, Scochem, Titan, WuXi LabNetwork, and Energy Chemical, and used without further purification unless otherwise specified. Flash chromatography was performed on a CombiFlashRf 150 (ISCO) via column with silica gel particles of 100-200 mesh or a Biotage via column with silica gel particles of 200-300 mesh. HPLC was performed on an Agilent 1100 Liquid Chromatography (Agilent, USA) or a Shimadzu LC 20/20A or a Shimadzu LC-20AD/LH-40/FRC-40/GX-281 (Shimadzu, Japan). Supercritical fluid chromatography was performed on a Waters Prep SFC 150 AP/150 Mgn/80Q/200/350 system or a Waters Prep SFC Thar 80 (Waters, USA) or a Shimadzu-PRE-UC (Shimadzu, Japan). Analytical and preparative thin layer chromatography plates (TLC) were GF 254 (0.25, 0.5 mm thickness, Anhui Liangchen Silicon Material Co., Ltd., China) or GF 254 (0.15-0.2 mm thickness, Shanghai Anbang Company or Yucheng Chemical (Shanghai) Co., Ltd, China). Nuclear magnetic resonance (NMR) spectra were obtained on a Brucker AV-400 NMR or Bruker AVIII 500 MHz NMR (Bruker, Switzerland). Chemical shifts were reported in parts per million (ppm, 6) downfield from tetramethylsilane. Mass spectra were given with electrospray ionization (ESI) from a Waters LCT TOF Mass Spectrometer (Waters, USA). LC-MS was performed on an Agilent Prime-6125B/Agilent LC1260-MS6150/Agilent LC1260-MS6125B/Agilent LC1200-MS6110 (Agilent, USA) or a Shimadzu LC20-MS2020. Microwave reactions were run on an Initiator 2.5 Microwave Synthesizer (Biotage, Sweden).

### Intermediate Pyrazolo[1,5-a]pyridine-2-carbaldehyde

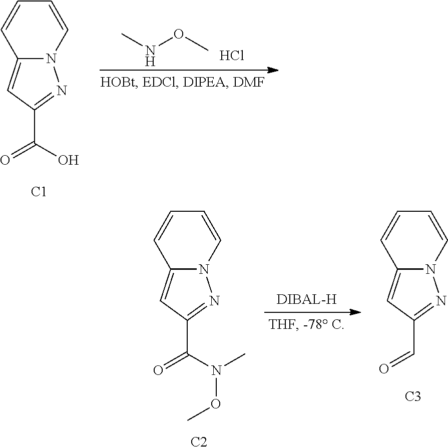

### Step 1: Preparation of N-methoxy-N-methyl-pyrazolo[1,5-a]pyridine-2-carboxamide (C2)

To a solution of pyrazolo[1,5-a]pyridine-2-carboxylic acid (C1) (12.5 g, 77.1 mmol) and N-methoxymethanamine (9.42 g, 96.6 mmol, 1.25 eq, HCl) in DMF (200 mL) was added EDCI (22.17 g, 116 mmol, 1.5 eq), DIPEA (29.89 g, 231 mmol, 40.3 mL, 3 eq) and HOBt (15.63 g, 116 mmol, 1.5 eq). The reaction mixture was stirred at rt for 12 hrs. Reaction progress was checked using TLC (PE:EtOAc=1:1). The reaction mixture was combined with the reaction mixture from another reaction performed using 12.5 g of C1 and concentrated to dryness. The residue was dissolved in DCM (200 mL), and the organic layer washed with sat. aq. Na₂CO₃ solution (100 mL) and brine (100 mL), dried over Na₂SO₄, filtered, and concentrated to dryness. The residue was purified by column chromatography on silica gel (PE:EtOAc=20:1 to 1:1) to give C2 (30.5 g, 96% yield). ¹H NMR (400 MHz, CDCl₃) δ 8.47-8.54 (m, 1H), 7.59 (d, 1H), 7.16 (ddd, 1H), 7.02 (s, 1H), 6.86 (td, 1H), 3.81 (s, 3H), 3.51 (s, 3H).

### Step 2: Preparation of pyrazolo[1,5-a]pyridine-2-carbaldehyde (C3)

To a solution of N-methoxy-N-methyl-pyrazolo[1,5-a]pyridine-2-carboxamide (C2) (15 g, 73.1 mmol) in THF (150 mL) was added DIBAL-H (1 M, 146.2 mL, 2 eq) dropwise at −78° C. under N₂, and then the reaction mixture was stirred at −78° C. for 2 hrs under N₂. Reaction progress was tracked using TLC (PE:EtOAc=1:1). The reaction mixture was combined with the reaction mixture from another reaction performed using 15 g of C₂ and was quenched by the addition of sat. aq. NH₄Cl solution (200 mL) slowly and stirred for 15 min. 1 M HCl solution was added until a clear solution was observed. The aqueous portion was extracted with EtOAc (250 mL×3), and the combined organic layer was washed with water (100 mL) and brine (100 mL), dried over Na₂SO₄, filtered, and concentrated to dryness. The residue was purified by column chromatography on silica gel (PE:EtOAc=20:1 to 1:1) to give C3 (14.2 g). ¹H NMR (400 MHz, DMSO-d₆) δ 10.14 (s, 1H), 8.81 (dd, 1H), 7.84 (d, 1H), 7.34 (t, 1H), 7.10-7.15 (m, 2H).

Intermediate 3-methylpyrazolo[1,5-a]pyridine-2-carbaldehyde was prepared following the general procedure described above for C3, starting with 3-methylpyrazolo[1,5-a]pyridine-2-carboxylic acid.

Intermediate 3-bromopyrazolo[1,5-a]pyridine-2-carbaldehyde was prepared following the general procedure described above for C3, starting with 3-bromopyrazolo[1,5-a]pyridine-2-carboxylic acid.

Intermediate 4-chloropyrazolo[1,5-a]pyridine-2-carbaldehyde was prepared following the general procedure described above for C3, starting with 4-chloropyrazolo[1,5-a]pyridine-2-carboxylic acid.

Intermediate 6-bromopyrazolo[1,5-a]pyridine-2-carbaldehyde was prepared following the general procedure described above for C3, starting with 6-bromopyrazolo[1,5-a]pyridine-2-carboxylic acid.

Intermediate 6-chloropyrazolo[1,5-a]pyridine-2-carbaldehyde was prepared following the general procedure described above for C3, starting with 6-chloropyrazolo[1,5-a]pyridine-2-carboxylic acid.

Intermediate 7-methylpyrazolo[1,5-a]pyridine-2-carbaldehyde was prepared following the general procedure described above for C3, starting with 7-methylpyrazolo[1,5-a]pyridine-2-carboxylic acid.

### Intermediate 4-fluoropyrazolo[1,5-a]pyridine-2-carbaldehyde

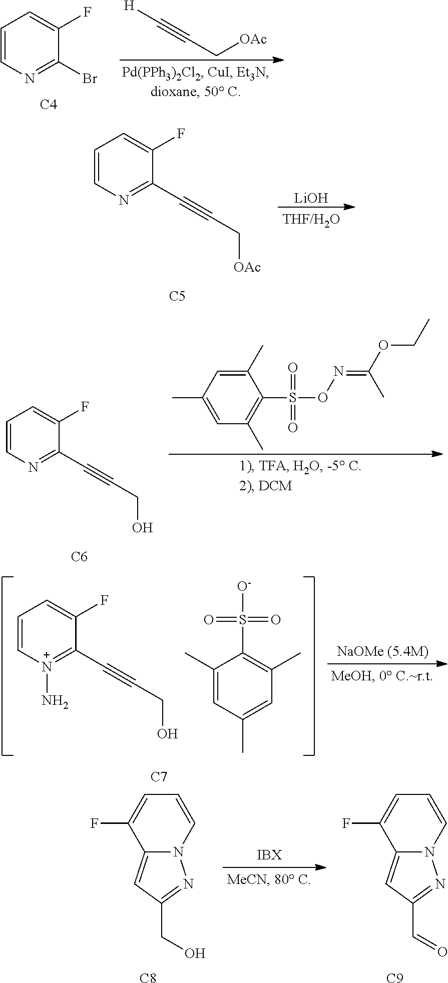

### Step 1: Preparation of 3-(3-fluoro-2-pyridyl)prop-2-yn-lyl acetate (C5)

A mixture of 2-bromo-3-fluoro-pyridine (C4) (30 g, 170 mmol), prop-2-yn-lyl acetate (23.41 g, 239 mmol, 1.4 eq), Pd(PPh₃)₂Cl₂ (5.98 g, 8.52 mmol, 0.05 eq), CuI (1.62 g, 8.52 mmol, 0.05 eq) and TEA (51.75 g, 511 mmol, 71.2 mL, 3 eq) in dioxane (300 mL) was degassed and purged with N₂ 3 times, and then the reaction mixture was stirred at 50° C. for 6 hrs under N₂ atmosphere. Reaction progress was checked using TLC (PE:EtOAc=5:1). The reaction mixture was concentrated to dryness. EtOAc (600 mL) was added to the residue, and the organic portion washed with water (300 mL) and brine (300 mL), dried over Na₂SO₄, filtered, and concentrated to dryness. The residue was purified by flash silica gel chromatography (ISCO®; 120 g SepaFlash® Silica Flash Column, eluent of 0-20% EtOAc/PE, gradient @120 mL/min) to give C5 (23.4 g, 71% yield). ¹H NMR (400 MHz, CDCl₃) δ 8.43 (d, 1H), 7.46 (td, 1H), 7.32 (dt, 1H), 4.99 (s, 2H), 2.16 (s, 3H).

### Step 2: Preparation of 3-(3-fluoro-2-pyridyl)prop-2-yn-1-ol (C6)

To a solution of 3-(3-fluoro-2-pyridyl)prop-2-ynyl acetate (C5) (23.4 g, 121 mmol) in THE (300 mL) and H₂O (150 mL) and was added LiOH·H₂O (5.34 g, 127 mmol, 1.05 eq) at 0° C. The reaction mixture was stirred at rt for 2 hrs. Reaction progress was checked using TLC (PE:EtOAc=1:1). EtOAc (600 mL) was added to the reaction mixture, and the organic layer washed with H₂O (400 mL) and brine (400 mL), dried over Na₂SO₄, filtered and concentrated to dryness to afford C6 (17.5 g), which was used without further purification. ¹H NMR (400 MHz, DMSO-d₆) δ 8.34-8.53 (m, 1H), 7.72-7.88 (m, 1H), 7.50 (dt, 1H), 5.54 (t, 1H), 4.38 (d, 2H).

### Step 3: Preparation of 1-amino-3-fluoro-2-(3-hydroxyprop-1-yn-1-yl)pyridin-1-ium2,4,6-trimethylbenzenesulfonate (C7)

To a mixture of H₂O (35 mL, 1.94 mol, 17.3 eq) and TFA (308.0 g, 2.70 mol, 200 mL, 24.0 eq) was added ethyl (1E)-N-(2,4,6-trimethylphenyl)sulfonyloxyethanimidate (33.70 g, 118 mmol, 1.05 eq) at 0° C., and the reaction mixture was stirred at 0° C. for 2 hrs. The reaction was quenched with ice-water (40 mL). The precipitate was filtered and washed with water (20 mL×2). The precipitate was then dissolved in DCM (200 mL) and dried over Na₂SO₄ and filtered. To the filtrate was added 3-(3-fluoro-2-pyridyl)prop-2-yn-1-ol (C6) (17 g, 112 mmol, 1 eq) at 0° C. The reaction mixture was warmed to rt for 16 hrs. Reaction progress was checked using TLC (PE:EtOAc=1:1). TBME (400 mL) was slowly added to the reaction mixture, and the precipitate was collected by filtration. The precipitate was rinsed with TBME (200 mL×2) to afford C7 (26 g), which was used without further purification. ¹H NMR (400 MHz, DMSO-d₆) δ 8.66-8.88 (m, 3H), 8.32 (t, 1H), 8.01 (dt, 1H), 6.75 (s, 2H), 4.58 (s, 2H), 3.17 (s, 1H), 2.49 (s, 6H), 2.17 (s, 3H).

### Step 4: Preparation of (4-fluoropyrazolo[1,5-a]pyridin-2-yl) methanol (C8)

To a solution of 1-amino-3-fluoro-2-(3-hydroxyprop-1-yn-1-yl)pyridin-1-ium 2,4,6-trimethylbenzenesulfonate (C7) (26 g, 71.0 mmol) in MeOH (250 mL) was added NaOMe (5.4 M, 26.3 mL, 2 eq) at 0° C. The reaction mixture was stirred at rt for 1 hr. The reaction progress was tracked by TLC (PE:EtOAc=1:1). The reaction mixture was quenched by the addition of ice-cold H₂O (200 mL), concentrated to remove most of the MeOH, and extracted with EtOAc (200 mL×3). The combined organic layer was washed with brine (400 mL), dried over Na₂SO₄, filtered, and concentrated to dryness to give C8 (7.5 g), which was used without further purification. ¹H NMR (400 MHz, CDCl₃) δ 8.27 (d, 1H), 6.62-6.88 (m, 3H), 4.94 (s, 2H).

### Step 5: Preparation of 4-fluoropyrazolo[1,5-a]pyridine-2-carbaldehyde (C9)

To a solution of (4-fluoropyrazolo[1,5-a]pyridin-2-yl)methanol (C8) (7.5 g, 45.1 mmol) in MeCN (150 mL) was added IBX (15.17 g, 54.2 mmol, 1.2 eq). The reaction mixture was stirred at 80° C. for 3 hrs. Reaction progress was checked using TLC (PE:EtOAc=5:1). The reaction mixture was filtered and concentrated to dryness. The residue was purified by flash silica gel chromatography (ISCO®; 40 g SepaFlash® Silica Flash Column, Eluent of 0-4% EtOAc/PE, gradient @40 mL/min) to give C9 (4 g, 54% yield). ¹H NMR (400 MHz, CDCl₃) δ 10.22 (s, 1H), 8.33-8.41 (m, 1H), 7.20 (d, 1H), 6.85-6.98 (m, 2H).

Intermediate 4-methylpyrazolo[1,5-a]pyridine-2-carbaldehyde was prepared following the general procedure described above for C9, starting with 2-bromo-3-methylpyridine.

Intermediate 4-(trifluoromethyl)pyrazolo[1,5-a]pyridine-2-carbaldehyde was prepared following the general procedure described above for C9, starting with 2-bromo-3-(trifluoromethyl)pyridine.

Intermediate 4-methoxypyrazolo[1,5-a]pyridine-2-carbaldehyde was prepared following the general procedure described above for C9, starting with 2-bromo-3-methoxypyridine.

Intermediate 5-methylpyrazolo[1,5-a]pyridine-2-carbaldehyde was prepared following the general procedure described above for C9, starting with 2-bromo-4-methylpyridine.

Intermediate 5-fluoropyrazolo[1,5-a]pyridine-2-carbaldehyde was prepared following the general procedure described above for C9, starting with 2-bromo-4-fluoropyridine.

Intermediate 5-(trifluoromethyl)pyrazolo[1,5-a]pyridine-2-carbaldehyde was prepared following the general procedure described above for C9, starting with 4-(trifluoromethyl)pyridine.

Intermediate 6-methylpyrazolo[1,5-a]pyridine-2-carbaldehyde was prepared following the general procedure described above for C9, starting with 2-bromo-5-methylpyridine.

Intermediate 6-fluoropyrazolo[1,5-a]pyridine-2-carbaldehyde was prepared following the general procedure described above for C9, starting with 2-bromo-5-fluoropyridine.

Intermediate 6-(trifluoromethyl)pyrazolo[1,5-a]pyridine-2-carbaldehyde was prepared following the general procedure described above for C9, starting with 2-bromo-5-(trifluoromethyl)pyridine.

Intermediate 7-(trifluoromethyl)pyrazolo[1,5-a]pyridine-2-carbaldehyde

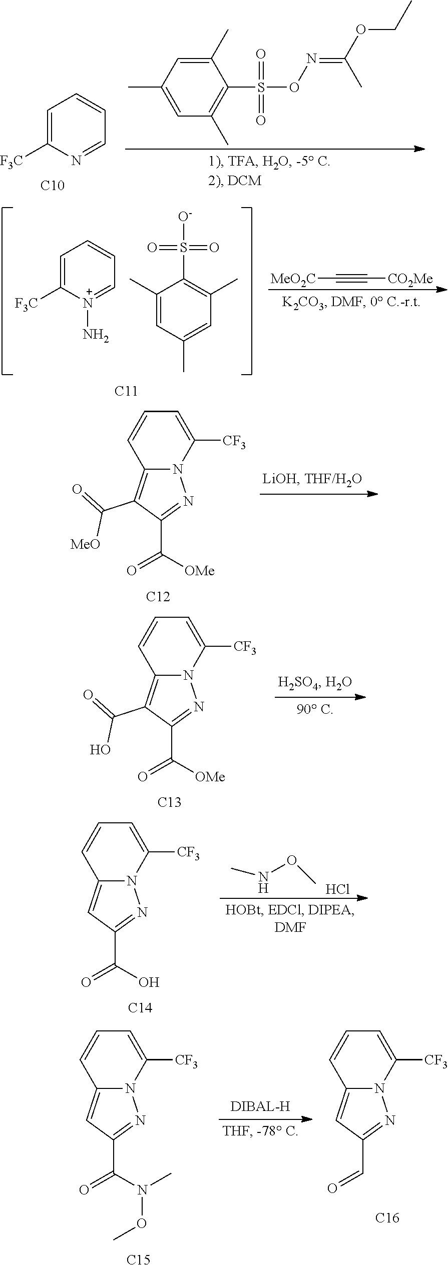

### Step 1: Preparation of 1-amino-2-(trifluoromethyl)pyridin-1-ium 2,4,6-trimethylbenzenesulfonate (C11)

To a mixture of TFA (215.60 g, 1.89 mol, 140 mL, 27.8 eq) and H₂O (20.00 g, 1.11 mol, 20.0 mL, 16.3 eq) was added ethyl (1E)-N-(2,4,6-trimethylphenyl) sulfonyloxyethanimidate (21.34 g, 74.8 mmol, 1.1 eq) at 0° C., and the reaction mixture was stirred at 0° C. for 2 hrs. The reaction was quenched by the addition of ice-water (100 mL), and the precipitate was filtered and rinsed with water (50 mL×2). The precipitate was then dissolved in DCM (140 mL), dried over Na₂SO₄ and filtered. To the filtrate was added 2-(trifluoromethyl)pyridine (C10) (10 g, 68.0 mmol, 7.87 mL) at 0° C. The reaction mixture was warmed to rt and stirred for 16 hrs. TBME (100 mL) was slowly added to the reaction mixture, and the precipitate was collected and rinsed with TBME (40 mL×3), and then dried in vacuo to give C11 (8.5 g, 35% yield), which was used without further purification. ¹H NMR (400 MHz, DMSO-d₆) δ 9.06 (d, 1H), 8.40-8.60 (m, 2H), 8.20-8.26 (m, 1H), 8.12 (s, 2H), 6.60 (s, 2H), 2.34 (s, 6H), 2.02 (s, 3H).

### Step 2: Preparation of dimethyl 7-(trifluoromethyl)pyrazolo[1,5-a]pyridine-2,3-dicarboxylate (C12)

To a mixture of 1-amino-2-(trifluoromethyl)pyridin-1-ium 2,4,6-trimethylbenzenesulfonate (C11) (8.5 g, 23.5 mmol) and K₂CO₃ (6.49 g, 46.9 mmol, 2 eq) in DMF (100 mL) was added dimethyl but-2-ynedioate (6.66 g, 46.9 mmol, 2 eq) at 0° C., The reaction mixture was warmed to rt and stirred for 16 hrs. Reaction progress was tracked by TLC (PE:EtOAc=1:1). Water (200 mL) was added, and the solution was stirred for 30 mins. The resulting precipitate was collected by vacuum filtration, rinsed with water, and dried. The residue was purified by flash silica gel chromatography (ISCO®; 80 g SepaFlash® Silica Flash Column, eluent of 0-40% EtOAc:PE @60 mL/min) to give C12 (2.85 g, 40% yield). ¹H NMR (400 MHz, DMSO-d₆) δ 8.40 (d, 1H), 7.74-7.96 (m, 2H), 3.85-3.96 (m, 6H).

### Step 3: Preparation of 2-(methoxycarbonyl)-7-(trifluoromethyl)pyrazolo[1,5-a]pyridine-3-carboxylic acid (C13)

To a solution of dimethyl 7-(trifluoromethyl)pyrazolo[1,5-a]pyridine-2,3-dicarboxylate (C12) (2.85 g, 9.43 mmol) in THF (20 mL) was added LiOH·H₂O (1.58 g, 37.7 mmol, 4 eq) in H₂O (20 mL), and the reaction mixture was stirred at rt for 16 hrs. Reaction progress was tracked using TLC (PE:EtOAc=1:1). The reaction mixture was concentrated in vacuo to remove most of THF, and the aqueous phase was adjusted to pH ˜2 with 4 M HCl. The precipitate was collected by filtration and then dried in vacuo to give C13 (2.25 g), which was used without further purification. ¹H NMR (400 MHz, DMSO-d₆) δ 8.43 (d, 1H), 7.85 (d, 1H), 7.71-7.76 (m, 1H).

### Step 4: Preparation of 7-(trifluoromethyl) pyrazolo[1,5-a]pyridine-2-carboxylic acid (C14)

A mixture of 2-(methoxycarbonyl)-7-(trifluoromethyl)pyrazolo[1,5-a]pyridine-3-carboxylic acid (C13) (2.25 g, 8.21 mmol) in H₂SO₄ (41.40 g, 422 mmol, 22.5 mL, 51.4 eq) and H₂O (11.25 g, 624 mmol, 11.3 mL, 76.1 eq) was stirred at 90° C. for 16 hrs. Reaction progress was tracked using LC-MS. The reaction mixture was cooled to rt, and water (60 mL) was added. The precipitate was collected by filtration and then dried in vacuo to give C14 (1.7 g), which was used without further purification. ¹H NMR (400 MHz, DMSO-d₆) δ 8.14 (d, 1H), 7.71 (d, 1H), 7.41-7.49 (m, 1H), 7.32 (s, 1H).

### Step 5: Preparation of N-methoxy-N-methyl-7-(trifluoromethyl)pyrazolo[1,5-a]pyridine-2-carboxamide (C15)

To a solution of 7-(trifluoromethyl)pyrazolo[1,5-a]pyridine-2-carboxylic acid (C14) (8.00 g, 34.8 mmol) and N-methoxymethanamine (10.17 g, 104 mmol, 3 eq, HCl) in DMF (80 mL) was added HOBt (7.05 g, 52.1 mmol, 1.5 eq), EDCI (10.00 g, 52.1 mmol, 1.5 eq) and DIPEA (13.48 g, 104 mmol, 18.2 mL, 3 eq). The mixture was stirred at rt for 16 hrs. Reaction progress was tracked using TLC (DCM:MeOH=10:1). DCM (150 mL) was added, and the organic portion washed with sat. aq. Na₂CO₃ solution (100 mL×2) and brine (100 mL), dried over Na₂SO₄, filtered, and concentrated to dryness. The residue was purified by flash silica gel chromatography (ISCO®; 80 g SepaFlash® Silica Flash Column, eluent of 0-10% MeOH:DCM @60 mL/min) to give C15 (6 g, 60% yield). ¹H NMR (400 MHz, DMSO-d₆) δ 8.11 (d, 1H), 7.67 (d, 1H), 7.37-7.49 (m, 1H), 7.21 (s, 1H), 3.77 (s, 3H), 2.50-2.52 (m, 3H); LCMS: m/z 274.1 [M+H]⁺.

### Step 6: Preparation of 7-(trifluoromethyl)pyrazolo[1,5-a]pyridine-2-carbaldehyde (C16)

To a solution of N-methoxy-N-methyl-7-(trifluoromethyl)pyrazolo[1,5-a]pyridine-2-carboxamide (C15) (6 g, 22.0 mmol) in THF (60 mL) was added DIBAL-H (1 M, 65.9 mL, 3 eq) dropwise at −78° C. under N₂, and then the reaction mixture was stirred at −78° C. for 2 hrs under N₂. Reaction progress was tracked using TLC (PE:EtOAc=5:1). The reaction mixture was quenched by the addition of sat. aq. NH₄Cl solution (150 mL), and then 4 M HCl solution (80 mL) was added. The aqueous portion was extracted with EtOAc (100 mL×2), and the combined organic layer was washed with water (80 mL) and brine (80 mL). The organic layer was dried over Na₂SO₄, filtered, and evaporated to dryness. The residue was purified by flash silica gel chromatography (ISCO®; 40 g SepaFlash® Silica Flash Column, eluent of 0-30% EtOAc:PE @40 mL/min) to give C16 (3.95 g, 68% yield). ¹H NMR (400 MHz, DMSO-d₆) δ 10.20 (s, 1H), 8.20 (d, 1H), 7.79 (d, 1H), 7.49 (dd, 1H), 7.40 (s, 1H).

Intermediate 7-chloropyrazolo[1,5-a]pyridine-2-carbaldehyde was prepared following the general procedure described above for C16, starting with 2-chloropyridine.

Intermediate 5-(trifluoromethyl)pyrazolo[1,5-a]pyridine-2-carbaldehyde was prepared following the general procedure described above for C16, starting with 4-(trifluoromethyl)pyridine.

Intermediate 7-fluoropyrazolo[1,5-a]pyridine-2-carbaldehyde

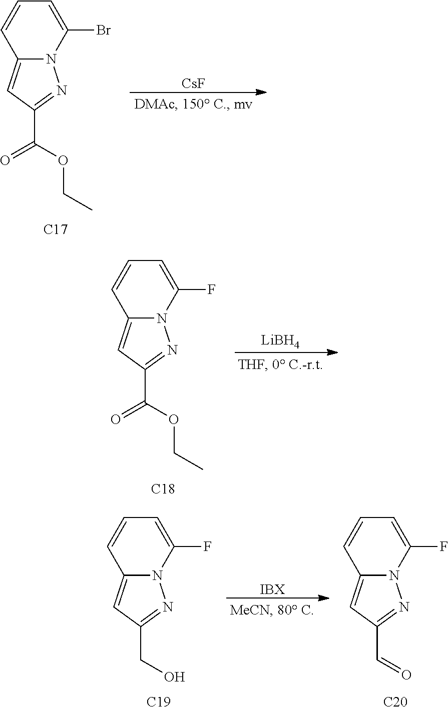

### Step 1: Preparation of ethyl 7-fluoropyrazolo[1,5-a]pyridine-2-carboxylate (C18)

To a solution of ethyl 7-bromopyrazolo[1,5-a]pyridine-2-carboxylate (C17) (1 g, 3.72 mmol) in DMAc (8 mL) was added CsF (1.69 g, 11.2 mmol, 411 μL, 3 eq), the reaction mixture was stirred at 150° C. for 1 hr under microwave. Reaction progress was tracked using LCMS. DCM (60 mL) was added to the reaction mixture, and the organic portion washed with water (30 mL) and brine (30 mL), dried over Na₂SO₄, filtered, and concentrated to dryness. The residue was purified by column chromatography (SiO₂, PE/EtOAc=1/0 to 4/1) to give C18 (450 mg, 29% yield). ¹H NMR (400 MHz, CDCl₃) δ 7.48 (d, 1H), 7.18-7.26 (m, 2H), 6.64 (ddd, 1H), 4.52 (q, 2H), 1.48 (t, 3H); LCMS: m/z 208.8 [M+H]⁺.

### Step 2: Preparation of (7-fluoropyrazolo[1,5-a]pyridin-2-yl)methanol (C19)

To a solution of ethyl 7-fluoropyrazolo[1,5-a]pyridine-2-carboxylate (C18) (450 mg, 2.16 mmol) in THF (4 mL) and EtOH (2 mL) was added LiBH₄ (260 mg, 11.9 mmol, 5.5 eq) at 0° C. The reaction mixture was stirred at rt for 2 hrs. Reaction progress was tracked using LCMS. The reaction mixture was quenched by the addition of sat. aq. NH₄Cl solution (10 mL), and the aqueous portion extracted with EtOAc (30 mL). The organic layer was washed with water (20 mL) and brine (20 mL), dried over Na₂SO₄, filtered, and concentrated to dryness to give C19 (360 mg), which was used without further purification. LCMS: m/z 167.0 [M+H]⁺.

### Step 3: Preparation of 7-fluoropyrazolo[1,5-a]pyridine-2-carbaldehyde (C20)

To a solution of (7-fluoropyrazolo[1,5-a]pyridin-2-yl)methanol (C19) (360 mg, 2.17 mmol) in MeCN (5 mL) was added IBX (971 mg, 3.47 mmol, 1.6 eq), and the reaction mixture was stirred at 80° C. for 1 hr. Reaction progress was tracked using LCMS. The reaction mixture was filtrated and concentrated to dryness to give C20 (355 mg), which was used without further purification. ¹H NMR (400 MHz, CDCl₃) δ 10.26-10.46 (m, 1H), 7.52 (dd, 1H), 7.22-7.28 (m, 1H), 7.16 (d, 1H), 6.69 (ddd, 1H); LCMS: m/z 165.0 [M+H]⁺.

### Preparation of Intermediate 4-(pyrazolo[1,5-a]pyridin-2-yl)-4,5,6,7-tetrahydro-1H-imidazo[4,5-c]pyridine

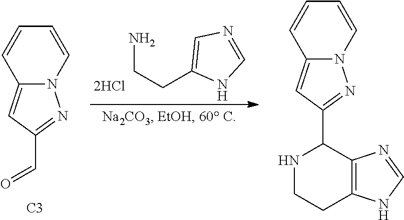

A mixture of pyrazolo[1,5-a]pyridine-2-carbaldehyde (C3) (9.2 g, 63.0 mmol), 2-(1H-imidazol-5-yl)ethanamine (15.06 g, 81.8 mmol, 2HCl salt) and K₂CO₃ (17.40 g, 126 mmol) in EtOH (300 mL) was stirred at 80° C. for 16 hrs under N₂. Reaction progress was checked using LCMS. The reaction mixture was combined with another reaction mixture performed using 5 g of C3 and filtered, and the filtrate was concentrated to dryness. The residue was purified by column chromatography on silica gel (DCM:MeOH=20:1 to 10:1) to give 4-pyrazolo[1,5-a]pyridin-2-yl-4,5,6,7-tetrahydro-1H-imidazo[4,5-c]pyridine (11.4 g). ¹H NMR (400 MHz, CD₃OD) δ 8.48 (d, 1H), 7.54-7.62 (m, 2H), 7.09-7.29 (m, 1H), 6.82-6.90 (m, 1H), 6.44 (s, 1H), 5.28 (s, 1H), 3.28 (dt, 1H), 3.04-3.12 (m, 1H), 2.73-2.80 (m, 2H); LC-MS: m/z 240.2 (M+H)⁺.

### Preparation of Intermediate 4-pyrazolo[1,5-a]pyridin-2-yl-1-tetrahydropyran-2-yl-4,5,6,7-tetrahydroimidazo[4,5-c]pyridine according to General Scheme 3

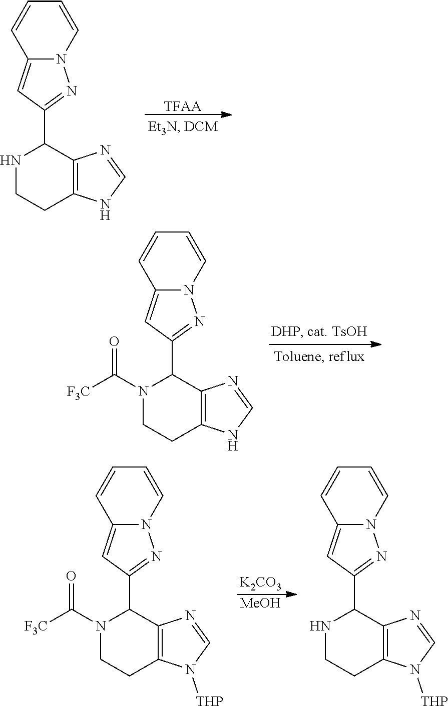

### Step 1: Preparation of 2,2,2-trifluoro-1-(4-pyrazolo[1,5-a]pyridin-2-yl-1,4,6,7-tetrahydroimidazo[4,5-c]pyridin-5-yl)ethenone

To a solution of 4-pyrazolo[1,5-a]pyridin-2-yl-4,5,6,7-tetrahydro-1H-imidazo[4,5-c]pyridine (1.2 g, 5.02 mmol) in DCM (25 mL), Et₃N (2.03 g, 20.1 mmol, 2.79 mL) and TFAA (2.74 g, 13.0 mmol, 1.81 mL) were added dropwise at 0° C. After addition was completed, the reaction mixture was stirred at rt for 15 hrs. DCM (50 mL) was added to the reaction mixture, and the organic portion washed with water (30 mL) and brine (30 mL), dried over Na₂SO₄, filtered, and concentrated to dryness. The residue was purified by flash silica gel chromatography (ISCO®; 4 g SepaFlash® Silica Flash Column, Eluent 0-10% DCM/MeOH @20 mL/min) to give 2,2,2-trifluoro-1-(4-pyrazolo[1,5-a]pyridin-2-yl-1,4,6,7-tetrahydroimidazo[4,5-c]pyridin-5-yl)ethanone (1.23 g, 69.6% yield). ¹H NMR (400 MHz, DMSO-d₆) δ 8.61-8.66 (m, 1H), 7.61-7.66 (m, 2H), 7.18-7.23 (m, 1H), 6.87 (td, 1H), 6.60 (s, 1H), 6.51-6.54 (m, 1H), 4.09 (br d, 1H), 3.67-3.77 (m, 0.2H), 3.38-3.45 (m, 0.8H), 2.69-2.85 (m, 2H); LCMS: m/z 336.1 [M+H]⁺.

### Step 2: Preparation of 2,2,2-trifluoro-1-(4-pyrazolo[1,5-a]pyridin-2-yl-1-tetrahydropyran-2-yl-6,7-dihydro-4H-imidazo[4,5-c]pyridin-5-yl)ethenone

To a solution of 2,2,2-trifluoro-1-(4-pyrazolo[1,5-a]pyridin-2-yl-1,4,6,7-tetrahydroimidazo[4,5-c]pyridin-5-yl)ethanone (1.23 g, 3.67 mmol) in toluene (16 mL) were added TsOH (63.2 mg, 367 μmol) and 3,4-dihydro-2H-pyran (926 mg, 11.0 mmol, 1.01 mL). The reaction mixture was stirred at 100° C. for 15 hrs. The reaction mixture was cooled to rt, and DCM was added (200 mL). The organic portion was washed with sat. aq. Na₂CO₃ solution (60 mL) and brine (50 mL), dried over Na₂SO₄, filtered, and concentrated to dryness. The residue was purified by column chromatography (SiO₂, DCM:MeOH=100:1 to 10:1) to give 2,2,2-trifluoro-1-(4-pyrazolo[1,5-a]pyridin-2-yl-1-tetrahydropyran-2-yl-6,7-dihydro-4H-imidazo[4,5-c]pyridin-5-yl)ethanone (1.1 g, 70.5% yield). ¹H NMR (400 MHz, DMSO-d₆) δ 8.58-8.66 (m, 1H), 7.80 (d, 1H), 7.63 (d, 1H), 7.20 (dd, 1H), 6.83-6.89 (m, 1H), 6.48-6.57 (m, 2H), 5.18-5.32 (m, 1H), 4.05-4.16 (m, 1H), 3.68-3.79 (m, 1H), 3.56-3.66 (m, 1H), 3.37-3.47 (m, 1H), 2.71-2.90 (m, 2H), 1.95 (br d, 2H), 1.42-1.54 (m, 4H); LCMS: m/z 420.2 [M+H]⁺.

### Step 3: 4-pyrazolo[1,5-a]pyridin-2-yl-1-tetrahydropyran-2-yl-4,5,6,7-tetrahydroimidazo[4,5-c]pyridine

To a solution of 2,2,2-trifluoro-1-(4-pyrazolo[1,5-a]pyridin-2-yl-1-tetrahydropyran-2-yl-6,7-dihydro-4H-imidazo[4,5-c]pyridin-5-yl)ethanone (1.1 g, 2.62 mmol) in MeOH (10 mL) was added K₂CO₃ (1.63 g, 11.8 mmol). The reaction mixture was stirred at 60° C. for 14 hrs. The reaction mixture was cooled to rt and filtered, and the filtrate was concentrated to dryness. The residue was purified by column chromatography (SiO₂, DCM:MeOH=100:1 to 10:1) to give 4-pyrazolo[1,5-a]pyridin-2-yl-1-tetrahydropyran-2-yl-4,5,6,7-tetrahydroimidazo[4,5-c]pyridine (580 mg, 67.8% yield). ¹H NMR (400 MHz, DMSO-d₆) δ 8.57 (br d, 1H), 7.54-7.63 (m, 2H), 7.13 (dd, 1H), 6.80 (br t, 1H), 6.33 (d, 1H), 5.14-5.24 (m, 1H), 5.03 (br s, 1H), 3.95 (br d, 1H), 3.56-3.67 (m, 1H), 3.05-3.14 (m, 1H), 2.84-3.00 (m, 2H), 1.87-2.03 (m, 3H), 1.59-1.80 (m, 2H), 1.54 (br d, 2H); LCMS: m/z 324.2 [M+H]⁺.

### Preparation of Intermediate methyl 5-(4-(4-fluoropyrazolo[1,5-a]pyridin-2-yl)-1,4,6,7-tetrahydro-5H-imidazo[4,5-c]pyridin-5-yl)pyrazine-2-carboxylate and enantiomers methyl (R)-5-(4-(4-fluoropyrazolo[1,5-a]pyridin-2-yl)-1,4,6,7-tetrahydro-5H-imidazo[4,5-c]pyridin-5-yl)pyrazine-2-carboxylate and methyl (S)-5-(4-(4-fluoropyrazolo[1,5-a]pyridin-2-yl)-1,4,6,7-tetrahydro-5H-imidazo[4,5-c]pyridin-5-yl)pyrazine-2-carboxylate

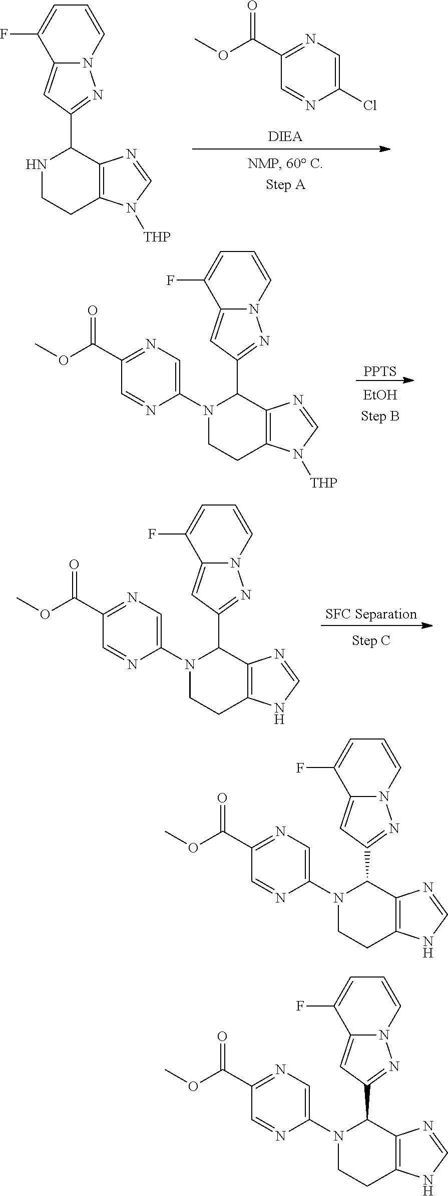

### Step A. Synthesis of methyl 5-(4-(4-fluoropyrazolo[1,5-a]pyridin-2-yl)-1-(tetrahydro-2H-pyran-2-yl)-1,4,6,7-tetrahydro-5H-imidazo[4,5-c]pyridin-5-yl)pyrazine-2-carboxylate

To a solution of 4-(4-fluoropyrazolo[1,5-a]pyridin-2-yl)-1-(3,4,5,6-tetrahydro-2H-pyran-2-yl)-4,5,6,7-tetrahydroimidazo[5,4-c]pyridine (10.0 g, 29.3 mmol) in NMP (100 mL) was added methyl 5-chloropyrazine-2-carboxylate (10.1 g, 58.6 mmol) and DIEA (14.5 mL, 87.9 mmol). The reaction mixture was stirred at 60° C. for 18 hrs under N₂. The reaction mixture was cooled to rt, and EtOAc (100 mL) and water (100 mL) were added. The aqueous portion was extracted with EtOAc (100 mL×3). The combined organic layer was washed with brine (100 mL×5), dried over Na₂SO₄, filtered, and concentrated to dryness. The residue was purified with silica gel (0-5% MeOH in DCM) to afford 5-(4-(4-fluoropyrazolo[1,5-a]pyridin-2-yl)-1-(tetrahydro-2H-pyran-2-yl)-1,4,6,7-tetrahydro-5H-imidazo[4,5-c]pyridin-5-yl)pyrazine-2-carboxylate (11 g, 79% yield). LC-MS: m/z 478.2 [M+H]⁺.

### Step B. Synthesis of methyl 5-(4-(4-fluoropyrazolo[1,5-a]pyridin-2-yl)-1,4,6,7-tetrahydro-5H-imidazo[4,5-c]pyridin-5-yl)pyrazine-2-carboxylate

To a solution of methyl 5-[4-(4-fluoropyrazolo[1,5-a]pyridin-2-yl)-1-(3,4,5,6-tetrahydro-2H-pyran-2-yl)-4,5,6,7-tetrahydroimidazo[5,4-c]pyridin-5-yl]pyrazine-2-carboxylate (11.0 g, 23.0 mmol) in EtOH (100 mL) was added PPTS (17.4 g, 69.1 mmol). The reaction mixture was stirred at 85° C. for 18 hrs. The reaction mixture was concentrated to dryness, and the residue purified with prep-HPLC with a C18 column (mobile phase: 0-44% MeCN in deionized water (0.1% NH₄HCO₃)) to afford methyl 5-(4-(4-fluoropyrazolo[1,5-a]pyridin-2-yl)-1,4,6,7-tetrahydro-5H-imidazo[4,5-c]pyridin-5-yl)pyrazine-2-carboxylate (7.5 g, 83% yield). LC-MS: m/z 394.1 [M+H]⁺.

### Step C: SFC Separation

Methyl 5-(4-(4-fluoropyrazolo[1,5-a]pyridin-2-yl)-1,4,6,7-tetrahydro-5H-imidazo[4,5-c]pyridin-5-yl)pyrazine-2-carboxylate (6.5 g, 16.5 mmol) was separated by SFC (column: CHIRALPAK IH (4.6*100 mm, 5 μm); mobile phase: [CO₂-MeOH(0.05% DEA)]; B %:40%, isocratic elution mode) to afford Enantiomer #1 (3 g, 46% yield, Rt=1.374 min) and Enantiomer #2 (2.8 g, 43% yield, Rt=2.184 min).

Enantiomer #1, methyl (R)-5-(4-(4-fluoropyrazolo[1,5-a]pyridin-2-yl)-1,4,6,7-tetrahydro-5H-imidazo[4,5-c]pyridin-5-yl)pyrazine-2-carboxylate: ¹H NMR (400 MHz, DMSO-d₆) δ 12.04 (s, 1H), 8.70 (s, 1H), 8.64 (s, 1H), 8.53 (d, 1H), 7.59 (s, 1H), 7.20-7.01 (m, 1H), 6.93-6.77 (m, 2H), 6.74 (s, 1H), 5.05-4.64 (m, 1H), 3.82 (s, 3H), 3.66-3.51 (m, 1H), 2.97-2.83 (m, 1H), 2.82-2.70 (m, 1H); LC-MS: m/z 394.1 [M+H]⁺; 100% ee.

Enantiomer #2, methyl (S)-5-(4-(4-fluoropyrazolo[1,5-a]pyridin-2-yl)-1,4,6,7-tetrahydro-5H-imidazo[4,5-c]pyridin-5-yl)pyrazine-2-carboxylate: ¹H NMR (400 MHz, DMSO-d) δ 12.04 (s, 1H), 8.70 (s, 1H), 8.64 (s, 1H), 8.53 (d, 1H), 7.59 (s, 1H), 7.20-7.01 (m, 1H), 6.93-6.77 (m, 2H), 6.74 (s, 1H), 5.05-4.64 (m, 1H), 3.82 (s, 3H), 3.66-3.51 (m, 1H), 2.97-2.83 (m, 1H), 2.82-2.70 (m, 1H); LC-MS: m/z 394.1 [M+H]⁺; 100% ee.

### Preparation of Examples 89 and 90 According to General Scheme 2, Method C2

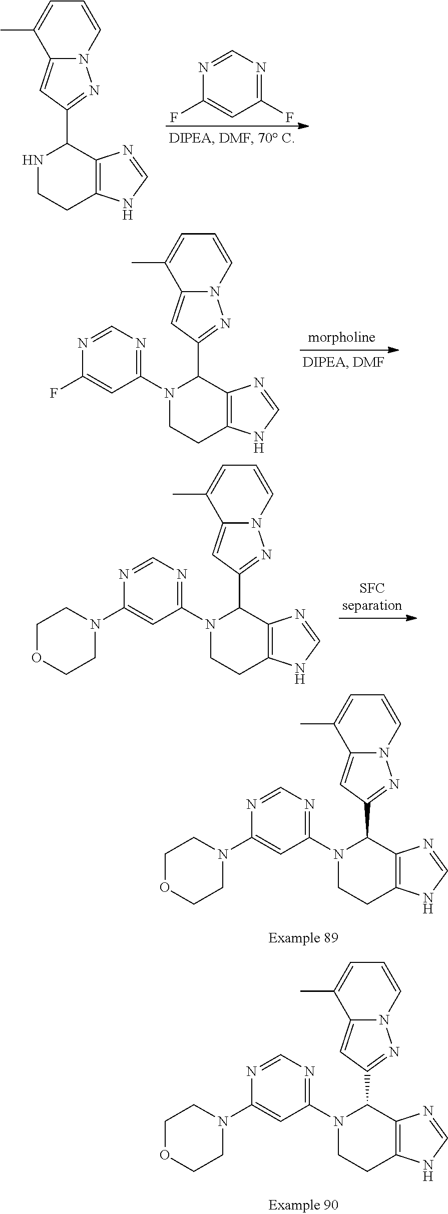

### Step 1: Preparation of 5-(6-fluoropyrimidin-4-yl)-4-(4-methylpyrazolo[1,5-a]pyridin-2-yl)-1,4,6,7-tetrahydroimidazo[4,5-c]pyridine

To a solution of 4-(4-methylpyrazolo[1,5-a]pyridin-2-yl)-4,5,6,7-tetrahydro-1H-imidazo [4,5-c]pyridine (500 mg, 1.97 mmol) in DMF (5 mL) was added DIPEA (765 mg, 5.92 mmol, 1.03 mL) and 4,6-difluoropyrimidine (275 mg, 2.37 mmol). The reaction mixture was stirred at 70° C. for 14 hrs, and then allowed to cool to rt. Sat. aq. Na₂CO₃ solution (15 mL) was added, and the aqueous portion extracted with DCM (30 mL×3). The combined organic layer was dried over Na₂SO₄, filtered, and concentrated to dryness. The residue was purified by flash silica gel chromatography (ISCO®; 20 g SepaFlash® Silica Flash Column, Eluent of 0-4.2% MeOH/DCM @35 mL/min) to give 5-(6-fluoropyrimidin-4-yl)-4-(4-methylpyrazolo[1,5-a]pyridin-2-yl)-1,4,6,7-tetrahydroimidazo[4,5-c]pyridine (426 mg, 43.9% yield). ¹H NMR (400 MHz, METHANOL-d₄) δ 8.25-8.35 (m, 2H), 7.67 (s, 1H), 6.97 (br d, 2H), 6.76 (t, 1H), 6.65 (s, 1H), 6.53 (s, 1H), 4.49-4.82 (m, 1H), 3.55-3.66 (m, 1H), 2.87-2.99 (m, 1H), 2.69-2.82 (m, 1H), 2.42 (s, 3H); LCMS: m/z 350.1 [M+H]⁺.

### Step 2: Preparation of 4-[6-[4-(4-methylpyrazolo[1,5-a]pyridin-2-yl)-1,4,6,7-tetrahydroimidazo[4,5-c]pyridin-5-yl]pyrimidin-4-yl]morpholine

To a solution of 5-(6-fluoropyrimidin-4-yl)-4-(4-methylpyrazolo[1,5-a]pyridin-2-yl)-1,4,6,7-tetrahydroimidazo[4,5-c]pyridine (120 mg, 343 μmol) and morpholine (32.9 mg, 378 μmol, 33.3 μL) in DMF (2 mL) was added DIPEA (133 mg, 1.03 mmol, 179 μL). The reaction mixture was stirred at rt for 15 hrs and then purified by prep-HPLC (column: Phenomenex C18 75*30 mm*3 um; mobile phase: [water(NH₃H₂O+NH₄HCO₃)-ACN];gradient:25%-55% B over 7 min) to give 4-[6-[4-(4-methylpyrazolo[1,5-a]pyridin-2-yl)-1,4,6,7-tetrahydroimidazo[4,5-c]pyridin-5-yl]pyrimidin-4-yl]morpholine (60 mg, 41.9% yield). LCMS: m/z 417.2 [M+H]⁺.

### Step 3: SFC Separation

4-[6-[4-(4-methylpyrazolo[1,5-a]pyridin-2-yl)-1,4,6,7-tetrahydroimidazo [4,5-c]pyridin-5-yl]pyrimidin-4-yl]morpholine (60 mg) was separated by SFC (column: DAICEL CHIRALPAK IG (250 mm*30 mm, 10 μm); mobile phase: [CO₂-EtOH(0.1% NH₃H₂O)];B %:60%, isocratic elution mode) to give Enantiomer #1 (13.4 mg, Rt=0.873 min, 22.0% yield) and Enantiomer #2 (14.6 mg, Rt=1.470 min, 23.8% yield).

Enantiomer #1 (Example 89), (R)-4-(6-(4-(4-methylpyrazolo[1,5-a]pyridin-2-yl)-1,4,6,7-tetrahydro-5H-imidazo[4,5-c]pyridin-5-yl)pyrimidin-4-yl)morpholine: ¹H NMR (400 MHz, METHANOL-d₄) δ 8.30 (d, 1H), 8.14 (s, 1H), 7.61 (s, 1H), 6.95 (d, 1H), 6.75 (br t, 2H), 6.48 (s, 1H), 6.15 (s, 1H), 4.62-4.70 (m, 1H), 3.70-3.81 (m, 4H), 3.47-3.59 (m, 5H), 2.82-2.99 (m, 1H), 2.69 (br dd, 1H), 2.41 (s, 3H); LCMS: m/z 417.2 [M+H]⁺; SFC: 99.4% ee.

Enantiomer #2 (Example 90), (S)-4-(6-(4-(4-methylpyrazolo[1,5-a]pyridin-2-yl)-1,4,6,7-tetrahydro-5H-imidazo[4,5-c]pyridin-5-yl)pyrimidin-4-yl)morpholine: ¹H NMR (400 MHz, METHANOL-d₄) δ 8.30 (d, 1H), 8.14 (s, 1H), 7.62 (s, 1H), 6.96 (d, 1H), 6.75 (t, 2H), 6.48 (s, 1H), 6.15 (s, 1H), 4.62-4.71 (m, 1H), 3.66-3.84 (m, 4H), 3.46-3.59 (m, 5H), 2.86-2.98 (m, 1H), 2.69 (br dd, 1H), 2.41 (s, 3H); LCMS: m/z 417.2 [M+H]⁺; SFC: 98.7% ee.

### Preparation of Examples 117, 118, 119, and 120 according to General Scheme 2, Method C1

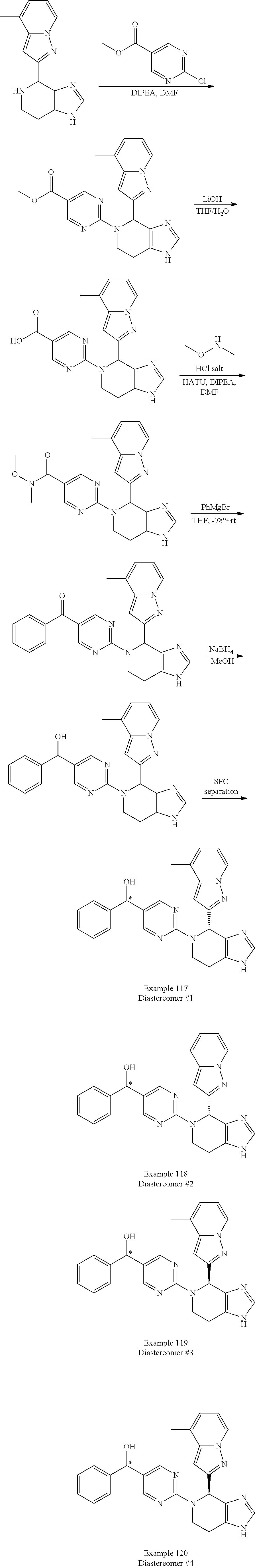

### Step 1: Preparation of methyl 2-[4-(4-methylpyrazolo[1,5-a]pyridin-2-yl)-1,4,6,7-tetrahydroimidazo[4,5-c]pyridin-5-yl]pyrimidine-5-carboxylate

To a solution of 4-(4-methylpyrazolo[1,5-a]pyridin-2-yl)-4,5,6,7-tetrahydro-1H-imidazo [4,5-c]pyridine (1 g, 3.95 mmol) and methyl 2-chloropyrimidine-5-carboxylate (749 mg, 4.34 mmol) in DMF (10 mL) was added DIPEA (1.53 g, 11.8 mmol, 2.06 mL). The reaction mixture was stirred at rt for 12 hrs. DCM (100 mL) was added to the reaction mixture, and the organic portion washed with H₂O (60 mL) and brine (60 ml), dried over Na₂SO₄, filtered, and concentrated to dryness. The residue was purified by flash silica gel chromatography (ISCO®; 12 g SepaFlash® Silica Flash Column, Eluent of 0-10% MeOH/DCM @30 mL/min) to give methyl 2-[4-(4-methylpyrazolo[1,5-a]pyridin-2-yl)-1,4,6,7-tetrahydroimidazo[4,5-c]pyridin-5-yl]pyrimidine-5-carboxylate (1.37 g, 87.6% yield). LCMS: m/z 390.2 [M+H]⁺.

### Step 2: Preparation of 2-[4-(4-methylpyrazolo[1,5-a]pyridin-2-yl)-1,4,6,7-tetrahydroimidazo[4,5-c]pyridin-5-yl]pyrimidine-5-carboxylic acid

To a solution of methyl 2-[4-(4-methylpyrazolo[1,5-a]pyridin-2-yl)-1,4,6,7-tetrahydroimidazo [4,5-c]pyridin-5-yl]pyrimidine-5-carboxylate (1.37 g, 3.52 mmol) in THF (20 mL) was added LiOH·H₂O (295 mg, 7.04 mmol) in H₂O (10 mL), and the reaction mixture was stirred at rt for 1 hr. The reaction mixture was concentrated under reduced pressure, and 4M HCl was added to the aqueous phase until it was approximately pH 5. The precipitate was filtered and dried in vacuo to afford 2-[4-(4-methylpyrazolo[1,5-a]pyridin-2-yl)-1,4,6,7-tetrahydroimidazo[4,5-c]pyridin-5-yl]pyrimidine-5-carboxylic acid (1.32 g). LCMS: m/z 376.2 [M+H]⁺.

### Step 3: Preparation of N-methoxy-N-methyl-2-[4-(4-methylpyrazolo[1,5-a]pyridin-2-yl)-1,4,6,7-tetrahydroimidazo[4,5-c]pyridin-5-yl]pyrimidine-5-carboxamide

To a solution of 2-[4-(4-methylpyrazolo[1,5-a]pyridin-2-yl)-1,4,6,7-tetrahydroimidazo[4,5-c]pyridin-5-yl]pyrimidine-5-carboxylic acid (1.3 g, 3.46 mmol) and N-methoxymethanamine (676 mg, 6.93 mmol, HCl salt) in DMF (10 mL) was added HATU (1.98 g, 5.19 mmol) and DIPEA (1.79 g, 13.9 mmol, 2.41 mL). The reaction mixture was stirred at rt for 1 hr. DCM (100 mL) was added to the reaction mixture, and the organic portion washed with sat. aq. NaHCO₃ solution (50 mL) and brine (50 mL), dried over Na₂SO₄, filtered, and concentrated to dryness. The residue was purified by flash silica gel chromatography (ISCO®; 24 g SepaFlash® Silica Flash Column, Eluent of 0-10% MeOH/DCM @30 mL/min) to give N-methoxy-N-methyl-2-[4-(4-methylpyrazolo[1,5-a]pyridin-2-yl)-1,4,6,7-tetrahydroimidazo[4,5-c]pyridin-5-yl]pyrimidine-5-carboxamide (0.8 g, 50.2% yield). ¹H NMR (400 MHz, DMSO-d₆) δ 8.74-8.76 (m, 2H), 8.45 (d, 1H), 7.70 (s, 1H), 7.11 (s, 1H), 6.97 (d, 1H), 6.73-6.77 (m, 1H), 6.53 (s, 1H), 5.06 (br dd, 1H), 3.63 (s, 3H), 3.51 (br d, 1H), 3.26 (s, 3H), 2.70-2.82 (m, 2H), 2.38 (s, 3H); LCMS: m/z 419.3 [M+H]⁺.

### Step 4: Preparation of [2-[4-(4-methylpyrazolo[1,5-a]pyridin-2-yl)-1,4,6,7-tetrahydroimidazo[4,5-c]pyridin-5-yl]pyrimidin-5-yl]-phenyl-methanone

A solution of N-methoxy-N-methyl-2-[4-(4-methylpyrazolo[1,5-a]pyridin-2-yl)-1,4,6,7-tetrahydroimidazo[4,5-c]pyridin-5-yl]pyrimidine-5-carboxamide (800 mg, 1.91 mmol) in THF (20 mL) was degassed and purged with N₂ for 3 times, and then bromo(phenyl)magnesium (1 M, 9.56 mL) was added dropwise at −78° C. The reaction mixture was stirred at rt for 2 hrs. Sat. aq. NH₄Cl solution (80 mL) was added, and the aqueous portion was extracted with EtOAc (100 mL). The organic layer was washed with water (40 mL) and brine (40 mL), dried over Na₂SO₄, filtered, and concentrated to dryness. The residue was purified by flash silica gel chromatography (ISCO®; 12 g SepaFlash® Silica Flash Column, Eluent of 0-9% MeOH/DCM @25 mL/min) to give [2-[4-(4-methylpyrazolo[1,5-a]pyridin-2-yl)-1,4,6,7-tetrahydroimidazo[4,5-c]pyridin-5-yl]pyrimidin-5-yl]-phenyl-methanone (0.5 g, 54.4% yield). LCMS: m/z 436.2 [M+H]⁺.

### Step 5: Preparation of [2-[4-(4-methylpyrazolo[1,5-a]pyridin-2-yl)-1,4,6,7-tetrahydroimidazo[4,5-c]pyridin-5-yl]pyrimidin-5-yl]-phenyl-methanol

To a solution of [2-[4-(4-methylpyrazolo[1,5-a]pyridin-2-yl)-1,4,6,7-tetrahydroimidazo [4,5-c]pyridin-5-yl]pyrimidin-5-yl]-phenyl-methanone (0.3 g, 689 μmol) in MeOH (10 mL) was added NaBH₄ (52.1 mg, 1.38 mmol), and the reaction mixture was stirred at rt for 1 hr. Water (30 mL) was added at 0° C., and the aqueous portion extracted with DCM (40 mL×2). The combined organic layer was washed with brine (30 mL), dried over Na₂SO₄, filtered, and concentrated to dryness. The residue was purified by flash silica gel chromatography (ISCO®; 12 g SepaFlash® Silica Flash Column, Eluent of 0-10% MeOH/DCM @30 mL/min) to give [2-[4-(4-methylpyrazolo[1,5-a]pyridin-2-yl)-1,4,6,7-tetrahydroimidazo[4,5-c]pyridin-5-yl]pyrimidin-5-yl]-phenyl-methanol (260 mg, 80.5% yield). LCMS: m/z 438.2 [M+H]⁺.

### Step 6: SFC Separation

[2-[4-(4-methylpyrazolo[1,5-a]pyridin-2-yl)-1,4,6,7-tetrahydroimidazo[4,5-c]pyridin-5-yl]pyrimidin-5-yl]-phenyl-methanol (0.26 g) was separated by SFC (column: DAICEL CHIRALPAK IG (250 mm*30 mm, 10 μm); mobile phase: [CO₂-MeOH(0.1% NH₃H₂O)];B %:60%, isocratic elution mode) to give a mixture of Diastereomer #1 and Diastereomer #2 (80 mg, Rt=1.379 min), Diastereomer #3 (38.1 mg, Rt=1.712 min, 14.5% yield) and Diastereomer #4 (39.9 mg, Rt=2.924 min, 14.6% yield). The mixture of Diastereomer #1 and Diastereomer #2 was further separated by SFC (column: DAICEL CHIRALPAK AD (250 mm*30 mm, 10 μm); mobile phase: [CO₂-i-PrOH (0.1% NH₃H₂O)]; B %:45%, isocratic elution mode) to give Diastereomer #1 (30 mg, Rt=3.906 min, 37.2% yield) and Diastereomer #2 (30 mg, Rt=5.283 min, 37.2% yield).

Diastereomer #1 (Example 117), (2-((S)-4-(4-methylpyrazolo[1,5-a]pyridin-2-yl)-1,4,6,7-tetrahydro-5H-imidazo[4,5-c]pyridin-5-yl)pyrimidin-5-yl)(phenyl)methanol: ¹H NMR (400 MHz, METHANOL-d₄) δ 8.31-8.37 (m, 2H), 8.23-8.30 (m, 1H), 7.62 (s, 1H), 7.33-7.45 (m, 4H), 7.24-7.31 (m, 1H), 7.18 (s, 1H), 6.95 (d, 1H), 6.74 (t, 1H), 6.51 (s, 1H), 5.74 (s, 1H), 5.01 (dd, 1H), 3.41-3.50 (m, 1H), 2.83-2.95 (m, 1H), 2.68 (dd, 1H), 2.42 (s, 3H); LCMS: m/z 438.2 [M+H]⁺; SFC: 100% ee.

Diastereomer #2 (Example 118), (2-((S)-4-(4-methylpyrazolo[1,5-a]pyridin-2-yl)-1,4,6,7-tetrahydro-5H-imidazo[4,5-c]pyridin-5-yl)pyrimidin-5-yl)(phenyl)methanol: ¹H NMR (400 MHz, METHANOL-d₄) δ 8.34 (s, 2H), 8.27 (d, 1H), 7.62 (s, 1H), 7.34-7.45 (m, 4H), 7.23-7.31 (m, 1H), 7.18 (s, 1H), 6.95 (d, 1H), 6.74 (t, 1H), 6.51 (s, 1H), 5.74 (s, 1H), 5.01 (br dd, 1H), 3.42-3.51 (m, 1H), 2.83-2.95 (m, 1H), 2.68 (dd, 1H), 2.41 (s, 3H); LCMS: m/z 438.2 [M+H]⁺; SFC: 98.8% ee.

Diastereomer #3 (Example 119), (2-((R)-4-(4-methylpyrazolo[1,5-a]pyridin-2-yl)-1,4,6,7-tetrahydro-5H-imidazo[4,5-c]pyridin-5-yl)pyrimidin-5-yl)(phenyl)methanol: ¹H NMR (400 MHz, METHANOL-d₄) δ 8.31-8.38 (m, 2H), 8.27 (d, 1H), 7.62 (s, 1H), 7.33-7.45 (m, 4H), 7.26-7.31 (m, 1H), 7.18 (s, 1H), 6.90-6.98 (m, 1H), 6.70-6.77 (m, 1H), 6.46-6.53 (m, 1H), 5.74 (s, 1H), 4.98-5.03 (m, 1H), 3.41-3.51 (m, 1H), 2.82-2.94 (m, 1H), 2.63-2.72 (m, 1H), 2.41 (s, 3H); LCMS: m/z 438.2 [M+H]⁺; SFC: 88.4% ee.

Diastereomer #4 (Example 120), (2-((R)-4-(4-methylpyrazolo[1,5-a]pyridin-2-yl)-1,4,6,7-tetrahydro-5H-imidazo[4,5-c]pyridin-5-yl)pyrimidin-5-yl)(phenyl)methanol: ¹H NMR (400 MHz, METHANOL-d₄) δ 8.32 (s, 2H), 8.24 (d, 1H), 7.61 (s, 1H), 7.31-7.42 (m, 4H), 7.21-7.29 (m, 1H), 7.16 (s, 1H), 6.89-6.95 (m, 1H), 6.68-6.74 (m, 1H), 6.49 (s, 1H), 5.68-5.74 (m, 1H), 4.99 (br dd, 1H), 3.38-3.49 (m, 1H), 2.81-2.92 (m, 1H), 2.66 (br dd, 1H), 2.39 (s, 3H); LCMS: m/z 438.2 [M+H]⁺; SFC: 94.9% ee.

### Preparation of Examples 107, 108, 109, and 110 according to General Scheme 2, Method C1

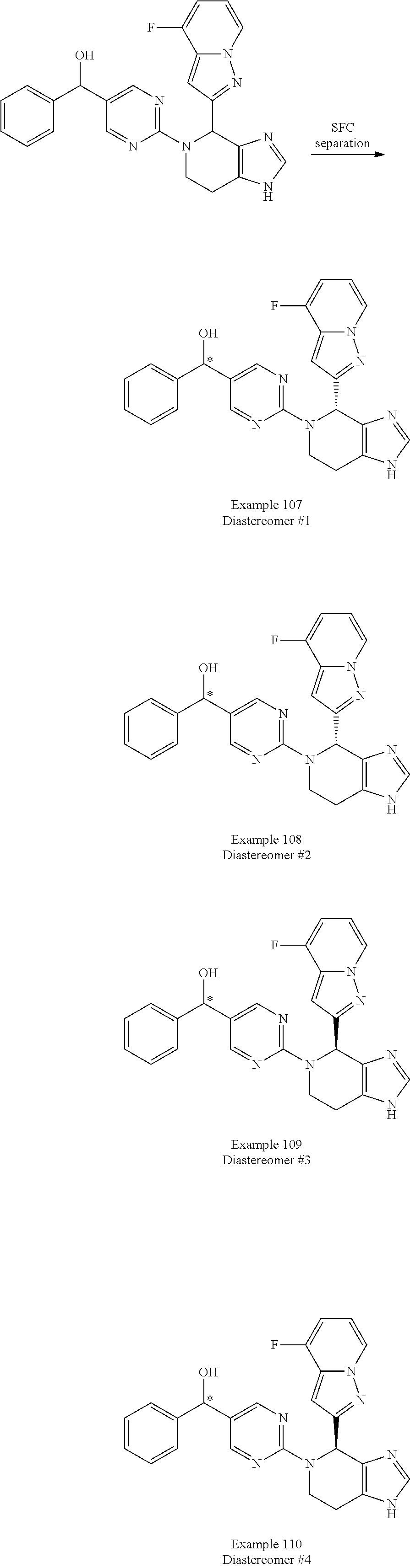 [SMILES: OC(c1ccccc1)c1cnc(N2CCc3[nH]cnc3C2c2cc3c(F)cccn3n2)nc1 ; OC(c1ccccc1)c1cnc(N2CCc3[nH]cnc3[C@H]2c2cc3c(F)cccn3n2)nc1 ; OC(c1ccccc1)c1cnc(N2CCc3[nH]cnc3[C@H]2c2cc3c(F)cccn3n2)nc1 ; OC(c1ccccc1)c1cnc(N2CCc3[nH]cnc3[C@@H]2c2cc3c(F)cccn3n2)nc1 ; OC(c1ccccc1)c1cnc(N2CCc3[nH]cnc3[C@@H]2c2cc3c(F)cccn3n2)nc1]

Racemate [2-[4-(4-fluoropyrazolo[1,5-a]pyridin-2-yl)-1,4,6,7-tetrahydroimidazo[4,5-c]pyridin-5-yl]pyrimidin-5-yl]-phenyl-methanol was prepared in accordance with the procedures described above for Examples 117, 118, 119, and 120, starting with 4-(4-fluoropyrazolo[1,5-a]pyridin-2-yl)-4,5,6,7-tetrahydro-1H-imidazo [4,5-c]pyridine.

Racemate [2-[4-(4-fluoropyrazolo[1,5-a]pyridin-2-yl)-1,4,6,7-tetrahydroimidazo[4,5-c]pyridin-5-yl]pyrimidin-5-yl]-phenyl-methanol (182 mg) was separated by SFC (column: DAICEL CHIRALPAK IG (250 mm*30 mm, 10 μm); mobile phase: [CO₂-MeOH(0.1% NH₃H₂O)];B %:55%, isocratic elution mode) to give a mixture of Diastereomer #1 and Diastereomer #2 (70 mg, Rt=1.102 min), Diastereomer #3 (25.5 mg, Rt=1.514 min, 13.7% yield), and Diastereomer #4 (30.6 mg, Rt=2.302 min, 15.9% yield). The mixture of Diastereomer #1 and Diastereomer #2 was further separated by SFC (column: DAICEL CHIRALPAK AD (250 mm*30 mm, 10 μm); mobile phase: [CO₂-MeOH (0.1% NH₃H₂O)]; B %:35%, isocratic elution mode) to give Diastereomer #1 (16.1 mg, Rt=2.390 min, 22.8% yield) and Diastereomer #2 (23.8 mg, Rt=2.846 min, 33.2% yield).

Diastereomer #1 (Example 107), (2-((S)-4-(4-fluoropyrazolo[1,5-a]pyridin-2-yl)-1,4,6,7-tetrahydro-5H-imidazo[4,5-c]pyridin-5-yl)pyrimidin-5-yl)(phenyl)methanol: ¹H NMR (400 MHz, METHANOL-d₄) δ 8.23 (s, 2H), 8.18 (d, 1H), 7.51 (s, 1H), 7.22-7.33 (m, 4H), 7.13-7.20 (m, 1H), 7.04 (s, 1H), 6.81 (dd, 1H), 6.67 (td, 1H), 6.53 (s, 1H), 5.62 (s, 1H), 4.92 (dd, 1H), 3.29-3.38 (m, 1H), 2.71-2.81 (m, 1H), 2.57 (br dd, 1H); LCMS: m/z 442.2 [M+H]⁺; SFC: 95.0% ee.

Diastereomer #2 (Example 108), (2-((S)-4-(4-fluoropyrazolo[1,5-a]pyridin-2-yl)-1,4,6,7-tetrahydro-5H-imidazo[4,5-c]pyridin-5-yl)pyrimidin-5-yl)(phenyl)methanol: ¹H NMR (400 MHz, METHANOL-d₄) δ 8.35 (s, 2H), 8.28-8.32 (m, 1H), 7.63 (s, 1H), 7.35-7.44 (m, 4H), 7.26-7.31 (m, 1H), 7.16 (s, 1H), 6.93 (dd, 1H), 6.76-6.82 (m, 1H), 6.65 (s, 1H), 5.74 (s, 1H), 5.04 (br dd, 1H), 3.41-3.50 (m, 1H), 2.83-2.94 (m, 1H), 2.69 (br dd, 1H); LCMS: m/z 442.2 [M+H]⁺; SFC: 90.0% ee.

Diastereomer #3 (Example 109), (2-((R)-4-(4-fluoropyrazolo[1,5-a]pyridin-2-yl)-1,4,6,7-tetrahydro-5H-imidazo[4,5-c]pyridin-5-yl)pyrimidin-5-yl)(phenyl)methanol: ¹H NMR (400 MHz, METHANOL-d₄) δ 8.35 (s, 2H), 8.30 (d, 1H), 7.63 (s, 1H), 7.34-7.45 (m, 4H), 7.25-7.31 (m, 1H), 7.16 (s, 1H), 6.93 (dd, 1H), 6.79 (td, 1H), 6.65 (s, 1H), 5.74 (s, 1H), 5.04 (dd, 1H), 3.41-3.51 (m, 1H), 2.84-2.94 (m, 1H), 2.69 (dd, 1H); LCMS: m/z 442.2 [M+H]⁺; SFC: 93.8% ee.

Diastereomer #4 (Example 110), (2-((R)-4-(4-fluoropyrazolo[1,5-a]pyridin-2-yl)-1,4,6,7-tetrahydro-5H-imidazo[4,5-c]pyridin-5-yl)pyrimidin-5-yl)(phenyl)methanol: ¹H NMR (400 MHz, METHANOL-d₄) δ 8.35 (s, 2H), 8.30 (d, 1H), 7.63 (s, 1H), 7.34-7.45 (m, 4H), 7.25-7.31 (m, 1H), 7.16 (s, 1H), 6.93 (dd, 1H), 6.75-6.83 (m, 1H), 6.65 (s, 1H), 5.74 (s, 1H), 5.04 (br dd, 1H), 3.42-3.51 (m, 1H), 2.84-2.94 (m, 1H), 2.65-2.73 (m, 1H); LCMS: m/z 442.1 [M+H]⁺; SFC: 92.6% ee.

### Preparation of Examples 125 and 126 According to General Schemes 4A and 4B

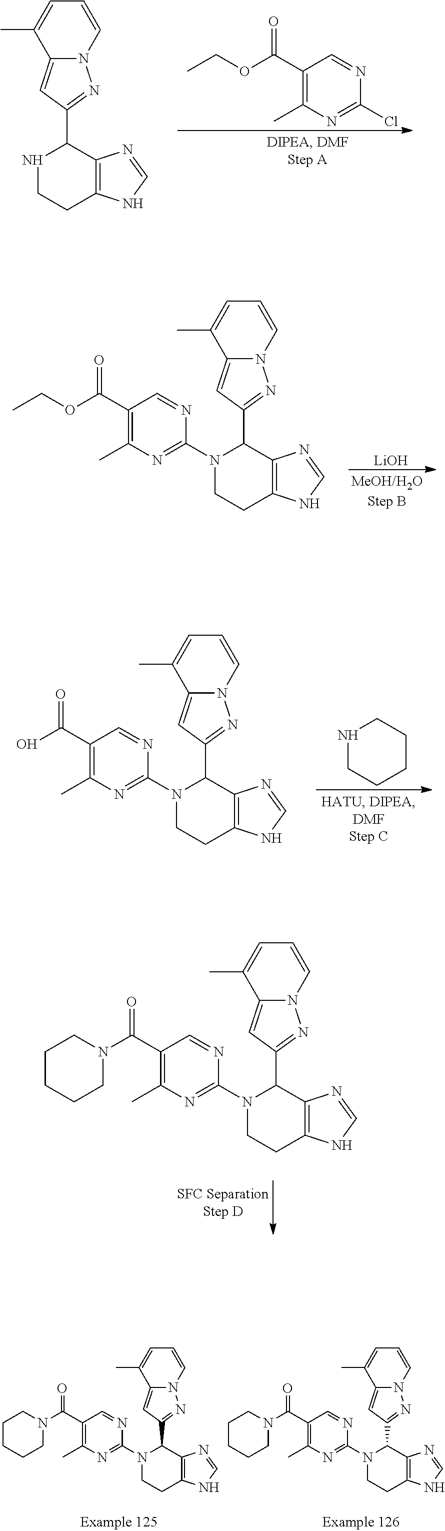

### Step A. Preparation of ethyl 4-methyl-2-(4-(4-methylpyrazolo[1,5-a]pyridin-2-yl)-1,4,6,7-tetrahydro-5H-imidazo[4,5-c]pyridin-5-yl)pyrimidine-5-carboxylate

To a solution of 4-(4-methylpyrazolo[1,5-a]pyridin-2-yl)-4,5,6,7-tetrahydro-1H-imidazo[4,5-c]pyridine (230 mg, 908 μmol) and 2-chloro-4-methyl-5-pyrimidinecarboxylic acid ethyl ester (219 mg, 173 μL, 1.09 mmol) in DMF (4.1 mL) was added DIPEA (176 mg, 237 μL, 1.36 mmol), and the reaction mixture stirred at rt overnight. Water (20 mL) was added to the reaction mixture, and the aqueous portion extracted with EtOAc (2×15 mL). The combined organic layer was dried over Na₂SO₄, filtered, and concentrated to dryness. The residue was purified by column chromatography (Biotage 10 gr SNAP column, 18 mL/min, 0-100% EtOAc in heptane, then 0-20% MeOH in EtOAc) to give ethyl 4-methyl-2-(4-(4-methylpyrazolo[1,5-a]pyridin-2-yl)-1,4,6,7-tetrahydro-5H-imidazo[4,5-c]pyridin-5-yl)pyrimidine-5-carboxylate (106 mg, 71% yield). ¹H NMR (400 MHz, METHANOL-d₄) δ 8.85 (s, 1H), 8.25 (d, 1H), 7.63 (d, 1H), 7.31 (s, 1H), 6.93 (dt, 1H), 6.72 (td, 1H), 6.53 (s, 1H), 5.19 (s, 1H), 4.31 (qd, 2H), 3.52-3.45 (m, 1H), 2.92-2.85 (m, 1H), 2.72 (dd, 1H), 2.68 (s, 3H), 2.40 (s, 4H), 1.38-1.34 (m, 3H); LCMS: m/z 418.2 [M+H]⁺.

### Step B. Preparation of 4-methyl-2-(4-(4-methylpyrazolo[1,5-a]pyridin-2-yl)-1,4,6,7-tetrahydro-5H-imidazo[4,5-c]pyridin-5-yl)pyrimidine-5-carboxylic acid, lithium salt

A solution of ethyl 4-methyl-2-(4-(4-methylpyrazolo[1,5-a]pyridin-2-yl)-1,4,6,7-tetrahydro-5H-imidazo[4,5-c]pyridin-5-yl)pyrimidine-5-carboxylate (343 mg, 822 μmol) in methanol (2.9 mL) and water (0.8 mL) was cooled to 0° C., and lithium hydroxide monohydrate (103 mg, 68.5 μL, 2.46 mmol) added. The reaction mixture was stirred at rt overnight and concentrated to dryness to afford 4-methyl-2-(4-(4-methylpyrazolo[1,5-a]pyridin-2-yl)-1,4,6,7-tetrahydro-5H-imidazo[4,5-c]pyridin-5-yl)pyrimidine-5-carboxylic acid, lithium salt (320 mg, 100% yield). LCMS: m/z 390.1 [M+H]⁺.

### Step C. Preparation of (4-methyl-2-(4-(4-methylpyrazolo[1,5-a]pyridin-2-yl)-1,4,6,7-tetrahydro-5H-imidazo[4,5-c]pyridin-5-yl)pyrimidin-5-yl)(piperidin-1-yl)methanone

To a solution of 4-methyl-2-(4-(4-methylpyrazolo[1,5-a]pyridin-2-yl)-1,4,6,7-tetrahydro-5H-imidazo[4,5-c]pyridin-5-yl)pyrimidine-5-carboxylic acid (100 mg, 257 μmol) in DMF (1.2 mL) were added HATU (205 mg, 539 μmol) and DIPEA (39.8 mg, 53.7 μL, 308 μmol). The reaction mixture was stirred at rt for 10 min, and then piperidine (45.9 mg, 53.1 μL, 539 μmol) was added. The reaction mixture was stirred at rt overnight. Water (25 mL) was added to the reaction mixture, and the aqueous portion extracted with EtOAc (2×20 mL). The combined organic layer was dried over Na₂SO₄, filtered, and concentrated to dryness. The residue was purified by column chromatography (Biotage 10 gr Sfar column, 18 mL/min, 0-20% MeOH in DCM) and further purified by prep-HPLC (Column: XSelect CSH C18 (5 μm, 30×100 mm), mobile phase: 21-26% MeCN in H₂O [with 0.1% formic acid]) to afford (4-methyl-2-(4-(4-methylpyrazolo[1,5-a]pyridin-2-yl)-1,4,6,7-tetrahydro-5H-imidazo[4,5-c]pyridin-5-yl)pyrimidin-5-yl)(piperidin-1-yl)methanone (85 mg, 73% yield). ¹H NMR (400 MHz, METHANOL-d₄) δ 8.24 (d, 1H), 8.20 (d, 1H), 7.67 (s, 1H), 7.25 (s, 1H), 6.92 (dd, 1H), 6.71 (t, 1H), 6.51 (s, 1H), 5.12 (dd, 1H), 3.70 (s, 2H), 3.46 (td, 1H), 3.35 (d, 2H), 2.88 (ddd, 1H), 2.69 (dd, 1H), 2.39-2.36 (m, 6H), 1.72-1.54 (m, 6H); LCMS: m/z 457.2 [M+H]⁺.

### Step D. SFC Separation

[4-methyl-2-[4-(4-methylpyrazolo[1,5-a]pyridin-2-yl)-1,4,6,7-tetrahydroimidazo[4,5-c]pyridin-5-yl]pyrimidin-5-yl]-(1-piperidyl)methanone (85 mg) was separated by SFC (column: ChiralCel OD, 250×30 mm, 10 μm; mobile phase: [CO₂-i-PrOH (0.1% NH₃H₂O)]; B %:35%, isocratic elution mode) to give Enantiomer #1 (18.8 mg, Rt=1.770 min, 22.1% yield) and Enantiomer #2 (17.8 mg, Rt=2.510 min, 20.9% yield).

Enantiomer #1 (Example 125), (R)-(4-methyl-2-(4-(4-methylpyrazolo[1,5-a]pyridin-2-yl)-1,4,6,7-tetrahydro-5H-imidazo[4,5-c]pyridin-5-yl)pyrimidin-5-yl)(piperidin-1-yl)methanone: ¹H NMR (400 MHz, METHANOL-d₄) δ 8.16-8.31 (m, 2H), 7.65 (s, 1H), 7.27 (s, 1H), 6.96 (d, 1H), 6.75 (t, 1H), 6.54 (s, 1H), 5.13 (br d, 1H), 3.60-3.83 (m, 2H), 3.35-3.55 (m, 3H), 2.83-2.97 (m, 1H), 2.71 (dd, 1H), 2.40 (d, 6H), 1.45-1.79 (m, 6H); LCMS: m/z 457.2 [M+H]⁺; SFC: 100% ee.

Enantiomer #2 (Example 126), (S)-(4-methyl-2-(4-(4-methylpyrazolo[1,5-a]pyridin-2-yl)-1,4,6,7-tetrahydro-5H-imidazo[4,5-c]pyridin-5-yl)pyrimidin-5-yl)(piperidin-1-yl)methanone: ¹H NMR (400 MHz, METHANOL-d₄) δ 8.17-8.33 (m, 2H), 7.65 (s, 1H), 7.27 (br s, 1H), 6.97 (d, 1H), 6.76 (t, 1H), 6.54 (s, 1H), 5.13 (br d, 1H), 3.73 (br s, 2H), 3.35-3.53 (m, 3H), 2.82-2.99 (m, 1H), 2.71 (dd, 1H), 2.41 (d, 6H), 1.53-1.75 (m, 6H); LCMS: m/z 457.2 [M+H]⁺; SFC: 98.4% ee.

### Preparation of Examples 129 and 130 According to General Scheme 4B

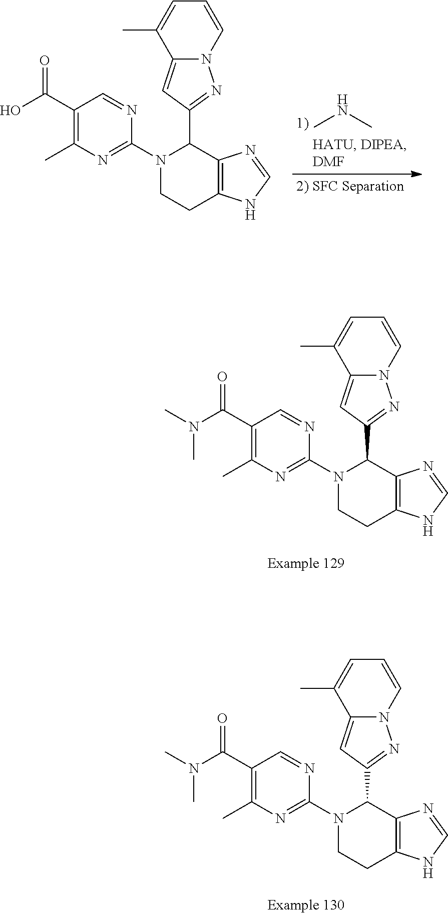

### Step 1. Preparation of N,N,4-trimethyl-2-(4-(4-methylpyrazolo[1,5-a]pyridin-2-yl)-1,4,6,7-tetrahydro-5H-imidazo[4,5-c]pyridin-5-yl)pyrimidine-5-carboxamide

To a solution of 4-methyl-2-(4-(4-methylpyrazolo[1,5-a]pyridin-2-yl)-1,4,6,7-tetrahydro-5H-imidazo[4,5-c]pyridin-5-yl)pyrimidine-5-carboxylic acid (150 mg, 385 μmol) in DMF (1.5 mL) were added HATU (308 mg, 809 μmol) and DIPEA (59.7 mg, 80.5 μL, 462 μmol). The reaction mixture was stirred at rt for 10 min, and then dimethylamine (36.5 mg, 404 μL, 809 μmol) was added. The reaction mixture was stirred at rt overnight. Water (25 mL) was added to the reaction mixture, and the aqueous portion extracted with EtOAc (2×20 mL). The combined organic layer was dried over Na₂SO₄, filtered, and concentrated to dryness. The residue was purified by column chromatography (Biotage 10 gr Sfar column, 18 mL/min, 0-20% MeOH in DCM) and then further purified by prep-HPLC (Column: XSelect CSH C18 (5 μm, 30×100 mm), mobile phase: 18-23% MeCN in H₂O [with 0.1% formic acid]) to afford N,N,4-trimethyl-2-(4-(4-methylpyrazolo[1,5-a]pyridin-2-yl)-1,4,6,7-tetrahydro-5H-imidazo[4,5-c]pyridin-5-yl)pyrimidine-5-carboxamide (67 mg, 42% yield). ¹H NMR (400 MHz, METHANOL-d₄) δ 8.27-8.22 (m, 2H), 7.63 (s, 1H), 7.25 (s, 1H), 6.94 (dq, 1H), 6.73 (t, 1H), 6.52 (s, 1H), 5.12 (dd, 1H), 3.47 (td, 1H), 3.10 (s, 3H), 2.99 (s, 3H), 2.89 (ddd, 1H), 2.69 (dd, 1H), 2.40 (s, 3H), 2.36 (d, 3H); LCMS: m/z 417.2 [M+H]⁺.

### Step 2. SFC Separation

N,N,4-trimethyl-2-(4-(4-methylpyrazolo[1,5-a]pyridin-2-yl)-1,4,6,7-tetrahydro-5H-imidazo[4,5-c]pyridin-5-yl)pyrimidine-5-carboxamide (63 mg) was separated by SFC (column: ChiralCel OD, 250×30 mm, 10 μm; mobile phase: [CO₂-i-PrOH(0.1% NH₃H₂O)]; B %:35%, isocratic elution mode) to give Enantiomer #1 (12.1 mg, Rt=1.693 min, 19.2% yield) and Enantiomer #2 (10.2 mg, Rt=2.350 min, 16.2% yield).

Enantiomer #1 (Example 129), (R)—N,N,4-trimethyl-2-(4-(4-methylpyrazolo[1,5-a]pyridin-2-yl)-1,4,6,7-tetrahydro-5H-imidazo[4,5-c]pyridin-5-yl)pyrimidine-5-carboxamide: ¹H NMR (400 MHz, METHANOL-d₄) δ 8.18-8.31 (m, 2H), 7.65 (s, 1H), 7.25 (s, 1H), 6.95 (d, 1H), 6.74 (t, 1H), 6.53 (s, 1H), 5.12 (br d, 1H), 3.46 (td, 1H), 3.11 (s, 3H), 2.99 (s, 3H), 2.83-2.95 (m, 1H), 2.70 (dd, 1H), 2.41 (s, 3H), 2.36 (s, 3H); LCMS: m/z 417.2 [M+H]⁺; SFC: 100% ee.

Enantiomer #2 (Example 130), (S)—N,N,4-trimethyl-2-(4-(4-methylpyrazolo[1,5-a]pyridin-2-yl)-1,4,6,7-tetrahydro-5H-imidazo[4,5-c]pyridin-5-yl)pyrimidine-5-carboxamide: ¹H NMR (400 MHz, METHANOL-d₄) δ 8.16-8.35 (m, 2H), 7.64 (s, 1H), 7.25 (s, 1H), 6.95 (d, 1H), 6.74 (t, 1H), 6.52 (s, 1H), 5.12 (br d, 1H), 3.40-3.52 (m, 1H), 3.11 (s, 3H), 2.99 (s, 3H), 2.83-2.94 (m, 1H), 2.70 (dd, 1H), 2.41 (s, 3H), 2.36 (s, 3H); LCMS: m/z 417.2 [M+H]⁺; SFC: 96.7% ee.

### Preparation of Examples 238 and 239 According to General Scheme 2, Method A, and General Scheme 5

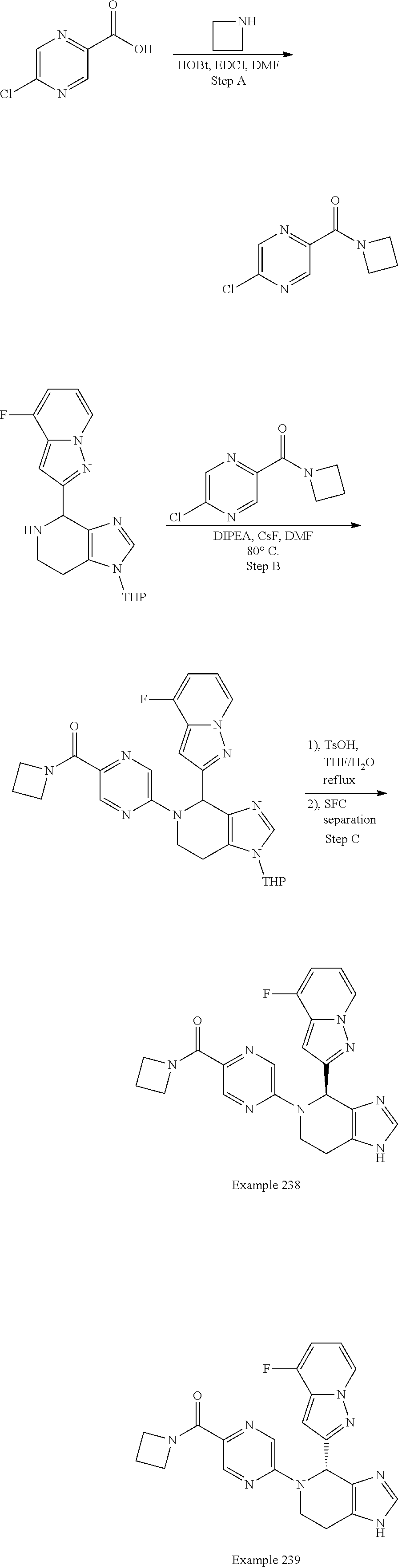

### Step A: Synthesis of azetidin-1-yl-(5-chloropyrazin-2-yl) methanone

To a solution of 5-chloropyrazine-2-carboxylic acid (1 g, 6.31 mmol) and azetidine (540 mg, 9.46 mmol, 639 μL) in DMF (20 mL) was added EDCI (1.81 g, 9.46 mmol) and HOBt (1.28 g, 9.46 mmol), and the reaction mixture was stirred at rt for 12 hrs under N₂ atmosphere. EtOAc (100 mL) was added to the reaction mixture, and the organic layer was washed by water (50 mL) and brine (50 mL), dried over Na₂SO₄, filtered, and concentrated to dryness. The residue was purified by flash silica gel chromatography (ISCO®; 12 g SepaFlash® Silica Flash Column, Eluent of 0-10% MeOH:DCM @20 mL/min) to give azetidin-1-yl-(5-chloropyrazin-2-yl) methanone (720 mg, 57.8% yield). ¹H NMR (400 MHz, CDCl₃) δ 9.10 (d, 1H), 8.53 (d, 1H), 4.68 (t, 2H), 4.28 (t, 2H), 2.40 (quin, 2H); LCMS: m/z 198.1 [M+H]⁺.

### Step B: Synthesis of azetidin-1-yl-[5-[4-(4-fluoropyrazolo[1,5-a]pyridin-2-yl)-1-tetrahydropyran-2-yl-6,7-dihydro-4H-imidazo[4,5-c]pyridin-5-yl]pyrazin-2-yl]methanone

To a solution of 4-(4-fluoropyrazolo[1,5-a]pyridin-2-yl)-1-tetrahydropyran-2-yl-4,5,6,7-tetrahydroimidazo[4,5-c]pyridine (1.5 g, 4.39 mmol) and azetidin-1-yl-(5-chloropyrazin-2-yl)methanone (1.04 g, 5.27 mmol) in DMF (10 mL) was added DIPEA (1.70 g, 13.2 mmol, 2.30 mL) and CsF (1.33 g, 8.79 mmol,), and the reaction mixture was stirred at 80° C. for 12 hrs under N₂ atmosphere. The reaction mixture was cooled to rt, and EtOAc (100 mL) was added. The organic layer was washed by water (50 mL) and brine (50 mL), dried over Na₂SO₄, filtered, and concentrated to dryness. The residue was purified by flash silica gel chromatography (ISCO®; 20 g SepaFlash® Silica Flash Column, Eluent of 0-5% MeOH:DCM @40 mL/min) to give azetidin-1-yl-[5-[4-(4-fluoropyrazolo[1,5-a]pyridin-2-yl)-1-tetrahydropyran-2-yl-6,7-dihydro-4H-imidazo[4,5-c]pyridin-5-yl]pyrazin-2-yl]methanone (2.2 g, 84.7% yield). LCMS: m/z 503.2 [M+H]⁺.

### Step C: Synthesis of azetidin-1-yl-[5-[4-(4-fluoropyrazolo[1,5-a]pyridin-2-yl)-1,4,6,7-tetrahydroimidazo[4,5-c]pyridin-5-yl]pyrazin-2-yl]methanone

To a solution of azetidin-1-yl-[5-[4-(4-fluoropyrazolo[1,5-a]pyridin-2-yl)-1-tetrahydropyran-2-yl-6,7-dihydro-4H-imidazo[4,5-c]pyridin-5-yl]pyrazin-2-yl]methanone (2.2 g, 4.38 mmol) in THF (20 mL) and H₂O (10 mL) was added TsOH (1.51 g, 8.76 mmol), and the reaction mixture was stirred at 80° C. for 12 hrs. The reaction mixture was concentrated in vacuum, and EtOAc (150 mL) was added to the residue. The organic layer was washed by sat. aq. NaHCO₃ solution (50 mL) and brine (50 mL), dried over Na₂SO₄, filtered, and concentrated to dryness. The residue was purified by flash silica gel chromatography (ISCO®; 20 g SepaFlash® Silica Flash Column, Eluent of 0-5% MeOH:DCM @30 mL/min) to give azetidin-1-yl-[5-[4-(4-fluoropyrazolo[1,5-a]pyridin-2-yl)-1,4,6,7-tetrahydroimidazo[4,5-c]pyridin-5-yl]pyrazin-2-yl]methanone (1.1 g, 58.9% yield). LCMS: m/z 419.2 [M+H]⁺.

### Step D: SFC Separation

Azetidin-1-yl-[5-[4-(4-fluoropyrazolo[1,5-a]pyridin-2-yl)-1,4,6,7-tetrahydroimidazo[4,5-c]pyridin-5-yl]pyrazin-2-yl]methanone (1 g) was separated by SFC (column: DAICEL CHIRALPAK AD (250 mm*50 mm, 10 μm); mobile phase: [CO₂-i-PrOH(0.1% NH₃H₂O)]; B %:55%, isocratic elution mode) to give Enantiomer #1 (269 mg, Rt=0.927 min, 26.4% yield) and Enantiomer #2 (275 mg, Rt=1.981 min, 27.0% yield).

Enantiomer #1 (Example 238), (R)-azetidin-1-yl(5-(4-(4-fluoropyrazolo[1,5-a]pyridin-2-yl)-1,4,6,7-tetrahydro-5H-imidazo[4,5-c]pyridin-5-yl)pyrazin-2-yl)methanone: ¹H NMR (400 MHz, METHANOL-d₄) δ 8.69 (d, 1H), 8.45 (d, 1H), 8.33 (d, 1H), 7.64 (s, 1H), 6.77-6.97 (m, 3H), 6.66 (s, 1H), 4.81-4.86 (m, 1H), 4.70 (t, 2H), 4.19 (t, 2H), 3.55-3.70 (m, 1H), 2.89-3.04 (m, 1H), 2.78 (dd, 1H), 2.36 (quin, 2H); LCMS: m/z 419.2 [M+H]⁺; SFC: 100% ee.

Enantiomer #2 (Example 239), (S)-azetidin-1-yl(5-(4-(4-fluoropyrazolo[1,5-a]pyridin-2-yl)-1,4,6,7-tetrahydro-5H-imidazo[4,5-c]pyridin-5-yl)pyrazin-2-yl)methanone: ¹H NMR (400 MHz, METHANOL-d₄) δ 8.70 (d, 1H), 8.46 (s, 1H), 8.33 (d, 1H), 7.64 (s, 1H), 6.72-7.00 (m, 3H), 6.66 (s, 1H), 4.83 (br d, 1H), 4.70 (t, 2H), 4.19 (t, 2H), 3.54-3.69 (m, 1H), 2.90-3.02 (m, 1H), 2.78 (br dd, 1H), 2.37 (quin, 2H); LCMS: m/z 419.2 [M+H]⁺; SFC: 99.5% ee.

### Preparation of Examples 240, 241, 242, and 243 according to General Scheme 2, Method A

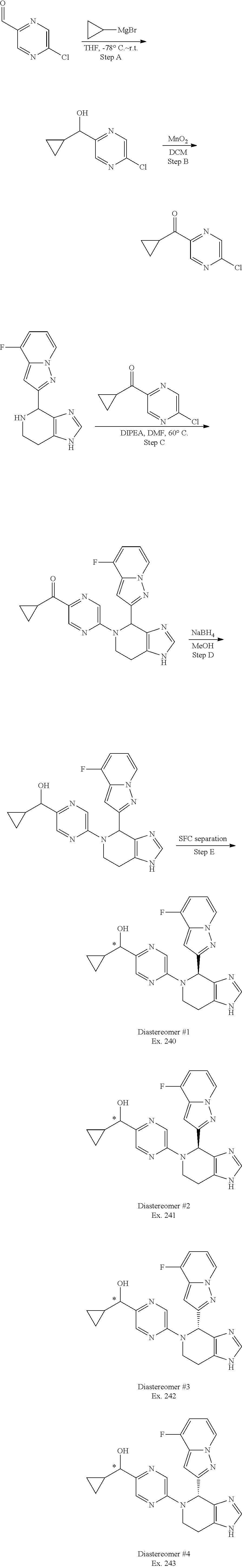

### Step A: Preparation of (5-chloropyrazin-2-yl)-cyclopropyl-methanol

To a solution of 5-chloropyrazine-2-carbaldehyde (1 g, 7.02 mmol) in THF (10 mL) was added bromo(cyclopropyl)magnesium (1 M, 14.03 mL, 2 eq), the reaction mixture was stirred at −78° C. for 2 hrs under N₂ atmosphere. Then the mixture was stirred at rt for another 2 hrs under N₂ atmosphere. Sat. aq. NH₄Cl solution (80 mL) was added to the reaction mixture at 0° C., and then the aqueous portion extracted with EtOAc (20 mL×3). The combined organic layer was dried over Na₂SO₄, filtered, and concentrated to dryness. The residue was purified by flash silica gel chromatography (ISCO®; 12 g SepaFlash® Silica Flash Column, Eluent of 0-15% EtOAc/PE, gradient @30 mL/min) to give (5-chloropyrazin-2-yl)-cyclopropyl-methanol (340 mg, 24.7% yield). ¹H NMR (400 MHz, CDCl₃) δ 8.39-8.71 (m, 2H), 4.19 (dd, 1H), 3.20 (br d, 1H), 1.19 (qt, 1H), 0.52-0.71 (m, 4H); LCMS: m/z 166.9 [M−18]⁺.

### Step B: Preparation of (5-chloropyrazin-2-yl)-cyclopropyl-methanone

To a solution of (5-chloropyrazin-2-yl)-cyclopropyl-methanol (340 mg, 1.84 mmol) in DCM (30 mL) was added MnO₂ (1.60 g, 18.4 mmol). The reaction mixture was stirred at rt for 14 hrs and then filtered and concentrated to dryness. The residue was purified by flash silica gel chromatography (ISCO®; 4 g SepaFlash® Silica Flash Column, Eluent of 0-5.8% EtOAc/PE, gradient @20 mL/min) to give (5-chloropyrazin-2-yl)-cyclopropyl-methanone (285 mg, 84.8% yield). LCMS: m/z 182.9 [M+H]⁺.

### Step C: Preparation of cyclopropyl-[5-[4-(4-fluoropyrazolo[1,5-a]pyridin-2-yl)-1,4,6,7-tetrahydroimidazo[4,5-c]pyridin-5-yl]pyrazin-2-yl]methanone

To a solution of 4-(4-fluoropyrazolo[1,5-a]pyridin-2-yl)-4,5,6,7-tetrahydro-1H-imidazo [4,5-c]pyridine (200 mg, 777 μmol) and (5-chloropyrazin-2-yl)-cyclopropyl-methanone (170 mg, 933 μmol) in DMF (3 mL) was added DIPEA (301 mg, 2.33 mmol, 406 L), the reaction mixture was stirred at 60° C. for 12 hrs under N₂ atmosphere. The reaction mixture was cooled to rt, and DCM (40 mL) was added. The organic layer was washed by water (20 mL) and brine (20 mL), dried over Na₂SO₄, filtered, and concentrated to dryness. The residue was purified by flash silica gel chromatography (ISCO®; 12 g SepaFlash® Silica Flash Column, Eluent of 0-5% MeOH/DCM@20 mL/min) to give cyclopropyl-[5-[4-(4-fluoropyrazolo[1,5-a]pyridin-2-yl)-1,4,6,7-tetrahydroimidazo[4,5-c]pyridin-5-yl]pyrazin-2-yl]methanone (150 mg, 42.1% yield). LCMS: m/z 404.2 [M+H]⁺.

### Step D: Preparation of cyclopropyl-[5-[4-(4-fluoropyrazolo[1,5-a]pyridin-2-yl)-1,4,6,7-tetrahydroimidazo[4,5-c]pyridin-5-yl]pyrazin-2-yl]methanol

To a solution of cyclopropyl-[5-[4-(4-fluoropyrazolo[1,5-a]pyridin-2-yl)-1,4,6,7-tetrahydroimidazo[4,5-c]pyridin-5-yl]pyrazin-2-yl]methanone (150 mg, 372 μmol) in MeOH (10 mL) was added NaBH₄ (28.1 mg, 744 μmol) at 0° C., then the reaction mixture was stirred at rt for 2 hrs under N₂ atmosphere. Water (20 mL) was added dropwise to the reaction mixture, and the aqueous portion extracted with DCM (50 mL×2). The combined organic layer was dried over Na₂SO₄, filtered, and concentrated to dryness. The residue was purified by flash silica gel chromatography (ISCO®; 12 g SepaFlash® Silica Flash Column, Eluent of 0-5% MeOH:DCM @20 mL/min) to afford cyclopropyl-[5-[4-(4-fluoropyrazolo[1,5-a]pyridin-2-yl)-1,4,6,7-tetrahydroimidazo[4,5-c]pyridin-5-yl]pyrazin-2-yl]methanol (140 mg, 80.3% yield). LCMS: m/z 406.3 [M+H]⁺.

### Step E: SFC Separation

Cyclopropyl-[5-[4-(4-fluoropyrazolo[1,5-a]pyridin-2-yl)-1,4,6,7-tetrahydroimidazo[4,5-c]pyridin-5-yl]pyrazin-2-yl]methanol (100 mg) was separated by SFC (column: DAICEL CHIRALPAK IG (250 mm*30 mm, 10 μm); mobile phase: [CO₂-i-PrOH (0.1% NH₃H₂O)]; B %:60%, isocratic elution mode) to give Diastereomer #1 (19.4 mg, Rt=1.797 min, 13.2% yield), Diastereomer #2 (19.4 mg, Rt=2.358 min, 13.5% yield), Diastereomer #3 (17.8 mg, Rt=3.249 min, 12.2% yield) and Diastereomer #4 (24.5 mg, Rt=4.504 min, 14.6% yield).

Diastereomer #1 (Example 240), cyclopropyl(5-((R)-4-(4-fluoropyrazolo[1,5-a]pyridin-2-yl)-1,4,6,7-tetrahydro-5H-imidazo[4,5-c]pyridin-5-yl)pyrazin-2-yl)methanol. ¹H NMR (400 MHz, METHANOL-d₄) δ 8.38 (d, 1H), 8.32 (d, 1H), 8.25 (d, 1H), 7.62 (s, 1H), 6.93 (dd, 1H), 6.79 (td, 2H), 6.63 (s, 1H), 4.68 (br dd, 1H), 3.98 (d, 1H), 3.61 (ddd, 1H), 2.87-3.04 (m, 1H), 2.74 (br dd, 1H), 1.21-1.36 (m, 1H), 0.54-0.68 (m, 1H), 0.41-0.53 (m, 2H), 0.27-0.40 (m, 1H); LCMS: m/z 406.2 [M+H]⁺; SFC: 100% ee.

Diastereomer #2 (Example 241), cyclopropyl(5-((R)-4-(4-fluoropyrazolo[1,5-a]pyridin-2-yl)-1,4,6,7-tetrahydro-5H-imidazo[4,5-c]pyridin-5-yl)pyrazin-2-yl)methanol. ¹H NMR (400 MHz, METHANOL-d₄) δ 8.38 (d, 1H), 8.32 (d, 1H), 8.25 (d, 1H), 7.62 (s, 1H), 6.93 (dd, 1H), 6.73-6.85 (m, 2H), 6.63 (s, 1H), 4.68 (br dd, 1H), 3.98 (d, 1H), 3.61 (ddd, 1H), 2.88-3.02 (m, 1H), 2.75 (br dd, 1H), 1.20-1.31 (m, 1H), 0.55-0.65 (m, 1H), 0.43-0.53 (m, 2H), 0.29-0.39 (m, 1H); LCMS: m/z 406.2 [M+H]⁺; SFC: 94.3% ee.

Diastereomer #3 (Example 242), cyclopropyl(5-((S)-4-(4-fluoropyrazolo[1,5-a]pyridin-2-yl)-1,4,6,7-tetrahydro-5H-imidazo[4,5-c]pyridin-5-yl)pyrazin-2-yl)methanol. ¹H NMR (400 MHz, METHANOL-d₄) δ 8.38 (d, 1H), 8.32 (d, 1H), 8.25 (d, 1H), 7.62 (s, 1H), 6.92 (dd, 1H), 6.73-6.85 (m, 2H), 6.63 (s, 1H), 4.68 (dd, 1H), 3.98 (d, 1H), 3.61 (ddd, 1H), 2.87-3.03 (m, 1H), 2.74 (dd, 1H), 1.19-1.32 (m, 1H), 0.55-0.66 (m, 1H), 0.42-0.53 (m, 2H), 0.28-0.39 (m, 1H); LCMS: m/z 406.2 [M+H]⁺; SFC: 96.9% ee.

Diastereomer #4 (Example 243), cyclopropyl(5-((S)-4-(4-fluoropyrazolo[1,5-a]pyridin-2-yl)-1,4,6,7-tetrahydro-5H-imidazo[4,5-c]pyridin-5-yl)pyrazin-2-yl)methanol. ¹H NMR (400 MHz, METHANOL-d₄) δ 8.38 (d, 1H), 8.32 (d, 1H), 8.25 (d, 1H), 7.64 (s, 1H), 6.92 (dd, 1H), 6.75-6.83 (m, 2H), 6.63 (s, 1H), 4.68 (dd, 1H), 3.98 (d, 1H), 3.61 (ddd, 1H), 2.89-3.03 (m, 1H), 2.74 (dd, 1H), 1.26-1.31 (m, 1H), 0.55-0.67 (m, 1H), 0.43-0.52 (m, 2H), 0.30-0.39 (m, 1H); LCMS: m/z 406.2 [M+H]⁺; SFC: 88.0% ee.

### Preparation of Examples 252 and 253 According to General Scheme 2, Method C1

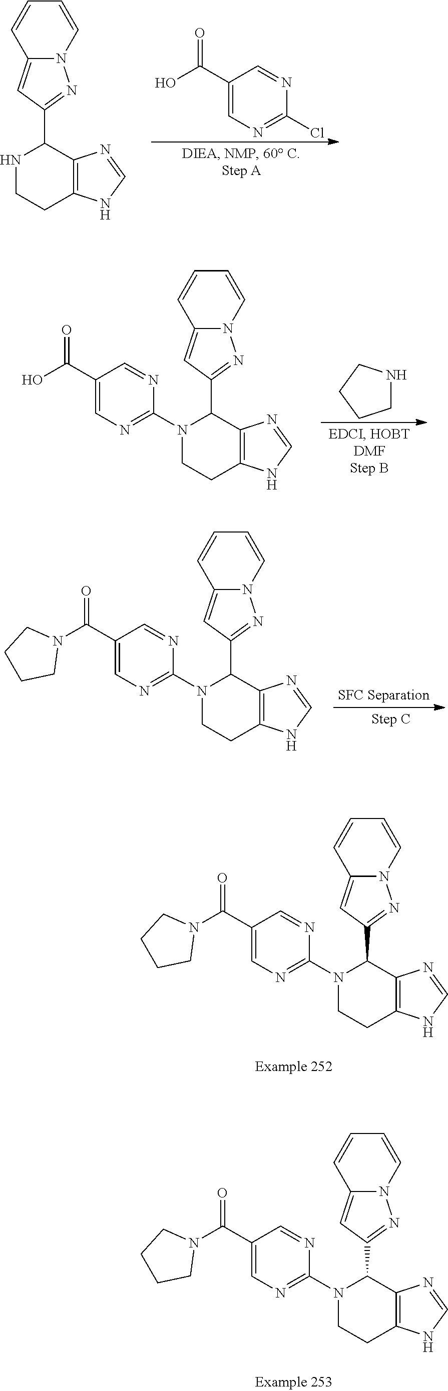

### Step A. Synthesis of 2-(4-(pyrazolo[1,5-a]pyridin-2-yl)-1,4,6,7-tetrahydro-5H-imidazo[4,5-c]pyridin-5-yl)pyrimidine-5-carboxylic acid

To a mixture of 2-chloropyrimidine-5-carboxylic acid (500 mg, 3.15 mmol) and 4-(pyrazolo[1,5-a]pyridin-2-yl)-4,5,6,7-tetrahydro-1H-imidazo[5,4-c]pyridine (755 mg, 3.15 mmol) in NMP (5 mL) was added DIEA (1.22 g, 9.46 mmol). The reaction mixture was stirred at 60° C. for 3 hrs. The reaction mixture was cooled to rt, and ice water (10 mL) was added. The aqueous portion was extracted with DCM (3×20 mL). The combined organic layer was washed with brine (10 mL), dried over Na₂SO₄, and concentrated to dryness. The residue was purified by Prep-HPLC (mobile phase: ACN-H₂O+0.1% NH₄HCO₃) to obtain 2-(4-(pyrazolo[1,5-a]pyridin-2-yl)-1,4,6,7-tetrahydro-5H-imidazo[4,5-c]pyridin-5-yl)pyrimidine-5-carboxylic acid (150 mg, 13% yield). LC-MS: m/z 362.1 [M+H]⁺.

### Step B. Synthesis of (2-(4-(pyrazolo[1,5-a]pyridin-2-yl)-1,4,6,7-tetrahydro-5H-imidazo[4,5-c]pyridin-5-yl)pyrimidin-5-yl)(pyrrolidin-1-yl)methanone

To a mixture of 2-(4-(pyrazolo[1,5-a]pyridin-2-yl)-1,4,6,7-tetrahydro-5H-imidazo[4,5-c]pyridin-5-yl)pyrimidine-5-carboxylic acid (100 mg, 0.28 mmol) and tetrahydropyrrole (0.07 mL, 0.83 mmol) in DMF (1.5 mL) was added HOBt (74.8 mg, 0.55 mmol) and EDCI (106 mg, 0.55 mmol). The reaction mixture was stirred at rt for 2 hrs. Ice water (10 mL) was added, and the aqueous portion extracted with DCM (3×20 mL). The combined organic layer was washed with brine (10 mL), dried over Na₂SO₄, and concentrated to dryness. The residue was purified by Prep-HPLC (mobile phase: ACN-H₂O+0.1% NH₄HCO₃) to obtain (2-(4-(pyrazolo[1,5-a]pyridin-2-yl)-1,4,6,7-tetrahydro-5H-imidazo[4,5-c]pyridin-5-yl)pyrimidin-5-yl)(pyrrolidin-1-yl)methanone (80.0 mg, 69% yield). LC-MS: m/z 415.2 [M+H]⁺.

### Step C: SFC Separation

(2-(4-(pyrazolo[1,5-a]pyridin-2-yl)-1,4,6,7-tetrahydro-5H-imidazo[4,5-c]pyridin-5-yl)pyrimidin-5-yl)(pyrrolidin-1-yl)methanone (40 mg, 0.1 mmol) was separated by SFC (Column: ChiralPak IB (4.6*100 mm, 3 μm); mobile phase: [CO₂-MeOH(0.05% DEA)]; B %:30%; isocratic elution mode) to obtain Enantiomer #1 (14.7 mg, Rt=3.581 min, 37% yield) and Enantiomer #2 (19.7 mg, Rt=4.518 min, 49% yield).

Enantiomer #1 (Example 252), (R)-(2-(4-(pyrazolo[1,5-a]pyridin-2-yl)-1,4,6,7-tetrahydro-5H-imidazo[4,5-c]pyridin-5-yl)pyrimidin-5-yl)(pyrrolidin-1-yl)methanone: ¹H NMR (400 MHz, DMSO-d₆) δ 12.03 (s, 1H), 8.64 (s, 2H), 8.60 (d, 1H), 7.60 (d, 1H), 7.56 (s, 1H), 7.21-7.13 (m, 1H), 7.06 (s, 1H), 6.82 (t, 1H), 6.53 (s, 1H), 5.04 (dd, 1H), 3.57-3.43 (m, 5H), 2.79-2.65 (m, 2H), 1.84 (s, 4H); LC-MS: m/z 415.2 [M+H]⁺; SFC: 100% ee.

Enantiomer #2 (Example 253), (S)-(2-(4-(pyrazolo[1,5-a]pyridin-2-yl)-1,4,6,7-tetrahydro-5H-imidazo[4,5-c]pyridin-5-yl)pyrimidin-5-yl)(pyrrolidin-1-yl)methanone: ¹H NMR (400 MHz, DMSO-d₆) δ 11.89 (s, 1H), 8.64 (s, 2H), 8.60 (d, 1H), 7.60 (d, 1H), 7.54 (s, 1H), 7.20-7.13 (m, 1H), 7.03 (s, 1H), 6.81 (t, 1H), 6.53 (s, 1H), 5.04 (dd, 1H), 3.56-3.45 (m, 5H), 2.79-2.68 (m, 2H), 1.84 (s, 4H); LC-MS: m/z 415.2 [M+H]⁺; SFC: 96.0% ee.

### Preparation of Examples 254 and 255 According to General Scheme 2, Method Cl, and General Scheme 5

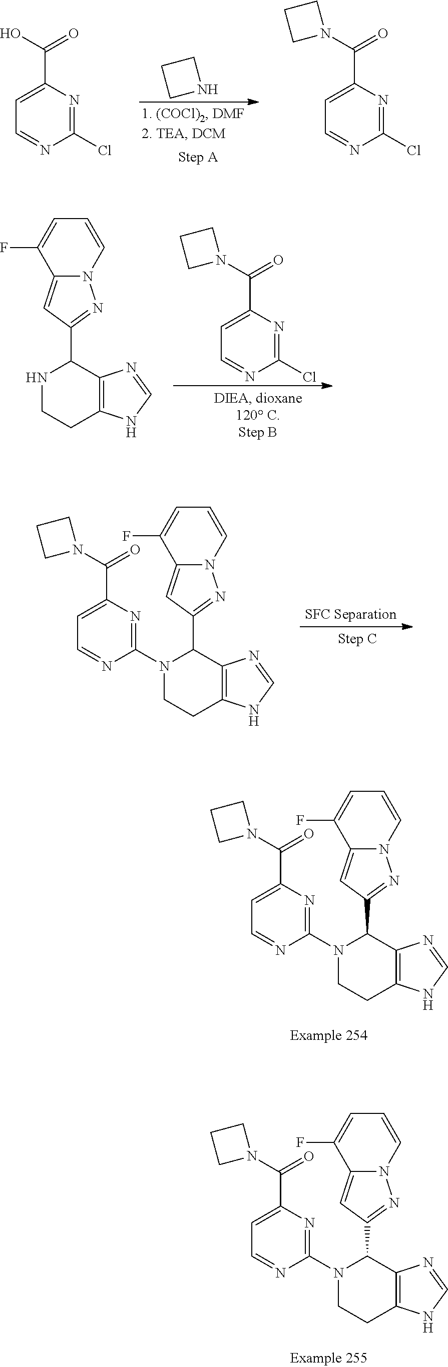

### Step A. Synthesis of azetidin-1-yl(2-chloropyrimidin-4-yl)methanone

To a cold (0° C.) solution of 2-chloropyrimidine-4-carboxylic acid (1 g, 6.31 mmol) and DMF (0.1 mL, 1.89 mmol) in DCM (10 mL) was added oxalyl chloride (1.20 g, 9.46 mmol), and the reaction mixture was stirred at 0° C. for 30 min. The reaction mixture was concentrated under reduced pressure, and DCM (10 mL), azetidine (396 mg, 6.94 mmol) and TEA (3.5 mL, 25.2 mmol) were added. The reaction mixture was stirred at rt for 1 hr, and ice water (10 mL) was added. The aqueous portion was extracted with DCM (3×20 mL). The combined organic layer was washed with brine (10 mL), dried over Na₂SO₄, filtered, and concentrated to dryness. The residue was purified by flash column chromatography on silica gel to obtain azetidin-1-yl(2-chloropyrimidin-4-yl)methanone (300 mg, 23% yield). LC-MS: m/z 198.0, 200.0 [M+H]⁺.

### Step B. Synthesis of azetidin-1-yl(2-(4-(4-fluoropyrazolo[1,5-a]pyridin-2-yl)-1,4,6,7-tetrahydro-5H-imidazo[4,5-c]pyridin-5-yl)pyrimidin-4-yl)methanone

To a mixture of (azetidin-1-yl)(2-chloropyrimidin-4-yl)methanone (300 mg, 1.52 mmol) and 4-(4-fluoropyrazolo[1,5-a]pyridin-2-yl)-4,5,6,7-tetrahydro-1H-imidazo[5,4-c]pyridine (391 mg, 1.52 mmol) in dioxane (3 mL) was added DIEA (589 mg, 4.56 mmol). The reaction mixture was stirred at 120° C. for 3 hrs. The reaction mixture was cooled to rt, and water (10 mL) was added. The aqueous portion was extracted with DCM (3×20 mL). The combined organic layer was washed with brine (10 mL), dried over Na₂SO₄, filtered, and concentrated to dryness. The residue was concentrated and purified by Prep-HPLC (mobile phase: ACN-H₂O+0.1% NH₄HCO₃) to obtain azetidin-1-yl(2-(4-(4-fluoropyrazolo[1,5-a]pyridin-2-yl)-1,4,6,7-tetrahydro-5H-imidazo[4,5-c]pyridin-5-yl)pyrimidin-4-yl)methanone (280 mg, 41% yield). LC-MS: m/z 419.1 [M+H]⁺.

### Step C. SFC Separation

Azetidin-1-yl(2-(4-(4-fluoropyrazolo[1,5-a]pyridin-2-yl)-1,4,6,7-tetrahydro-5H-imidazo[4,5-c]pyridin-5-yl)pyrimidin-4-yl)methanone (75.0 mg) was separated by SFC (Column: ChiralPak AS, (4.6*100 mm, 3 μm); mobile phase: [CO₂-MeOH(0.05% DEA)]; B %: 30%, isocratic elution mode) to give Enantiomer #1 (26.2 mg, Rt=1.617 min) and Enantiomer #2 (25.9 mg, Rt=3.884 min).

Enantiomer #1 (Example 254), (R)-azetidin-1-yl(2-(4-(4-fluoropyrazolo[1,5-a]pyridin-2-yl)-1,4,6,7-tetrahydro-5H-imidazo[4,5-c]pyridin-5-yl)pyrimidin-4-yl)methanone: ¹H NMR (400 MHz, DMSO-d₆) δ 12.00 (s, 1H), 8.60 (d, 1H), 8.54 (d, 1H), 7.56 (s, 1H), 7.16-7.04 (m, 2H), 6.97 (s, 1H), 6.83 (dd, 1H), 6.65 (s, 1H), 5.02-4.91 (m, 1H), 4.64 (s, 2H), 4.06 (t, 2H), 3.47 (t, 1H), 2.87-2.59 (m, 2H), 2.29 (s, 2H); LC-MS: m/z 419.2 [M+H]⁺; SFC: 100% ee.

Enantiomer #2 (Example 255), (S)-azetidin-1-yl(2-(4-(4-fluoropyrazolo[1,5-a]pyridin-2-yl)-1,4,6,7-tetrahydro-5H-imidazo[4,5-c]pyridin-5-yl)pyrimidin-4-yl)methanone: ¹H NMR (400 MHz, DMSO-d₆) δ 12.00 (s, 1H), 8.60 (d, 1H), 8.54 (d, 1H), 7.56 (s, 1H), 7.09 (dd, 2H), 6.97 (s, 1H), 6.83 (dd, 1H), 6.65 (s, 1H), 5.00-4.92 (m, 1H), 4.64 (s, 2H), 4.06 (t, 2H), 3.47 (t, 1H), 2.88-2.62 (m, 2H), 2.29 (s, 2H); LC-MS: m/z 419.2 [M+H]⁺; SFC: 100% ee.

### Preparation of Examples 258 and 259 According to General Scheme 2, Method B, and General Scheme 5

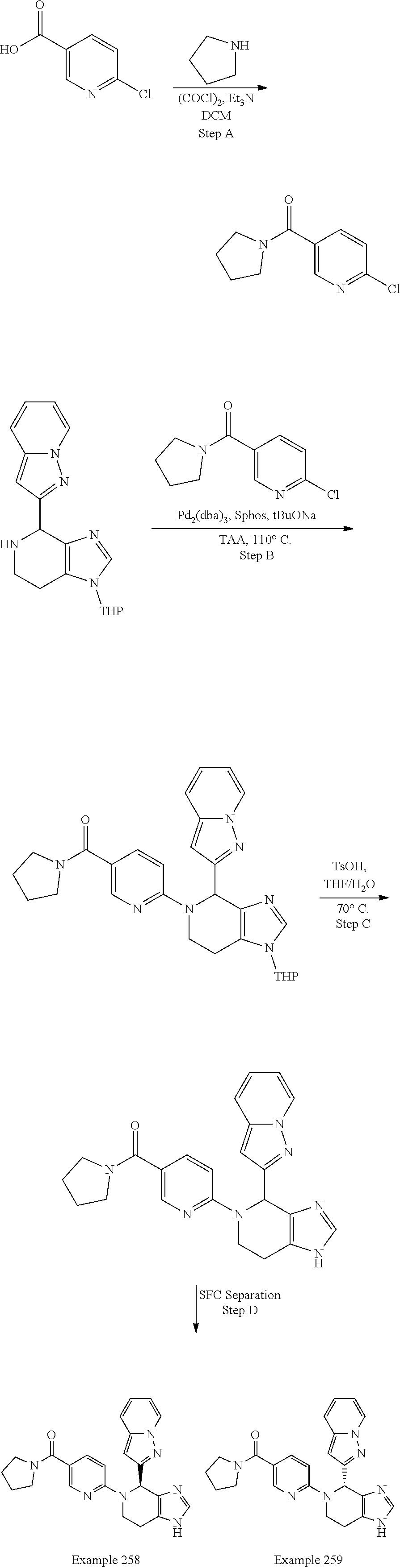

### Step A. Synthesis of (6-chloropyridin-3-yl)(pyrrolidin-1-yl)methanone

Oxalyl chloride (1.0 g, 7.62 mmol), followed by DMF (2 drops), was added to a solution of 6-chloropyridine-3-carboxylic acid (1.0 g, 6.35 mmol) in DCM (10 mL). The reaction mixture was stirred at rt for 1 hr, and the solvent was removed under reduced pressure. The residue was taken up in DCM (10 mL), and tetrahydropyrrole (0.5 g, 6.98 mmol) and Et₃N (1.0 g, 9.52 mmol) were added. The mixture was stirred at rt for 1 hr, and then concentrated under reduced pressure. EtOAc (50 mL) and water (30 mL) were added to the residue, and the aqueous portion extracted with EtOAc (30 mL×3). The combined organic layer was washed with brine (20 mL) and concentrated to dryness. The residue was purified by flash column chromatography on silica gel (0-29% EtOAc in PE) to afford (6-chloropyridin-3-yl)(tetrahydro-1H-pyrrol-1-yl)methanone (890 mg, 66% yield). ¹H NMR (400 MHz, DMSO-d₆) δ 8.77-8.36 (m, 1H), 8.01 (dd, 1H), 7.60 (d, 1H), 3.48 (t, 2H), 3.41 (t, 2H), 1.92-1.76 (m, 4H); LC-MS: m/z 210.9, 212.9 [M+H]⁺.

### Step B. Synthesis of (6-(4-(pyrazolo[1,5-a]pyridin-2-yl)-1-(tetrahydro-2H-pyran-2-yl)-1,4,6,7-tetrahydro-5H-imidazo[4,5-c]pyridin-5-yl)pyridin-3-yl)(pyrrolidin-1-yl)methanone

To the solution of (6-chloropyridin-3-yl)(tetrahydro-1H-pyrrol-1-yl)methanone (586 mg, 2.78 mmol) in 2-methylbutan-2-ol (6 mL) was added 4-(pyrazolo[1,5-a]pyridin-2-yl)-1-(3,4,5,6-tetrahydro-2H-pyran-2-yl)-4,5,6,7-tetrahydroimidazo[5,4-c]pyridine (600 mg, 1.86 mmol), Sphos (152 mg, 0.37 mmol), sodium tert-butoxide (535 mg, 5.57 mmol), and Pd₂(dba)₃ (170 mg, 0.19 mmol). The reaction mixture was stirred at 110° C. for 16 hrs under nitrogen, and allowed to cool to rt. The reaction mixture was poured into water (30 mL), and the aqueous portion extracted with EtOAc (30 mL×3). The combined organic layer was washed with brine (20 mL) and concentrated to dryness. The residue was purified by flash column chromatography on silica gel (0-5% MeOH in DCM) to afford (6-(4-(pyrazolo[1,5-a]pyridin-2-yl)-1-(tetrahydro-2H-pyran-2-yl)-1,4,6,7-tetrahydro-5H-imidazo[4,5-c]pyridin-5-yl)pyridin-3-yl)(pyrrolidin-1-yl)methanone (217 mg, 23% yield). ¹H NMR (400 MHz, DMSO-d₆) δ 8.64-8.59 (m, 1H), 8.36 (s, 1H), 7.73 (d, 2H), 7.60 (d, 1H), 7.19-7.13 (m, 1H), 7.05 (d, 1H), 6.85-6.78 (m, 1H), 6.68-6.61 (m, 1H), 6.54 (s, 1H), 5.30-5.16 (m, 1H), 4.84-4.72 (m, 1H), 4.00-3.90 (m, 1H), 3.64-3.44 (m, 6H), 2.88-2.70 (m, 2H), 2.08-1.76 (m, 10H); LC-MS: m/z 498.2 [M+H]⁺.

### Step C. Synthesis of (6-(4-(pyrazolo[1,5-a]pyridin-2-yl)-1,4,6,7-tetrahydro-5H-imidazo[4,5-c]pyridin-5-yl)pyridin-3-yl)(pyrrolidin-1-yl)methanone

To the solution of (6-(4-(pyrazolo[1,5-a]pyridin-2-yl)-1-(tetrahydro-2H-pyran-2-yl)-1,4,6,7-tetrahydro-5H-imidazo[4,5-c]pyridin-5-yl)pyridin-3-yl)(pyrrolidin-1-yl)methanone (217 mg, 0.44 mmol) in THF (3 mL) was added p-toluenesulfonic acid (376 mg, 2.18 mmol) and H₂O (0.3 mL). The reaction mixture was stirred at 70° C. for 12 hrs, and the solvent removed under reduced pressure. DCM (10 mL) and water (10 mL) were added to the residue, and the pH of aqueous portion was adjusted to a pH of 7 to 8 by the addition of sat. aq. sodium bicarbonate solution. The aqueous portion was extracted with DCM (30 mL×3). The combined organic layer was washed with brine (20 mL) and concentrated to dryness. The residue was purified by prep-HPLC to give (6-(4-(pyrazolo[1,5-a]pyridin-2-yl)-1,4,6,7-tetrahydro-5H-imidazo[4,5-c]pyridin-5-yl)pyridin-3-yl)(pyrrolidin-1-yl)methanone (107 mg, 59% yield). LC-MS: m/z 414.3 [M+H]⁺.

### Step D. SFC Separation

(6-(4-(pyrazolo[1,5-a]pyridin-2-yl)-1,4,6,7-tetrahydro-5H-imidazo[4,5-c]pyridin-5-yl)pyridin-3-yl)(pyrrolidin-1-yl)methanone (107 mg) was separated by SFC (column: ChiralPak IB (250×30 mm, 5 μm); mobile phase: [CO₂-MEOH(0.1% NH₃H₂O)]; B %: 35%; isocratic elution mode) to give Enantiomer #1 (20.1 mg, Rt=3.701 min) and Enantiomer #2 (17.9 mg, Rt=5.332 min).

Enantiomer #1 (Example 258), (R)-(6-(4-(pyrazolo[1,5-a]pyridin-2-yl)-1,4,6,7-tetrahydro-5H-imidazo[4,5-c]pyridin-5-yl)pyridin-3-yl)(pyrrolidin-1-yl)methanone: ¹H NMR (400 MHz, DMSO-d₆) δ 11.96 (s, 1H), 8.60 (d, 1H), 8.35 (d, 1H), 7.73 (dd, 1H), 7.60 (t, 1H), 7.51 (s, 1H), 7.23-7.10 (m, 1H), 7.03 (d, 1H), 6.98-6.57 (m, 2H), 6.50 (s, 1H), 4.85-4.45 (m, 1H), 3.58-3.40 (m, 5H), 2.89-2.75 (m, 1H), 2.65-2.55 (m, 1H), 1.82 (s, 4H); LC-MS: m/z 414.2 [M+H]⁺; SFC: 100% ee.

Enantiomer #2 (Example 259), (S)-(6-(4-(pyrazolo[1,5-a]pyridin-2-yl)-1,4,6,7-tetrahydro-5H-imidazo[4,5-c]pyridin-5-yl)pyridin-3-yl)(pyrrolidin-1-yl)methanone: ¹HNMR (400 MHz, DMSO-d₆) δ 11.96 (s, 1H), 8.60 (d, 1H), 8.36 (s, 1H), 7.73 (d, 1H), 7.60 (d, 1H), 7.51 (s, 1H), 7.21-7.10 (m, 1H), 7.03 (d, 1H), 6.98-6.56 (m, 2H), 6.50 (s, 1H), 4.99-4.50 (m, 1H), 3.58-3.42 (m, 5H), 2.88-2.76 (m, 1H), 2.66-2.58 (s, 1H), 1.82 (s, 4H); LC-MS: m/z 414.2 [M+H]⁺; SFC: 100% ee.

### Preparation of Examples 269 and 270 According to General Schemes 4A and 4B

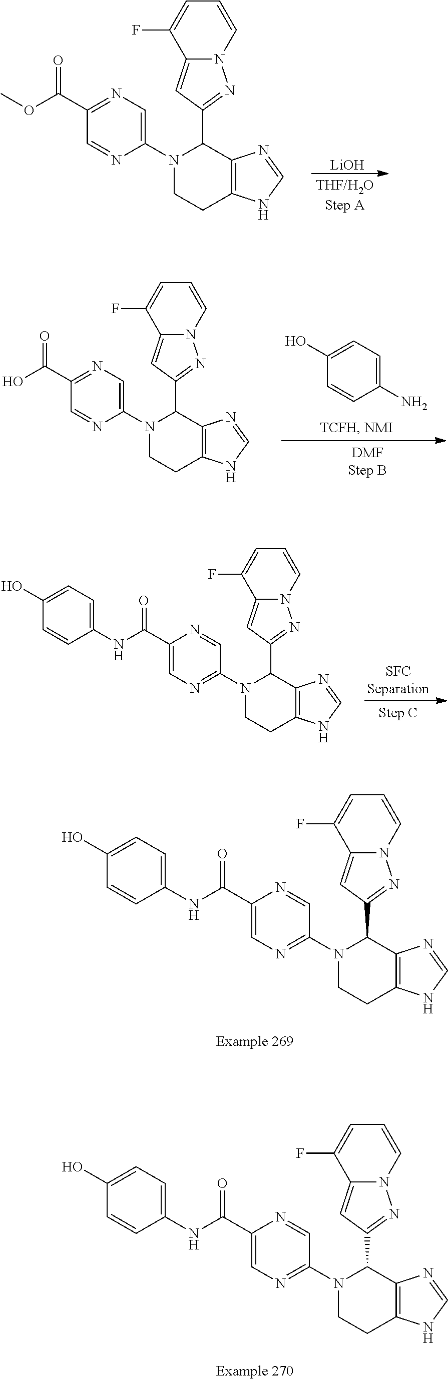

### Step A. Synthesis of 5-(4-(4-fluoropyrazolo[1,5-a]pyridin-2-yl)-1,4,6,7-tetrahydro-5H-imidazo[4,5-c]pyridin-5-yl)pyrazine-2-carboxylic acid

To a solution of methyl 5-[4-(4-fluoropyrazolo[1,5-a]pyridin-2-yl)-4,5,6,7-tetrahydro-1H-imidazo[5,4-c]pyridin-5-yl]pyrazine-2-carboxylate (2.0 g, 5.08 mmol) in THF (15 mL) was added LiOH (10.0 mL, 15.0 mmol, 1.5 mol/mL). The reaction mixture was stirred at rt for 1 hr, and then concentrated. The pH of the reaction mixture was adjusted to a pH of 6 by the addition of HCl (1N) and then concentrated to dryness to afford 5-(4-(4-fluoropyrazolo[1,5-a]pyridin-2-yl)-1,4,6,7-tetrahydro-5H-imidazo[4,5-c]pyridin-5-yl)pyrazine-2-carboxylic acid (1.9 g, 99% yield), which was used without further purification. ¹H NMR (400 MHz, DMSO-d₆) δ 15.01 (s, 1H), 12.91 (s, 1H), 9.05 (s, 1H), 8.75 (d, 1H), 8.68 (d, 1H), 8.58 (d, 1H), 7.27 (s, 1H), 7.17 (dd, 1H), 6.96-6.87 (m, 2H), 4.81 (dd, 1H), 3.58 (d, 1H), 3.05-2.96 (m, 1H), 2.93-2.85 (m, 1H); LC-MS: m/z 380.2 [M+H]⁺.

### Step B. Synthesis of 5-(4-(4-fluoropyrazolo[1,5-a]pyridin-2-yl)-1,4,6,7-tetrahydro-5H-imidazo[4,5-c]pyridin-5-yl)-N-(4-hydroxyphenyl)pyrazine-2-carboxamide

To a solution of 5-(4-(4-fluoropyrazolo[1,5-a]pyridin-2-yl)-1,4,6,7-tetrahydro-5H-imidazo[4,5-c]pyridin-5-yl)pyrazine-2-carboxylic acid (60.0 mg, 0.16 mmol) in DMF (1 mL) was added 4-aminophenol (34.5 mg, 0.32 mmol), [chloro(dimethylamino)methylidene]-dimethylammonium-hexafluoro-λ5-phosphanuide (TCFH) (53.3 mg, 0.19 mmol) and 1-methylimidazole (NMI) (39.0 mg, 0.47 mmol). The reaction mixture was stirred at rt for 1 hr, and then poured into water (5 mL). The aqueous portion was extracted with EtOAc (5 mL×3). The combined organic layer was washed with brine (3 mL×3), dried over Na₂SO₄, filtered, and concentrated to dryness. The residue was purified with prep-HPLC (C18 column (mobile phase: 0-36% MeCN in deionized water (0.1% NH₄HCO₃)) to afford 5-(4-(4-fluoropyrazolo[1,5-a]pyridin-2-yl)-1,4,6,7-tetrahydro-5H-imidazo[4,5-c]pyridin-5-yl)-N-(4-hydroxyphenyl)pyrazine-2-carboxamide (40.0 mg, 54% yield). LC-MS: m/z 471.2 [M+H]⁺.

### Step C. SFC Separation

5-(4-(4-fluoropyrazolo[1,5-a]pyridin-2-yl)-1,4,6,7-tetrahydro-5H-imidazo[4,5-c]pyridin-5-yl)-N-(4-hydroxyphenyl)pyrazine-2-carboxamide (30 mg, 0.06 mmol) was separated by SFC (column: CHIRALPAK AD (100*4.6 mm, 5 μm); mobile phase: [CO₂-MeOH(0.05% DEA)]; B %:40%, isocratic elution mode) to afford Enantiomer #1 (5.9 mg, Rt=2.296 min, 20% yield) and Enantiomer #2 (6.0 mg, Rt=4.659 min, 20% yield).

Enantiomer #1 (Example 269), (R)-5-(4-(4-fluoropyrazolo[1,5-a]pyridin-2-yl)-1,4,6,7-tetrahydro-5H-imidazo[4,5-c]pyridin-5-yl)-N-(4-hydroxyphenyl)pyrazine-2-carboxamide: ¹H NMR (400 MHz, DMSO-d₆) δ 12.06 (s, 1H), 9.96 (s, 1H), 9.23 (s, 1H), 8.74 (s, 1H), 8.57 (s, 1H), 8.54 (d, 1H), 7.61 (d, 3H), 7.15-7.05 (m, 1H), 6.91-6.77 (m, 2H), 6.75-6.66 (m, 3H), 4.94 (d, 1H), 3.58 (m, 1H), 2.87 (m, 1H), 2.75 (m, 1H); LC-MS: m/z 471.2 [M+H]⁺; 100% ee.

Enantiomer #2 (Example 270), (S)-5-(4-(4-fluoropyrazolo[1,5-a]pyridin-2-yl)-1,4,6,7-tetrahydro-5H-imidazo[4,5-c]pyridin-5-yl)-N-(4-hydroxyphenyl)pyrazine-2-carboxamide: ¹H NMR (400 MHz, DMSO-d₆) δ 12.06 (s, 1H), 9.96 (s, 1H), 9.23 (s, 1H), 8.74 (s, 1H), 8.57 (s, 1H), 8.54 (d, 1H), 7.61 (d, 3H), 7.15-7.05 (m, 1H), 6.91-6.77 (m, 2H), 6.75-6.66 (m, 3H), 4.94 (d, 1H), 3.58 (m, 1H), 2.87 (m, 1H), 2.75 (m, 1H); LC-MS: m/z 471.2 [M+H]⁺; 99.4% ee.

### Preparation of Examples 279 and 280 According to General Schemes 4A and 4B

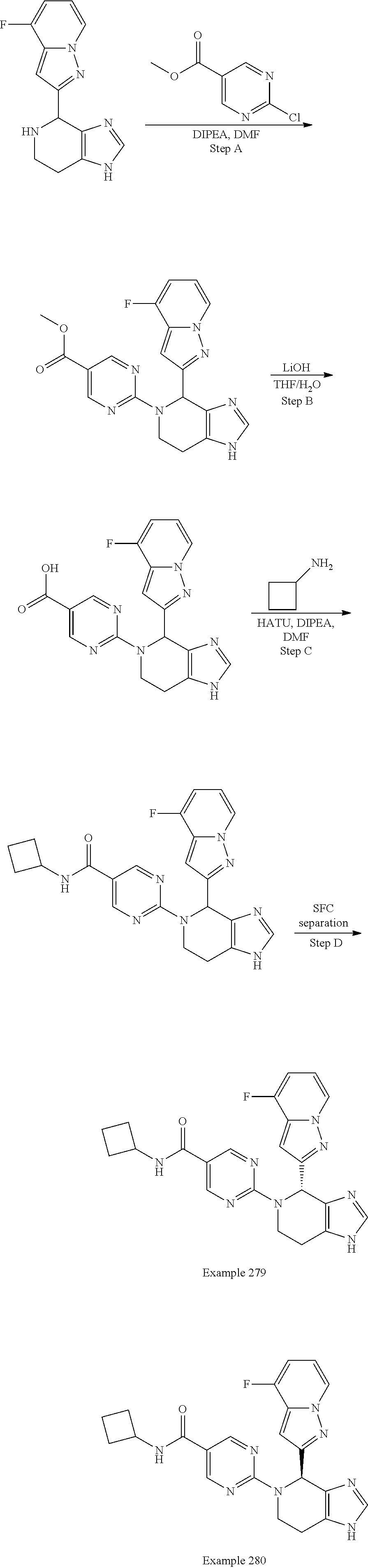

### Step A: Synthesis of methyl 2-[4-(4-fluoropyrazolo[1,5-a]pyridin-2-yl)-1,4,6,7-tetrahydroimidazo[4,5-c]pyridin-5-yl]pyrimidine-5-carboxylate

To a solution of 4-(4-fluoropyrazolo[1,5-a]pyridin-2-yl)-4,5,6,7-tetrahydro-1H-imidazo [4,5-c]pyridine (500 mg, 1.94 mmol) in DMF (5 mL) was added DIPEA (754 mg, 5.83 mmol, 1.02 mL) and methyl 2-chloropyrimidine-5-carboxylate (369 mg, 2.14 mmol). The reaction mixture was stirred at rt for 14 hrs. Water (30 mL) was added to the reaction mixture, and the aqueous portion extracted with EtOAc (20 mL×3). The combined organic layer was washed with brine (20 mL), dried over Na₂SO₄, filtered, and concentrated to dryness. The residue was purified by flash silica gel chromatography (ISCO®; 12 g SepaFlash® Silica Flash Column, Eluent of 0-4% MeOH/DCM @30 mL/min) to give methyl 2-[4-(4-fluoropyrazolo[1,5-a]pyridin-2-yl)-1,4,6,7-tetrahydroimidazo[4,5-c]pyridin-5-yl]pyrimidine-5-carboxylate (580 mg, 69.8% yield). LCMS: m/z 394.2 [M+H]⁺.

### Step B: Synthesis of 2-[4-(4-fluoropyrazolo[1,5-a]pyridin-2-yl)-1,4,6,7-tetrahydroimidazo [4,5-c]pyridin-5-yl]pyrimidine-5-carboxylic acid

To a solution of methyl 2-[4-(4-fluoropyrazolo[1,5-a]pyridin-2-yl)-1,4,6,7-tetrahydroimidazo [4,5-c]pyridin-5-yl]pyrimidine-5-carboxylate (580 mg, 1.47 mmol) in THF (5 mL) and H₂O (5 mL) was added LiOH·H₂O (68.1 mg, 1.62 mmol). The reaction mixture was stirred at 15° C. for 14 hrs and then concentrated under reduced pressure. The pH of the reaction mixture was adjusted to a pH of approximately 4 by adding 2 M HCl. A precipitate was formed and collected by filtration and then dried under reduced pressure to give 2-[4-(4-fluoropyrazolo[1,5-a]pyridin-2-yl)-1,4,6,7-tetrahydroimidazo [4,5-c]pyridin-5-yl]pyrimidine-5-carboxylic acid (520 mg, 87.4% yield). ¹H NMR (400 MHz, DMSO-d₆) δ 8.88 (s, 2H), 8.44-8.61 (m, 2H), 7.28 (s, 1H), 7.14 (dd, 1H), 6.88 (td, 1H), 6.80 (s, 1H), 5.12 (br dd, 1H), 3.46 (br dd, 1H), 2.79-2.89 (m, 2H); LCMS: m/z 380.1 [M+H]⁺.

### Step C: Synthesis of N-cyclobutyl-2-(4-(4-fluoropyrazolo[1,5-a]pyridin-2-yl)-1,4,6,7-tetrahydro-5H-imidazo[4,5-c]pyridin-5-yl)pyrimidine-5-carboxamide

To a solution of 2-[4-(4-fluoropyrazolo[1,5-a]pyridin-2-yl)-1,4,6,7-tetrahydroimidazo[4,5-c]pyridin-5-yl]pyrimidine-5-carboxylic acid (100 mg, 264 μmol) and cyclobutanamine (28.1 mg, 395 μmol, 33.9 μL) in DMF (2 mL) was added HATU (120 mg, 316 μmol) and DIPEA (102 mg, 791 μmol, 138 μL). The reaction mixture was stirred at rt for 14 hrs. The reaction mixture was then purified by prep-HPLC (column: Boston Prime C18 (150*30 mm*5 μm); mobile phase: [water(NH₃H₂O+NH₄HCO₃)-ACN]; gradient: 30%-60% B over 11 min) to give N-cyclobutyl-2-(4-(4-fluoropyrazolo[1,5-a]pyridin-2-yl)-1,4,6,7-tetrahydro-5H-imidazo[4,5-c]pyridin-5-yl)pyrimidine-5-carboxamide (90 mg). LCMS: m/z 433.2 [M+H]⁺.

### Step D: SFC Separation

2-[4-(4-fluoropyrazolo[1,5-a]pyridin-2-yl)-1,4,6,7-tetrahydroimidazo[4,5-c]pyridin-5-yl]pyrimidine-5-carboxylic acid (100 mg, 264 μmol) was separated by SFC (column: DAICEL CHIRALPAK IC (250 mm*30 mm, 10 μm); mobile phase: [CO₂-EtOH (0.1% NH₃H₂O)]; B %:60%, isocratic elution mode) to give Enantiomer #1 (35.8 mg, Rt=1.099 min, 31.3% yield) and Enantiomer #2 (36.5 mg, Rt=1.989 min, 31.7% yield).

Enantiomer #1 (Example 279), (S)—N-cyclobutyl-2-(4-(4-fluoropyrazolo[1,5-a]pyridin-2-yl)-1,4,6,7-tetrahydro-5H-imidazo[4,5-c]pyridin-5-yl)pyrimidine-5-carboxamide: ¹H NMR (400 MHz, METHANOL-d₄) δ 8.82 (s, 2H), 8.29 (br d, 1H), 7.64 (s, 1H), 7.24 (s, 1H), 6.92 (dd, 1H), 6.74-6.83 (m, 1H), 6.67 (s, 1H), 5.17 (br dd, 1H), 4.47 (quin, 1H), 3.42-3.58 (m, 1H), 2.81-2.96 (m, 1H), 2.65-2.78 (m, 1H), 2.25-2.43 (m, 2H), 1.99-2.20 (m, 2H), 1.67-1.89 (m, 2H); LCMS: m/z 433.2 [M+H]⁺; SFC: 100% ee.

Enantiomer #2 (Example 280), (R)—N-cyclobutyl-2-(4-(4-fluoropyrazolo[1,5-a]pyridin-2-yl)-1,4,6,7-tetrahydro-5H-imidazo[4,5-c]pyridin-5-yl)pyrimidine-5-carboxamide: ¹H NMR (400 MHz, METHANOL-d₄) δ 8.82 (s, 2H), 8.29 (d, 1H), 7.64 (s, 1H), 7.24 (s, 1H), 6.92 (dd, 1H), 6.79 (td, 1H), 6.67 (s, 1H), 5.17 (dd, 1H), 4.47 (quin, 1H), 3.45-3.57 (m, 1H), 2.83-2.97 (m, 1H), 2.69-2.79 (m, 1H), 2.27-2.40 (m, 2H), 2.04-2.16 (m, 2H), 1.68-1.91 (m, 2H); LCMS: m/z 433.3 [M+H]⁺; SFC: 99.8% ee.

### Synthesis of Example 281 According to General Scheme 7

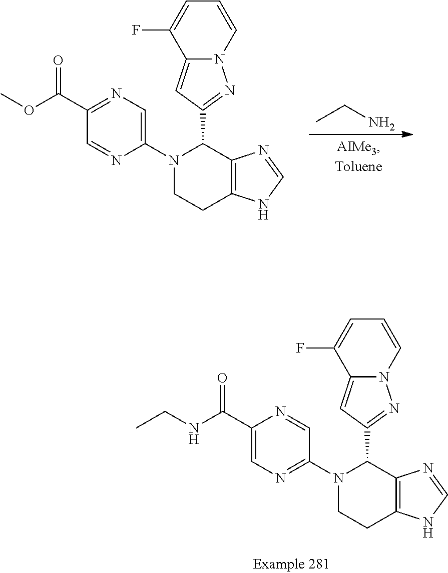

### Synthesis of (S)—N-ethyl-5-(4-(4-fluoropyrazolo[1,5-a]pyridin-2-yl)-1,4,6,7-tetrahydro-5H-imidazo[4,5-c]pyridin-5-yl)pyrazine-2-carboxamide

To a mixture of methyl (S)-5-(4-(4-fluoropyrazolo[1,5-a]pyridin-2-yl)-1,4,6,7-tetrahydro-5H-imidazo[4,5-c]pyridin-5-yl)pyrazine-2-carboxylate (40.0 mg, 0.10 mmol) and ethylamine (0.1 mL, 2.0 mmol) in toluene (1 mL) was added trimethylaluminum (0.2 mL, 0.20 mmol, 1.0 M in hexane). The reaction mixture was stirred at 100° C. for 18 hrs and then cooled to rt. Water (10 mL) was added to the reaction mixture, and the aqueous portion extracted with DCM (3×20 mL). The combined organic layer was washed with brine (10 mL), dried over Na₂SO₄, filtered, and concentrated to dryness. The residue was purified by Prep-HPLC (mobile phase: ACN-H₂O+0.1% NH₄HCO₃) to obtain Example 675 (28.0 mg, 69% yield). ¹H NMR (400 MHz, DMSO-d₆) δ 12.03 (s, 1H), 8.63 (s, 1H), 8.56-8.49 (m, 2H), 8.38 (t, 1H), 7.58 (s, 1H), 7.10 (dd, 1H), 6.84 (dd, 2H), 6.72 (s, 1H), 4.90 (d, 1H), 3.58-3.51 (m, 1H), 3.27-3.24 (m, 2H), 2.93-2.81 (m, 1H), 2.79-2.65 (m, 1H), 1.09 (t, 3H); LC-MS: m/z 407.2 [M+H]⁺; >99% ee (based on the enantiopurity of the chiral intermediate (S)-5-(4-(4-fluoropyrazolo[1,5-a]pyridin-2-yl)-1,4,6,7-tetrahydro-5H-imidazo[4,5-c]pyridin-5-yl)pyrazine-2-carboxylate).

### Preparation of Examples 283 and 284 According to General Schemes 4A and 4B

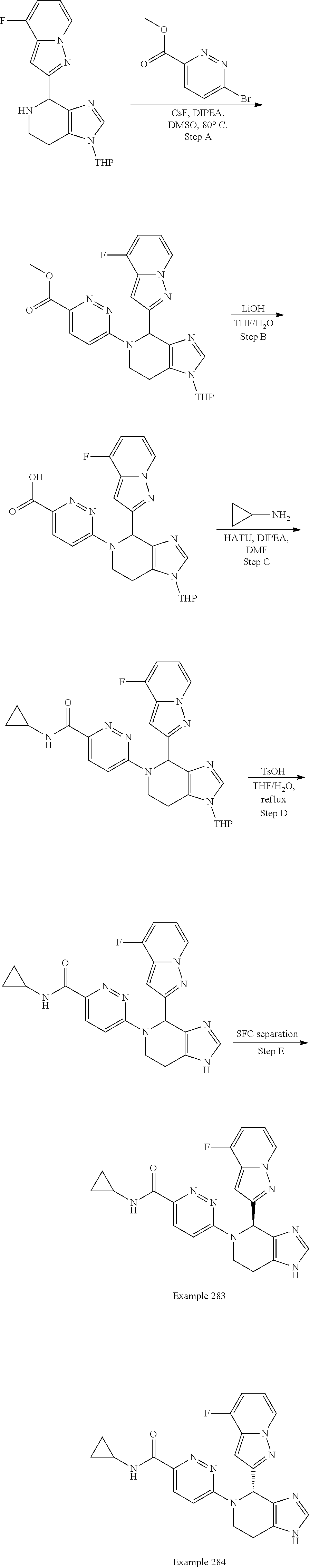

### Step A: Synthesis of methyl 6-[4-(4-fluoropyrazolo [1,5-a]pyridin-2-yl)-1-tetrahydropyran-2-yl-6,7-dihydro-4H-imidazo[4,5-c]pyridin-5-yl]pyridazine-3-carboxylate

To a solution of 4-(4-fluoropyrazolo[1,5-a]pyridin-2-yl)-1-tetrahydropyran-2-yl-4,5,6,7-tetrahydroimidazo[4,5-c]pyridine (3 g, 8.79 mmol) and methyl 6-bromopyridazine-3-carboxylate (2.29 g, 10.6 mmol) in DMSO (30 mL) was added CsF (2.67 g, 17.6 mmol and DIPEA (3.41 g, 26.4 mmol, 4.59 mL), and the reaction mixture was stirred at 80° C. for 12 hrs under N₂ atmosphere. EtOAc (200 mL) was added to the reaction mixture, and the organic layer washed with water (100 mL) and brine (100 mL), dried over Na₂SO₄, filtered, and concentrated to dryness. The residue was purified by flash silica gel chromatography (ISCO®; 20 g SepaFlash® Silica Flash Column, Eluent of 0-5% MeOH:DCM @30 mL/min) to give methyl 6-[4-(4-fluoropyrazolo [1,5-a]pyridin-2-yl)-1-tetrahydropyran-2-yl-6,7-dihydro-4H-imidazo[4,5-c]pyridin-5-yl]pyridazine-3-carboxylate (4 g). LCMS: m/z 478.3 [M+H]⁺.

### Step B: Synthesis of 6-[4-(4-fluoropyrazolo[1,5-a]pyridin-2-yl)-1-tetrahydropyran-2-yl-6,7-dihydro-4H-imidazo[4,5-c]pyridin-5-yl]pyridazine-3-carboxylic acid

To a solution of methyl 6-[4-(4-fluoropyrazolo[1,5-a]pyridin-2-yl)-1-tetrahydropyran-2-yl-6,7-dihydro-4H-imidazo[4,5-c]pyridin-5-yl]pyridazine-3-carboxylate (4 g, 8.38 mmol) in THF (30 mL) was added LiOH·H₂O (422 mg, 10.1 mmol) in H₂O (30 mL), and the reaction mixture was stirred at rt for 2 hrs. The reaction mixture was concentrated in vacuum, and 4 M HCl solution was added to the reaction mixture until the pH of the mixture was approximately equal to pH 6. A precipitate formed and was collected by filtration, dried in vacuum, and then purified by flash silica gel chromatography (ISCO®; 20 g SepaFlash® Silica Flash Column, Eluent of 0-20% MeOH:DCM @40 mL/min) to give 6-[4-(4-fluoropyrazolo[1,5-a]pyridin-2-yl)-1-tetrahydropyran-2-yl-6,7-dihydro-4H-imidazo[4,5-c]pyridin-5-yl]pyridazine-3-carboxylic acid (3.4 g, 80.0% yield). LCMS: m/z 464.2 [M+H]⁺.

### Step C: Synthesis of N-cyclopropyl-6-[4-(4-fluoropyrazolo[1,5-a]pyridin-2-yl)-1-tetrahydropyran-2-yl-6,7-dihydro-4H-imidazo[4,5-c]pyridin-5-yl]pyridazine-3-carboxamide

To a solution of 6-[4-(4-fluoropyrazolo[1,5-a]pyridin-2-yl)-1-tetrahydropyran-2-yl-6,7-dihydro-4H-imidazo[4,5-c]pyridin-5-yl]pyridazine-3-carboxylic acid (3.4 g, 7.34 mmol) in DMF (30 mL) was added DIPEA (2.84 g, 22.0 mmol, 3.83 mL), HATU (4.18 g, 11.0 mmol) and cyclopropanamine (2.09 g, 36.7 mmol, 2.54 mL). The reaction mixture was stirred at 30° C. for 12 hrs under N₂ atmosphere. EtOAc (200 mL) was added to the reaction mixture, and the organic layer washed by water (100 mL) and brine (100 mL), dried with Na₂SO₄, filtered, and concentrated to dryness. The residue was purified by flash silica gel chromatography (ISCO®; 40 g SepaFlash® Silica Flash Column, Eluent of 0-5% MeOH:DCM @40 mL/min) to give N-cyclopropyl-6-[4-(4-fluoropyrazolo[1,5-a]pyridin-2-yl)-1-tetrahydropyran-2-yl-6,7-dihydro-4H-imidazo[4,5-c]pyridin-5-yl]pyridazine-3-carboxamide (2.2 g, 56.1% yield). LCMS: m/z 503.2 [M+H]⁺.

### Step D: Synthesis of N-cyclopropyl-6-[4-(4-fluoropyrazolo[1,5-a]pyridin-2-yl)-1,4,6,7-tetrahydroimidazo[4,5-c]pyridin-5-yl]pyridazine-3-carboxamide

To a solution of N-cyclopropyl-6-[4-(4-fluoropyrazolo[1,5-a]pyridin-2-yl)-1-tetrahydropyran-2-yl-6,7-dihydro-4H-imidazo[4,5-c]pyridin-5-yl]pyridazine-3-carboxamide (3.4 g, 6.77 mmol) in THF (25 mL) and H₂O (5 mL) was added TsOH (2.33 g, 13.5 mmol). The reaction mixture was stirred at 80° C. for 12 hrs and then concentrated in vacuum. EtOAc (200 mL) was added to the residue, and the organic layer washed with sat. aq. NaHCO₃ solution (60 mL) and brine (60 mL), dried over Na₂SO₄, filtered, and concentrated to dryness. The residue was purified by prep-HPLC (column: Boston Prime C18 (150*30 mm*5 μm); mobile phase: [water(NH₄HCO₃)-ACN]; gradient:20%-50% B over 11 min) to give N-cyclopropyl-6-[4-(4-fluoropyrazolo[1,5-a]pyridin-2-yl)-1,4,6,7-tetrahydroimidazo[4,5-c]pyridin-5-yl]pyridazine-3-carboxamide (1.3 g, 45.9% yield). LCMS: m/z 419.1 [M+H]⁺.

### Step E: SFC Separation

N-cyclopropyl-6-[4-(4-fluoropyrazolo[1,5-a]pyridin-2-yl)-1,4,6,7-tetrahydroimidazo[4,5-c]pyridin-5-yl]pyridazine-3-carboxamide (1.3 g) was separated by SFC (column: DAICEL CHIRALPAK AD (250 mm*30 mm, 10 μm); mobile phase: [CO₂-i-PrOH(0.1% NH₃H₂O)]; B %:55%, isocratic elution mode) to give Enantiomer #1 (276 mg, Rt=0.831 min, 9.3% yield) and Enantiomer #2 (234 mg, Rt=2.076 min, 8.3% yield).

Enantiomer #1 (Example 283), (R)—N-cyclopropyl-6-(4-(4-fluoropyrazolo[1,5-a]pyridin-2-yl)-1,4,6,7-tetrahydro-5H-imidazo[4,5-c]pyridin-5-yl)pyridazine-3-carboxamide: ¹H NMR (400 MHz, METHANOL-d₄) δ 8.32 (d, 1H), 7.96 (d, 1H), 7.65 (s, 1H), 7.55 (d, 1H), 6.88-6.99 (m, 2H), 6.79 (td, 1H), 6.69 (s, 1H), 4.78 (br dd, 1H), 3.68 (ddd, 1H), 2.93-3.04 (m, 1H), 2.88 (tt, 1H), 2.79 (br dd, 1H), 0.79-0.87 (m, 2H), 0.64-0.71 (m, 2H); LCMS: m/z 419.2 [M+H]⁺; SFC: 99.6% ee.

Enantiomer #2 (Example 284), (S)—N-cyclopropyl-6-(4-(4-fluoropyrazolo[1,5-a]pyridin-2-yl)-1,4,6,7-tetrahydro-5H-imidazo[4,5-c]pyridin-5-yl)pyridazine-3-carboxamide: ¹H NMR (400 MHz, METHANOL-d₄) δ 8.32 (d, 1H), 7.96 (d, 1H), 7.65 (s, 1H), 7.55 (d, 1H), 6.93 (dd, 2H), 6.79 (td, 1H), 6.69 (s, 1H), 4.74-4.83 (m, 1H), 3.69 (ddd, 1H), 2.94-3.05 (m, 1H), 2.88 (tt, 1H), 2.79 (br dd, 1H), 0.79-0.86 (m, 2H), 0.65-0.71 (m, 2H); LCMS: m/z 419.2 [M+H]⁺; SFC: 100% ee.

### Preparation of Examples 316 and 317 According to General Scheme 8

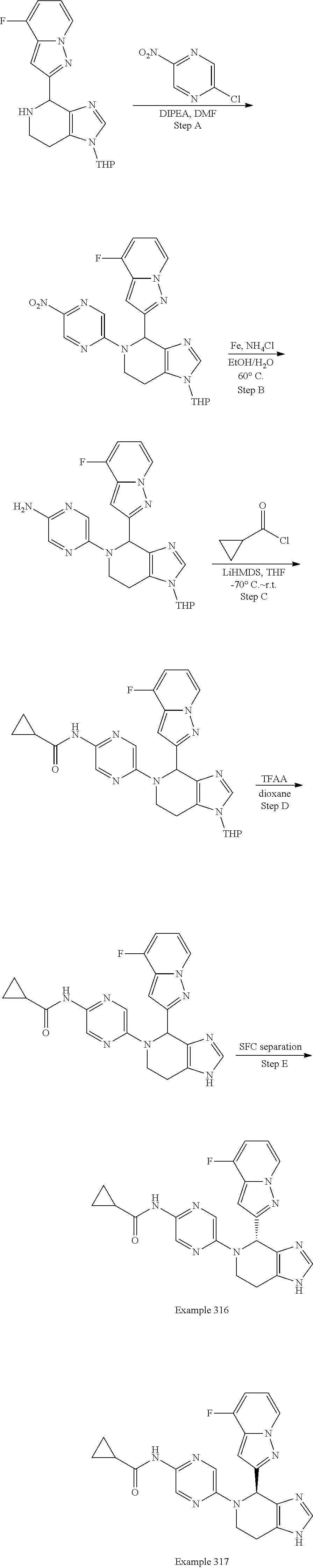

### Step A: Preparation of 4-(4-fluoropyrazolo[1,5-a]pyridin-2-yl)-5-(5-nitropyrazin-2-yl)-1-tetrahydropyran-2-yl-6,7-dihydro-4H-imidazo[4,5-c]pyridine

To a solution of 2-chloro-5-nitro-pyrazine (280 mg, 1.76 mmol) and 4-(4-fluoropyrazolo [1,5-a]pyridin-2-yl)-1-tetrahydropyran-2-yl-4,5,6,7-tetrahydroimidazo[4,5-c]pyridine (500 mg, 1.46 mmol) in DMF (3 mL) was added DIPEA (946 mg, 7.32 mmol, 1.28 mL). The reaction mixture was stirred at rt for 15 hrs. EtOAc (30 mL) was added to the reaction mixture, and the organic layer was washed with sat. aq. Na₂CO₃ solution (30 mL) and brine (30 mL), dried over Na₂SO₄, filtered, and concentrated to dryness. The residue was purified by flash silica gel chromatography (ISCO®; 20 g SepaFlash® Silica Flash Column, Eluent of 0-10% DCM/MeOH @20 mL/min) to afford 4-(4-fluoropyrazolo[1,5-a]pyridin-2-yl)-5-(5-nitropyrazin-2-yl)-1-tetrahydropyran-2-yl-6,7-dihydro-4H-imidazo[4,5-c]pyridine (540 mg, 66.7% yield). LCMS: m/z 465.2 [M+H]⁺.

### Step B: Preparation of 5-[4-(4-fluoropyrazolo[1,5-a]pyridin-2-yl)-1-tetrahydropyran-2-yl-6,7-dihydro-4H-imidazo[4,5-c]pyridin-5-yl]pyrazin-2-amine

To a solution of 4-(4-fluoropyrazolo[1,5-a]pyridin-2-yl)-5-(5-nitropyrazin-2-yl)-1-tetrahydropyran-2-yl-6,7-dihydro-4H-imidazo[4,5-c]pyridine (540 mg, 1.16 mmol) in EtOH (5 mL) was added Fe (519 mg, 9.30 mmol) and NH₄Cl (498 mg, 9.30 mmol) in H₂O (1 mL). The reaction mixture was stirred at 60° C. for 2 hrs, and then cooled to rt. The reaction mixture was filtered, and the filtrate was concentrated to dryness. The residue was purified by flash silica gel chromatography (ISCO®; 4 g SepaFlash® Silica Flash Column, Eluent of 0-10% DCM/MeOH @20 mL/min) to afford 5-[4-(4-fluoropyrazolo[1,5-a]pyridin-2-yl)-1-tetrahydropyran-2-yl-6,7-dihydro-4H-imidazo[4,5-c]pyridin-5-yl]pyrazin-2-amine (320 mg, 61.2% yield). LCMS: m/z 435.2 [M+H]⁺.

### Step C: Preparation of N-[5-[4-(4-fluoropyrazolo[1,5-a]pyridin-2-yl)-1-tetrahydropyran-2-yl-6,7-dihydro-4H-imidazo[4,5-c]pyridin-5-yl]pyrazin-2-yl]cyclopropanecarboxamide

To a solution of 5-[4-(4-fluoropyrazolo[1,5-a]pyridin-2-yl)-1-tetrahydropyran-2-yl-6,7-dihydro-4H-imidazo[4,5-c]pyridin-5-yl]pyrazin-2-amine (270 mg, 621 μmol) in THF (3 mL) was added LiHMDS (1 M, 746 μL) dropwise at −70° C. under N₂. The reaction mixture was stirred for 30 mins at −70° C. and then cyclopropanecarbonyl chloride (97.4 mg, 932 μmol, 84.6 L) was added dropwise. The reaction mixture was stirred at rt for 13.5 hrs, and then sat. aq. NH₄Cl solution (20 mL) was added to the reaction mixture. The aqueous portion was extracted with DCM (3×20 mL), and the combined organic layer concentrated to dryness. The residue was purified by flash silica gel chromatography (ISCO®; 12 g SepaFlash® Silica Flash Column, Eluent of 0-5% EtOAc/PE, gradient @25 mL/min) to afford N-[5-[4-(4-fluoropyrazolo[1,5-a]pyridin-2-yl)-1-tetrahydropyran-2-yl-6,7-dihydro-4H-imidazo[4,5-c]pyridin-5-yl]pyrazin-2-yl]cyclopropanecarboxamide (220 mg, 70.4% yield). LCMS: m/z 503.3 [M+H]⁺.

### Step D: Preparation of N-(5-(4-(4-fluoropyrazolo[1,5-a]pyridin-2-yl)-1,4,6,7-tetrahydro-5H-imidazo [4,5-c]pyridin-5-yl)pyrazin-2-yl)cyclopropanecarboxamide

To a solution of N-[5-[4-(4-fluoropyrazolo[1,5-a]pyridin-2-yl)-1-tetrahydropyran-2-yl-6,7-dihydro-4H-imidazo[4,5-c]pyridin-5-yl]pyrazin-2-yl]cyclopropanecarboxamide (220 mg, 438 μmol) in dioxane (4.5 mL) was added TFAA (919 mg, 4.38 mmol, 609 μL). The reaction mixture was stirred at rt for 14 hrs. Sat. aq. NaHCO₃ solution was added to the reaction mixture until the pH of the mixture was approximately pH 9, and then water (10 mL) was added. The aqueous portion was extracted with DCM (3×15 mL), and the combined organic layer was washed with brine (40 mL), dried over Na₂SO₄, filtered, and concentrated to dryness. The residue was purified by flash silica gel chromatography (ISCO®; 4 g SepaFlash® Silica Flash Column, Eluent of 0-5% MeOH/DCM @20 mL/min) to afford N-(5-(4-(4-fluoropyrazolo[1,5-a]pyridin-2-yl)-1,4,6,7-tetrahydro-5H-imidazo [4,5-c]pyridin-5-yl)pyrazin-2-yl)cyclopropanecarboxamide (110 mg, 50.9% yield). LCMS: m/z 419.2 [M+H]⁺.

### Step E: SFC Separation

N-(5-(4-(4-fluoropyrazolo[1,5-a]pyridin-2-yl)-1,4,6,7-tetrahydro-5H-imidazo[4,5-c]pyridin-5-yl)pyrazin-2-yl)cyclopropanecarboxamide (100 mg) was separated by SFC (Column: DAICEL CHIRALPAK IC (250 mm*30 mm, 10 μm); mobile phase: [CO₂-ACN/MeOH (0.1% NH₃H₂O)]; B %:55%, isocratic elution mode) to give Enantiomer #1 (37 mg, Rt=1.096 min, 76.6% yield) and Enantiomer #2 (39.2 mg, Rt=2.026 min, 85.4% yield).

Enantiomer #1 (Example 316), (S)—N-(5-(4-(4-fluoropyrazolo[1,5-a]pyridin-2-yl)-1,4,6,7-tetrahydro-5H-imidazo[4,5-c]pyridin-5-yl)pyrazin-2-yl)cyclopropanecarboxamide: ¹H NMR (400 MHz, METHANOL-d₄) δ 8.77 (s, 1H), 8.32 (d, 1H), 8.21 (d, 1H), 7.62 (s, 1H), 6.92 (dd, 1H), 6.78 (td, 1H), 6.66-6.75 (m, 1H), 6.61 (s, 1H), 4.55-4.66 (m, 1H), 3.59 (ddd, 1H), 2.89-3.03 (m, 1H), 2.72 (br dd, 1H), 1.79-1.88 (m, 1H), 0.94-0.98 (m, 2H), 0.83-0.88 (m, 2H); LCMS: m/z 419.2 [M+H]⁺; SFC: 99.9% ee.

Enantiomer 2 (Example 317), (R)—N-(5-(4-(4-fluoropyrazolo[1,5-a]pyridin-2-yl)-1,4,6,7-tetrahydro-5H-imidazo[4,5-c]pyridin-5-yl)pyrazin-2-yl)cyclopropanecarboxamide: ¹H NMR (400 MHz, METHANOL-d₄) δ 8.77 (s, 1H), 8.32 (d, 1H), 8.21 (d, 1H), 7.62 (s, 1H), 6.92 (dd, 1H), 6.78 (td, 1H), 6.66-6.75 (m, 1H), 6.61 (s, 1H), 4.55-4.66 (m, 1H), 3.59 (ddd, 1H), 2.89-3.03 (m, 1H), 2.72 (br dd, 1H), 1.79-1.88 (m, 1H), 0.94-0.98 (m, 2H), 0.83-0.88 (m, 2H); LCMS: m/z 419.2 [M+H]⁺; SFC: 99.9% ee.

### Preparation of Examples 349 and 350 According to General Scheme 2, Method A

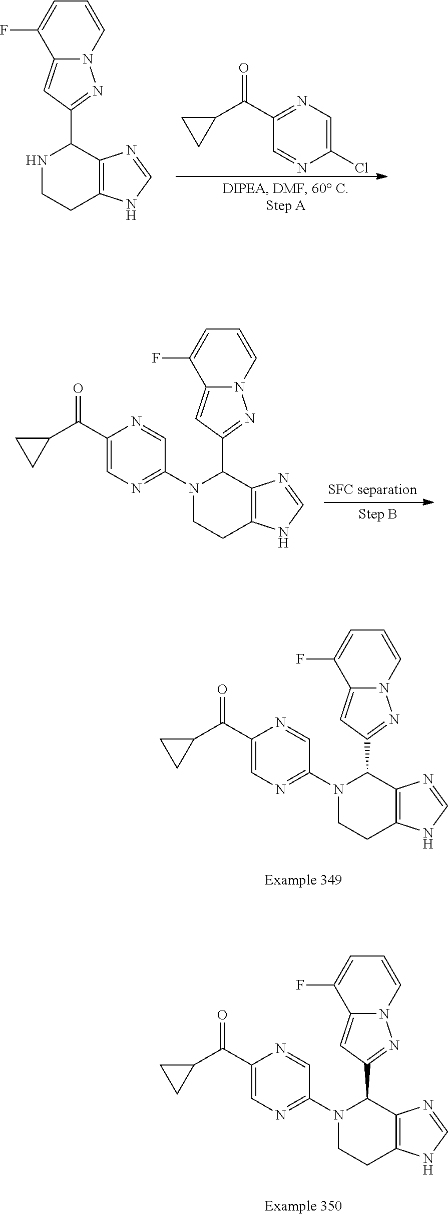 [SMILES: Fc1cccn2nc(C3NCCc4[nH]cnc43)cc12 ; O=C(c1cnc(Cl)cn1)C1CC1 ; O=C(c1cnc(N2CCc3[nH]cnc3C2c2cc3c(F)cccn3n2)cn1)C1CC1 ; O=C(c1cnc(N2CCc3[nH]cnc3[C@H]2c2cc3c(F)cccn3n2)cn1)C1CC1 ; O=C(c1cnc(N2CCc3[nH]cnc3[C@@H]2c2cc3c(F)cccn3n2)cn1)C1CC1]

### Step A. Preparation of cyclopropyl-[5-[4-[4-(trifluoromethyl)pyrazolo[1,5-a]pyridin-2-yl]-1,4,6,7-tetrahydroimidazo[4,5-c]pyridin-5-yl]pyrazin-2-yl]methanone

To a solution of 4-[4-(trifluoromethyl)pyrazolo[1,5-a]pyridin-2-yl]-4,5,6,7-tetrahydro-1H-imidazo[4,5-c]pyridine (200 mg, 651 μmol) and (5-chloropyrazin-2-yl)-cyclopropyl-methanone (178 mg, 976 μmol) in DMF (2 mL) was added DIPEA (421 mg, 3.25 mmol, 567 μL). The reaction mixture was stirred at 60° C. for 15 hrs. EtOAc (80 mL) was added to the reaction mixture. The organic layer washed with sat. aq. Na₂CO₃ solution (30 mL) and brine (30 mL), dried over Na₂SO₄, filtered and concentrated to dryness. The residue was purified by prep-HPLC (column: Boston Prime C18 (150*30 mm*5 μm); mobile phase: [water(NH₃H₂O+NH₄HCO₃)-ACN]; gradient:35%-65% B over 11 min) to give cyclopropyl-[5-[4-[4-(trifluoromethyl)pyrazolo [1,5-a]pyridin-2-yl]-1,4,6,7-tetrahydroimidazo[4,5-c]pyridin-5-yl]pyrazin-2-yl]methanone (100 mg, 33.7% yield). LCMS: m/z 454.1 [M+H]⁺.

### Step B: SFC Separation

Cyclopropyl-[5-[4-[4-(trifluoromethyl)pyrazolo[1,5-a]pyridin-2-yl]-1,4,6,7-tetrahydroimidazo[4,5-c]pyridin-5-yl]pyrazin-2-yl]methanone (100 mg) was separated by SFC (column: DAICEL CHIRALPAK IC (250 mm*30 mm, 10 μm); mobile phase: [CO₂-MeOH (0.1% NH₃H₂O)]; B %:55%, isocratic elution mode) to give Enantiomer #1 (42.9 mg, Rt=1.327 min, 39.0% yield) and Enantiomer #2 (39.3 mg, Rt=2.331 min, 38.3% yield).

Enantiomer #1 (Example 349), (S)-cyclopropyl(5-(4-(4-(trifluoromethyl)pyrazolo[1,5-a]pyridin-2-yl)-1,4,6,7-tetrahydro-5H-imidazo[4,5-c]pyridin-5-yl)pyrazin-2-yl)methanone: ¹H NMR (400 MHz, METHANOL-d₄) δ 8.75 (d, 1H), 8.69 (d, 1H), 8.57 (d, 1H), 7.67 (s, 1H), 7.61 (d, 1H), 6.94-7.03 (m, 2H), 6.71 (s, 1H), 4.91-4.96 (m, 1H), 3.60-3.69 (m, 1H), 3.33-3.36 (m, 1H), 2.95-3.05 (m, 1H), 2.82 (dd, 1H), 1.09-1.13 (m, 2H), 1.03-1.08 (m, 2H); LCMS: m/z 454.2 [M+H]⁺; SFC: 100% ee.

Enantiomer #2 (Example 350), (R)-cyclopropyl(5-(4-(4-(trifluoromethyl)pyrazolo[1,5-a]pyridin-2-yl)-1,4,6,7-tetrahydro-5H-imidazo[4,5-c]pyridin-5-yl)pyrazin-2-yl)methanone: ¹H NMR (400 MHz, METHANOL-d₄) δ 8.74 (d, 1H), 8.68 (d, 1H), 8.56 (d, 1H), 7.67 (s, 1H), 7.61 (d, 1H), 6.92-7.03 (m, 2H), 6.71 (s, 1H), 4.91-4.96 (m, 1H), 3.59-3.71 (m, 1H), 3.32-3.36 (m, 1H), 2.94-3.06 (m, 1H), 2.82 (dd, 1H), 1.10 (dt, 2H), 1.02-1.08 (m, 2H); LCMS: m/z 454.2 [M+H]⁺; SFC: 100% ee.

The compounds of Table 1 were characterized using proton NMR and LCMS and the enantiomeric excess determined. See Table 3.

TABLE 3

| | | LCMS: | |
|---|---|---|---|
| Ex. # | ¹H NMR | m/z | % ee |
| 89 | Data provided above | | |
| 90 | Data provided above | | |
| 91 | (400 MHz, METHANOL-d₄) δ 8.05-8.28 (m, 2 H), 7.61 (s, 1 | 493.2 | 100% |
| | H), 7.43-7.50 (m, 2 H), 7.24-7.42 (m, 3 H), 6.94 (d, 1 H), | [M + H]⁺ | |
| | 6.72 (t, 2 H), 6.47 (s, 1 H), 6.22 (s, 1 H), 4.49-4.74 (m, 2 H), | | |
| | 4.34 (br d, 1 H), 4.08-4.23 (m, 2 H), 3.80 (td, 1 H), 3.39-3.60 | | |
| | (m, 1 H), 3.03-3.20 (m, 1 H), 2.77-2.98 (m, 2 H), 2.69 (br dd, | | |
| | 1 H), 2.38 (s, 3 H) | | |
| 92 | (400 MHz, METHANOL-d₄) δ 8.09-8.39 (m, 2 H), 7.61 (s, 1 | 432.2 | 100% |
| | H), 7.41-7.49 (m, 2 H), 7.25-7.39 (m, 3 H), 6.95 (d, 1 H), | [M + H]⁺ | |
| | 6.61-6.87 (m, 2 H), 6.48 (s, 1 H), 6.19 (s, 1 H), 4.46-4.76 (m, | | |
| | 2 H), 4.35 (br d, 1 H), 4.04-4.27 (m, 2 H), 3.78 (td, 1 H), | | |
| | 3.43-3.62 (m, 1 H), 3.09 (td, 1 H), 2.82-2.98 (m, 2 H), 2.69 | | |
| | (br dd, 1 H), 2.40 (s, 3 H) | | |
| 93 | (400 MHz, METHANOL-d₄) δ 8.34 (d, 1 H), 8.14 (s, 1 H), | 421.1 | 100% |
| | 7.62 (s, 1 H), 6.93 (dd, 1 H), 6.65-6.86 (m, 2 H), 6.61 (s, 1 | [M + H]⁺ | |
| | H), 6.14 (s, 1 H), 4.66 (br dd, 1 H), 3.75 (t, 4 H), 3.42-3.62 | | |
| | (m, 5 H), 2.84-2.99 (m, 1 H), 2.70 (dd, 1 H) | | |
| 94 | (400 MHz, METHANOL-d₄) δ 8.34 (d, 1 H), 8.14 (s, 1 H), | 421.1 | 98.6% |
| | 7.63 (s, 1 H), 6.93 (dd, 1 H), 6.66-6.85 (m, 2 H), 6.61 (s, 1 | [M + H]⁺ | |
| | H), 6.14 (s, 1 H), 4.62-4.73 (m, 1 H), 3.75 (t, 4 H), 3.35-3.68 | | |
| | (m, 5 H), 2.85-2.99 (m, 1 H), 2.70 (dd, 1 H) | | |
| 95 | (400 MHz, METHANOL-d₄) δ 8.25 (d, 1 H), 8.15 (s, 1 H), | 497.2 | 100% |
| | 7.62 (s, 1 H), 7.42-7.51 (m, 2 H), 7.26-7.41 (m, 3 H), 6.92 | [M + H]⁺ | |
| | (dd, 1 H), 6.77 (td, 2 H), 6.59 (s, 1 H), 6.21 (s, 1 H), | | |
| | 4.63-4.72 (m, 1 H), 4.55 (dd, 1 H), 4.35 (br d, 1 H), 4.08-4.25 | | |
| | (m, 2 H), 3.80 (td, 1 H), 3.45-3.56 (m, 1 H), 3.10 (td, 1 H), | | |
| | 2.79-2.99 (m, 2 H), 2.69 (br dd, 1 H) | | |
| 96 | (400 MHz, METHANOL-d₄) δ 8.30 (d, 1 H), 8.15 (s, 1 H), | 497.2 | 99.5% |
| | 7.61 (s, 1 H), 7.41-7.49 (m, 2 H), 7.26-7.40 (m, 3 H), 6.93 | [M + H]⁺ | |
| | (dd, 1 H), 6.66-6.85 (m, 2 H), 6.62 (s, 1 H), 6.19 (s, 1 H), | | |
| | 4.70 (br d, 1 H), 4.53 (dd, 1 H), 4.36 (br d, 1 H), 4.08-4.23 | | |
| | (m, 2 H), 3.79 (td, 1 H), 3.44-3.59 (m, 1 H), 3.10 (td, 1 H), | | |
| | 2.80-2.98 (m, 2 H), 2.70 (br dd, 1 H) | | |
| 97 | (400 MHz, METHANOL-d₄) δ 8.68 (d, 1 H), 8.33 (d, 1 H), | 440.2 | 100% |
| | 8.04 (br d, 2 H), 7.59-7.66 (m, 2 H), 7.37-7.54 (m, 2 H), | [M + H]⁺ | |
| | 7.03-7.17 (m, 2 H), 6.95 (dd, 1 H), 6.81 (td, 1 H), 6.62 (br s, 1 | | |
| | H), 5.06-5.20 (m, 1 H), 3.48-3.61 (m, 1 H), 2.86-2.98 (m, 1 | | |
| | H), 2.66-2.80 (m, 1 H) | | |
| 98 | (400 MHz, METHANOL-d₄) δ 8.55 (d, 1 H), 8.20 (d, 1 H), | 440.2 | 99.2% |
| | 7.91 (br d, 2 H), 7.48-7.54 (m, 2 H), 7.33 (br s, 2 H), | [M + H]⁺ | |
| | 6.91-7.03 (m, 2 H), 6.82 (dd, 1 H), 6.68 (td, 1 H), 6.49 (br s, 1 | | |
| | H), 4.96-5.04 (m, 1 H), 3.36-3.47 (m, 1 H), 2.75-2.85 (m, 1 | | |
| | H), 2.60 (br d, 1 H) | | |
| 99 | (400 MHz, METHANOL-d₄) δ 8.86 (s, 2 H), 8.34 (br d, 1 H), | 440.2 | 99.8% |
| | 7.74-7.83 (m, 2 H), 7.63-7.71 (m, 2 H), 7.53-7.60 (m, 2 H), | [M + H]⁺ | |
| | 7.34 (br s, 1 H), 6.96 (dd, 1 H), 6.78-6.86 (m, 1 H), 6.73 (s, 1 | | |
| | H), 5.28 (dd, 1 H), 3.57 (td, 1 H), 2.89-3.00 (m, 1 H), 2.80 (br | | |
| | dd, 1 H) | | |
| 100 | (400 MHz, METHANOL-d₄) δ 8.86 (s, 2 H), 8.34 (d, 1 H), | 440.2 | 99.4% |
| | 7.79 (d, 2 H), 7.64-7.71 (m, 2 H), 7.54-7.60 (m, 2 H), 7.34 (s, | [M + H]⁺ | |
| | 1 H), 6.96 (dd, 1 H), 6.79-6.87 (m, 1 H), 6.73 (s, 1 H), 5.28 | | |
| | (dd, 1 H), 3.57 (td, 1 H), 2.90-3.01 (m, 1 H), 2.76-2.86 (m, 1 H) | | |
| 101 | (400 MHz, METHANOL-d₄) δ 8.22-8.41 (m, 2 H), 7.62 (s, 1 | 442.2 | 99.7% |
| | H), 7.44 (br d, 2 H), 7.20-7.31 (m, 3 H), 7.13 (s, 1 H), | [M + H]⁺ | |
| | 6.84-6.96 (m, 2 H), 6.77 (td, 1 H), 6.26-6.56 (m, 1 H), 5.60 (s, | | |
| | 1 H), 5.08 (br dd, 1 H), 3.41-3.52 (m, 1 H), 2.78-2.91 (m, 1 H), | | |
| | 2.64-2.74 (m, 1 H) | | |
| 102 | (400 MHz, METHANOL-d₄) δ 8.25-8.40 (m, 2 H), 7.63 (s, 1 | 442.1 | 94.4% |
| | H), 7.44 (d, 2 H), 7.21-7.32 (m, 3 H), 7.16 (s, 1 H), 6.93 (dd, | [M + H]⁺ | |
| | 1 H), 6.76-6.83 (m, 2 H), 6.64 (s, 1 H), 5.61 (s, 1 H), 5.09 (br | | |
| | dd, 1 H), 3.39-3.53 (m, 1 H), 2.84-2.94 (m, 1 H), 2.70 (dd, 1 H) | | |
| 103 | (400 MHz, METHANOL-d₄) δ 8.23-8.39 (m, 2 H), 7.62 (s, 1 | 442.2 | 98.2% |
| | H), 7.43 (br d, 2 H), 7.19-7.31 (m, 3 H), 7.13 (s, 1 H), | [M + H]⁺ | |
| | 6.84-6.96 (m, 2 H), 6.77 (td, 1 H), 6.42 (br d, 1 H), 5.60 (s, 1 | | |
| | H), 5.08 (br dd, 1 H), 3.40-3.52 (m, 1 H), 2.79-2.91 (m, 1 H), | | |
| | 2.64-2.74 (m, 1 H) | | |
| 104 | (400 MHz, METHANOL-d₄) δ 8.26-8.39 (m, 2 H), 7.62 (s, 1 | 442.1 | 97.2% |
| | H), 7.44 (d, 2 H), 7.23-7.32 (m, 3 H), 7.15 (s, 1 H), 6.94 (dd, | [M + H]⁺ | |
| | 1 H), 6.76-6.83 (m, 2 H), 6.64 (s, 1 H), 5.62 (s, 1 H), 5.09 (br | | |
| | dd, 1 H), 3.41-3.51 (m, 1 H), 2.84-2.96 (m, 1 H), 2.67-2.75 | | |
| | (m, 1 H) | | |
| 105 | (400 MHz, METHANOL-d₄) δ 8.67 (d, 1 H), 8.29 (d, 1 H), | 436.1 | 100% |
| | 8.04 (br d, 2 H), 7.59-7.66 (m, 2 H), 7.37-7.53 (m, 2 H), | [M + H]⁺ | |
| | 7.03-7.22 (m, 2 H), 6.97 (d, 1 H), 6.75 (t, 1 H), 6.48 (br s, 1 H), | | |
| | 5.04-5.12 (m, 1 H), 3.49-3.57 (m, 1 H), 2.86-2.99 (m, 1 H), | | |
| | 2.71 (br d, 1 H), 2.41 (s, 3 H) | | |
| 106 | (400 MHz, METHANOL-d₄) δ 8.68 (d, 1 H), 8.29 (d, 1 H), | 436.2 | 98.9% |
| | 8.05 (br d, 2 H), 7.60-7.65 (m, 2 H), 7.45 (br s, 2 H), | [M + H]⁺ | |
| | 7.03-7.18 (m, 2 H), 6.98 (d, 1 H), 6.76 (t, 1 H), 6.48 (br s, 1 H), | | |
| | 5.04-5.16 (m, 1 H), 3.46-3.59 (m, 1 H), 2.87-2.97 (m, 1 H), | | |
| | 2.67-2.75 (m, 1 H), 2.42 (s, 3 H) | | |
| 107 | Data provided above | | |
| 108 | Data provided above | | |
| 109 | Data provided above | | |
| 110 | Data provided above | | |
| 111 | (400 MHz, METHANOL-d₄) δ 8.78-8.92 (m, 2 H), 8.30 (br d, | 436.2 | 97.5% |
| | 1 H), 7.78 (br d, 2 H), 7.63-7.71 (m, 2 H), 7.53-7.60 (m, 2 H), | [M + H]⁺ | |
| | 7.35 (s, 1 H), 6.95-7.01 (m, 1 H), 6.77 (t, 1 H), 6.58 (s, 1 H), | | |
| | 5.24 (br dd, 1 H), 3.57 (td, 1 H), 2.90-3.00 (m, 1 H), 2.79 (br | | |
| | dd, 1 H), 2.44 (s, 3 H) | | |
| 112 | (400 MHz, METHANOL-d₄) δ 8.79-8.93 (m, 2 H), 8.30 (d, 1 | 436.2 | 97.3% |
| | H), 7.79 (d, 2 H), 7.63-7.71 (m, 2 H), 7.54-7.61 (m, 2 H), | [M + H]⁺ | |
| | 7.35 (s, 1 H), 6.99 (d, 1 H), 6.78 (t, 1 H), 6.59 (s, 1 H), 5.25 | | |
| | (br dd, 1 H), 3.58 (td, 1 H), 2.90-3.00 (m, 1 H), 2.75-2.83 (m, | | |
| | 1 H), 2.44 (s, 3 H) | | |
| 113 | (400 MHz, METHANOL-d₄) δ 8.35 (d, 1 H), 8.20-8.29 (m, 1 | 438.2 | 100% |
| | H), 7.62 (s, 1 H), 7.46 (br d, 2 H), 7.21-7.33 (m, 3 H), 7.18 (s, | [M + H]⁺ | |
| | 1 H), 6.94 (br d, 1 H), 6.85 (d, 1 H), 6.70-6.76 (m, 1 H), | | |
| | 6.22-6.46 (m, 1 H), 5.61 (s, 1 H), 5.05 (br dd, 1 H), 3.41-3.51 (m, | | |
| | 1 H), 2.79-2.91 (m, 1 H), 2.63-2.75 (m, 1 H), 2.36 (s, 3 H) | | |
| 114 | (400 MHz, METHANOL-d₄) δ 8.34 (d, 1 H), 8.22-8.30 (m, 1 | 438.2 | 94.5% |
| | H), 7.63 (s, 1 H), 7.45 (br d, 2 H), 7.21 (br s, 3 H), 7.17-7.20 | [M + H]⁺ | |
| | (m, 1 H), 6.95 (br d, 1 H), 6.71-6.82 (m, 2 H), 6.51 (s, 1 H), | | |
| | 5.62 (s, 1 H), 5.07 (br dd, 1 H), 3.40-3.53 (m, 1 H), 2.83-2.97 | | |
| | (m, 1 H), 2.64-2.77 (m, 1 H), 2.41 (s, 3 H) | | |
| 115 | (400 MHz, METHANOL-d₄) δ 8.36 (d, 1 H), 8.24-8.28 (m, 1 | 438.2 | 96.5% |
| | H), 7.63 (s, 1 H), 7.46 (br d, 2 H), 7.21-7.32 (m, 3 H), | [M + H]⁺ | |
| | 7.16-7.20 (m, 1 H), 6.95 (d, 1 H), 6.86 (d, 1 H), 6.71-6.77 (m, | | |
| | 1 H), 6.19-6.50 (m, 1 H), 5.61 (s, 1 H), 5.02-5.07 (m, 1 H), 3.46 | | |
| | (td, 1 H), 2.78-2.92 (m, 1 H), 2.64-2.74 (m, 1 H), 2.37 (s, 3 H) | | |
| 116 | (400 MHz, METHANOL-d₄) δ 8.34 (d, 1 H), 8.28 (br d, 1 H), | 438.2 | 96.7% |
| | 7.64 (s, 1 H), 7.45 (br d, 2 H), 7.27 (dt, 3 H), 7.19 (s, 1 H), | [M + H]⁺ | |
| | 6.96 (br d, 1 H), 6.72-6.82 (m, 2 H), 6.51 (s, 1 H), 5.62 (s, 1 | | |
| | H), 5.04-5.09 (m, 1 H), 3.42-3.50 (m, 1 H), 2.84-2.95 (m, 1 | | |
| | H), 2.70 (br dd, 1 H), 2.42 (s, 3 H) | | |
| 117 | Data provided above | | |
| 118 | Data provided above | | |
| 119 | Data provided above | | |
| 120 | Data provided above | | |
| 121 | (400 MHz, METHANOL-d₄) δ 8.21 (d, 1 H), 8.12 (s, 1 H), | 461.2 | 99.2% |
| | 7.79 (s, 1 H), 7.17 (s, 1 H), 6.84 (dd, 1 H), 6.70 (td, 1 H), 6.58 | [M + H]⁺ | |
| | (s, 1 H), 5.02-5.11 (m, 1 H), 3.54-3.70 (m, 2 H), 3.31-3.39 | | |
| | (m, 1 H), 3.26 (br d, 2 H), 2.76-2.86 (m, 1 H), 2.63 (dd, 1 H), | | |
| | 2.27 (s, 3 H), 1.53-1.64 (m, 4 H), 1.45 (br s, 2 H) | | |
| 122 | (400 MHz, METHANOL-d₄) δ 8.17-8.22 (m, 1 H), 8.07-8.13 | 461.2 | 100% |
| | (m, 1 H), 7.53 (s, 1 H), 7.12 (br s, 1 H), 6.82 (dd, 1 H), 6.69 | [M + H]⁺ | |
| | (td, 1 H), 6.56 (s, 1 H), 5.00-5.08 (m, 1 H), 3.52-3.73 (m, 2 | | |
| | H), 3.32-3.39 (m, 1 H), 3.26 (br s, 2 H), 2.72-2.83 (m, 1 H), | | |
| | 2.57-2.65 (m, 1 H), 2.27 (s, 3 H), 1.52-1.63 (m, 4 H), 1.45 (br | | |
| | d, 2 H) | | |
| 123 | (400 MHz, METHANOL-d₄) δ 8.25-8.39 (m, 2 H), 7.65 (s, 1 | 447.2 | 100% |
| | H), 7.23 (s, 1 H), 6.93 (dd, 1 H), 6.79 (td, 1 H), 6.66 (s, 1 H), | [M + H]⁺ | |
| | 5.03-5.22 (m, 1 H), 3.59 (t, 2 H), 3.41-3.51 (m, 1 H), 3.36 (t, | | |
| | 2 H), 2.82-2.93 (m, 1 H), 2.71 (dd, 1 H), 2.39 (s, 3 H), | | |
| | 1.86-2.03 (m, 4 H) | | |
| 124 | (400 MHz, METHANOL-d₄) δ 8.15-8.41 (m, 2 H), 7.67 (s, 1 | 447.2 | 98.2% |
| | H), 7.23 (br s, 1 H), 6.93 (dd, 1 H), 6.74-6.83 (m, 1 H), 6.66 | [M + H]⁺ | |
| | (s, 1 H), 5.14 (br dd, 1 H), 3.59 (t, 2 H), 3.41-3.52 (m, 1 H), | | |
| | 3.36 (t, 2 H), 2.82-2.97 (m, 1 H), 2.71 (dd, 1 H), 2.39 (s, 3 H), | | |
| | 1.86-2.11 (m, 4 H) | | |
| 125 | Data provided above | | |
| 126 | Data provided above | | |
| 127 | (400 MHz, METHANOL-d₄) δ 8.21-8.35 (m, 2 H), 7.65 (s, 1 | 443.2 | 100% |
| | H), 7.25 (s, 1 H), 6.94 (d, 1 H), 6.73 (t, 1 H), 6.52 (s, 1 H), | [M + H]⁺ | |
| | 5.12 (br d, 1 H), 3.58 (t, 2 H), 3.41-3.53 (m, 1 H), 3.35 (t, 2 | | |
| | H), 2.82-2.95 (m, 1 H), 2.70 (dd, 1 H), 2.40 (d, 6 H), | | |
| | 1.87-2.02 (m, 4 H) | | |
| 128 | (400 MHz, METHANOL-d₄) δ 8.21-8.33 (m, 2 H), 7.68 (s, 1 H), | 443.2 | 98.0% |
| | 7.26 (s, 1 H), 6.95 (d, 1 H), 6.74 (t, 1 H), 6.53 (s, 1 H), 5.12 (br | [M + H]⁺ | |
| | d, 1 H), 3.59 (t, 2 H), 3.42-3.52 (m, 1 H), 3.36 (t, 2 H), 2.82-2.95 | | |
| | (m, 1 H), 2.70 (dd, 1 H), 2.40 (d, 6 H), 1.89-2.02 (m, 4 H) | | |
| 129 | Data provided above | | |
| 130 | Data provided above | | |
| 131 | (400 MHz, METHANOL-d₄) δ 8.30 (d, 1 H), 8.24 (s, 1 H), 7.79 | 421.2 | 100% |
| | (s, 1 H), 7.26 (s, 1 H), 6.93 (dd, 1 H), 6.79 (td, 1 H), 6.67 (s, 1 | [M + H]⁺ | |
| | H), 5.16 (br dd, 1 H), 3.40-3.52 (m, 1 H), 3.11 (s, 3 H), 2.99 | | |
| | (s, 3 H), 2.84-2.95 (m, 1 H), 2.67-2.78 (m, 1 H), 2.37 (s, 3 H) | | |
| 132 | (400 MHz, METHANOL-d₄) δ 8.30 (d, 1 H), 8.24 (s, 1 H), | 421.2 | 97.2% |
| | 7.77 (s, 1 H), 7.25 (s, 1 H), 6.93 (dd, 1 H), 6.79 (td, 1 H), 6.67 | [M + H]⁺ | |
| | (s, 1 H), 5.16 (br dd, 1 H), 3.41-3.52 (m, 1 H), 3.11 (s, 3 H), | | |
| | 2.99 (s, 3 H), 2.85-2.95 (m, 1 H), 2.72 (dd, 1 H), 2.36 (s, 3 H) | | |
| 204 | (400 MHz, METHANOL-d₄) δ 8.35 (d, 1 H), 7.92 (s, 1 H), | 432.2 | 100% |
| | 7.67-7.76 (m, 1 H), 7.18 (d, 1 H), 7.06 (d, 1 H), 6.92-7.00 (m, 2 | [M + H]⁺ | |
| | H), 6.78-6.87 (m, 1 H), 6.64 (s, 1 H), 4.61 (br dd, 1 H), 3.53-3.74 | | |
| | (m, 5 H), 2.89-3.05 (m, 1 H), 2.74 (dd, 1 H), 1.81-2.03 (m, 4 H) | | |
| 205 | (400 MHz, METHANOL-d₄) δ 8.33 (d, 1 H), 7.66-7.76 (m, 2 | 432.2 | 99.2% |
| | H), 7.16 (d, 1 H), 7.05 (d, 1 H), 6.89-6.99 (m, 2 H), 6.77-6.85 | [M + H]⁺ | |
| | (m, 1 H), 6.62 (s, 1 H), 4.60 (br dd, 1 H), 3.55-3.69 (m, 5 H), | | |
| | 2.88-3.03 (m, 1 H), 2.72 (dd, 1 H), 1.82-2.01 (m, 4 H) | | |
| 216 | (400 MHz, METHANOL-d₄) δ 9.00 (s, 2 H), 8.27-8.40 (m, 1 | 404.2 | 99.6% |
| | H), 7.67 (s, 1 H), 7.27-7.41 (m, 1 H), 6.96 (dd, 1 H), 6.77-6.88 | [M + H]⁺ | |
| | (m, 1 H), 6.67-6.77 (m, 1 H), 5.23-5.29 (m, 1 H), 3.56 | | |
| | (td, 1 H), 2.87-2.98 (m, 1 H), 2.71-2.84 (m, 2 H), 1.17 (br s, 2 | | |
| | H), 1.06-1.12 (m, 2 H) | | |
| 217 | (400 MHz, METHANOL-d₄) δ 9.01-9.12 (m, 2 H), 8.33 (d, 1 | 404.2 | 99.3% |
| | H), 7.67 (s, 1 H), 7.32 (s, 1 H), 6.96 (dd, 1 H), 6.82 (td, 1 H), | [M + H]⁺ | |
| | 6.67-6.74 (m, 1 H), 5.26 (dd, 1 H), 3.56 (td, 1 H), 2.88-3.00 (m, | | |
| | 1 H), 2.72-2.82 (m, 2 H), 1.14-1.18 (m, 2 H), 1.06-1.11 (m, 2 H) | | |
| 218 | (400 MHz, METHANOL-d₄) δ 8.33-8.40 (m, 1 H), 8.28-8.32 | 442.2 | 99.8% |
| | (m, 1 H), 8.20-8.26 (m, 1 H), 7.58-7.67 (m, 1 H), 7.37-7.45 | [M + H]⁺ | |
| | (m, 2 H), 7.28-7.35 (m, 2 H), 7.18-7.27 (m, 1 H), 6.88-6.98 | | |
| | (m, 1 H), 6.73-6.85 (m, 2 H), 6.58-6.68 (m, 1 H), 5.70-5.78 | | |
| | (m, 1 H), 4.63-4.72 (m, 1 H), 3.52-3.65 (m, 1 H), 2.87-3.01 | | |
| | (m, 1 H), 2.67 (s, 1 H) | | |
| 219 | (400 MHz, METHANOL-d₄) δ 8.34-8.39 (m, 1 H), 8.27-8.33 | 442.2 | 94.6% |
| | (m, 1 H), 8.21-8.25 (m, 1 H), 7.59-7.67 (m, 1 H), 7.37-7.45 | [M + H]⁺ | |
| | (m, 2 H), 7.28-7.35 (m, 2 H), 7.18-7.27 (m, 1 H), 6.87-6.97 | | |
| | (m, 1 H), 6.74-6.83 (m, 2 H), 6.62 (s, 1 H), 5.70-5.78 (m, 1 | | |
| | H), 4.61-4.73 (m, 1 H), 3.52-3.65 (m, 1 H), 2.86-3.01 (m, 1 | | |
| | H), 2.65-2.78 (m, 1 H) | | |
| 220 | (400 MHz, METHANOL-d₄) δ 8.34-8.38 (m, 1 H), 8.27-8.33 | 442.2 | 100% |
| | (m, 1 H), 8.20-8.26 (m, 1 H), 7.58-7.69 (m, 1 H), 7.37-7.44 | [M + H]⁺ | |
| | (m, 2 H), 7.28-7.35 (m, 2 H), 7.20-7.27 (m, 1 H), 6.87-6.98 | | |
| | (m, 1 H), 6.74-6.85 (m, 2 H), 6.56-6.67 (m, 1 H), 5.76 (s, 1 | | |
| | H), 4.63-4.70 (m, 1 H), 3.53-3.64 (m, 1 H), 2.89-3.00 (m, 1 | | |
| | H), 2.68-2.79 (m, 1 H) | | |
| 221 | (400 MHz, METHANOL-d₄) δ 8.34-8.38 (m, 1 H), 8.27-8.33 | 442.2 | 97.7% |
| | (m, 1 H), 8.17-8.25 (m, 1 H), 7.55-7.69 (m, 1 H), 7.37-7.44 | [M + H]⁺ | |
| | (m, 2 H), 7.27-7.35 (m, 2 H), 7.16-7.26 (m, 1 H), 6.92 (dd, 1 | | |
| | H), 6.72-6.83 (m, 2 H), 6.56-6.66 (m, 1 H), 5.71-5.82 (m, 1 | | |
| | H), 4.62-4.70 (m, 1 H), 3.51-3.65 (m, 1 H), 2.87-3.00 (m, 1 | | |
| | H), 2.67-2.78 (m, 1 H) | | |
| 222 | (400 MHz, METHANOL-d₄) δ 8.37 (s, 2 H), 8.31 (d, 1 H), | 420.2 | 99.7% |
| | 7.71 (s, 1 H), 7.16-7.22 (m, 1 H), 6.90-6.98 (m, 1 H), 6.77 (s, | [M + H]⁺ | |
| | 1 H), 6.66 (s, 1 H), 5.05 (dd, 1 H), 4.44 (d, 1 H), 3.42-3.50 | | |
| | (m, 1 H), 2.85-2.97 (m, 1 H), 2.62-2.74 (m, 2 H), 2.00-2.20 | | |
| | (m, 2 H), 1.75-1.94 (m, 4 H) | | |
| 223 | (400 MHz, METHANOL-d₄) δ 8.33-8.41 (m, 2 H), 8.26 (d, 1 | 420.2 | 94.0% |
| | H), 7.64 (s, 1 H), 7.15 (s, 1 H), 6.91 (dd, 1 H), 6.77 (td, 1 H), | [M + H]⁺ | |
| | 6.63 (s, 1 H), 5.03 (br dd, 1 H), 4.42 (d, 1 H), 3.39-3.51 (m, 1 | | |
| | H), 2.81-2.93 (m, 1 H), 2.59-2.73 (m, 2 H), 1.97-2.19 (m, 2 | | |
| | H), 1.73-1.93 (m, 4 H) | | |
| 224 | (400 MHz, METHANOL-d₄) δ 8.38 (s, 2 H), 8.32 (d, 1 H), | 420.2 | 92.2% |
| | 7.81 (s, 1 H), 7.15-7.27 (m, 1 H), 6.91-7.02 (m, 1 H), 6.76-6.86 | [M + H]⁺ | |
| | (m, 1 H), 6.60-6.72 (m, 1 H), 5.03-5.10 (m, 1 H), 4.45 | | |
| | (d, 1 H), 3.41-3.50 (m, 1 H), 2.84-2.98 (m, 1 H), 2.62-2.76 | | |
| | (m, 2 H), 1.99-2.17 (m, 2 H), 1.73-1.94 (m, 4 H) | | |
| 225 | (400 MHz, METHANOL-d₄) δ 8.38 (s, 2 H), 8.31 (d, 1 H), | 420.2 | 99.2% |
| | 7.74 (s, 1 H), 7.19 (s, 1 H), 6.91-6.99 (m, 1 H), 6.76-6.84 (m, | [M + H]⁺ | |
| | 1 H), 6.66 (s, 1 H), 5.06 (dd, 1 H), 4.44 (d, 1 H), 3.41-3.53 | | |
| | (m, 1 H), 2.85-2.96 (m, 1 H), 2.63-2.75 (m, 2 H), 1.99-2.19 | | |
| | (m, 2 H), 1.74-1.95 (m, 4 H) | | |
| 226 | (400 MHz, METHANOL-d₄) δ 8.42-8.51 (m, 2 H), 8.29 (d, 1 | 406.2 | 76.5% |
| | H), 7.62 (s, 1 H), 7.16 (s, 1 H), 6.89-6.95 (m, 1 H), 6.78 (td, 1 | [M + H]⁺ | |
| | H), 6.64 (s, 1 H), 5.04 (dd, 1 H), 3.89 (d, 1 H), 3.45 (ddd, 1 | | |
| | H), 2.83-2.97 (m, 1 H), 2.64-2.73 (m, 1 H), 1.13-1.22 (m, 1 | | |
| | H), 0.60-0.69 (m, 1 H), 0.45-0.58 (m, 2 H), 0.28-0.36 (m, 1 H) | | |
| 227 | (400 MHz, METHANOL-d₄) δ 8.45-8.52 (m, 2 H), 8.32 (d, 1 H), | 406.2 | 56.7% |
| | 7.64 (s, 1 H), 7.18 (s, 1 H), 6.89-6.98 (m, 1 H), 6.76-6.85 (m, 1 | [M + H]⁺ | |
| | H), 6.63-6.70 (m, 1 H), 5.06 (dd, 1 H), 3.91 (d, 1 H), 3.48 (ddd, | | |
| | 1 H), 2.85-2.98 (m, 1 H), 2.65-2.79 (m, 1 H), 1.15-1.25 (m, 1 | | |
| | H), 0.63-0.71 (m, 1 H), 0.47-0.61 (m, 2 H), 0.28-0.39 (m, 1 H) | | |
| 228 | (400 MHz, METHANOL-d₄) δ 8.43-8.49 (m, 2 H), 8.30 (d, 1 H), | 406.2 | 97.2% |
| | 7.62 (s, 1 H), 7.13-7.18 (m, 1 H), 6.92 (dd, 1 H), 6.74-6.82 (m, | [M + H]⁺ | |
| | 1 H), 6.65 (s, 1 H), 5.04 (dd, 1 H), 3.89 (d, 1 H), 3.46 (ddd, | | |
| | 1 H), 2.84-2.95 (m, 1 H), 2.65-2.76 (m, 1 H), 1.14-1.23 (m, 1 | | |
| | H), 0.60-0.69 (m, 1 H), 0.46-0.59 (m, 2 H), 0.26-0.36 (m, 1 H) | | |
| 229 | (400 MHz, METHANOL-d₄) δ 8.46 (s, 2 H), 8.30 (d, 1 H), 7.63 | 406.2 | 95.7% |
| | (s, 1 H), 7.17 (s, 1 H), 6.88-6.99 (m, 1 H), 6.78 (td, 1 H), 6.65 (s, | [M + H]⁺ | |
| | 1 H), 5.05 (dd, 1 H), 3.88-3.93 (m, 1 H), 3.42-3.51 (m, 1 H), | | |
| | 2.84-2.95 (m, 1 H), 2.66-2.76 (m, 1 H), 1.14-1.24 (m, 1 H), | | |
| | 0.60-0.69 (m, 1 H), 0.46-0.58 (m, 2 H), 0.27-0.36 (m, 1 H) | | |
| 230 | (400 MHz, METHANOL-d₄) δ 8.32 (dd, 1 H), 8.10-8.18 (m, | 454.2 | 100% |
| | 2 H), 7.60 (s, 1 H), 7.43-7.52 (m, 2 H), 7.23 (t, 2 H), 7.12 (dd, | [M + H]⁺ | |
| | 1 H), 7.03-7.09 (m, 1 H), 6.88 (dd, 1 H), 6.72 (td, 1 H), 6.59 | | |
| | (s, 1 H), 6.46 (s, 1 H), 3.76-3.86 (m, 1 H), 3.67 (td, 1 H), | | |
| | 2.79-2.89 (m, 1 H), 2.70-2.78 (m, 1 H) | | |
| 231 | (400 MHz, METHANOL-d₄) δ 8.32 (dd, 1 H), 8.11-8.18 (m, | 454.2 | 98.0% |
| | 2 H), 7.60 (s, 1 H), 7.48 (d, 2 H), 7.24 (t, 2 H), 7.12 (dd, 1 H), | [M + H]⁺ | |
| | 7.03-7.09 (m, 1 H), 6.88 (dd, 1 H), 6.73 (td, 1 H), 6.59 (s, 1 | | |
| | H), 6.46 (s, 1 H), 3.81 (dt, 1 H), 3.66 (ddd, 1 H), 2.79-2.89 | | |
| | (m, 1 H), 2.70-2.77 (m, 1 H) | | |
| 232 | (400 MHz, METHANOL-d₄) δ 8.87 (d, 1 H), 8.56 (s, 1 H), 8.35 | 455.2 | 100% |
| | (d, 1 H), 7.74 (d, 2 H), 7.68 (s, 1 H), 7.37 (t, 2 H), 7.11-7.20 (m, | [M + H]⁺ | |
| | 1 H), 6.89-7.02 (m, 2 H), 6.83 (td, 1 H), 6.71 (s, 1 H), 4.94-5.03 | | |
| | (m, 1 H), 3.59-3.72 (m, 1 H), 2.94-3.08 (m, 1 H), 2.83 (br dd, 1 H) | | |
| 233 | (400 MHz, METHANOL-d₄) δ 8.87 (d, 1 H), 8.56 (s, 1 H), 8.35 | 455.2 | 100% |
| | (d, 1 H), 7.74 (d, 2 H), 7.68 (s, 1 H), 7.37 (t, 2 H), 7.11-7.20 (m, 1 | [M + H]⁺ | |
| | H), 6.96 (dd, 2 H), 6.83 (td, 1 H), 6.71 (s, 1 H), 4.94-5.01 (m, 1 | | |
| | H), 3.58-3.74 (m, 1 H), 2.94-3.09 (m, 1 H), 2.83 (br dd, 1 H) | | |
| 234 | (400 MHz, METHANOL-d₄) δ 8.13-8.26 (m, 3 H), 7.51 (s, 1 H), | 472.2 | 99.7% |
| | 7.17-7.23 (m, 2 H), 7.04 (s, 1 H), 6.78-6.84 (m, 3 H), 6.68 (td, 1 | [M + H]⁺ | |
| | H), 6.48-6.58 (m, 1 H), 5.58 (s, 1 H), 4.90-4.93 (m, 1 H), 3.68 (s, | | |
| | 3 H), 3.30-3.38 (m, 1 H), 2.70-2.84 (m, 1 H), 2.52-2.65 (m, 1 H) | | |
| 235 | (400 MHz, METHANOL-d₄) δ 8.16-8.26 (m, 3 H), 7.53 (s, 1 H), | 472.2 | 98.9% |
| | 7.20 (d, 2 H), 7.02-7.07 (m, 1 H), 6.77-6.85 (m, 3 H), 6.63-6.71 | [M + H]⁺ | |
| | (m, 1 H), 6.49-6.56 (m, 1 H), 5.57 (s, 1 H), 4.91 (br s, 1 H), 3.68 | | |
| | (s, 3 H), 3.29-3.37 (m, 1 H), 2.72-2.83 (m, 1 H), 2.53-2.62 (m, 1 H) | | |
| 236 | (400 MHz, METHANOL-d₄) δ 8.14-8.27 (m, 3 H), 7.52 (s, 1 | 472.2 | 95.7% |
| | H), 7.20 (d, 2 H), 7.04 (s, 1 H), 6.77-6.85 (m, 3 H), 6.67 (td, 1 | [M + H]⁺ | |
| | H), 6.53 (s, 1 H), 5.58 (s, 1 H), 4.92 (dd, 1 H), 3.68 (s, 3 H), | | |
| | 3.28-3.38 (m, 1 H), 2.71-2.84 (m, 1 H), 2.57 (dd, 1 H) | | |
| 237 | (400 MHz, METHANOL-d₄) δ 8.13-8.28 (m, 3 H), 7.61 (s, 1 | 472.2 | 80.3% |
| | H), 7.20 (d, 2 H), 7.06 (s, 1 H), 6.74-6.87 (m, 3 H), 6.61-6.72 | [M + H]⁺ | |
| | (m, 1 H), 6.53 (s, 1 H), 5.58 (s, 1 H), 4.93 (dd, 1 H), 3.68 (s, 3 | | |
| | H), 3.28-3.39 (m, 1 H), 2.73-2.82 (m, 1 H), 2.53-2.64 (m, 1 H) | | |
| 238 | Data provided above | | |
| 239 | Data provided above | | |
| 240 | Data provided above | | |
| 241 | Data provided above | | |
| 242 | Data provided above | | |
| 243 | Data provided above | | |
| 244 | (400 MHz, METHANOL-d₄) δ 8.33 (d, 1 H), 7.90 (d, 1 H), 7.71 | 419.2 | 100% |
| | (s, 1 H), 7.55 (d, 1 H), 6.90-7.07 (m, 2 H), 6.76-6.86 (m, 1 H), | [M + H]⁺ | |
| | 6.69 (s, 1 H), 4.80 (br d, 1 H), 4.74 (br t, 2 H), 4.19-4.30 (m, 2 H), | | |
| | 3.67 (ddd, 1 H), 2.91-3.05 (m, 1 H), 2.79 (dd, 1 H), 2.40 (dt, 2 H) | | |
| 245 | (400 MHz, METHANOL-d₄) δ 8.33 (d, 1 H), 7.90 (d, 1 H), 7.65 | 419.2 | 100% |
| | (s, 1 H), 7.55 (d, 1 H), 6.85-7.00 (m, 2 H), 6.80 (td, 1 H), 6.69 (s, | [M + H]⁺ | |
| | 1 H), 4.82 (br s, 1 H), 4.74 (br t, 2 H), 4.23 (t, 2 H), 3.67 (ddd, 1 | | |
| | H), 2.92-3.06 (m, 1 H), 2.79 (dd, 1 H), 2.40 (quin, 2 H) | | |
| 246 | (400 MHz, METHANOL-d₄) δ 8.74 (d, 1 H), 8.33 (d, 1 H), | 419.2 | 100% |
| | 7.64 (s, 1 H), 7.48 (d, 1 H), 6.93 (dd, 1 H), 6.80 (td, 1 H), | [M + H]⁺ | |
| | 6.70 (s, 1 H), 6.51 (s, 1 H), 3.81-4.37 (m, 6 H), 3.10-3.23 (m, | | |
| | 1 H), 2.73 (dd, 1 H), 2.10-2.35 (m, 2 H) | | |
| 247 | (400 MHz, METHANOL-d₄) δ 8.74 (d, 1 H), 8.33 (d, 1 H), | 419.2 | 99.4% |
| | 7.65 (s, 1 H), 7.48 (d, 1 H), 6.93 (dd, 1 H), 6.79 (td, 1 H), | [M + H]⁺ | |
| | 6.70 (s, 1 H), 6.52 (s, 1 H), 3.82-4.35 (m, 6 H), 3.11-3.24 (m, | | |
| | 1 H), 2.73 (dd, 1 H), 2.12-2.29 (m, 2 H) | | |
| 248 | (400 MHz, DMSO-d₆) δ 12.27 (s, 1H), 8.74-8.67 (m, 1H), | 401.2 | 100% |
| | 8.58 (dd, 2H), 7.69-7.55 (m, 2H), 7.17-7.05 (m, 3H), 6.81 (t, | [M + H]⁺ | |
| | 1H), 6.52 (s, 1H), 5.31-5.06 (m, 1H), 3.54-3.46 (m, 1H), | | |
| | 2.85-2.68 (m, 3H), 0.83-0.61 (m, 4H) | | |
| 249 | (400 MHz, DMSO-d₆) δ 11.92 (s, 1H), 8.81-8.66 (m, 1H), | 400.8 | 98.1% |
| | 8.57 (dd, 2H), 7.66-7.50 (m, 2H), 7.30-6.93 (m, 3H), 6.80 (t, | [M + H]⁺ | |
| | 1H), 6.52 (s, 1H), 5.33-5.06 (m, 1H), 3.53-3.42 (m, 1H), | | |
| | 2.85-2.68 (m, 3H), 0.82-0.62 (m, 4H) | | |
| 250 | (400 MHz, METHANOL-d₄) δ 8.68 (d, 1 H), 8.39-8.47 (m, 2 | 401.2 | 99.9% |
| | H), 7.63 (s, 1 H), 7.53 (d, 1 H), 7.10-7.18 (m, 1 H), 6.74-6.90 | [M + H]⁺ | |
| | (m, 2 H), 6.52 (s, 1 H), 4.80 (br dd, 1 H), 4.68 (br t, 2 H), 4.17 | | |
| | (br t, 2 H), 3.55-3.67 (m, 1 H), 2.87-3.02 (m, 1 H), 2.69-2.80 | | |
| | (m, 1 H), 2.35 (quin, 2 H) | | |
| 251 | (400 MHz, METHANOL-d₄) δ 8.70 (d, 1 H), 8.45 (s, 2 H), 7.63 | 401.2 | 99.2% |
| | (s, 1 H), 7.56 (d, 1 H), 7.13-7.21 (m, 1 H), 6.83 (br t, 2 H), 6.53 | [M + H]⁺ | |
| | (s, 1 H), 4.80-4.83 (m, 1 H), 4.71 (t, 2 H), 4.19 (t, 2 H), 3.63 | | |
| | (ddd, 1 H), 2.91-3.01 (m, 1 H), 2.77 (br dd, 1 H), 2.37 (quin, 2 H) | | |
| 252 | Data provided above | | |
| 253 | Data provided above | | |
| 254 | Data provided above | | |
| 255 | Data provided above | | |
| 256 | (400 MHz, DMSO-d₆) δ 12.03 (s, 1H), 8.80-8.69 (m, 1H), | 419.2 | 100% |
| | 8.60 (d, 1H), 8.49 (d, 1H), 7.55 (s, 1H), 7.13-7.04 (m, 2H), | [M + H]⁺ | |
| | 6.81 (dd, 1H), 6.70 (s, 1H), 5.33-5.08 (m, 1H), 3.59-3.52 (m, | | |
| | 1H), 2.86-2.65 (m, 3H), 0.83-0.63 (m, 4H) | | |
| 257 | (400 MHz, DMSO-d₆) δ 11.95 (s, 1H), 8.81-8.69 (m, 1H), | 418.8 | 100% |
| | 8.60 (d, 1H), 8.49 (d, 1H), 7.56 (s, 1H), 7.12-7.06 (m, 2H), | [M + H]⁺ | |
| | 6.81 (dd, 1H), 6.70 (s, 1H), 5.33-5.09 (m, 1H), 3.55-3.41 (m, | | |
| | 1H), 2.87-2.70 (m, 3H), 0.76-0.67 (m, 4H) | | |
| 258 | Data provided above | | |
| 259 | Data provided above | | |
| 260 | (400 MHz, DMSO-d₆) δ 12.06 (s, 1H), 8.61 (d, 1H), 8.50 (d, | 415.2 | 100% |
| | 2H), 7.62 (d, 1H), 7.58 (s, 1H), 7.25-7.09 (m, 1H), 6.83 (t, | [M + H]⁺ | |
| | 1H), 6.78 (s, 1H), 6.56 (s, 1H), 4.82 (d, 1H), 3.73 (t, 2H), | | |
| | 3.59-3.47 (m, 3H), 2.90-2.68 (m, 2H), 1.87-1.80 (m, 4H). | | |
| 261 | (400 MHz, DMSO-d₆) δ 11.99 (s, 1H), 8.61 (d, 1H), 8.50 (d, | 415.2 | 96.3% |
| | 2H), 7.62 (d, 1H), 7.55 (s, 1H), 7.24-7.12 (m, 1H), 6.83 (t, | [M + H]⁺ | |
| | 1H), 6.73 (s, 1H), 6.56 (s, 1H), 4.82 (d, 1H), 3.73 (t, 2H), | | |
| | 3.59-3.47 (m, 3H), 2.93-2.64 (m, 2H), 1.89-1.78 (m, 4H) | | |
| 262 | (400 MHz, DMSO-d₆) δ 12.03 (m, 1H), 8.58 (d, 1H), 7.78 (d, | 401.3 | 100% |
| | 1H), 7.61 (d, 1H), 7.54 (d, 2H), 7.17 (t, 1H), 6.83 (m, 1H), | [M + H]⁺ | |
| | 6.69 (s, 1H), 6.56 (s, 1H), 5.13-4.63 (m, 1H), 4.62-4.47 (m, | | |
| | 2H), 4.07 (t, 2H), 3.60 (m, 1H), 2.87 (m, 1H), 2.78-2.69 (m, | | |
| | 1H), 2.31-2.23 (m, 2H) | | |
| 263 | (400 MHz, DMSO-d₆) δ 12.04 (m, 1H), 8.60 (d, 1H), 7.79 (d, | 401.2 | 98.9% |
| | 1H), 7.62 (d, 1H), 7.55 (d, 2H), 7.26-7.15 (m, 1H), 6.89-6.77 | [M + H]⁺ | |
| | (m, 1H), 6.70 (s, 1H), 6.55 (s, 1H), 5.09-4.66 (m, 1H), 4.58 | | |
| | (m, 2H), 4.15-3.99 (m, 2H), 3.65-3.50 (m, 1H), 2.94-2.81 (m, | | |
| | 1H), 2.73 (m, 1H), 2.28 (m, 2H) | | |
| 264 | (400 MHz, DMSO-d₆) δ 12.03 (m, 1H), 8.78 (d, 1H), 8.66-8.47 | 401.1 | 100% |
| | (m, 1H), 7.84 (d, 1H), 7.61 (d, 1H), 7.56 (d, 2H), 7.17 (t, | [M + H]⁺ | |
| | 1H), 6.80 (m, 2H), 6.55 (s, 1H), 4.79 (m, 1H), 3.61 (t, 1H), | | |
| | 2.89 (m, 2H), 2.75 (s, 1H), 0.67 (m, 4H) | | |
| 265 | (400 MHz, DMSO-d₆) δ 12.02 (m, 1H), 8.78 (d, 1H), 8.58 (d, | 401.1 | 98.3% |
| | 1H), 7.84 (d, 1H), 7.61 (d, 1H), 7.58-7.52 (m, 2H), 7.16 (t, | [M + H]⁺ | |
| | 1H), 6.81 (t, 1H), 6.75 (s, 1H), 6.54 (s, 1H), 4.80 (m, 1H), | | |
| | 3.62 (t, 1H), 2.89 (m, 2H), 2.73 (m, 1H), 0.66 (m, 4H) | | |
| 266 | (400 MHz, DMSO-d₆) δ 12.01 (s, 1H), 8.62 (d, 1H), 8.48 (d, | 415.1 | 99.3% |
| | 1H), 8.43 (d, 1H), 8.30 (d, 1H), 7.56 (s, 1H), 6.96 (d, 1H), 6.75 | [M + H]⁺ | |
| | (t, 2H), 6.53 (s, 1H), 4.90 (s, 1H), 3.60-3.52 (m, 1H), 2.88-2.81 | | |
| | (m, 2H), 2.74-2.71 (m, 1H), 2.37 (s, 3H), 0.70-0.54 (m, 4H) | | |
| 267 | (400 MHz, DMSO-d₆) δ 12.04 (s, 1H), 9.76 (s, 1H), 8.73 (s, | 483.2 | 98.4% |
| | 1H), 8.60-8.57 (m, 2H), 7.59 (s, 1H), 7.10 (s, 3H), 6.87-6.76 | [M + H]⁺ | |
| | (m, 3H), 4.97 (dd, 1H), 3.62-3.56 (m, 1H), 2.91-2.89 (m, 1H), | | |
| | 2.79-2.68 (m, 2H), 2.16 (s, 6H) | | |
| 268 | (400 MHz, DMSO-d₆) δ 12.04 (s, 1H), 9.76 (s, 1H), 8.73 (s, | 483.2 | 94.1% |
| | 1H), 8.60-8.55 (m, 2H), 7.59 (s, 1H), 7.10 (s, 3H), 6.87-6.76 | [M + H]⁺ | |
| | (m, 3H), 4.95 (d, 1H), 3.62-3.56 (m, 1H), 2.91-2.89 (m, 1H), | | |
| | 2.79-2.68 (m, 2H), 2.16 (s, 6H) | | |
| 269 | Data provided above | | |
| 270 | Data provided above | | |
| 271 | (400 MHz, DMSO-d₆) δ 12.27 (s, 1H), 8.63 (d, 1H), 8.54-8.50 | 421.2 | 100% |
| | (m, 2H), 8.02 (d, 1H), 7.65 (s, 1H), 7.11 (dd, 1H), 6.93-6.75 (m, | [M + H]⁺ | |
| | 2H), 6.73 (s, 1H), 4.90 (dd, 1H), 4.13-4.04 (m, 1H), 3.70-3.44 | | |
| | (m, 1H), 2.98-2.81 (m, 1H), 2.79-2.63 (m, 1H), 1.16 (dd, 6H) | | |
| 272 | (400 MHz, DMSO-d₆) δ 12.46 (s, 1H), 8.64 (d, 1H), 8.54-8.50 | 421.2 | 100% |
| | (m, 2H), 8.02 (d, 1H), 7.72 (s, 1H), 7.11 (dd, 1H), 6.87-6.82 (m, | [M + H]⁺ | |
| | 2H), 6.73 (s, 1H), 4.89 (dd, 1H), 4.13-4.04 (m, 1H), 3.64-3.49 (m, | | |
| | 1H), 2.95-2.80 (m, 1H), 2.79-2.66 (m, 1H), 1.16 (dd, 6H) | | |
| 273 | (400 MHz, DMSO-d₆) δ 12.01 (s, 1H), 8.62 (s, 1H), 8.53-8.48 | 433.2 | ≥99%* |
| | (m, 3H), 7.57 (s, 1H), 7.10 (dd, 1H), 6.83 (dd, 1H), 6.75 (s, | [M + H]⁺ | |
| | 1H), 6.72 (s, 1H), 5.02-4.75 (m, 1H), 4.53-4.36 (m, 1H), | | |
| | 3.65-3.49 (m, 1H), 2.97-2.80 (m, 1H), 2.77-2.64 (m, 1H), | | |
| | 2.21-2.09 (m, 4H), 1.69-1.58 (m, 2H) | | |
| 274 | (400 MHz, DMSO-d₆) δ 12.06 (s, 1H), 8.90 (d, 1H), 8.63 (s, | 469.2 | ≥99%* |
| | 1H), 8.52 (d, 2H), 7.59 (s, 1H), 7.10 (dd, 1H), 6.87-6.82 (m, | [M + H]⁺ | |
| | 2H), 6.72 (s, 1H), 4.92 (dd, 1H), 4.30 (dt, 1H), 3.66-3.47 (m, | | |
| | 1H), 2.93-2.81 (m, 5H), 2.77-2.65 (m, 1H) | | |
| 275 | (400 MHz, DMSO-d₆) δ 8.64 (d, 1H), 8.53 (d, 1H), 8.49 (s, 1H), | 433.2 | 100% |
| | 8.44 (s, 1H), 7.98 (s, 1H), 7.12 (dd, 1H), 6.91 (s, 1H), 6.88-6.83 | [M + H]⁺ | |
| | (m, 1H), 6.75 (s, 1H), 4.87 (d, 1H), 3.62-3.47 (m, 2H), 2.93-2.83 | | |
| | (m, 1H), 2.76 (dd, 1H), 1.35 (s, 3H), 0.75 (t, 2H), 0.59 (q, 2H) | | |
| 276 | (400 MHz, DMSO-d₆) δ 8.65 (d, 1H), 8.54 (d, 1H), 8.50 (s, 1H), | 433.2 | 100% |
| | 8.46 (s, 1H), 8.27 (s, 1H), 7.14 (dd, 1H), 7.00 (s, 1H), 6.88-6.83 | [M + H]⁺ | |
| | (m, 1H), 6.78 (s, 1H), 4.85 (d, 1H), 3.61-3.46 (m, 2H), 2.97-2.87 | | |
| | (m, 1H), 2.80 (dd, 1H), 1.35 (s, 3H), 0.75 (t, 2H), 0.59 (q, 2H) | | |
| 277 | (400 MHz, METHANOL-d₄) δ 8.80 (s, 2 H), 8.29 (br d, 1 H), | 419.2 | 100% |
| | 7.63 (s, 1 H), 7.23 (s, 1 H), 6.92 (dd, 1 H), 6.73-6.83 (m, 1 H), | [M + H]⁺ | |
| | 6.67 (s, 1 H), 5.17 (br dd, 1 H), 3.45-3.58 (m, 1 H), 2.79-2.97 (m, | | |
| | 2 H), 2.74 (br dd, 1 H), 0.74-0.86 (m, 2 H), 0.57-0.65 (m, 2 H) | | |
| 278 | (400 MHz, METHANOL-d₄) δ 8.80 (s, 2 H), 8.29 (d, 1 H), | 419.2 | 99.7% |
| | 7.63 (s, 1 H), 7.23 (s, 1 H), 6.92 (dd, 1 H), 6.78 (td, 1 H), 6.66 | [M + H]⁺ | |
| | (s, 1 H), 5.16 (br dd, 1 H), 3.42-3.67 (m, 1 H), 2.78-2.95 (m, | | |
| | 2 H), 2.66-2.77 (m, 1 H), 0.74-0.84 (m, 2 H), 0.56-0.68 (m, 2 H) | | |
| 279 | Data provided above | | |
| 280 | Data provided above | | |
| 281 | Data provided above | | |
| 282 | (400 MHz, DMSO-d₆) δ 12.01 (s, 1H), 8.63-8.48 (m, 3H), | 435.1 | ≥99%* |
| | 8.38 (s, 1H), 7.57 (s, 1H), 7.11 (dd, 1H), 6.87-6.82 (m, 1H), | [M + H]⁺ | |
| | 6.73 (s, 1H), 5.69 (s, 1H), 4.93-4.79 (m, 1H), 4.72-4.66 (m, | | |
| | 1H), 4.51-4.45 (m, 1H), 4.26-4.19 (m, 2H), 3.79-3.74 (m, | | |
| | 1H), 3.58-3.54 (m, 1H), 2.90-2.82 (m, 1H), 2.75-2.68 (m, 1H) | | |
| 283 | Data provided above | | |
| 284 | Data provided above | | |
| 285 | (400 MHz, METHANOL-d₄) δ 8.78 (s, 2 H), 8.29 (d, 1 H), | 433.3 | 100% |
| | 7.64 (s, 1 H), 7.23 (s, 1 H), 6.92 (dd, 1 H), 6.73-6.83 (m, 1 H), | [M + H]⁺ | |
| | 6.67 (s, 1 H), 5.16 (br dd, 1 H), 3.42-3.55 (m, 1 H), 2.83-2.95 | | |
| | (m, 1 H), 2.68-2.79 (m, 1 H), 1.42 (s, 3 H), 0.79-0.87 (m, | | |
| | 2 H), 0.63-0.73 (m, 2 H) | | |
| 286 | (400 MHz, METHANOL-d₄) δ 8.79 (s, 2 H), 8.29 (d, 1 H), 7.64 | 433.2 | 100% |
| | (s, 1 H), 7.23 (s, 1 H), 6.93 (dd, 1 H), 6.79 (td, 1 H), 6.67 (s, 1 H), | [M + H]⁺ | |
| | 5.16 (dd, 1 H), 3.43-3.57 (m, 1 H), 2.84-2.96 (m, 1 H), 2.67-2.80 | | |
| | (m, 1 H), 1.42 (s, 3 H), 0.77-0.87 (m, 2 H), 0.62-0.75 (m, 2 H) | | |
| 287 | (400 MHz, DMSO-d₆) δ 12.02 (s, 1H), 8.66 (s, 1H), 8.53 (d, | 436.7 | 100% |
| | 2H), 8.30 (d, 1H), 7.58 (s, 1H), 7.11 (t, 1H), 6.85-6.73 (m, 3H), | [M + H]⁺ | |
| | 4.94-4.90 (m, 1H), 4.87-4.67 (m, 1H), 3.56 (t, 1H), 2.88-2.84 (m, | | |
| | 2H), 2.77-2.74 (m, 1H), 1.32-1.21 (m, 1H), 1.13-1.04 (m, 1H). | | |
| 288 | (400 MHz, DMSO-d₆) δ 12.03 (s, 1H), 8.66 (s, 1H), 8.53 | 436.7 | 99.1% |
| | (d, 2H), 8.30 (d, 1H), 7.58 (s, 1H), 7.11 (t, 1H), 6.86-6.73 (m, | [M + H]⁺ | |
| | 3H), 4.94-4.90 (m, 1H), 4.86-4.66 (m, 1H), 3.55 (t, 1H), | | |
| | 2.87-2.74 (m, 3H), 1.32-1.21 (m, 1H), 1.14-1.04 (m, 1H) | | |
| 289 | (400 MHz, DMSO-d₆) δ 12.00 (s, 1H), 8.61 (s, 1H), 8.54 | 449.3 | 100% |
| | (d, 1H), 8.49 (s, 1H), 7.56 (s, 1H), 7.10 (t, 1H), 6.84 (dd, 1H), | [M + H]⁺ | |
| | 6.76 (s, 1H), 6.73 (s, 1H), 5.62 (s, 1H), 4.88-4.83 (m, 1H), | | |
| | 4.43-4.25 (m, 2H), 3.87 (q, 2H), 3.63-3.48 (m, 1H), 2.92-2.67 | | |
| | (m, 2H), 1.38 (s, 3H) | | |
| 290 | (400 MHz, DMSO-d₆) δ 12.00 (s, 1H), 8.61 (s, 1H), 8.54 | 449.3 | 100% |
| | (d, 1H), 8.49 (s, 1H), 7.56 (s, 1H), 7.10 (t, 1H), 6.84 (dd, 1H), | [M + H]⁺ | |
| | 6.76 (s, 1H), 6.72 (s, 1H), 5.62 (s, 1H), 4.87-4.83 (m, 1H), | | |
| | 4.43-4.28 (m, 2H), 3.87 (q, 2H), 3.58-3.52 (m, 1H), 2.90-2.68 | | |
| | (m, 2H), 1.38 (s, 3H) | | |
| 291 | (400 MHz, METHANOL-d₄) δ 8.32 (d, 1 H), 7.94 (d, 1 H), | 433.2 | 100% |
| | 7.65 (s, 1 H), 7.56 (d, 1 H), 6.85-7.04 (m, 2 H), 6.80 (td, 1 H), | [M + H]⁺ | |
| | 6.71 (s, 1 H), 4.76-4.84 (m, 1 H), 4.48-4.58 (m, 1 H), 3.69 | | |
| | (ddd, 1 H), 2.92-3.07 (m, 1 H), 2.80 (br dd, 1 H), 2.36 (dtd, 2 | | |
| | H), 2.09-2.23 (m, 2 H), 1.74-1.86 (m, 2 H) | | |
| 292 | (400 MHz, METHANOL-d₄) δ 8.32 (d, 1 H), 7.95 (d, 1 H), | 433.2 | 98.7% |
| | 7.66 (s, 1 H), 7.56 (d, 1 H), 6.94 (dd, 2 H), 6.80 (td, 1 H), | [M + H]⁺ | |
| | 6.71 (s, 1 H), 4.74-4.84 (m, 1 H), 4.53 (quin, 1 H), 3.69 (ddd, | | |
| | 1 H), 2.93-3.07 (m, 1 H), 2.70-2.86 (m, 1 H), 2.27-2.45 (m, 2 | | |
| | H), 2.06-2.24 (m, 2 H), 1.72-1.88 (m, 2 H) | | |
| 293 | (400 MHz, METHANOL-d₄) δ 8.32 (d, 1 H), 7.95 (d, 1 H), 7.65 | 433.2 | 100% |
| | (s, 1 H), 7.55 (d, 1 H), 6.93 (dd, 2 H), 6.79 (td, 1 H), 6.69 (s, 1 H), | [M + H]⁺ | |
| | 4.73-4.83 (m, 1 H), 3.68 (ddd, 1 H), 2.92-3.07 (m, 1 H), 2.79 (br | | |
| | dd, 1 H), 1.45 (s, 3 H), 0.83-0.95 (m, 2 H), 0.64-0.78 (m, 2 H) | | |
| 294 | (400 MHz, METHANOL-d₄) δ 8.32 (d, 1 H), 7.95 (d, 1 H), 7.65 | 433.2 | 99.3% |
| | (s, 1 H), 7.55 (d, 1 H), 6.87-7.01 (m, 2 H), 6.80 (td, 1 H), 6.69 (s, | [M + H]⁺ | |
| | 1 H), 4.74-4.82 (m, 1 H), 3.68 (ddd, 1 H), 2.91-3.07 (m, 1 H), 2.79 | | |
| | (br dd, 1 H), 1.45 (s, 3 H), 0.84-0.90 (m, 2 H), 0.64-0.74 (m, 2 H) | | |
| 295 | (400 MHz, DMSO-d₆) δ 12.01 (s, 1H), 8.63 (d, 1H), 8.58-8.47 | 447.2 | ≥99%* |
| | (m, 2H), 8.08 (d, 1H), 7.57 (s, 1H), 7.16-6.99 (m, 1H), | [M + H]⁺ | |
| | 6.90-6.80 (m, 1H), 6.73 (d, 2H), 5.02-4.73 (m, 1H), 4.31-4.10 | | |
| | (m, 1H), 3.61-3.47 (m, 1H), 2.93-2.66 (m, 2H), 1.92-1.79 (m, | | |
| | 2H), 1.68 (s, 2H), 1.53 (s, 4H) | | |
| 296 | (400 MHz, DMSO-d₆) δ 11.99 (s, 1H), 8.53 (d, 1H), 8.48 (s, 1H), | 449.2 | ≥99%* |
| | 8.41 (s, 1H), 7.56 (s, 1H), 7.14-7.06 (m, 1H), 6.83 (dd, 1H), 6.73 | [M + H]⁺ | |
| | (s, 1H), 4.88-4.67 (m, 1H), 3.71-3.52 (m, 10H), 2.90-2.69 (m, 2H) | | |
| 297 | (400 MHz, DMSO-d₆) δ 12.02 (s, 1H), 8.63 (d, 1H), 8.54-8.52 | 434.7 | ≥99%* |
| | (m, 2H), 8.34 (t, 1H), 7.58 (s, 1H), 7.11 (dd, 1H), 6.84 | [M + H]⁺ | |
| | (dd, 1H), 6.77-6.73 (m, 2H), 4.92-4.89 (m, 1H), 3.55 (t, 1H), | | |
| | 3.26 (dd, 2H), 2.87-2.76 (m, 2H), 1.53-1.45 (m, 2H), 1.34-1.25 | | |
| | (m, 2H), 0.89 (t, 3H) | | |
| 298 | (400 MHz, DMSO-d₆) δ 12.09-12.02 (m, 1H), 8.64 (s, 1H), | 462.8 | ≥99%* |
| | 8.52 (s, 2H), 8.21 (d, 1H), 7.58 (s, 1H), 7.12-7.08 (m, 1H), | [M + H]⁺ | |
| | 6.86-6.81 (m, 1H), 6.75-6.73 (m, 2H), 4.96-4.82 (m, 1H), | | |
| | 4.01-3.96 (m, 1H), 3.86 (d, 2H), 3.55 (t, 1H), 3.41-3.37 (m, | | |
| | 2H), 2.88-2.68 (m, 2H), 1.69-1.61 (m, 4H) | | |
| 299 | (400 MHz, DMSO-d₆) δ 12.02 (s, 1H), 8.64 (d, 1H), 8.54-8.53 | 434.8 | ≥99%* |
| | (m, 2H), 8.33 (t, 1H), 7.58 (s, 1H), 7.13-7.08 (m, 1H), 6.87-6.82 | [M + H]⁺ | |
| | (m, 1H), 6.77-6.73 (m, 2H), 4.92-4.89 (m, 1H), 3.59-3.53 (m, | | |
| | 1H), 3.09 (t, 2H), 2.88-2.68 (m, 2H), 1.88-1.81 (m, 1H), 0.86 (d, 6H) | | |
| 300 | (400 MHz, DMSO-d₆) δ 12.01 (s, 1H), 9.06 (d, 1H), 8.62 (d, | 435.2 | ≥99%* |
| | 1H), 8.55-8.50 (m, 2H), 7.59 (s, 1H), 7.10 (dd, 1H), 6.89-6.75 | [M + H]⁺ | |
| | (m, 2H), 6.72 (s, 1H), 5.08-4.86 (m, 2H), 4.74-4.63 (m, 4H), | | |
| | 3.64-3.49 (m, 1H), 2.91-2.81 (m, 1H), 2.79-2.70 (m, 1H) | | |
| 301 | (400 MHz, DMSO-d₆) δ 12.01 (s, 1H), 8.62 (s, 1H), 8.54-8.49 | 449.1 | ≥99%* |
| | (m, 2H), 8.31 (s, 1H), 7.57 (s, 1H), 7.10 (dd, 1H), 6.83 (dd, | [M + H]⁺ | |
| | 2H), 6.72 (s, 1H), 4.89 (s, 1H), 4.71 (s, 1H), 3.55 (d, 1H), | | |
| | 3.49 (d, 2H), 2.86 (s, 1H), 2.75 (s, 1H), 0.75 (s, 4H) | | |
| 302 | (400 MHz, METHANOL-d₄) δ 8.62 (s, 1 H), 8.40 (s, 1 H), | 419.2 | 100% |
| | 8.33 (d, 1 H), 7.64 (s, 1 H), 6.93 (dd, 1 H), 6.77-6.86 (m, 2 | [M + H]⁺ | |
| | H), 6.61 (s, 1 H), 4.75 (br dd, 1 H), 3.70 (ddd, 1 H), 2.94-3.07 | | |
| | (m, 1 H), 2.79-2.91 (m, 2 H), 0.87 (dd, 2 H), 0.69-0.79 (m, 2 H) | | |
| 303 | (400 MHz, METHANOL-d₄) δ 8.63 (s, 1 H), 8.40 (s, 1 H), 8.34 | 419.2 | 99.5% |
| | (d, 1 H), 7.64 (s, 1 H), 6.94 (dd, 1 H), 6.76-6.87 (m, 2 H), 6.62 | [M + H]⁺ | |
| | (s, 1 H), 4.76 (br dd, 1 H), 3.71 (ddd, 1 H), 2.96-3.07 (m, 1 H), | | |
| | 2.77-2.91 (m, 2 H), 0.83-0.95 (m, 2 H), 0.61-0.79 (m, 2 H) | | |
| 304 | (400 MHz, DMSO-d₆) δ 12.01 (s, 1H), 8.64 (s, 1H), 8.53 (d, | 421.2 | 99.9% |
| | 2H), 8.36 (t, 1H), 7.58 (s, 1H), 7.18-7.06 (m, 1H), 6.88-6.80 | [M + H]⁺ | |
| | (m, 1H), 6.76 (s, 1H), 6.73 (s, 1H), 4.92 (s, 1H), 3.61-3.49 | | |
| | (m, 1H), 3.22 (d, 2H), 2.89-2.83 (m, 1H), 2.79-2.72 (m, 1H), | | |
| | 1.56-1.48 (m, 2H), 0.86 (t, 3H) | | |
| 305 | (400 MHz, DMSO-d₆) δ 12.01 (s, 1H), 8.63 (s, 1H), 8.53 (d, | 421.3 | 96.0% |
| | 2H), 8.35 (t, 1H), 7.58 (s, 1H), 7.17-7.05 (m, 1H), 6.91-6.79 | [M + H]⁺ | |
| | (m, 1H), 6.76 (s, 1H), 6.73 (s, 1H), 5.05-4.72 (m, 1H), 3.58-3.51 | | |
| | (m, 1H), 3.24-3.19 (m, 2H), 2.93-2.83 (m, 1H), 2.80-2.73 | | |
| | (m, 1H), 1.59-1.47 (m, 2H), 0.86 (t, 3H) | | |
| 306 | (400 MHz, DMSO-d₆) δ 12.00 (s, 1H), 8.55 (d, 1H), 8.49 (s, | 407.2 | 100% |
| | 1H), 8.38 (s, 1H), 7.57 (s, 1H), 7.20-7.05 (m, 1H), 6.84 (d, | [M + H]⁺ | |
| | 1H), 6.73 (s, 2H), 4.91-4.66 (m, 1H), 3.65-3.50 (m, 1H), 3.10 | | |
| | (s, 3H), 2.98 (s, 3H), 2.93-2.82 (m, 1H), 2.77-2.66 (m, 1H) | | |
| 307 | (400 MHz, DMSO-d₆) δ 12.00 (s, 1H), 8.55 (d, 1H), 8.49 (s, | 407.2 | 98.7% |
| | 1H), 8.38 (s, 1H), 7.57 (s, 1H), 7.20-7.05 (m, 1H), 6.84 (d, | [M + H]⁺ | |
| | 1H), 6.73 (s, 2H), 4.91-4.66 (m, 1H), 3.65-3.50 (m, 1H), 3.10 | | |
| | (s, 3H), 2.98 (s, 3H), 2.93-2.82 (m, 1H), 2.77-2.66 (m, 1H) | | |
| 308 | (400 MHz, DMSO-d₆) δ 12.00 (s, 1H), 8.54 (d, 1H), 8.46 (s, | 435.2 | 100% |
| | 1H), 8.36 (s, 1H), 7.56 (s, 1H), 7.16-7.05 (m, 1H), 6.90-6.75 | [M + H]⁺ | |
| | (m, 2H), 6.72 (s, 1H), 4.90-4.70 (m, 1H), 3.62-3.50 (m, 1H), | | |
| | 3.48-3.40 (m, 4H), 2.95-2.70 (m, 2H), 1.13 (t, 6H) | | |
| 309 | (400 MHz, DMSO-d₆) δ 12.04 (s, 1H), 8.61-8.51 (m, 1H), 8.47 | 435.3 | 99.7% |
| | (s, 1H), 8.37 (s, 1H), 7.56 (s, 1H), 7.20-7.05 (m, 1H), 6.93-6.80 | [M + H]⁺ | |
| | (m, 1H), 6.76-6.70 (m, 2H), 4.91-4.66 (m, 1H), 3.62-3.52 | | |
| | (m, 1H), 3.50-3.38 (m, 4H), 2.90-2.66 (m, 2H), 1.14 (t, 6H) | | |
| 310 | (400 MHz, DMSO-d₆) δ 12.05 (s, 1H), 8.63 (s, 1H), 8.58-8.38 | 477.2 | ≥99%* |
| | (m, 2H), 8.04-7.92 (m, 1H), 7.58 (s, 1H), 7.22-7.05 (m, 1H), | [M + H]⁺ | |
| | 7.02-6.73 (m, 2H), 6.72 (s, 1H), 4.86 (m, 1H), 4.55 (d, 1H), | | |
| | 3.76-3.66 (m, 2H), 3.60-3.50 (m, 1H), 2.95-2.70 (m, 2H), | | |
| | 1.88-1.73 (m, 4H), 1.50-1.36 (m, 2H), 1.30-1.16 (m, 2H). | | |
| 311 | (400 MHz, DMSO-d₆) δ 12.05 (s, 1H), 8.66-8.61 (m, 1H), | 477.3 | ≥99%* |
| | 8.59-8.42 (m, 2H), 7.93-7.83 (m, 1H), 7.57 (s, 1H), 7.17-7.05 | [M + H]⁺ | |
| | (m, 1H), 7.03-6.73 (m, 2H), 6.73 (s, 1H), 5.0-4.64 (m, 1H), | | |
| | 4.39 (d, 1H), 3.83-3.69 (m, 2H), 3.58-3.52 (m, 1H), 2.94-2.82 | | |
| | (m, 2H), 1.81-1.67 (m, 2H), 1.64-1.46 (m, 6H). | | |
| 312 | (400 MHz, DMSO-d₆) δ 12.12-11.97 (m, 1H), 8.61 (s, 1H), | 449.3[M + H]⁺ | 100% |
| | 8.53 (d, 1H), 8.49 (s, 1H), 7.56 (s, 1H), 7.13-7.06 (m, 1H), | | |
| | 6.87-6.81 (m, 1H), 6.80-6.68 (m, 2H), 4.86 (d, 1H), 4.71-4.65 | | |
| | (m, 1H), 4.31-4.19 (m, 3H), 3.83 (t, 1H), 3.56 (t, 1H), 3.22 (s, | | |
| | 3H), 2.86 (s, 1H), 2.77-2.67 (m, 1H) | | |
| 313 | (400 MHz, DMSO-d₆) δ 12.11-11.97 (m, 1H), 8.61 (s, 1H), | 449.2 | 100% |
| | 8.54 (d, 1H), 8.49 (s, 1H), 7.56 (s, 1H), 7.13-7.07 (m, 1H), | [M + H]⁺ | |
| | 6.83 (d, 1H), 6.79-6.69 (m, 2H), 4.85 (s, 1H), 4.71-4.66 (m, | | |
| | 1H), 4.31-4.20 (m, 3H), 3.83 (t, 1H), 3.56 (t, 1H), 3.22 (s, | | |
| | 3H), 2.86 (s, 1H), 2.76-2.66 (m, 1H) | | |
| 314 | (400 MHz, DMSO-d₆) δ 12.01 (s, 1H), 8.63 (d, 1H), 8.53-8.51 | 491.1 | ≥99%* |
| | (m, 2H), 8.02 (d, 1H), 7.57 (s, 1H), 7.12-7.08 (m, 1H), 6.84 (d, | [M + H]⁺ | |
| | 2H), 6.72 (s, 1H), 4.89 (s, 1H), 4.27 (s, 1H), 3.81-3.76 (m, 1H), | | |
| | 3.55 (s, 1H), 2.85 (d, 1H), 2.73 (d, 1H), 1.69 (d, 2H), 1.53 | | |
| | (d, 4H), 1.45 (d, 2H), 1.15 (s, 3H) | | |
| 315 | (400 MHz, DMSO-d₆) δ 12.01 (s, 1H), 8.63 (d, 1H), 8.54-8.50 | 491.2 | ≥99%* |
| | (m, 2H), 7.87 (d, 1H), 7.57 (s, 1H), 7.10 (dd, 1H), 6.84 (d, 2H), | [M + H]⁺ | |
| | 6.72 (s, 1H), 4.89 (s, 1H), 4.03 (s, 1H), 3.69 (d, 1H), 3.54 (d, | | |
| | 1H), 2.85 (d, 1H), 2.73 (d, 1H), 1.69 (t, 2H), 1.55 (d, 4H), | | |
| | 1.37 (t, 2H), 1.11 (s, 3H) | | |
| 316 | Data provided above | | |
| 317 | Data provided above | | |
| 318 | (400 MHz, DMSO-d₆) δ 11.99 (s, 1H), 8.54 (d, 1H), 8.47 (s, 1H), | 421.2 | 100% |
| | 8.36 (s, 1H), 7.56 (s, 1H), 7.14-7.05 (m, 1H), 6.88-6.79 (m, 1H), | [M + H]⁺ | |
| | 6.72 (s, 2H), 4.90-4.66 (m, 1H), 3.61-3.51 (m, 1H), 3.45 (d, 2H), | | |
| | 3.11-2.83 (m, 4H), 2.77-2.63 (m, 1H), 1.13 (t, 3H) | | |
| 319 | (400 MHz, DMSO-d₆) δ 11.99 (s, 1H), 8.55 (d, 1H), 8.47 (s, 1H), | 421.3 | 98.1% |
| | 8.37 (s, 1H), 7.56 (s, 1H), 7.16-7.06 (m, 1H), 6.89-6.79 (m, 1H), | [M + H]⁺ | |
| | 6.77-6.69 (m, 2H), 4.88-4.63 (m, 1H), 3.62-3.50 (m, 1H), 3.45 | | |
| | (d, 2H), 3.11-2.82 (m, 4H), 2.76-2.62 (m, 1H), 1.12 (t, 3H) | | |
| 320 | (400 MHz, DMSO-d₆) δ 12.02 (s, 1H), 8.65 (s, 1H), 8.53 (d, | 449.3 | ≥99%* |
| | 2H), 8.12 (t, 1H), 7.57 (s, 1H), 7.10 (dd, 1H), 6.87-6.76 (m, | [M + H]⁺ | |
| | 2H), 6.73 (s, 1H), 4.89 (s, 1H), 3.59-3.53 (m, 1H), 3.11 (d, | | |
| | 2H), 2.89-2.82 (m, 1H), 2.75-2.67 (m, 1H), 0.88 (s, 9H) | | |
| 321 | (400 MHz, DMSO-d₆) δ 12.02 (s, 1H), 8.65 (s, 1H), 8.57-8.48 | 379.2 | 100% |
| | (m, 2H), 7.74 (s, 1H), 7.58 (s, 1H), 7.37 (s, 1H), 7.18-7.03 | [M + H]⁺ | |
| | (m, 1H), 6.88-6.80 (m, 1H), 6.76 (s, 1H), 6.73 (s, 1H), 4.90 | | |
| | (s, 1H), 3.65-3.50 (m, 1H), 2.97-2.81 (m, 1H), 2.80-2.61 (m, 1H) | | |
| 322 | (400 MHz, METHANOL-d₄) δ 8.73 (d, 1 H), 8.67 (d, 1 H), 8.45 | 469.2 | 100% |
| | (s, 1 H), 7.66 (s, 1 H), 7.60 (d, 1 H), 6.81-7.09 (m, 2 H), 6.70 | [M + H]⁺ | |
| | (s, 1 H), 4.76-4.86 (m, 1 H), 3.54-3.68 (m, 1 H), 2.91-3.04 (m, | | |
| | 1 H), 2.73-2.88 (m, 2 H), 0.77-0.85 (m, 2 H), 0.60-0.68 (m, 2 H) | | |
| 323 | (400 MHz, METHANOL-d₄) δ 8.73 (d, 1 H), 8.66 (d, 1 H), 8.44 | 469.2 | 98.8% |
| | (d, 1 H), 7.66 (s, 1 H), 7.59 (d, 1 H), 6.81-7.03 (m, 2 H), 6.70 (s, | [M + H]⁺ | |
| | 1 H), 4.82-4.87 (m, 1 H), 3.56-3.67 (m, 1 H), 2.92-3.02 (m, 1 H), | | |
| | 2.74-2.87 (m, 2 H), 0.77-0.85 (m, 2 H), 0.61-0.67 (m, 2 H) | | |
| 324 | (400 MHz, METHANOL-d₄) δ 8.73 (d, 1 H), 8.33-8.53 (m, 2 | 435.2 | 100% |
| | H), 7.65 (s, 1 H), 7.27 (d, 1 H), 6.82 (t, 2 H), 6.65 (s, 1 H), | [M + H]⁺ | |
| | 4.85 (br s, 1 H), 3.55-3.68 (m, 1 H), 2.90-3.04 (m, 1 H), | | |
| | 2.74-2.89 (m, 2 H), 0.76-0.87 (m, 2 H), 0.59-0.71 (m, 2 H) | | |
| 325 | (400 MHz, METHANOL-d₄) δ 8.73 (d, 1 H), 8.37-8.48 (m, 2 | 435.2 | 97.5% |
| | H), 7.65 (s, 1 H), 7.26 (d, 1 H), 6.76-6.92 (m, 2 H), 6.65 (s, 1 | [M + H]⁺ | |
| | H), 4.84 (br s, 1 H), 3.61 (ddd, 1 H), 2.91-3.02 (m, 1 H), | | |
| | 2.74-2.87 (m, 2 H), 0.77-0.85 (m, 2 H), 0.59-0.68 (m, 2 H) | | |
| 326 | (400 MHz, METHANOL-d₄) δ 8.75 (d, 1 H), 8.58 (d, 1 H), 8.47 | 451.2 | 100% |
| | (d, 1 H), 7.67 (s, 1 H), 7.42 (d, 1 H), 6.81-7.13 (m, 3 H), 6.72 (s, | [M + H]⁺ | |
| | 1 H), 4.85 (br d, 1 H), 3.56-3.70 (m, 1 H), 2.92-3.07 (m, 1 H), | | |
| | 2.75-2.91 (m, 2 H), 0.79-0.88 (m, 2 H), 0.61-0.71 (m, 2 H) | | |
| 327 | (400 MHz, METHANOL-d₄) δ 8.75 (d, 1 H), 8.58 (d, 1 H), 8.47 | 451.2 | 98.7% |
| | (s, 1 H), 7.67 (s, 1 H), 7.42 (br d, 1 H), 6.82-7.13 (m, 3 H), 6.72 | [M + H]⁺ | |
| | (s, 1 H), 4.86 (br d, 1 H), 3.57-3.70 (m, 1 H), 2.93-3.05 (m, 1 H), | | |
| | 2.76-2.90 (m, 2 H), 0.80-0.87 (m, 2 H), 0.62-0.69 (m, 2 H) | | |
| 328 | (400 MHz, METHANOL-d₄) δ 8.71 (s, 1 H), 8.45 (s, 1 H), | 419.2 | 100% |
| | 7.63 (s, 1 H), 7.41 (d, 1 H), 7.15-7.24 (m, 1 H), 6.85 (br s, 1 | [M + H]⁺ | |
| | H), 6.65 (d, 1 H), 6.60 (br t, 1 H), 4.84 (br d, 1 H), 3.59-3.70 | | |
| | (m, 1 H), 2.90-3.02 (m, 1 H), 2.74-2.86 (m, 2 H), 0.76-0.84 | | |
| | (m, 2 H), 0.61-0.67 (m, 2 H) | | |
| 329 | (400 MHz, METHANOL-d₄) δ 8.72 (d, 1 H), 8.46 (s, 1 H), | 419.2 | 98.0% |
| | 7.63 (s, 1 H), 7.42 (d, 1 H), 7.21 (ddd, 1 H), 6.86 (br s, 1 H), | [M + H]⁺ | |
| | 6.66 (d, 1 H), 6.61 (t, 1 H), 4.85 (br d, 1 H), 3.61-3.71 (m, 1 | | |
| | H), 2.91-3.02 (m, 1 H), 2.75-2.87 (m, 2 H), 0.77-0.85 (m, 2 | | |
| | H), 0.61-0.67 (m, 2 H) | | |
| 330 | (400 MHz, DMSO-d₆) δ 12.05 (s, 1H), 10.68 (s, 1H), 8.80 (s, | 480.2 | ≥99%* |
| | 1H), 8.61 (s, 1H), 8.53 (d, 1H), 8.11 (d, 2H), 7.80 (d, 2H), 7.60 | [M + H]⁺ | |
| | (s, 1H), 7.22-7.02 (m, 1H), 6.94-6.78 (m, 2H), 6.76 (s, 1H), 5.02 | | |
| | (s, 1H), 3.68-3.54 (m, 1H), 3.00-2.83 (m, 1H), 2.83-2.71 (m, 1H) | | |
| 331 | (400 MHz, DMSO-d₆) δ 12.05 (s, 1H), 10.61 (s, 1H), 8.80 (s, | 480.3 | ≥99%* |
| | 1H), 8.61 (s, 1H), 8.53 (d, 1H), 8.35 (s, 1H), 8.24-8.15 (m, 1H), | [M + H]⁺ | |
| | 7.60 (s, 1H), 7.58-7.52 (m, 2H), 7.23-7.04 (m, 1H), 6.93-6.78 | | |
| | (m, 2H), 6.76 (s, 1H), 5.22-4.70 (m, 1H), 3.76-3.52 (m, | | |
| | 1H), 2.99-2.85 (m, 1H), 2.82-2.63 (m, 1H) | | |
| 332 | (400 MHz, DMSO-d₆) δ 12.07 (s, 1H), 9.93 (s, 1H), 8.80 (d, | 485.3 | ≥99%* |
| | 1H), 8.63 (s, 1H), 8.56 (d, 1H), 8.46-8.39 (m, 1H), 7.60 (s, | [M + H]⁺ | |
| | 1H), 7.15-7.06 (m, 3H), 7.02-6.96 (m, 1H), 6.94-6.81 (m, | | |
| | 2H), 6.77 (s, 1H), 5.11-4.77 (m, 1H), 3.92 (s, 3H), 3.68-3.55 | | |
| | (m, 1H), 2.97-2.83 (m, 1H), 2.82-2.70 (m, 1H) | | |
| 333 | (400 MHz, DMSO-d₆) δ 12.06 (s, 1H), 9.32 (s, 1H), 8.67 (s, | 444.1 | 99.2% |
| | 1H), 8.56-8.48 (m, 2H), 7.59 (s, 1H), 7.10 (dd, 1H), 6.91-6.78 | [M + H]⁺ | |
| | (m, 2H), 6.72 (s, 1H), 4.92 (s, 1H), 3.60-3.48 (m, 1H), 2.89-2.69 | | |
| | (m, 2H), 1.51 (dd, 2H), 1.29 (dd, 2H) | | |
| 334 | (400 MHz, METHANOL-d₄) δ 8.73 (d, 1 H), 8.44 (s, 1 H), | 431.2 | 100% |
| | 8.06 (d, 1 H), 7.64 (s, 1 H), 6.84 (br d, 1 H), 6.71-6.79 (m, 1 | [M + H]⁺ | |
| | H), 6.51-6.62 (m, 2 H), 4.82 (br d, 1 H), 3.93 (s, 3 H), 3.54-3.65 | | |
| | (m, 1 H), 2.90-3.03 (m, 1 H), 2.72-2.88 (m, 2 H), 0.76-0.85 | | |
| | (m, 2 H), 0.58-0.69 (m, 2 H) | | |
| 335 | (400 MHz, METHANOL-d₄) δ 8.73 (d, 1 H), 8.44 (d, 1 H), | 431.2 | 100% |
| | 8.06 (d, 1 H), 7.64 (s, 1 H), 6.84 (br s, 1 H), 6.75 (t, 1 H), | [M + H]⁺ | |
| | 6.46-6.60 (m, 2 H), 4.81 (br d, 1 H), 3.93 (s, 3 H), 3.53-3.68 | | |
| | (m, 1 H), 2.90-3.03 (m, 1 H), 2.73-2.88 (m, 2 H), 0.78-0.84 | | |
| | (m, 2 H), 0.62-0.67 (m, 2 H) | | |
| 336 | (400 MHz, METHANOL-d₄) δ 8.73 (d, 1 H), 8.45 (s, 1 H), | 467.2 | 100% |
| | 8.33 (d, 1 H), 7.65 (s, 1 H), 6.77-7.23 (m, 4 H), 6.64 (s, 1 H), | [M + H]⁺ | |
| | 4.80-4.84 (m, 1 H), 3.54-3.69 (m, 1 H), 2.91-3.04 (m, 1 H), | | |
| | 2.73-2.88 (m, 2 H), 0.77-0.87 (m, 2 H), 0.52-0.69 (m, 2 H) | | |
| 337 | (400 MHz, METHANOL-d₄) δ 8.73 (d, 1 H), 8.45 (d, 1 H), | 467.2 | 99.4% |
| | 8.33 (d, 1 H), 7.66 (s, 1 H), 6.79-7.20 (m, 4 H), 6.64 (s, 1 H), | [M + H]⁺ | |
| | 4.83 (br s, 1 H), 3.56-3.68 (m, 1 H), 2.91-3.03 (m, 1 H), | | |
| | 2.73-2.88 (m, 2 H), 0.77-0.87 (m, 2 H), 0.58-0.69 (m, 2 H) | | |
| 338 | (400 MHz, METHANOL-d₄) δ 8.71 (d, 1 H), 8.54 (s, 1 H), | 415.2 | 100% |
| | 7.63 (s, 1 H), 7.44 (d, 1 H), 7.09 (dd, 1 H), 6.77 (br s, 1 H), | [M + H]⁺ | |
| | 6.69 (d, 1 H), 6.48 (s, 1 H), 4.96 (br d, 1 H), 3.61-3.73 (m, 1 | | |
| | H), 2.91-3.02 (m, 1 H), 2.74-2.88 (m, 2 H), 2.65 (s, 3 H), | | |
| | 0.76-0.88 (m, 2 H), 0.52-0.70 (m, 2 H) | | |
| 339 | (400 MHz, METHANOL-d₄) δ 8.71 (d, 1 H), 8.54 (s, 1 H), | 415.3 | 95.0% |
| | 7.63 (s, 1 H), 7.44 (d, 1 H), 7.10 (dd, 1 H), 6.72-6.85 (m, 1 | [M + H]⁺ | |
| | H), 6.69 (d, 1 H), 6.48 (s, 1 H), 4.94-5.01 (m, 1 H), 3.67 (ddd, | | |
| | 1 H), 2.90-3.04 (m, 1 H), 2.75-2.89 (m, 2 H), 2.66 (s, 3 H), | | |
| | 0.77-0.85 (m, 2 H), 0.58-0.70 (m, 2 H) | | |
| 340 | (400 MHz, DMSO-d₆) δ 11.93 (s, 1H), 8.59-8.49 (m, 2H), | 418.1 | 100% |
| | 8.18 (d, 1H), 7.91 (dd, 1H), 7.52 (s, 1H), 7.11-7.03 (m, 2H), | [M + H]⁺ | |
| | 6.88-6.79 (m, 1H), 6.74-6.62 (m, 2H), 4.88-4.52 (m, 1H), | | |
| | 3.58-3.46 (m, 1H), 2.88-2.73 (m, 2H), 2.71-2.57 (m, 1H), | | |
| | 0.70-0.63 (m, 2H), 0.55-0.49 (m, 2H) | | |
| 341 | (400 MHz, DMSO-d₆) δ 11.93 (s, 1H), 8.60-8.48 (m, 2H), | 418.1 | 98.7% |
| | 8.18 (d, 1H), 7.91 (dd, 1H), 7.53 (s, 1 H), 7.12-7.03 (m, 2H), | [M + H]⁺ | |
| | 6.88-6.78 (m, 1H), 6.74-6.62 (m, 2H), 4.85-4.52 (m, 1H), | | |
| | 3.60-3.45 (m, 1H), 2.90-2.73 (m, 2H), 2.72-2.57 (m, 1H), | | |
| | 0.71-0.63 (m, 2H), 0.56-0.48 (m, 2H) | | |
| 342 | (400 MHz, METHANOL-d₄) δ 8.58 (s, 1 H), 8.29 (d, 1 H), | 419.2 | 100% |
| | 8.22 (s, 1 H), 7.64 (s, 1 H), 7.02 (s, 1 H), 6.94 (dd, 1 H), | [M + H]⁺ | |
| | 6.75-6.83 (m, 2 H), 4.16 (br dd, 1 H), 3.68-3.80 (m, 1 H), | | |
| | 3.09-3.21 (m, 1 H), 2.84 (tt, 1 H), 2.67 (br dd, 1 H), 0.74-0.83 | | |
| | (m, 2 H), 0.54-0.61 (m, 2 H) | | |
| 343 | (400 MHz, METHANOL-d₄) δ 8.58 (s, 1 H), 8.29 (d, 1 H), 8.22 | 419.2 | 99.7% |
| | (s, 1 H), 7.64 (s, 1 H), 7.02 (s, 1 H), 6.93 (dd, 1 H), 6.75-6.83 | [M + H]⁺ | |
| | (m, 2 H), 4.16 (br dd, 1 H), 3.68-3.78 (m, 1 H), 3.09-3.21 (m, | | |
| | 1 H), 2.84 (tt, 1 H), 2.62-2.72 (m, 1 H), 0.75-0.84 | | |
| | (m, 2 H), 0.51-0.62 (m, 2 H) | | |
| 344 | (400 MHz, DMSO-d₆) δ 11.97 (s, 1H), 8.57-8.50 (m, 1H), | 418.5 | 100% |
| | 8.44-8.39 (m, 1H), 8.28 (d, 1H), 7.83-7.76 (m, 1H), 7.56 | [M + H]⁺ | |
| | (dd, 1H), 7.53 (s, 1H), 7.15-7.04 (m, 1H), 6.90-6.79 (m, 1H), | | |
| | 6.70-6.65 (m, 1H), 6.36-6.13 (m, 1H), 4.24-4.14 (m, 1H), | | |
| | 3.77-3.64 (m, 1H), 2.98-2.67 (m, 3H), 0.68-0.57 (m, 4H) | | |
| 345 | (400 MHz, DMSO-d₆) δ 11.98 (s, 1H), 8.52 (d, 1H), 8.42 | 418.3 | 95.2% |
| | (d, 1H), 8.31-8.24 (m, 1H), 7.79 (d, 1H), 7.59-7.52 (m, 2H), | [M + H]⁺ | |
| | 7.18-7.05 (m, 1H), 6.88-6.79 (m, 1H), 6.67 (s, 1H), 6.39-6.13 | | |
| | (m, 1H), 4.25-4.13 (m, 1H), 3.76-3.61 (m, 1H), 2.98-2.68 (m, | | |
| | 3H), 0.68-0.57 (m, 4H) | | |
| 346 | (400 MHz, METHANOL-d₄) δ 8.73 (d, 1 H), 8.41-8.51 (m, 2 | 485.2 | 99.7% |
| | H), 7.66 (s, 1 H), 7.17 (d, 1 H), 6.80-6.91 (m, 2 H), 6.68 (s, 1 | [M + H]⁺ | |
| | H), 4.84 (br d, 1 H), 3.61 (ddd, 1 H), 2.92-3.02 (m, 1 H), | | |
| | 2.74-2.89 (m, 2 H), 0.78-0.85 (m, 2 H), 0.60-0.68 (m, 2 H) | | |
| 347 | (400 MHz, METHANOL-d₄) δ 8.73 (d, 1 H), 8.39-8.53 (m, 2 | 485.2 | 99.1% |
| | H), 7.66 (s, 1 H), 7.11-7.24 (m, 1 H), 6.79-6.98 (m, 2 H), 6.68 | [M + H]⁺ | |
| | (s, 1 H), 4.84 (br d, 1 H), 3.57-3.67 (m, 1 H), 2.91-3.02 (m, 1 | | |
| | H), 2.75-2.87 (m, 2 H), 0.77-0.85 (m, 2 H), 0.61-0.67 (m, 2 H) | | |
| 348 | (400 MHz, METHANOL-d₄) δ 8.83 (s, 1 H), 8.52 (s, 1 H), | 451.2 | 100% |
| | 8.28 (br d, 1 H), 7.61-7.76 (m, 3 H), 7.34 (t, 2 H), 7.06-7.17 | [M + H]⁺ | |
| | (m, 1 H), 6.87-6.99 (m, 2 H), 6.75 (t, 1 H), 6.55 (s, 1 H), | | |
| | 4.95-5.19 (m, 1 H), 3.59-3.73 (m, 1 H), 2.93-3.05 (m, 1 H), | | |
| | 2.74-2.90 (m, 1 H), 2.41 (s, 3 H) | | |
| 349 | Data provided above | | |
| 350 | Data provided above | | |
| 351 | (400 MHz, METHANOL-d₄) δ 8.73 (d, 1 H), 8.55 (d, 1 H), | 470.2 | 99.3% |
| | 8.47 (d, 1 H), 7.67 (s, 1 H), 7.17 (d, 1 H), 6.96 (br s, 1 H), | [M + H]⁺ | |
| | 6.87 (t, 1 H), 6.69 (s, 1 H), 4.91-4.94 (m, 1 H), 3.60-3.70 (m, | | |
| | 1 H), 3.32-3.40 (m, 1 H), 2.93-3.05 (m, 1 H), 2.81 (dd, 1 H), | | |
| | 1.02-1.12 (m, 4 H) | | |
| 352 | (400 MHz, METHANOL-d₄) δ 8.75 (d, 1 H), 8.56 (d, 1 H), | 470.2 | 97.5% |
| | 8.48 (d, 1 H), 7.69 (s, 1 H), 7.18 (d, 1 H), 6.98 (s, 1 H), 6.88 | [M + H]⁺ | |
| | (t, 1 H), 6.70 (s, 1 H), 4.92-4.94 (m, 1 H), 3.59-3.70 (m, 1 H), | | |
| | 3.32-3.37 (m, 1 H), 2.95-3.05 (m, 1 H), 2.77-2.87 (m, 1 H), | | |
| | 1.02-1.12 (m, 4 H) | | |
| 353 | (500 MHz, METHANOL-d₄) δ 8.76 (d, 1 H), 8.56 (s, 1 H), 8.09 | 416.2 | 99.1% |
| | (d, 1 H), 7.67 (s, 1 H), 6.95 (br s, 1 H), 6.78 (t, 1 H), 6.52-6.64 | [M + H]⁺ | |
| | (m, 2 H), 4.90-4.93 (m, 1 H), 3.95 (s, 3 H), 3.58-3.72 (m, 1 H), | | |
| | 3.34-3.37 (m, 1 H), 2.94-3.06 (m, 1 H), 2.82 (dd, 1 H), | | |
| | 1.02-1.16 (m, 4 H) | | |
| 354 | (500 MHz, METHANOL-d₄) δ 8.64 (d, 1 H), 8.44 (s, 1 H), | 416.2 | 98.5% |
| | 7.97 (d, 1 H), 7.55 (s, 1 H), 6.84 (br s, 1 H), 6.66 (t, 1 H), | [M + H]⁺ | |
| | 6.41-6.52 (m, 2 H), 4.80-4.93 (m, 1 H), 3.83 (s, 3 H), 3.54 | | |
| | (ddd, 1 H), 3.22-3.27 (m, 1 H), 2.82-2.94 (m, 1 H), 2.70 (dd, | | |
| | 1 H), 0.91-1.05 (m, 4 H) | | |
| 355 | (400 MHz, METHANOL-d₄) δ 8.73 (s, 1 H), 8.54 (s, 1 H), | 400.2 | 100% |
| | 8.29 (br d, 1 H), 7.66 (s, 1 H), 6.90-7.07 (m, 2 H), 6.75 (t, 1 | [M + H]⁺ | |
| | H), 6.55 (s, 1 H), 4.83-4.88 (m, 1 H), 3.61-3.71 (m, 1 H), | | |
| | 3.32-3.39 (m, 1 H), 2.93-3.04 (m, 1 H), 2.80 (br dd, 1 H), | | |
| | 2.41 (s, 3 H), 1.01-1.13 (m, 4 H) | | |
| 356 | (400 MHz, METHANOL-d₄) δ 8.74 (d, 1 H), 8.68 (d, 1 H), | 483.2 | 100% |
| | 8.56 (d, 1 H), 7.67 (s, 1 H), 7.61 (d, 1 H), 6.92-7.03 (m, 2 H), | [M + H]⁺ | |
| | 6.71 (s, 1 H), 4.91-4.96 (m, 1 H), 3.59-3.71 (m, 1 H), 3.32-3.36 | | |
| | (m, 1 H), 2.94-3.06 (m, 1 H), 2.82 (dd, 1 H), 1.10 (dt, 2 H), | | |
| | 1.02-1.08 (m, 2 H) | | |
| 357 | (400 MHz, METHANOL-d₄) δ 8.75 (d, 1 H), 8.69 (d, 1 H), | 483.2 | 99.7% |
| | 8.57 (d, 1 H), 7.67 (s, 1 H), 7.61 (d, 1 H), 6.94-7.03 (m, 2 H), | [M + H]⁺ | |
| | 6.71 (s, 1 H), 4.91-4.96 (m, 1 H), 3.60-3.69 (m, 1 H), 3.33-3.36 | | |
| | (m, 1 H), 2.95-3.05 (m, 1 H), 2.82 (dd, 1 H), 1.09-1.13 | | |
| | (m, 2 H), 1.03-1.08 (m, 2 H) | | |
| 358 | (400 MHz, METHANOL-d₄) δ 8.70 (d, 1 H), 8.38-8.53 (m, 2 | 501.2 | 100% |
| | H), 7.81 (s, 1 H), 7.65 (s, 1 H), 7.17 (d, 1 H), 6.77-6.93 (m, 2 | [M + H]⁺ | |
| | H), 6.67 (s, 1 H), 4.83 (br d, 1 H), 3.56-3.68 (m, 1 H), 2.91-3.03 | | |
| | (m, 1 H), 2.79 (dd, 1 H), 1.45 (s, 9 H) | | |
| 359 | (400 MHz, METHANOL-d₄) δ 8.70 (d, 1 H), 8.39-8.51 (m, 2 | 501.2 | 99.5% |
| | H), 7.81 (s, 1 H), 7.66 (s, 1 H), 7.17 (d, 1 H), 6.79-6.94 (m, 2 | [M + H]⁺ | |
| | H), 6.67 (s, 1 H), 4.83 (br d, 1 H), 3.57-3.67 (m, 1 H), 2.92-3.04 | | |
| | (m, 1 H), 2.79 (dd, 1 H), 1.46 (s, 9 H) | | |
| 360 | (500 MHz, METHANOL-d₄) δ 8.62 (d, 1 H), 8.47 (d, 1 H), | 445.2 | 100% |
| | 8.08 (d, 1 H), 7.65 (s, 1 H), 6.87 (s, 1 H), 6.77 (t, 1 H), 6.54-6.61 | [M + H]⁺ | |
| | (m, 2 H), 4.80 (br s, 1 H), 3.95 (s, 3 H), 3.88 (t, 2 H), 3.59-3.69 | | |
| | (m, 3 H), 2.93-3.04 (m, 1 H), 2.79 (dd, 1 H), 1.92-2.00 (m, 4 H) | | |
| 361 | (500 MHz, METHANOL-d₄) δ 8.62 (d, 1 H), 8.47 (d, 1 H), 8.08 | 445.2 | 100% |
| | (d, 1 H), 7.65 (s, 1 H), 6.87 (s, 1 H), 6.77 (t, 1 H), 6.53-6.61 (m, | [M + H]⁺ | |
| | 2 H), 4.80 (br d, 1 H), 3.95 (s, 3 H), 3.88 (t, 2 H), 3.61-3.67 (m, | | |
| | 3 H), 2.94-3.03 (m, 1 H), 2.79 (dd, 1 H), 1.93-1.98 (m, 4 H) | | |

In Table 3, “*” represents that the reported % ee is based on the enantiopurity of the chiral intermediate (S)-5-(4-(4-fluoropyrazolo[1,5-a]pyridin-2-yl)-1,4,6,7-tetrahydro-5H-imidazo[4,5-c]pyridin-5-yl)pyrazine-2-carboxylate used.

### Cell Based Phenylalanine Flux Assay

Cells expressing R408W PAH were made by transducing A375 cells with lentivirus encoding human PAH with the R408W mutation in pLVX-Puro, then selecting with puromycin until stable cell lines were generated. A375 R408W cells were seeded into 96 well plates in DMEM+10% FBS at a density of 40,000 cells/well one hour prior to compound addition. Compounds were resuspended in DMSO, and 2-fold serial dilutions were performed to generate a 10-point dose curve. Compounds were added to plated cells in a total volume of 100 μl, and a final DMSO concentration of 0.5%. Each compound was tested in duplicate. Following compound addition, cells were placed in a 5% CO₂, 37° C. tissue culture incubator for 24 hrs. After the incubation period, 20 μM sepiapterin and 800 μM ¹³C₉,¹⁵N-Phenylalanine were added. After 4 hours, cell media was removed. An aliquot of 10 μl of cell media was combined with 200 μl of extraction buffer (80% acetonitrile/20% H₂O) for each well. Determination of ¹³C-Tyrosine concentration was assessed by liquid chromatography mass spectrometry.

Specific compounds disclosed herein were tested in the foregoing assay and they were determined to have an AC₅₀ according to the following scores: (A) less than or equal to 0.500 μM, (B) greater than 0.500 and less than 1.000 μM, (C) greater than or equal to 1.000 and less than 5.000 μM, (D) greater than or equal to 5.000 μM, (E) no fit, and (NT) not tested, as shown below. Where a compound was tested multiple times, the average value of the tests is reported.

| Ex. No. | AC₅₀ |
|---|---|
| 89 | E |
| 90 | B |
| 91 | E |
| 92 | A |
| 93 | E |
| 94 | A |
| 95 | E |
| 96 | A |
| 97 | E |
| 98 | C |
| 99 | A |
| 100 | D |
| 101 | E |
| 102 | E |
| 103 | C |
| 104 | C |
| 105 | E |
| 106 | C |
| 107 | A |
| 108 | A |
| 109 | D |
| 110 | C |
| 111 | A |
| 112 | D |
| 113 | E |
| 114 | B |
| 115 | B |
| 116 | C |
| 117 | A |
| 118 | B |
| 119 | D |
| 120 | D |
| 121 | D |
| 122 | B |
| 123 | E |
| 124 | C |
| 125 | D |
| 126 | B |
| 127 | D |
| 128 | C |
| 129 | E |
| 130 | C |
| 131 | D |
| 132 | C |
| 204 | D |
| 205 | C |
| 216 | D |
| 217 | A |
| 218 | A |
| 219 | A |
| 220 | D |
| 221 | D |
| 222 | A |
| 223 | B |
| 224 | C |
| 225 | D |
| 226 | A |
| 227 | A |
| 228 | E |
| 229 | E |
| 230 | C |
| 231 | C |
| 232 | D |
| 233 | A |
| 234 | D |
| 235 | A |
| 236 | A |
| 237 | C |
| 238 | E |
| 239 | A |
| 240 | D |
| 241 | D |
| 242 | A |
| 243 | B |
| 244 | C |
| 245 | B |
| 246 | E |
| 247 | C |
| 248 | E |
| 249 | C |
| 250 | A |
| 251 | E |
| 252 | E |
| 253 | B |
| 254 | B |
| 255 | A |
| 256 | E |
| 257 | B |
| 258 | E |
| 259 | C |
| 260 | E |
| 261 | A |
| 262 | E |
| 263 | C |
| 264 | D |
| 265 | B |
| 266 | A |
| 267 | D |
| 268 | A |
| 269 | D |
| 270 | C |
| 271 | A |
| 272 | D |
| 273 | A |
| 274 | A |
| 275 | A |
| 276 | D |
| 277 | B |
| 278 | E |
| 279 | A |
| 280 | E |
| 281 | A |
| 282 | C |
| 283 | D |
| 284 | A |
| 285 | B |
| 286 | E |
| 287 | A |
| 288 | C |
| 289 | C |
| 290 | E |
| 291 | A |
| 292 | D |
| 293 | A |
| 294 | C |
| 295 | A |
| 296 | B |
| 297 | A |
| 298 | A |
| 299 | A |
| 300 | C |
| 301 | C |
| 302 | D |
| 303 | C |
| 304 | A |
| 305 | C |
| 306 | E |
| 307 | C |
| 308 | E |
| 309 | A |
| 310 | C |
| 311 | C |
| 312 | A |
| 313 | E |
| 314 | B |
| 315 | A |
| 316 | B |
| 317 | C |
| 318 | B |
| 319 | C |
| 320 | A |
| 321 | B |
| 322 | E |
| 323 | A |
| 324 | E |
| 325 | A |
| 326 | E |
| 327 | A |
| 328 | E |
| 329 | B |
| 330 | A |
| 331 | A |
| 332 | A |
| 333 | B |
| 334 | E |
| 335 | B |
| 336 | E |
| 337 | A |
| 338 | C |
| 339 | A |
| 340 | D |
| 341 | C |
| 342 | E |
| 343 | D |
| 344 | C |
| 345 | A |
| 346 | E |
| 347 | A |
| 348 | A |
| 349 | A |
| 350 | E |
| 351 | A |
| 352 | E |
| 353 | A |
| 354 | D |
| 355 | A |
| 356 | E |
| 357 | A |
| 358 | A |
| 359 | E |
| 360 | A |
| 361 | E |

Having now fully described the methods, compounds, and compositions of matter provided herein, it will be understood by those of skill in the art that the same can be performed within a wide and equivalent range of conditions, formulations, and other parameters without affecting the scope of the methods, compounds, and compositions provided herein or any embodiment thereof.

All patents, patent applications and publications cited herein are fully incorporated by reference herein in their entirety.

1. A compound of Formula I: 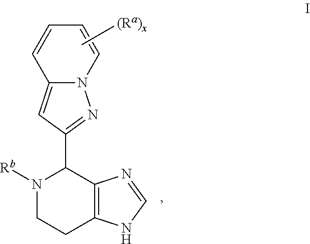
or tautomer thereof, or a pharmaceutically acceptable salt of the compound or the tautomer, wherein:
x is 0 to 5;
each R^{a} independently is halo, C₁₋₆alkyl, C₃₋₆cycloalkyl, C₁₋₆haloalkyl, C₁₋₆alkoxy or C₁₋₆haloalkoxy;
R^{b} is 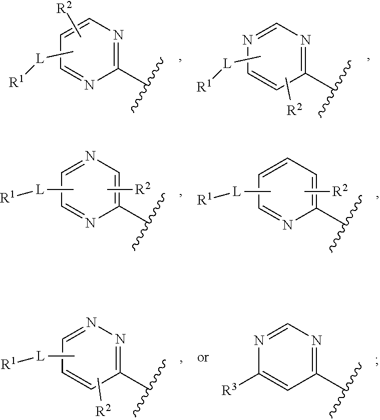
L is —C(O)—, —CH(OH)—, or —C(O)NH—;
R¹ is optionally substituted phenyl, optionally substituted 4-, 5-, or 6-membered heterocyclyl, —NR⁴R⁵, or optionally substituted C₃₋₆cycloalkyl;
R² is hydrogen, C₁₋₆alkyl, C₁₋₆haloalkyl, or halo;
R³ is optionally substituted 5- or 6-membered heterocyclyl; and
R⁴ and R⁵ are each independently hydrogen or C₁₋₆alkyl.

2. The compound of claim 1, or tautomer thereof, or a pharmaceutically acceptable salt of the compound or the tautomer, wherein R^{b} is

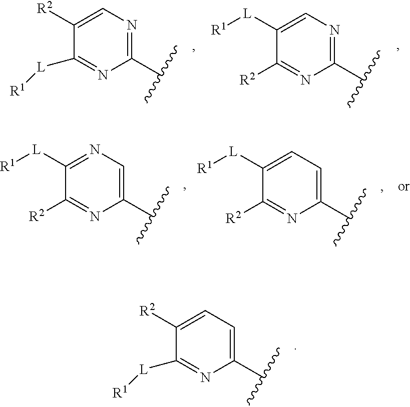

3.-10. (canceled)

11. The compound of claim 1, or tautomer thereof, or a pharmaceutically acceptable salt of the compound or the tautomer, wherein R¹ is optionally substituted phenyl, optionally substituted 4-, 5-, or 6-membered heterocyclyl, or optionally substituted C₃₋₆cycloalkyl.

12. The compound of claim 11, or tautomer thereof, or a pharmaceutically acceptable salt of the compound or the tautomer, wherein R¹ is phenyl, optionally substituted with one or more of C₁₋₆alkoxy, halo, C₁₋₆alkyl, or C₁₋₆haloalkyl.

13. The compound of claim 11, or tautomer thereof, or a pharmaceutically acceptable salt of the compound or the tautomer, wherein the 4-, 5-, or 6-membered heterocyclyl is optionally substituted azetidinyl, optionally substituted pyrrolidinyl, optionally substituted piperidinyl, optionally substituted piperazinyl, or optionally substituted morpholinyl.

14. The compound of claim 13, or tautomer thereof, or a pharmaceutically acceptable salt of the compound or the tautomer, wherein the 4-, 5-, or 6-membered heterocyclyl is optionally substituted with one or more of halo, C₁₋₆alkyl, OH, C₁₋₆hydroxyalkyl, or C₃₋₆cycloalkyl.

15.-17. (canceled)

18. The compound of claim 11, or tautomer thereof, or a pharmaceutically acceptable salt of the compound or the tautomer, wherein R¹ is C₃₋₆ cycloalkyl, optionally substituted with one or more of halo, OH, C₁₋₆alkyl, or C₁₋₆haloalkyl.

19. (canceled)

20. The compound of claim 18, or tautomer thereof, or a pharmaceutically acceptable salt of the compound or the tautomer, wherein the C₃₋₆cycloalkyl is cyclopropyl or cyclobutyl.

21. The compound of claim 1, or tautomer thereof, or a pharmaceutically acceptable salt of the compound or the tautomer, wherein L is —C(O)— or —CH(OH)— and R¹ is —NR⁴R⁵.

22. (canceled)

23. The compound of claim 1, or tautomer thereof, or a pharmaceutically acceptable salt of the compound or the tautomer, wherein R² is hydrogen or C₁₋₆alkyl.

24. (canceled)

25. The compound of claim 23, or tautomer thereof, or a pharmaceutically acceptable salt of the compound or the tautomer, wherein R² is hydrogen.

26.-31. (canceled)

32. The compound of claim 1, or tautomer thereof, or a pharmaceutically acceptable salt of the compound or the tautomer, wherein x is 0 or 1.

33. The compound of claim 32, or tautomer thereof, or a pharmaceutically acceptable salt of the compound or the tautomer, wherein R^{a} is F, Br, Cl, methyl, ethyl, isopropyl, methoxy, ethoxy, CF₃, CHF₂, OCF₃, OCHF₂, or cyclopropyl.

34.-38. (canceled)

39. A compound, or tautomer thereof, or a pharmaceutically acceptable salt of the compound or the tautomer, wherein the compound is selected from the following:

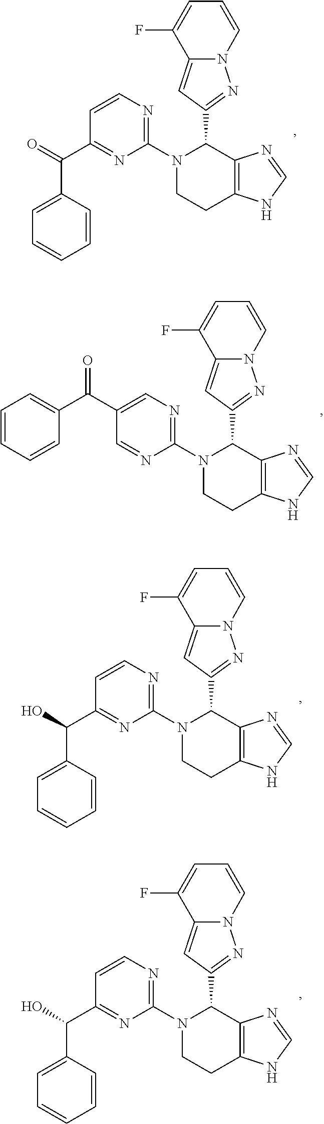 [SMILES: O=C(c1ccccc1)c1ccnc(N2CCc3[nH]cnc3[C@H]2c2cc3c(F)cccn3n2)n1 ; O=C(c1ccccc1)c1cnc(N2CCc3[nH]cnc3[C@H]2c2cc3c(F)cccn3n2)nc1 ; O[C@H](c1ccccc1)c1ccnc(N2CCc3[nH]cnc3[C@H]2c2cc3c(F)cccn3n2)n1 ; O[C@@H](c1ccccc1)c1ccnc(N2CCc3[nH]cnc3[C@H]2c2cc3c(F)cccn3n2)n1]

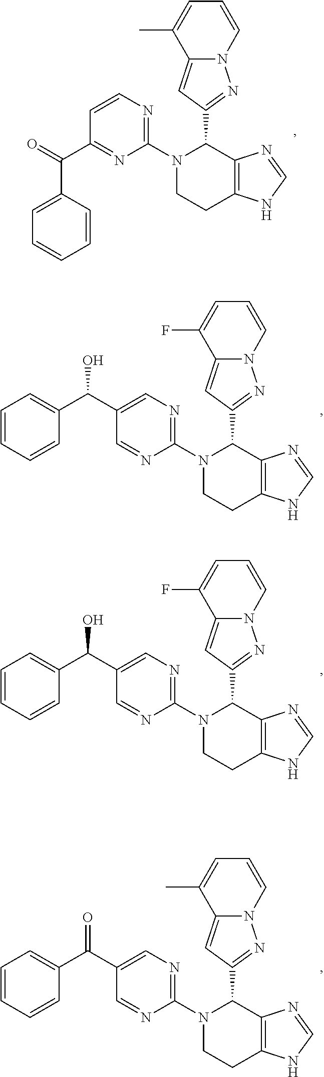 [SMILES: Cc1cccn2nc([C@@H]3c4nc[nH]c4CCN3c3nccc(C(=O)c4ccccc4)n3)cc12 ; O[C@@H](c1ccccc1)c1cnc(N2CCc3[nH]cnc3[C@H]2c2cc3c(F)cccn3n2)nc1 ; O[C@H](c1ccccc1)c1cnc(N2CCc3[nH]cnc3[C@H]2c2cc3c(F)cccn3n2)nc1 ; Cc1cccn2nc([C@@H]3c4nc[nH]c4CCN3c3ncc(C(=O)c4ccccc4)cn3)cc12]

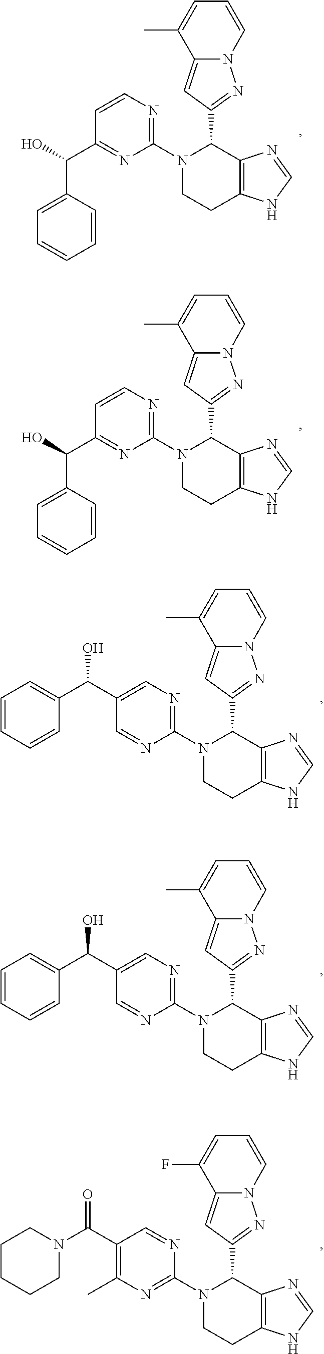 [SMILES: Cc1cccn2nc([C@@H]3c4nc[nH]c4CCN3c3nccc([C@@H](O)c4ccccc4)n3)cc12 ; Cc1cccn2nc([C@@H]3c4nc[nH]c4CCN3c3nccc([C@H](O)c4ccccc4)n3)cc12 ; Cc1cccn2nc([C@@H]3c4nc[nH]c4CCN3c3ncc([C@@H](O)c4ccccc4)cn3)cc12 ; Cc1cccn2nc([C@@H]3c4nc[nH]c4CCN3c3ncc([C@H](O)c4ccccc4)cn3)cc12 ; Cc1nc(N2CCc3[nH]cnc3[C@H]2c2cc3c(F)cccn3n2)ncc1C(=O)N1CCCCC1]

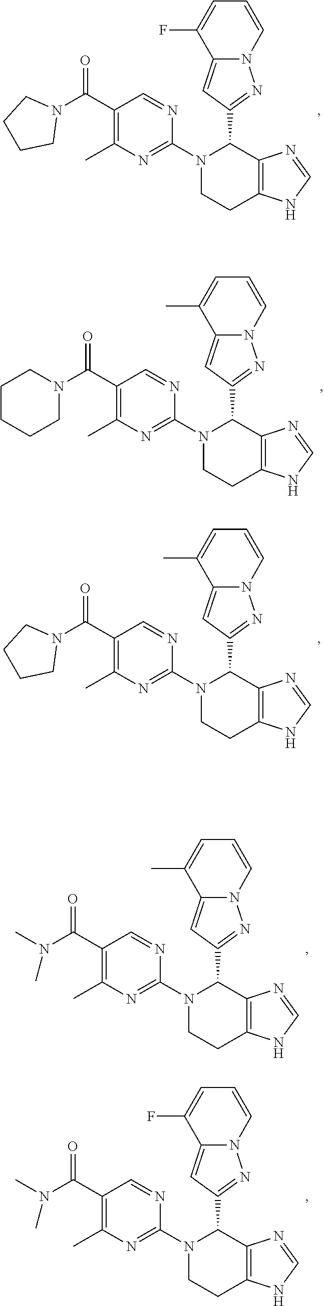

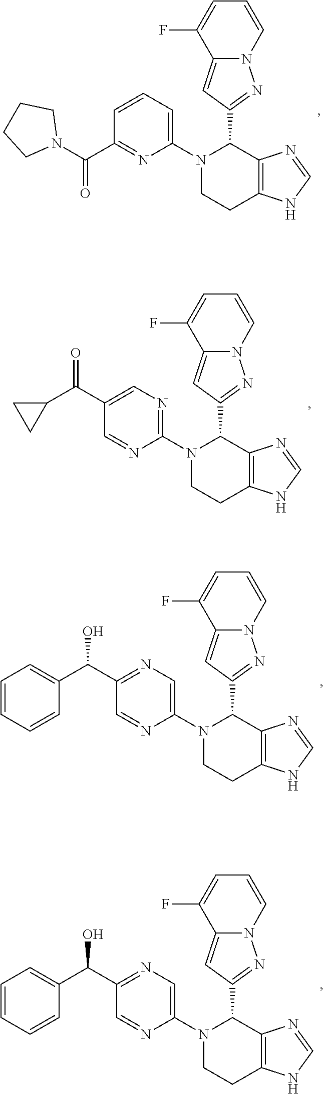 [SMILES: O=C(c1cccc(N2CCc3[nH]cnc3[C@H]2c2cc3c(F)cccn3n2)n1)N1CCCC1 ; O=C(c1cnc(N2CCc3[nH]cnc3[C@H]2c2cc3c(F)cccn3n2)nc1)C1CC1 ; O[C@@H](c1ccccc1)c1cnc(N2CCc3[nH]cnc3[C@H]2c2cc3c(F)cccn3n2)cn1 ; O[C@H](c1ccccc1)c1cnc(N2CCc3[nH]cnc3[C@H]2c2cc3c(F)cccn3n2)cn1]

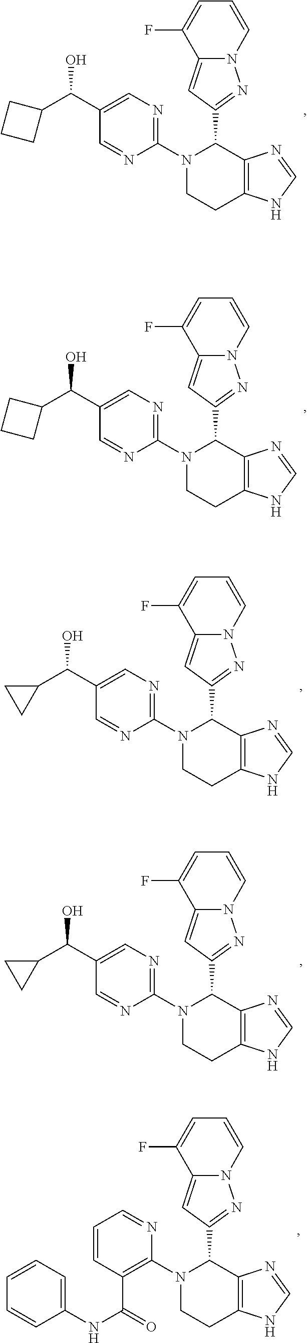 [SMILES: O[C@H](c1cnc(N2CCc3[nH]cnc3[C@H]2c2cc3c(F)cccn3n2)nc1)C1CCC1 ; O[C@@H](c1cnc(N2CCc3[nH]cnc3[C@H]2c2cc3c(F)cccn3n2)nc1)C1CCC1 ; O[C@H](c1cnc(N2CCc3[nH]cnc3[C@H]2c2cc3c(F)cccn3n2)nc1)C1CC1 ; O[C@@H](c1cnc(N2CCc3[nH]cnc3[C@H]2c2cc3c(F)cccn3n2)nc1)C1CC1 ; O=C(Nc1ccccc1)c1cccnc1N1CCc2[nH]cnc2[C@H]1c1cc2c(F)cccn2n1]

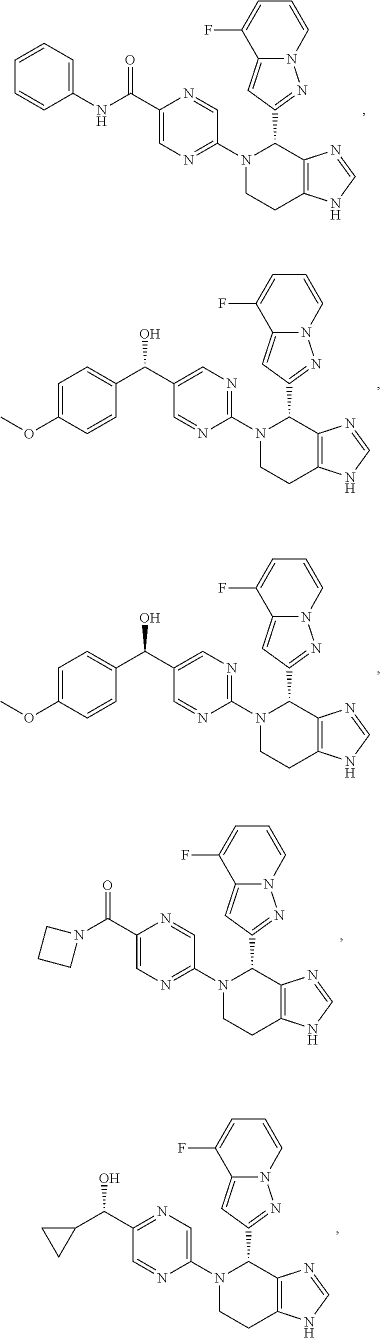 [SMILES: O=C(Nc1ccccc1)c1cnc(N2CCc3[nH]cnc3[C@H]2c2cc3c(F)cccn3n2)cn1 ; COc1ccc([C@H](O)c2cnc(N3CCc4[nH]cnc4[C@H]3c3cc4c(F)cccn4n3)nc2)cc1 ; COc1ccc([C@@H](O)c2cnc(N3CCc4[nH]cnc4[C@H]3c3cc4c(F)cccn4n3)nc2)cc1 ; O=C(c1cnc(N2CCc3[nH]cnc3[C@H]2c2cc3c(F)cccn3n2)cn1)N1CCC1 ; O[C@H](c1cnc(N2CCc3[nH]cnc3[C@H]2c2cc3c(F)cccn3n2)cn1)C1CC1]

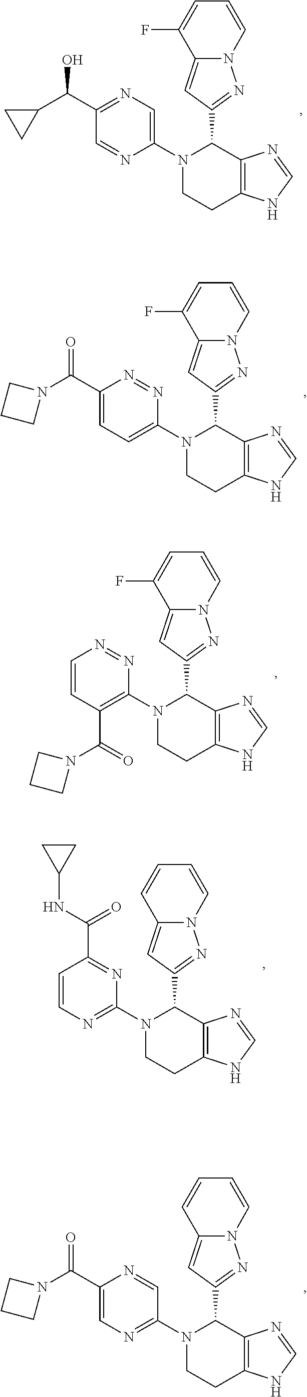 [SMILES: O[C@@H](c1cnc(N2CCc3[nH]cnc3[C@H]2c2cc3c(F)cccn3n2)cn1)C1CC1 ; O=C(c1ccc(N2CCc3[nH]cnc3[C@H]2c2cc3c(F)cccn3n2)nn1)N1CCC1 ; O=C(c1ccnnc1N1CCc2[nH]cnc2[C@H]1c1cc2c(F)cccn2n1)N1CCC1 ; O=C(NC1CC1)c1ccnc(N2CCc3[nH]cnc3[C@H]2c2cc3ccccn3n2)n1 ; O=C(c1cnc(N2CCc3[nH]cnc3[C@H]2c2cc3ccccn3n2)cn1)N1CCC1]

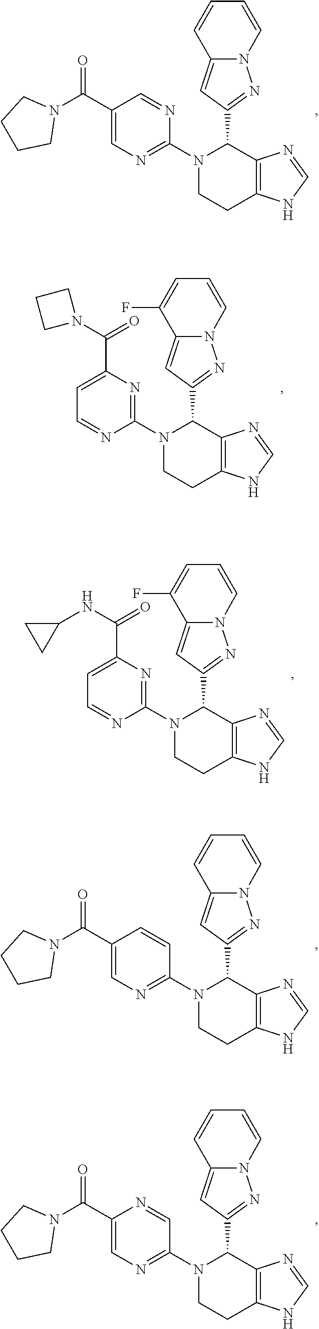 [SMILES: O=C(c1cnc(N2CCc3[nH]cnc3[C@H]2c2cc3ccccn3n2)nc1)N1CCCC1 ; O=C(c1ccnc(N2CCc3[nH]cnc3[C@H]2c2cc3c(F)cccn3n2)n1)N1CCC1 ; O=C(NC1CC1)c1ccnc(N2CCc3[nH]cnc3[C@H]2c2cc3c(F)cccn3n2)n1 ; O=C(c1ccc(N2CCc3[nH]cnc3[C@H]2c2cc3ccccn3n2)nc1)N1CCCC1 ; O=C(c1cnc(N2CCc3[nH]cnc3[C@H]2c2cc3ccccn3n2)cn1)N1CCCC1]

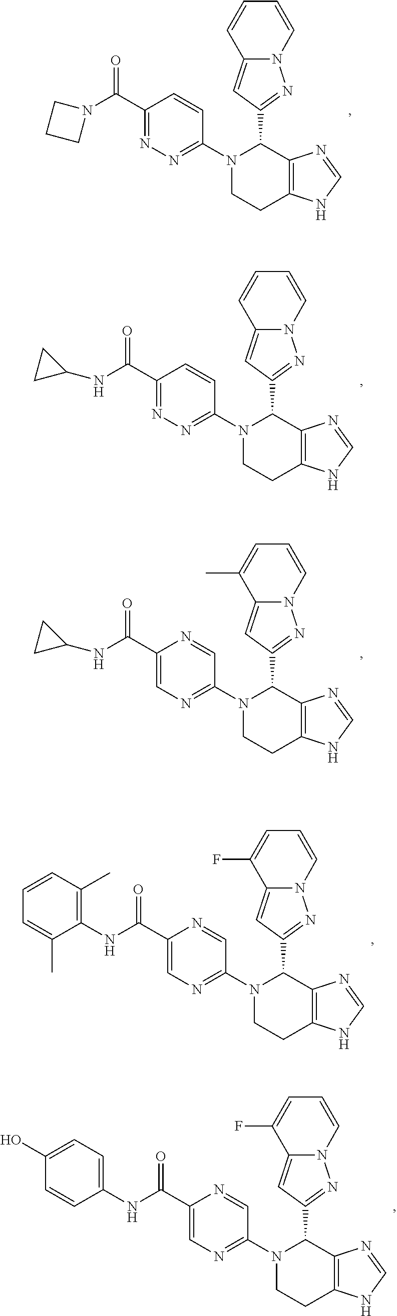 [SMILES: O=C(c1ccc(N2CCc3[nH]cnc3[C@H]2c2cc3ccccn3n2)nn1)N1CCC1 ; O=C(NC1CC1)c1ccc(N2CCc3[nH]cnc3[C@H]2c2cc3ccccn3n2)nn1 ; Cc1cccn2nc([C@@H]3c4nc[nH]c4CCN3c3cnc(C(=O)NC4CC4)cn3)cc12 ; Cc1cccc(C)c1NC(=O)c1cnc(N2CCc3[nH]cnc3[C@H]2c2cc3c(F)cccn3n2)cn1 ; O=C(Nc1ccc(O)cc1)c1cnc(N2CCc3[nH]cnc3[C@H]2c2cc3c(F)cccn3n2)cn1]

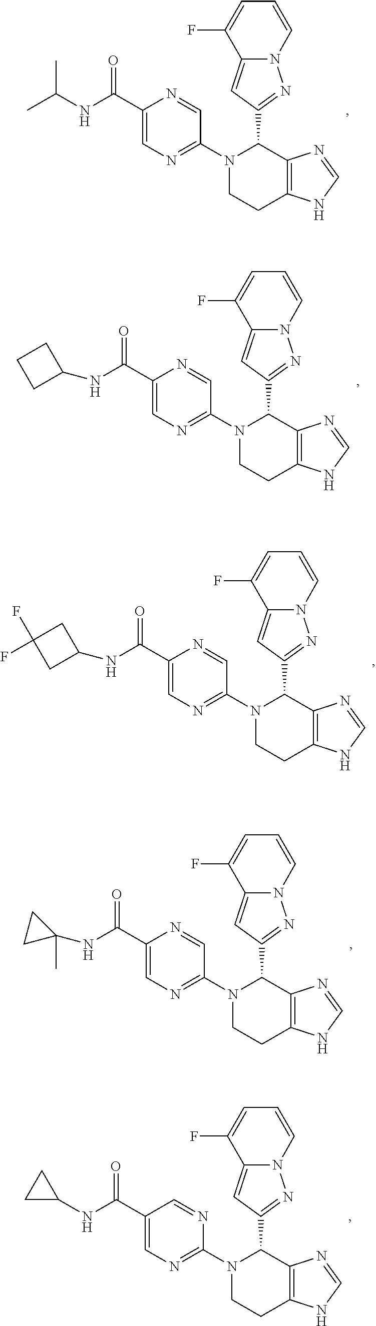 [SMILES: CC(C)NC(=O)c1cnc(N2CCc3[nH]cnc3[C@H]2c2cc3c(F)cccn3n2)cn1 ; O=C(NC1CCC1)c1cnc(N2CCc3[nH]cnc3[C@H]2c2cc3c(F)cccn3n2)cn1 ; O=C(NC1CC(F)(F)C1)c1cnc(N2CCc3[nH]cnc3[C@H]2c2cc3c(F)cccn3n2)cn1 ; CC1(NC(=O)c2cnc(N3CCc4[nH]cnc4[C@H]3c3cc4c(F)cccn4n3)cn2)CC1 ; O=C(NC1CC1)c1cnc(N2CCc3[nH]cnc3[C@H]2c2cc3c(F)cccn3n2)nc1]

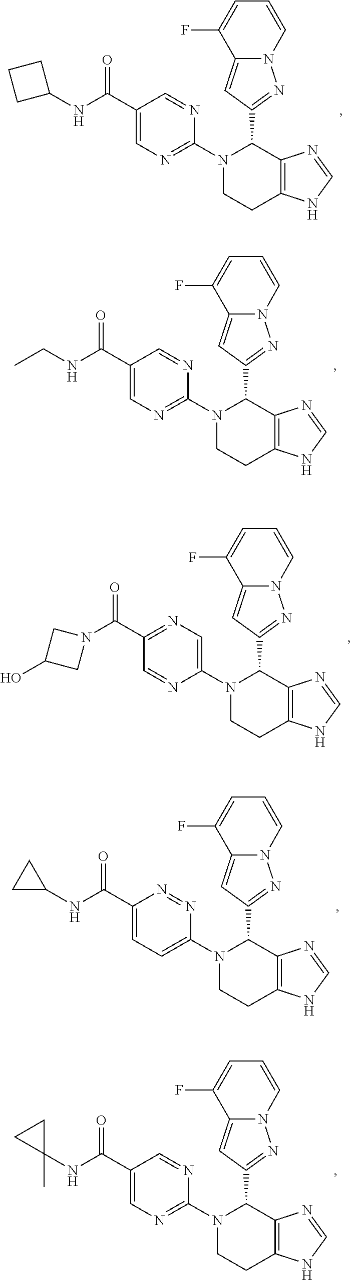 [SMILES: O=C(NC1CCC1)c1cnc(N2CCc3[nH]cnc3[C@H]2c2cc3c(F)cccn3n2)nc1 ; CCNC(=O)c1cnc(N2CCc3[nH]cnc3[C@H]2c2cc3c(F)cccn3n2)nc1 ; O=C(c1cnc(N2CCc3[nH]cnc3[C@H]2c2cc3c(F)cccn3n2)cn1)N1CC(O)C1 ; O=C(NC1CC1)c1ccc(N2CCc3[nH]cnc3[C@H]2c2cc3c(F)cccn3n2)nn1 ; CC1(NC(=O)c2cnc(N3CCc4[nH]cnc4[C@H]3c3cc4c(F)cccn4n3)nc2)CC1]

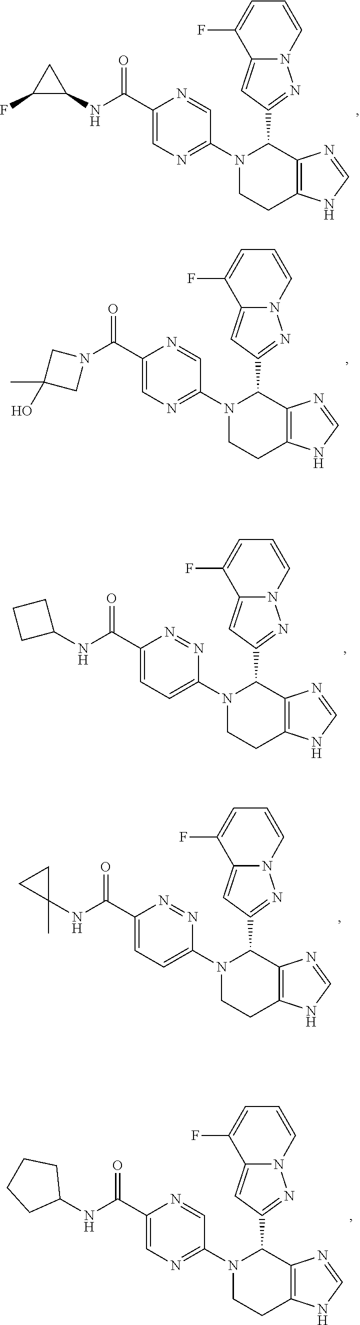

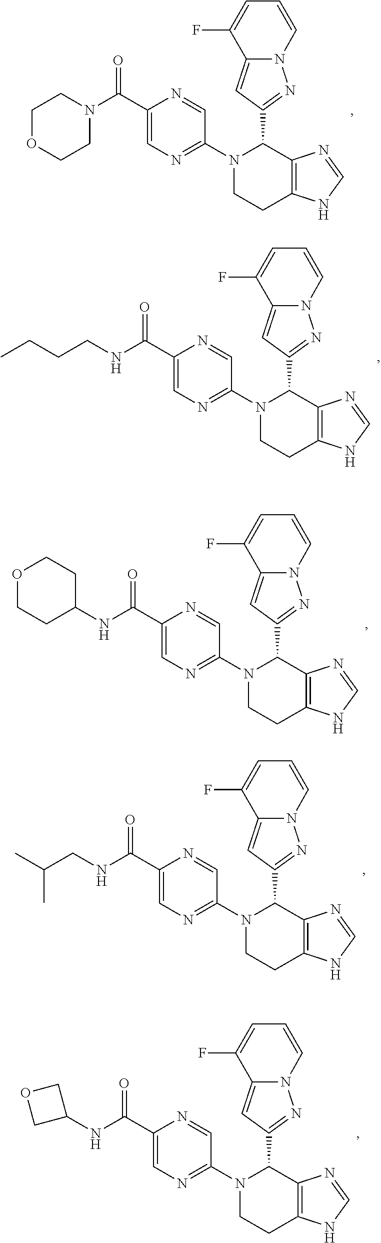

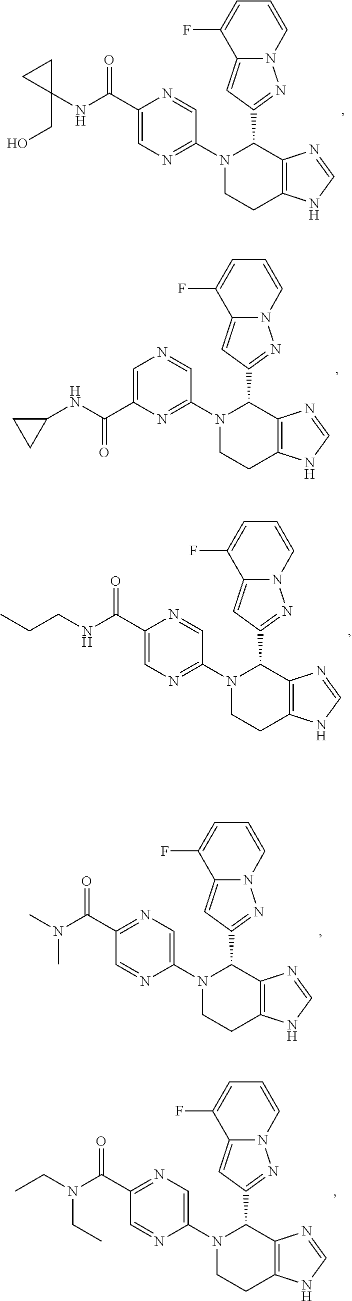 [SMILES: O=C(NC1(CO)CC1)c1cnc(N2CCc3[nH]cnc3[C@H]2c2cc3c(F)cccn3n2)cn1 ; O=C(NC1CC1)c1cncc(N2CCc3[nH]cnc3[C@H]2c2cc3c(F)cccn3n2)n1 ; CCCNC(=O)c1cnc(N2CCc3[nH]cnc3[C@H]2c2cc3c(F)cccn3n2)cn1 ; CN(C)C(=O)c1cnc(N2CCc3[nH]cnc3[C@H]2c2cc3c(F)cccn3n2)cn1 ; CCN(CC)C(=O)c1cnc(N2CCc3[nH]cnc3[C@H]2c2cc3c(F)cccn3n2)cn1]

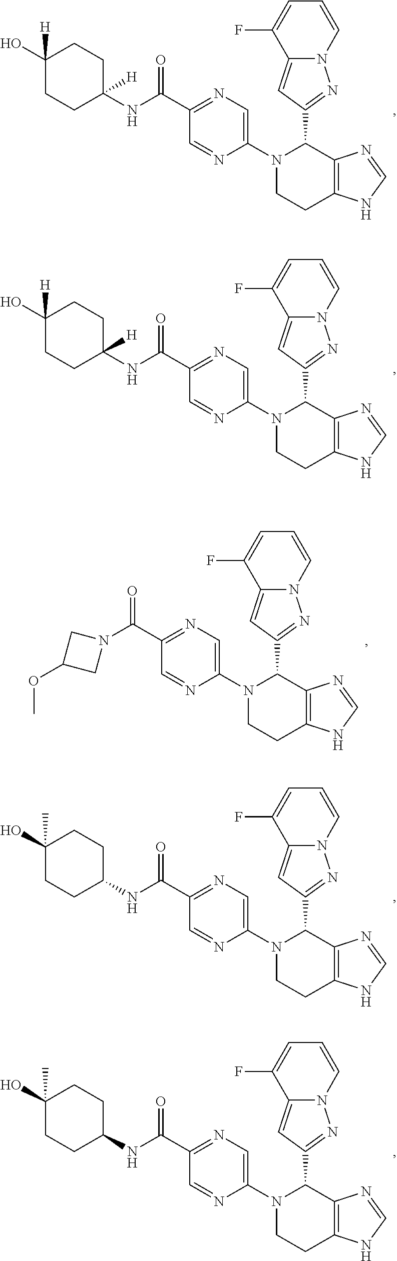 [SMILES: O=C(N[C@H]1CC[C@H](O)CC1)c1cnc(N2CCc3[nH]cnc3[C@H]2c2cc3c(F)cccn3n2)cn1 ; O=C(N[C@H]1CC[C@@H](O)CC1)c1cnc(N2CCc3[nH]cnc3[C@H]2c2cc3c(F)cccn3n2)cn1 ; COC1CN(C(=O)c2cnc(N3CCc4[nH]cnc4[C@H]3c3cc4c(F)cccn4n3)cn2)C1 ; C[C@]1(O)CC[C@H](NC(=O)c2cnc(N3CCc4[nH]cnc4[C@H]3c3cc4c(F)cccn4n3)cn2)CC1 ; C[C@]1(O)CC[C@@H](NC(=O)c2cnc(N3CCc4[nH]cnc4[C@H]3c3cc4c(F)cccn4n3)cn2)CC1]

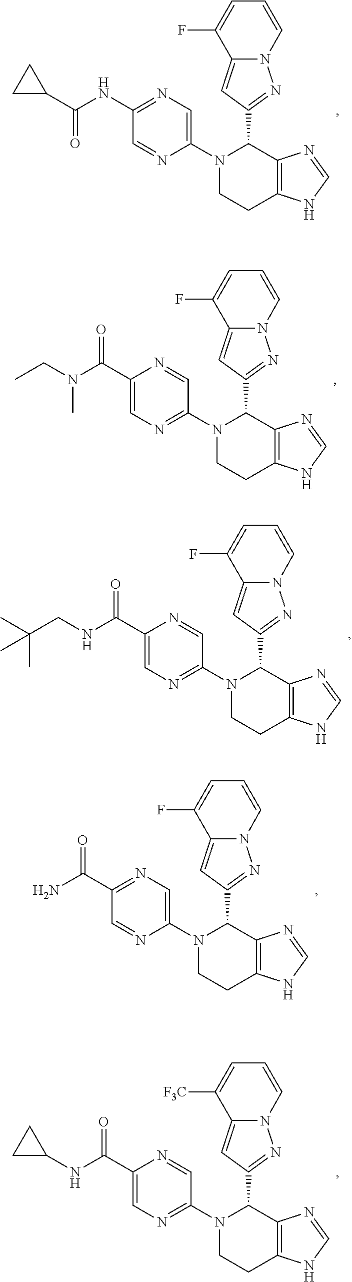

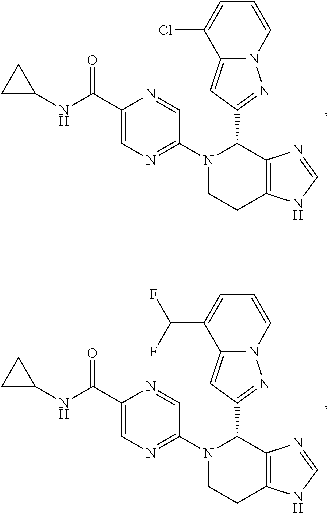 [SMILES: O=C(NC1CC1)c1cnc(N2CCc3[nH]cnc3[C@H]2c2cc3c(Cl)cccn3n2)cn1 ; O=C(NC1CC1)c1cnc(N2CCc3[nH]cnc3[C@H]2c2cc3c(C(F)F)cccn3n2)cn1]

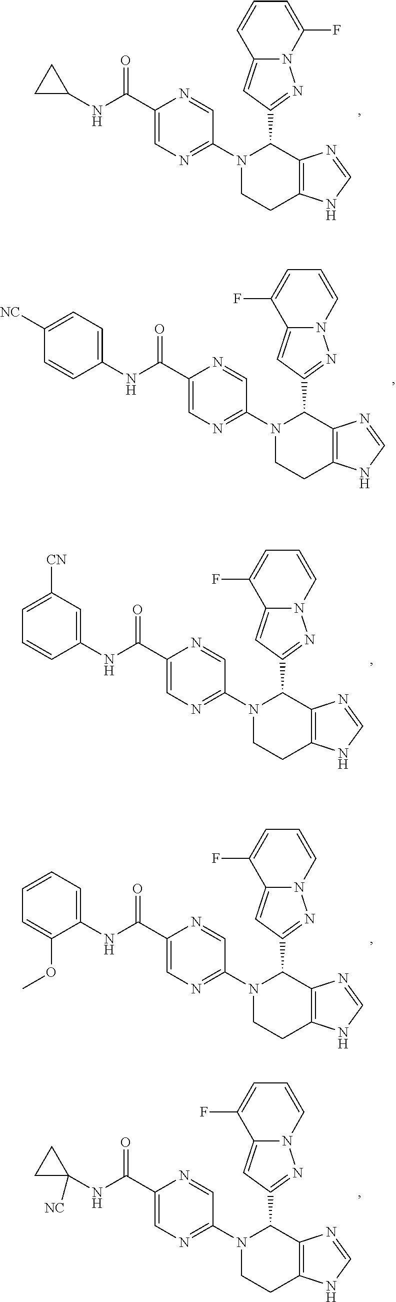

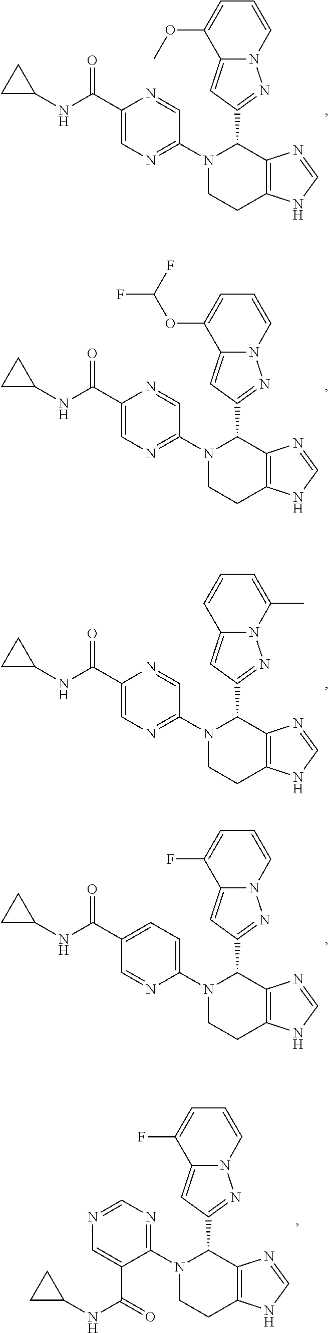 [SMILES: COc1cccn2nc([C@@H]3c4nc[nH]c4CCN3c3cnc(C(=O)NC4CC4)cn3)cc12 ; O=C(NC1CC1)c1cnc(N2CCc3[nH]cnc3[C@H]2c2cc3c(OC(F)F)cccn3n2)cn1 ; Cc1cccc2cc([C@@H]3c4nc[nH]c4CCN3c3cnc(C(=O)NC4CC4)cn3)nn12 ; O=C(NC1CC1)c1ccc(N2CCc3[nH]cnc3[C@H]2c2cc3c(F)cccn3n2)nc1 ; O=C(NC1CC1)c1cncnc1N1CCc2[nH]cnc2[C@H]1c1cc2c(F)cccn2n1]

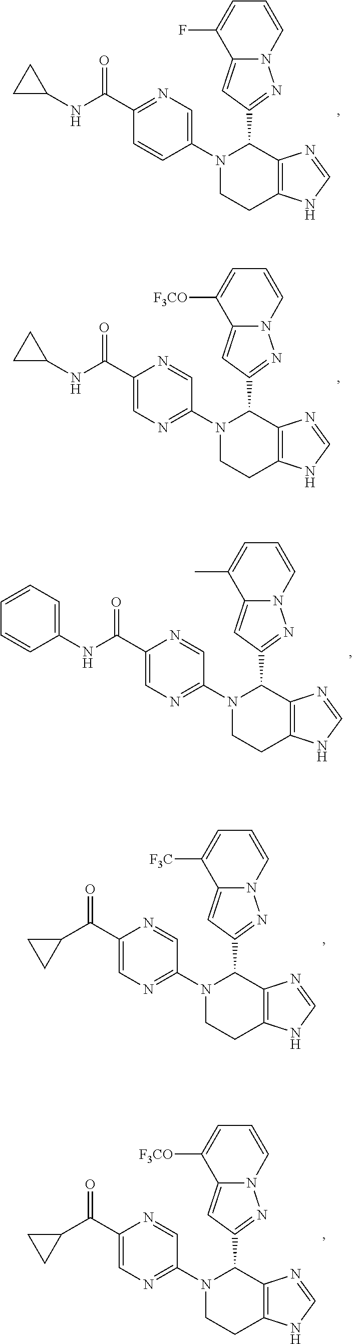 [SMILES: O=C(NC1CC1)c1ccc(N2CCc3[nH]cnc3[C@H]2c2cc3c(F)cccn3n2)cn1 ; O=C(NC1CC1)c1cnc(N2CCc3[nH]cnc3[C@H]2c2cc3c(OC(F)(F)F)cccn3n2)cn1 ; Cc1cccn2nc([C@@H]3c4nc[nH]c4CCN3c3cnc(C(=O)Nc4ccccc4)cn3)cc12 ; O=C(c1cnc(N2CCc3[nH]cnc3[C@H]2c2cc3c(C(F)(F)F)cccn3n2)cn1)C1CC1 ; O=C(c1cnc(N2CCc3[nH]cnc3[C@H]2c2cc3c(OC(F)(F)F)cccn3n2)cn1)C1CC1]

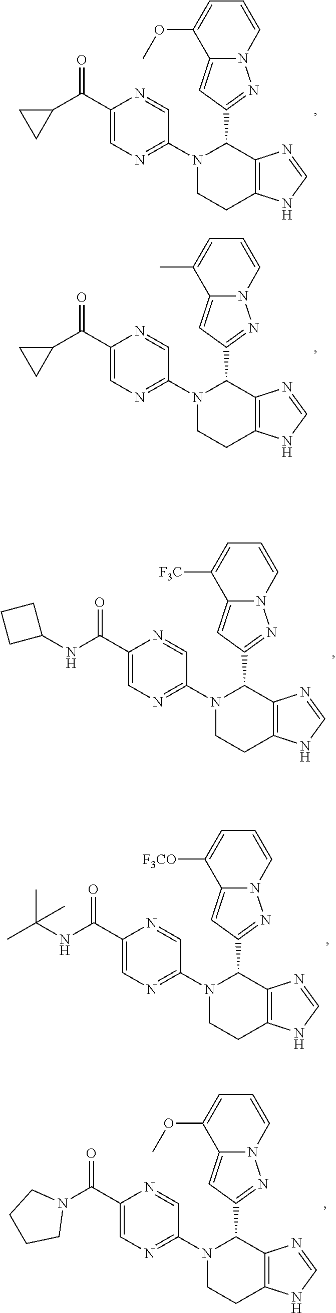

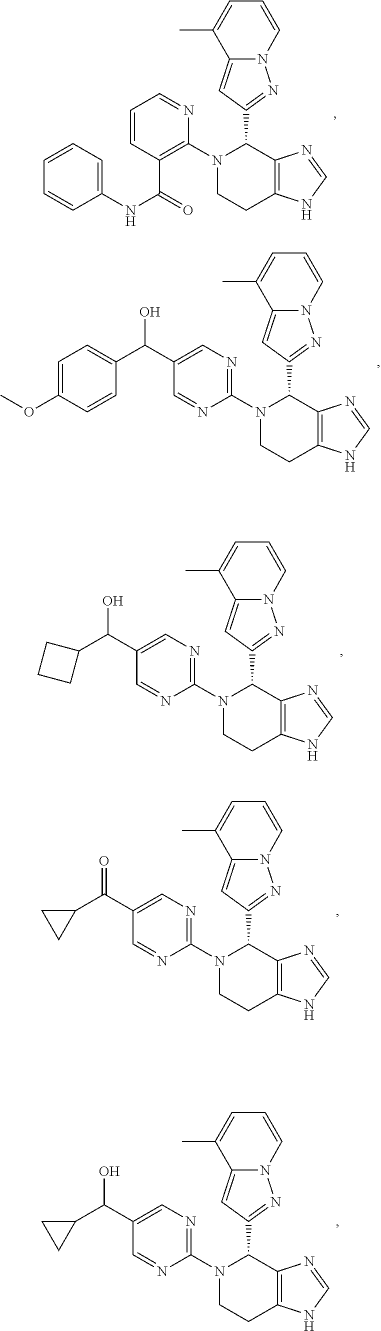 [SMILES: Cc1cccn2nc([C@@H]3c4nc[nH]c4CCN3c3ncccc3C(=O)Nc3ccccc3)cc12 ; COc1ccc(C(O)c2cnc(N3CCc4[nH]cnc4[C@H]3c3cc4c(C)cccn4n3)nc2)cc1 ; Cc1cccn2nc([C@@H]3c4nc[nH]c4CCN3c3ncc(C(O)C4CCC4)cn3)cc12 ; Cc1cccn2nc([C@@H]3c4nc[nH]c4CCN3c3ncc(C(=O)C4CC4)cn3)cc12 ; Cc1cccn2nc([C@@H]3c4nc[nH]c4CCN3c3ncc(C(O)C4CC4)cn3)cc12]

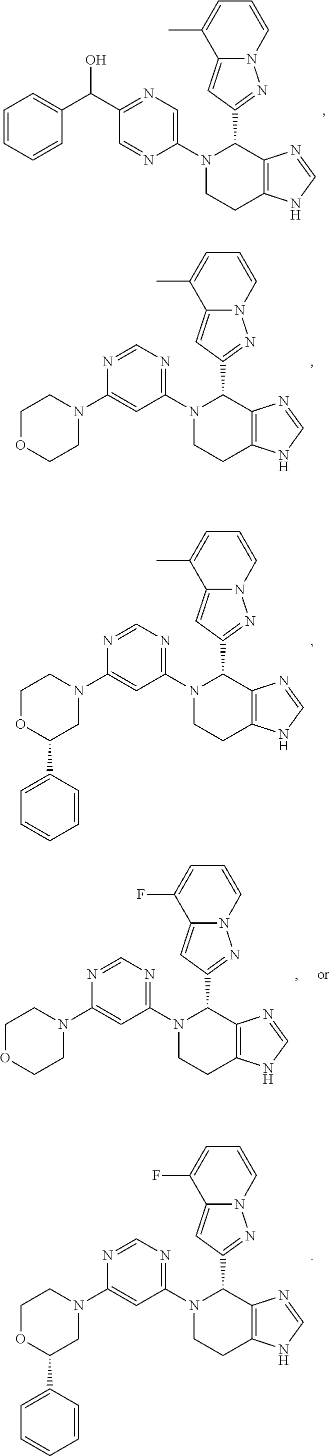

40. The compound of claim 1, wherein the compound is an S-enantiomer, or tautomer thereof, or a pharmaceutically acceptable salt of the compound or the tautomer.

41. (canceled)

42. A pharmaceutical composition comprising a compound of claim 1, or tautomer thereof, or a pharmaceutically acceptable salt of the compound or the tautomer, and a pharmaceutically acceptable excipient.

43. (canceled)

44. A method for stabilizing a mutant PAH protein, comprising contacting the protein with a compound of claim 1, or tautomer thereof, or a pharmaceutically acceptable salt of the compound or the tautomer.

45. (canceled)

46. (canceled)

47. The method of claim 44, wherein the mutant PAH protein contains at least one R408W mutation.

48. (canceled)

49. A method for reducing blood phenylalanine concentration in a subject suffering from phenylketonuria comprising administering a compound of claim 1, or tautomer thereof, or a pharmaceutically acceptable salt of the compound or the tautomer.

50. The method of claim 49, wherein the blood phenylalanine concentration is reduced to a concentration less than or equal to about 600 μM.

51.-53. (canceled)
